(19) United States
(12) Patent Application Publication
(10) Pub. No.: US 2006/0004011 A1
(43) Pub. Date: Jan. 5, 2006

(54) Indolyl pyrazinone derivatives useful for treating hyper-proliferative disorders and diseases associated with angiogenesis

(75) Inventors: Gaetan H Ladouceur, Branford, CT (US); Brian Bear, Milford, CT (US); Cheng Bi, West Haven, CT (US); David R Brittelli, Branford, CT (US); Michael J Burke, New Haven, CT (US); Gang Chen, North York (CA); James Cook, East Hampton, CT (US); Jacques Dumas, Bethany, CT (US); Robert Sibley, North Haven, CT (US); Michael R Turner, Milford, CT (US)

Correspondence Address: JEFFREY M. GREENMAN, BAYER PHARMACEUTICALS CORPORATION, 400 MORGAN LANE, WEST HAVEN, CT, 06516, US

(73) Assignee: Bayer Pharmaceuticals Corporation, West Haven, CT (US)

(21) Appl. No.: 10/534,215

(22) PCT Filed: Nov. 10, 2003
(86) PCT No.: PCT/US03/36003

§ 371 Date: May 6, 2005

Related U.S. Application Data
(60) Provisional application No. 60/484,202, filed on Jun. 30, 2003.

(51) Int. Cl. A61K 31/498; C07D 43/14

(52) U.S. Cl. 514/249; 544/353

(57) ABSTRACT

This invention relates to a compound of Formula I (I) and its use in treating hyper-proliferative disorders and diseases associated with angiogenesis.

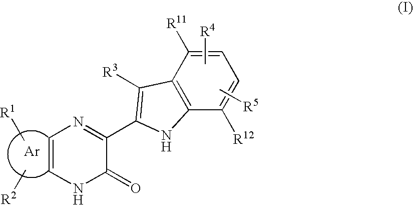

This application claims priority to U.S. Provisional Application No. 60/425490, filed Nov. 12, 2002, and to U.S. Provisional Application No. 60/460,915, filed Apr. 7, 2003, and to U.S. Provisional Application No. 60/484,202 filed Jun. 30, 2003.

### FIELD OF THE INVENTION

This invention relates to novel indolyl pyrazinone compounds, pharmaceutical compositions containing such compounds and the use of those compounds and compositions for the prevention and/or treatment of hyper-proliferative disorders and diseases associated with angiogenesis.

### DESCRIPTION OF THE INVENTION

Compounds of the Present Invention

One embodiment of this invention is a compound of Formula I 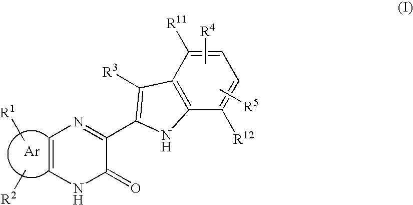
wherein 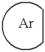
represents a 6 membered aromatic ring containing 0, 1 or 2 N atoms;
R¹ and R² are each independently selected from H, halo, CF₃, C(O)R⁹, 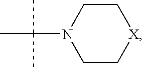
   (C₁-C₆)alkyl optionally substituted with up to two substituents selected from OH, (C₁-C₃)alkoxy, F, and phenyl,
   (C₁-C₆)alkoxy optionally substituted with one or two substituents each independently selected from 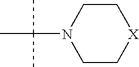
   and N[(C₁-C₃)alkyl]₂ where each alkyl is independently optionally substituted up to two times with (C₁-C₃)alkoxy,
   NH(C₁-C₃)alkyl where said alkyl is optionally substituted with up to two substitutents each selected independently from OH, F, (C₁-C₃)alkoxy, N[(C₁-C₃)alkyl]₂, NH(C₁-C₃)alkyl, phenyl, pyrrolidinyl, and 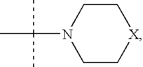
   N[(C₁-C₃)alkyl]₂ where each alkyl is independently optionally substituted with up to two substitutents each selected independently from OH, F, phenyl, and (C₁-C₃)alkoxy, said alkoxy being optionally substituted with 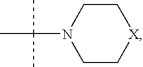
   pyrrolidinyl optionally substituted up to two times with N[(C₁-C₃)alkyl]₂,
   phenyl optionally substituted with up to two substitutents each selected independently from (C₁-C₃)alkyl, (C₁-C₃)alkoxy, halo, CF₃, and CN,
   with the proviso that when 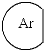
   contains 1 or 2 N atoms, R¹ and R² must each be H,
   and, R¹ and R² together with the adjacent C atoms to which they are attached form a ring selected from benzo, dioxolo and imidazo,
      said imidazo being optionally substituted up to two times with (C₁-C₃)alkyl,
   with the proviso that R¹ and R² together with the adjacent C atoms to
   which they are attached form a ring only when 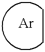
   contains no N atoms;
R³ is selected from H, (C₁-C₄)alkyl, OH, NO₂, NH₂, NH(C₁-C₄)alkyl, NHC(O)(C₁-C₄)alkyl and NHC(O)phenyl, said phenyl being optionally substituted with up to two substituents independently selected from (C₁-C₃)alkyl, (C₁-C₃)alkoxy, halo, CF₃, and CN;
R⁴ is selected from H, OH, halo, CN, C(O)R⁶, S(O)₂R⁷, OSi[(C₁-C₄)alkyl]₃, tetrazolyl, thienyl, pyrrolyl, pyrimidinyl, oxazolyl, furanyl,
   (C₁-C₆)alkyl, (C₂-C₆)alkenyl or (C₂-C₆)alkynyl, each optionally substituted with OH, F, OC(O)NHphenyl, NHC(O)(C₁-C₃)alkyl, C(O)NH₂, C(O)NH(C₁-C₃)alkyl, C(O)N[(C₁-C₃)alkyl]₂, 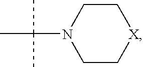
      (C₁-C₃)alkoxy optionally substituted up to two times with (C₁-C₃)alkoxy,
      NHC(O)NH(C₁-C₃)alkyl where said alkyl is optionally substituted with up to two substituents independently selected from OH, (C₁-C₃)alkoxy, F and phenyl,
      NHC(O)NHphenyl where said phenyl is optionally substituted with up to two substituents independently selected from (C₁-C₃)alkyl, (C₁-C₃)alkoxy, halo, CF₃, CN, and 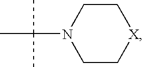
      NHC(O)N[(C₁-C₃)alkyl]₂ where each alkyl is independently optionally substituted up to two times with (C₁-C₃)alkoxy,
      NH-phenyl, said phenyl being optionally substituted with up to two substituents independently selected from (C₁-C₃)alkyl, (C₁-C₃)alkoxy, halo, CN, and 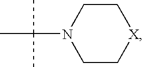
      N[(C₁-C₃)alkyl]₂ where each alkyl is independently optionally substituted up to two times with (C₁-C₃)alkoxy,
      phenyl optionally substituted with up to two substituents independently selected from (C₁-C₃)alkyl, (C₁-C₃)alkoxy, halo, CN, CF₃, and 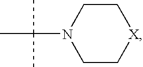
      pyrrolidinyl optionally substituted up to two times with N[(C₁-C₃)alkyl]₂,
   (C₁-C₆)alkoxy optionally substituted with up to two substituents independently selected from (C₁-C₃)alkoxy, pyrrolidinyl, 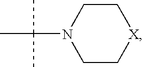
   and N[(C₁-C₃)alkyl]₂ where each alkyl is independently optionally substituted with up to two substituents independently selected from OH, F, (C₁-C₃)alkoxy and phenyl,
   N[(C₁-C₄)alkyl]₂ where each of said alkyl groups are independently optionally substituted with up to two substituents independently selected from OH, (C₁-C₃)alkyl, F, (C₁-C₃)alkoxy, and phenyl,
   oxadiazolyl optionally substituted up to two times with (C₁-C₃)alkyl,
   phenyl optionally substituted with up to two substituents independently selected from (C₁-C₃)alkoxy, CN, (C₁-C₃)alkyl, halo, 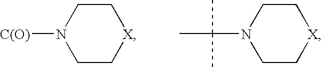
      C(O)(C₁-C₃)alkyl optionally substituted with up to two substituents independently selected from (C₁-C₃)alkoxy, OH, (C₁-C₃)alkoxy, F, and phenyl, and
      C(O)N[(C₁-C₃)alkyl]₂ where each of said alkyl groups are independently optionally substituted up to two times with (C₁-C₃)alkoxy,
   pyridyl optionally substituted with up to two substituents independently selected from (C₁-C₃)alkyl,
   C(O)N[(C₁-C₃)alkyl]₂ where each of said alkyl groups are independently optionally substituted up to two times with (C₁-C₃)alkoxy, and
   O-pyridyl optionally substituted with up to two substituents independently selected from CF₃, halo, and (C₁-C₃)alkyl;
R⁵ is selected from H, halo, CN, (C₁-C₆)alkoxy, and (C₁-C₆)alkyl;
R⁶ is selected from OH, NHR¹⁰, O—(C₃-C₆)cycloakyl, (C₁-C₃)alkoxy, O—(C₂-C₆)alkenyl, O—(C₃-C₆)alkynyl,
   (C₁-C₆)alkyl optionally substituted with up to two substituents independently selected from OH, (C₁-C₃)alkoxy, F, and phenyl,
   N[(C₁-C₄)alkyl]₂ where each of said alkyl groups are independently optionally substituted with up to two substituents independently selected from OH, CN, N[(C₁-C₄)alkyl]₂, (C₁-C₃)alkoxy, S(O)₂-phenyl, S(O)₂(C₁-C₃)alkyl, phenyl, furyl, tetrahydrofuryl, (C₃-C₆)cycloalkyl, and pyridyl,
   N[(C₁-C₃)alkyl]R⁸ where [(C₁-C₃)alkyl] is optionally substituted up to two times with (C₁-C₃)alkoxy,
   N[(C₃-C₆)cycloalkyl](C₁-C₃)alkyl where said alkyl is substituted with up to two substituents independently selected from (C₁-C₃)alkoxy, OH, CN, N[(C₁-C₄)alkyl]₂, S(O)₂-phenyl, S(O)₂(C₁-C₃)alkyl, phenyl, furyl, tetrahydrofuryl, (C₅-C₆)cycloalkyl, and pyridyl,
   pyrrolidinyl optionally substituted with up to two substituents independently selected from NH₂, NH(C₁-C₃)alkyl, N[(C₁-C₄)alkyl]₂, C(O)NH₂, NHC(O)(C₁-C₃)alkyl, NHS(O)₂(C₁-C₃)alkyl, pyridyl, N[(C₁-C₃)alkyl]C(O)NH(C₁-C₃)alkyl, N[(C₁-C₃)alkyl]C(O)(C₁-C₃)alkyl, and (C₁-C₃)alkyl optionally substituted with up to two substituents
      independently selected from N[(C₁-C₄)alkyl]₂, (C₁-C₃)alkoxy, and pyrrolidinyl,
   morpholinyl optionally substituted up to two times with (C₁-C₃)alkyl,
   thiomorpholinyl optionally substituted up to two times with (C₁-C₃)alkyl,
   piperazinyl optionally substituted with up to two substituents independently selected from pyrazinyl, C(O)NH₂, C(O)NH-phenyl, C(O)-furanyl, C(O)(C₁-C₃)alkyl, C(O)NH(C₁-C₃)alkyl, C(O)N[(C₁-C₃)alkyl]R⁸, S(O)₂(C₁-C₃)alkyl, S(O)₂-phenyl, 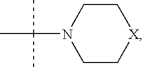
      pyridyl optionally substituted with up to two substituents independently selected from (C₁-C₃)alkyl, CN and CF₃,
      phenyl optionally substituted with up to two substituents independently selected from (C₁-C₃)alkyl, CN, halo, CF₃, and (C₁-C₃)alkoxy,
      (C₁-C₃)alkyl optionally substituted with up to two substituents independently selected from OH, F, phenyl, (C₁-C₃)alkoxy, N[(C₁-C₃)alkyl]₂, pyrrolinidyl, C(O)-pyrrolidinyl, 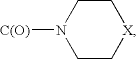 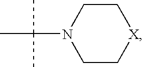
      and pyridyl optionally substituted up to two times with (C₁-C₃)alkoxy, and
   piperidinyl optionally substituted with up to two substituents independently selected from phenyl, pyridyl, pyrrolidinyl and oxo-dihydrobenzimidazolyl;
R⁷ is selected from NH₂, pyrrolidinyl, 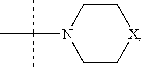
   NH(C₁-C₃)alkyl said alkyl being optionally substituted up to two times with (C₁-C₃)alkoxy,
   NH-phenyl said phenyl being optionally substituted with up to two substituents independently selected from (C₁-C₃)alkyl, CN, (C₁-C₄)alkoxy, halo and CF₃,
   N[(C₁-C₃)alkyl]₂ wherein each alkyl is independently optionally substituted up to two times with (C₁-C₄)alkoxy, and
   phenyl optionally substituted with up to two substituents independently selected from (C₁-C₃)alkyl, (C₁-C₃)alkoxy, halo, CF₃ and CN;
R⁸ is selected from (C₁-C₃)alkoxy, pyridyl, piperidinyl, pyranyl and
   phenyl, where each ring moiety is optionally substituted with up to two substituents independently selected from (C₁-C₃)alkoxy, and (C₁-C₃)alkyl;
R⁹ is selected from (C₁-C₃)alkyl, (C₁-C₃)alkoxy, OH, 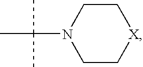
   phenyl optionally substituted with (C₁-C₃)alkyl, (C₁-C₃)alkoxy, halo, CF₃, and CN,
   N[(C₁-C₄)alkyl]₂ where each of said alkyl groups are independently optionally substituted with OH, CN, N[(C₁-C₄)alkyl]₂, (C₁-C₄)alkoxy, S(O)₂-phenyl, S(O)₂(C₁-C₃)alkyl, phenyl, furyl, tetrahydrofuryl, (C₃-C₆)cycloalkyl, and pyridyl, and
   pyrrolidinyl optionally substituted with N[(C₁-C₃)alkyl]₂,
   and, only when 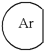
   contains no N atoms, R⁹ is also selected from pyridyl, thienyl, and NHR¹⁰;
R¹⁰ is selected from H, indolyl,
   (C₁-C₄)alkyl optionally substituted with up to two substituents independently selected from OH, F, phenyl, (C₁-C₄)alkoxy, NHC(O)(C₁-C₃)alkyl,
      S—(C₁-C₃)alkyl, benzimidazolyl, indolyl, thienyl, pyrazolyl, 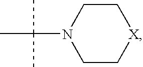
      N[(C₁-C₄)alkyl]₂ where each alkyl is independently optionally substituted with up to two substituents independently selected from OH, (C₁-C₃)alkoxy, F, and phenyl,
      phenyl optionally substituted with up to two substituents independently selected from (C₁-C₃)alkyl, (C₁-C₃)alkoxy, CN, halo, CF₃, S(O)₂(C₁-C₃)alkyl, S(O)₂phenyl, and S(O)₂NH₂,
      pyridyl optionally substituted up to two times with CF₃,
      imidazolyl optionally substituted up to two times with (C₁-C₃)alkyl,
      furyl optionally substituted up to two times with (C₁-C₄)alkyl, and
      pyrrolidinyl optionally substituted with up to two substituents independently selected from (C₁-C₄)alkoxy, (O), and
         (C₁-C₄)alkyl optionally substituted with up to two substituents independently selected from OH, (C₁-C₃)alkoxy, F, and phenyl,
   S(O)₂-phenyl optionally substituted with up to two substituents independently selected from (C₁-C₄)alkoxy, (C₁-C₃)alkyl, halo, and CN,
   pyrazolyl optionally substituted with up to two substituents independently selected from (C₁-C₄)alkyl, (C₃-C₆)cycloalkyl, and
      phenyl, said phenyl being optionally substituted with up to two substituents independently selected from (C₁-C₄)alkoxy, (C₁-C₄)alkyl, halo, CF₃, and CN,
   benzothiazolyl optionally substituted up to two times with (C₁-C₄)alkyl,
   thiazolyl, optionally substituted up to two times with (C₁-C₄)alkyl,
   thiadiazolyl, optionally substituted with up to two substituents independently selected from CF₃, (C₃-C₆)cycloalkyl, and (C₁-C₆)alkyl,
   phenyl optionally substituted with up to two substituents independently selected from CN, halo, CF₃, N[(C₁-C₄)alkyl]₂, indolyl, 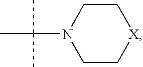
      O-pyridyl optionally substituted with C(O)NH(C₁-C₄)alkyl,
      (C₁-C₄)alkyl optionally substituted with up to two substituents independently selected from pyridyl, 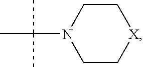
      OH,
         (C₁-C₃)alkoxy, F, and phenyl, and
         (C₁-C₄)alkoxy optionally substituted with N[(C₁-C₄)alkyl]₂ where one alkyl group is optionally substituted with phenyl, or
         (C₁-C₄)alkoxy optionally substituted with 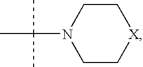
   pyridyl optionally substituted with phenoxy where said phenoxy is optionally substituted with up to two substituents independently selected from (C₁-C₄)alkyl and (C₁-C₄)alkoxy, and
   indazolyl optionally substituted up to two times with (C₁-C₄)alkyl;
R¹¹ and R¹² are each selected independently from H, F and Cl with the proviso that when one of R¹¹ and R¹² is F or Cl, the other must be H;
X is selected from O, S, CH₂, and NH, and
   when X is NH, the H on NH is optionally replaced with pyridyl, pyrazinyl, phenyl, or (C₁-C₄)alkyl optionally substituted with up to two substituents
      independently selected from OH, (C₁-C₃)alkoxy, N[(C₁-C₃)alkyl]₂,
      C(O)-pyrrolidinyl, N[(C₁-C₄)alkyl]₂, and phenyl said phenyl being optionally substituted with up to two substituents independently selected from CN and (C₁-C₃)alkoxy,
   and when X is O, S, or CH₂, the 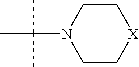
   moiety is optionally substituted by replacing any H atom in the 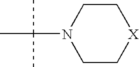
   moiety with (C₁-C₄)alkyl;
or a pharmaceutically acceptable salt or ester thereof.

The terms identified above have the following meaning throughout:

The term 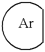
represents a 6 membered aromatic ring containing 0, 1 or 2 N atoms. That is, one embodiment of Ar is an aromatic ring containing 6 C atoms. Those 6 C atoms include the 2 C atoms that the Ar ring shares with the adjacent pyrazinone ring. This definition also includes the aromatic ring described above where 1 or 2 C atoms have been replaced with N atoms. The N atom(s) may be located at any position on the aromatic ring except they may not be located at the adjacent C atoms that are shared by the Ar ring and the adjacent pyrazinone ring. Examples of 6 membered aromatic N containing rings include pyrido, pyrimido, pyrazino, and pyridazo.

R¹ and R² are each independently attached to the Ar ring at any available C atom except that when R¹ and R² together form a ring, each of R¹ and R² are attached to adjacent C atoms that are shared with the Ar ring so that the R¹/R² ring is fused to the Ar ring through 2 adjacent C atoms that are shared between the R¹/R² ring and the Ar ring.

R⁴ is attached to the indolyl moiety of the core molecule at either the 5 or 6 atom of the indole moiety.

R⁵ is attached to the core molecule at the 5 or 6 atom on the indole moiety that is not occupied by the R⁴ group. That is, when R⁴ is attached to the 5 atom of the indoyl moiety, then R⁵ is attached to the 6 atom of the indolyl moiety, and visa versa.

The term “optionally substituted” means that, unless indicated otherwise, the moiety so modified may have from one to up to at least two of the substituents indicated. Each substituent may replace any H atom on the moiety so modified as long as the replacement is chemically possible and chemically stable. For example, a chemically unstable compound would be one where each of two substituents are bonded to a single C atom through each substituent's heteroatom. Another example of a chemically unstable compound would be one where an alkoxy group is bonded to the unsaturated carbon of an alkene to form an enol ether. When there are two substituents on any moiety, each substituent is chosen independently of the other substituent and so that, accordingly, the substituents can be the same or different.

The terms “(C₁-C₃)alkyl” and “(C₁-C₄)alkyl” and “(C₁-C₆)alkyl” mean linear or branched saturated carbon groups having from about 1 to about 3, about 4, or about 6 C atoms, respectively. Such groups include but are not limited to methyl, ethyl, n-propyl, isopropyl, and the like.

The term “(C₃-C₆)cycloalkyl” means a saturated monocyclic alkyl group of from 3 to about 6 carbon atoms and includes such groups as cyclopropyl, cyclobutyl, cyclopentyl, cyclohexyl.

The term “(C₂-C₆)alkenyl” means a linear or branched carbon group having from about 2 to about 6 C atoms wherein at least two adjacent C atoms in the alkenyl group are joined by a double bond, with the proviso that when a C atom is double bonded to one adjacent C atom, it must be single bonded to any other adjacent C atom. The alkenyl group is attached to the rest of the molecule through a single bond.

The term (C₂-C₆)alkynyl means means a linear or branched carbon group having from about 2 to about 6 C atoms wherein at least two adjacent C atoms in the alkynyl group are joined by a triple bond, with the proviso that when a C atom is triple bonded to one adjacent C atom, it must be single bonded to any other adjacent C atom. The alkynyl group is attached to the rest of the molecule through a single bond.

The terms “(C₁-C₃)alkoxy”, “(C₁-C₄)alkoxy” and “(C₁-C₆)alkoxy” mean a linear or branched saturated carbon group having from about 1 to about 3, about 4, or about 6 C atoms, respectively, said carbon group being attached to an O atom. The O atom is the point of attachment of the alkoxy substituent to the rest of the molecule. Such groups include but are not limited to methoxy, ethoxy, n-propoxy, isopropoxy, and the like.

The term “halo” means an atom selected from Cl, Br, F and I.

The term “phenoxy” means a phenyl ring attached to an O atom, the O atom being attached to the rest of the molecule.

When “(O)” is used in a chemical formula, it means ═O. That is, ═O means an O atom that is double bonded to the C or S atom to which it is attached.

The formula “N[C₁-C₃)alkyl]₂” means that each of the 2 possible alkyl groups attached to the N atom are selected independently from the other so that they may be the same or they may be different.

When a phenyl ring or a heterocycle is attached to the rest of the molecule, it is attached by replacing any H atom on the phenyl ring or on the heterocycle, respectively, with a bond to the rest of the molecule, as long as the replacement is chemically possible and chemically stable. 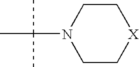
means morpholinyl, thiomorpholinyl, piperidinyl or piperazinyl. Each is optionally substituted as described above.

Representative compounds of the invention are shown by way of example in Table I.

TABLE 1

| | | LCMS | LCMS | Preparative |
|---|---|---|---|---|
| Ex. | | RT | Ion | Method(s) |
| No. | Structure | (min) | [M + H]⁺ | (Ex. No.) |
| 1 | 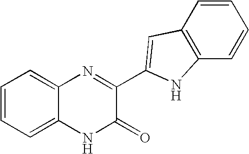 | 2.96 | 262.2 | 1 |
| 2 | 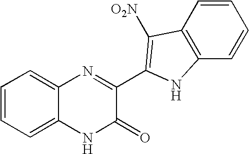 | 2.86 | 307.2 | 1, 2 |
| 3 | 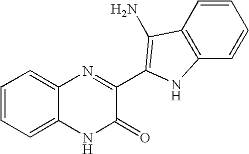 | 2.33 | 277.3 | 1, 2, 3 |
| 4 | 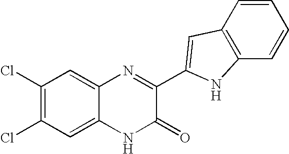 | 3.66 | 330.2 | 1, 2 |
| 5 | 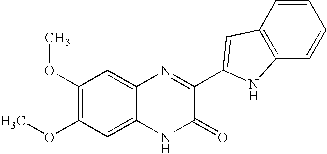 | 2.85 | 372.3 | 1, 2 |
| 6 | 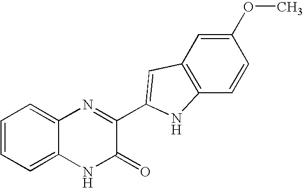 | 2.71 | 292.3 | 1 |
| 7 | 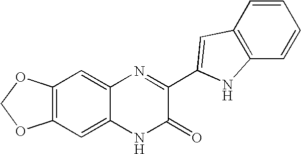 | 3.05 | 306.3 | 1 |
| 8 | 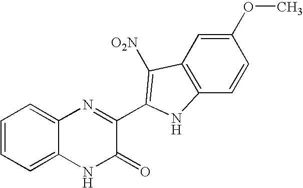 | 2.64 | 337.2 | 1, 2 |
| 9 | 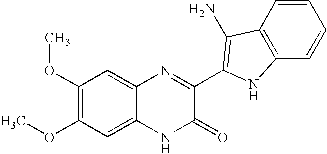 | 1.02 | 337.3 | 1, 2, 3 |
| 10 | 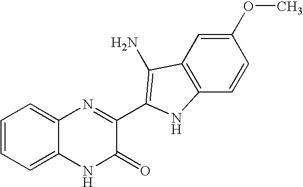 | 2.11 | 307.2 | 3 |
| 11 | 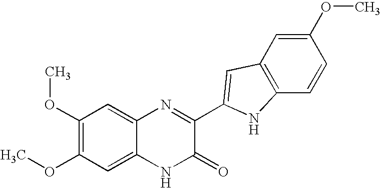 | 2.70 | 352.2 | 1 |
| 12 | 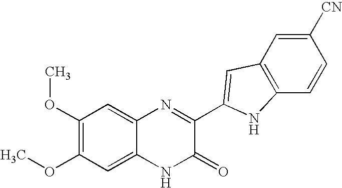 | 2.68 | 347.2 | 12 |
| 13 | 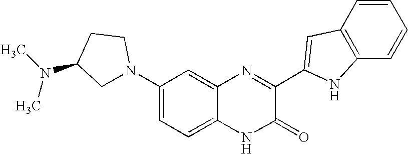 | 2.06 | 374.4 | 13 |
| 14 | 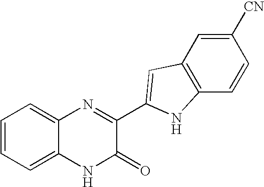 | 2.77 | 287.3 | 12 |
| 15 | 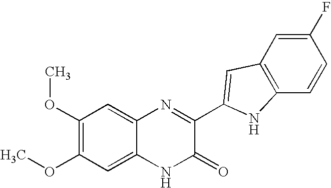 | 2.84 | 340.2 | 1 |
| 16 | 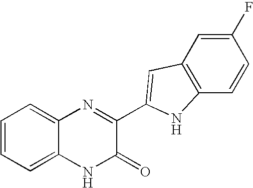 | 2.92 | 280.2 | 1 |
| 17 | 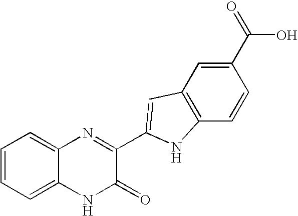 | 2.56 | 306.3 | 22 |
| 18 | 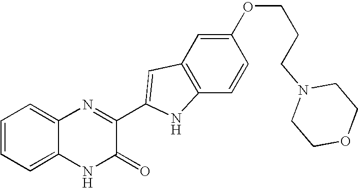 | 2.45 | 405.3 | 18 |
| 19 | 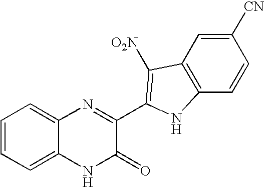 | 2.81 | 332.2 | 12, 2 |
| 20 | 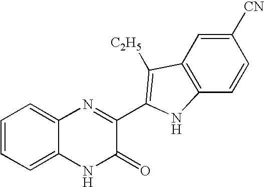 | 2.88 | 302.3 | 12, 2, 3 |
| 21 | 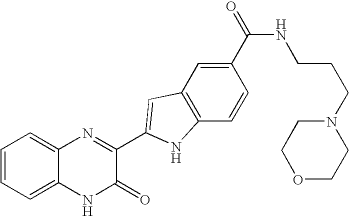 | 2.30 | 432.3 | 21 |
| 22 | 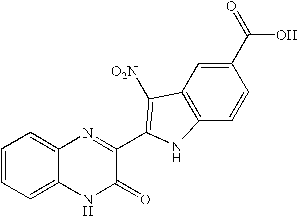 | 2.58 | 351.3 | 22 |
| 23 | 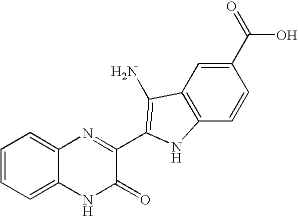 | 2.65 | 320.3 | 22, 3 |
| 24 | 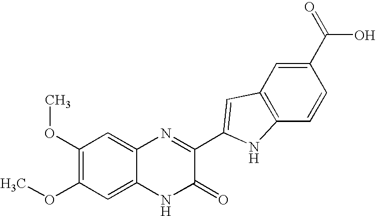 | 2.76 | 366.3 | 22 |
| 25 | 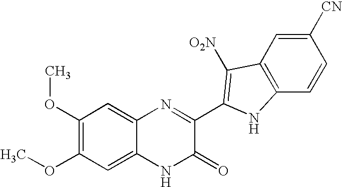 | 2.54 | 392.0 | 12, 2 |
| 26 | 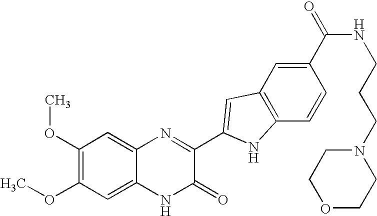 | 2.48 | 492.2 | 21 |
| 27 | 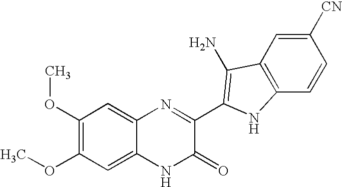 | 2.31 | 362.2 | 12, 2, 3 |
| 28 | 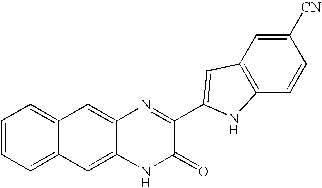 | 3.45 | 337.3 | 12 |
| 29 | 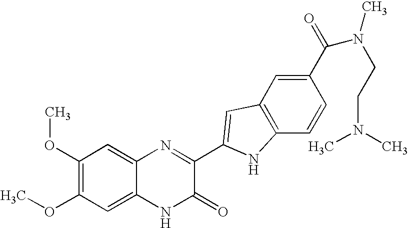 | 2.38 | 450.2 | 21 |
| 30 | 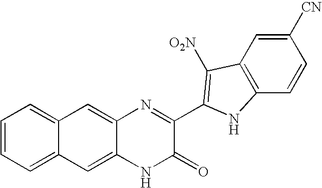 | 2.96 | 382.3 | 12, 2 |
| 31 | 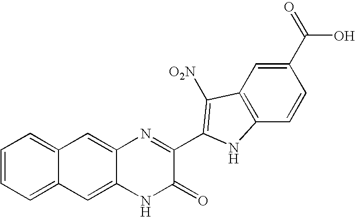 | 2.69 | 401.2 | 22 |
| 32 | 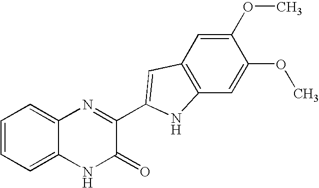 | 2.70 | 321.3 | 1 |
| 33 | 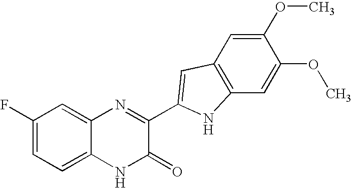 | 2.67 | 340.3 | 1 |
| 34 | 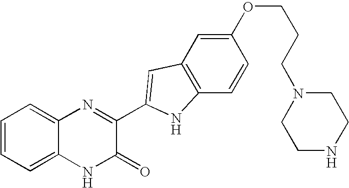 | 2.44 | 404.3 | 18 |
| 35 | 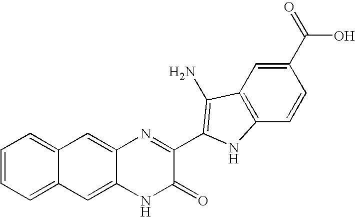 | 2.88 | 370.3 | 12, 22, 2, 3 |
| 36 | 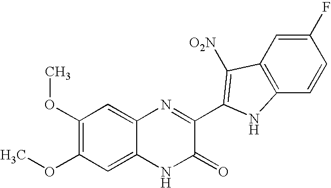 | 2.68 | 385.1 | 1, 2 |
| 37 | 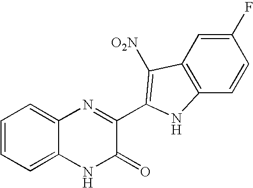 | 2.61 | 325.0 | 2 |
| 38 | 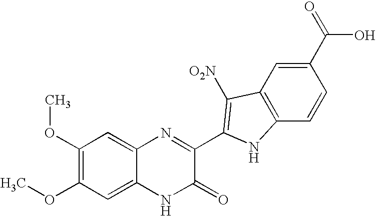 | 2.25 | 411.1 | 22 |
| 39 | 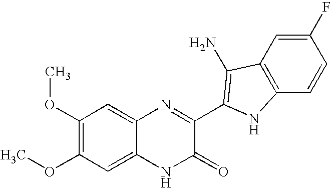 | 2.02 | 355.2 | 1, 2, 3 |
| 40 | 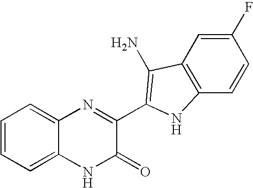 | 2.22 | 295.2 | 1, 2, 3 |
| 41 | 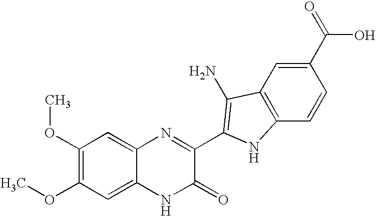 | 1.94 | 381.2 | 22, 2, 3 |
| 42 | 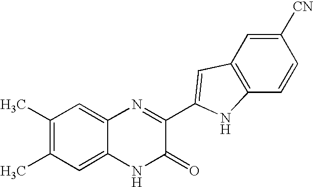 | 2.93 | 315.3 | 12 |
| 43 | 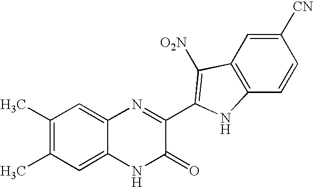 | 2.89 | 360.3 | 12, 2 |
| 44 | 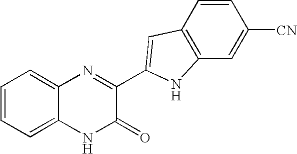 | 2.79 | 287.3 | 12 |
| 45 | 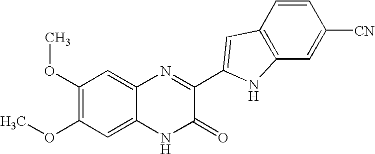 | 2.68 | 347.2 | 12 |
| 46 | 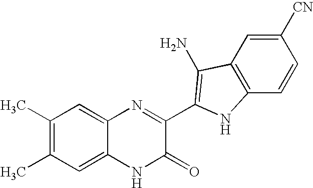 | 2.98 | 330.3 | 12, 2, 3 |
| 47 | 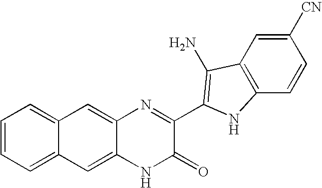 | 3.27 | 351.4 | 12, 2, 3 |
| 48 | 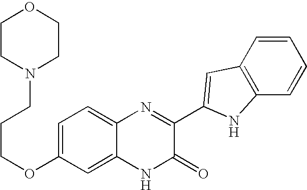 | 2.26 | 405.2 | 49 |
| 49 | 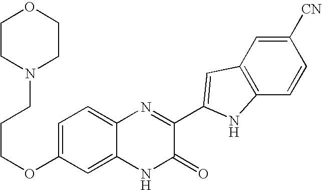 | 2.11 | 430.2 | 49 |
| 50 | 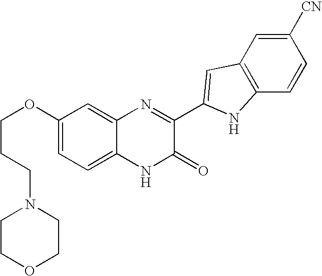 | 2.21 | 430.3 | 50 |
| 51 | 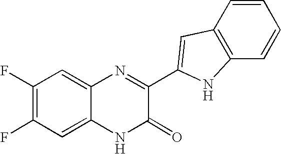 | 3.13 | 298.3 | 1 |
| 52 | 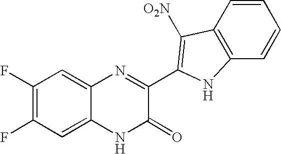 | 2.83 | 343.1 | 1, 2 |
| 53 | 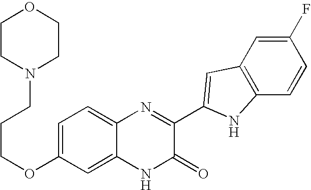 | 2.33 | 423.3 | 49 |
| 54 | 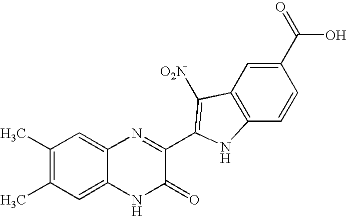 | 2.71 | 379.2 | 22 |
| 55 | 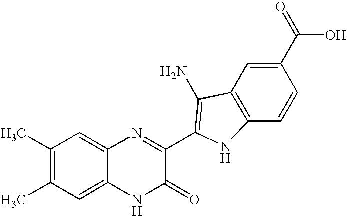 | 2.79 | 348.4 | 22, 2, 3 |
| 56 | 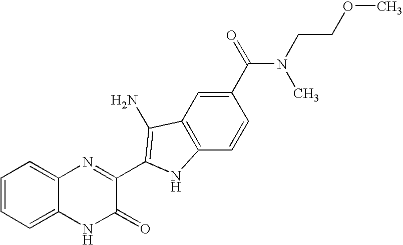 | 2.11 | 392.2 | 56 |
| 57 | 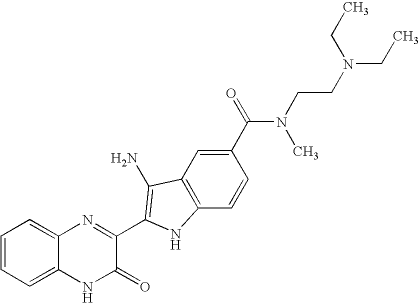 | 1.73 | 433.1 | 56 |
| 58 | 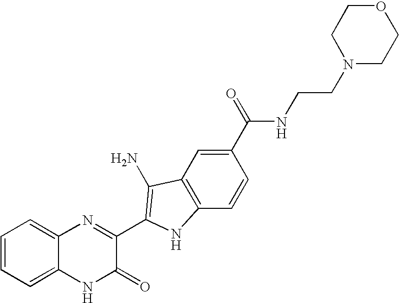 | 1.67 | 433.1 | 56 |
| 59 | 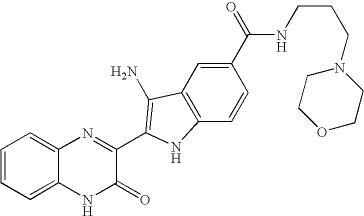 | 1.70 | 447.2 | 56 |
| 60 | 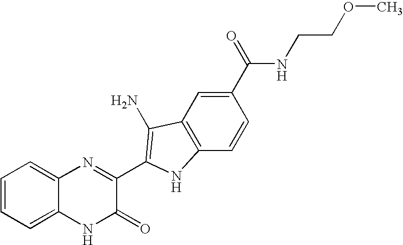 | 2.03 | 378.2 | 56 |
| 61 | 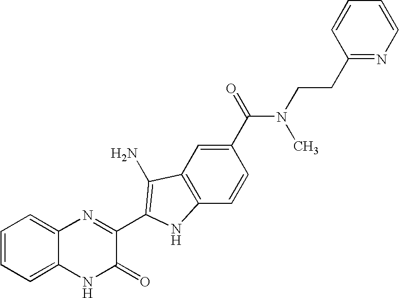 | 1.71 | 439.1 | 56 |
| 62 | 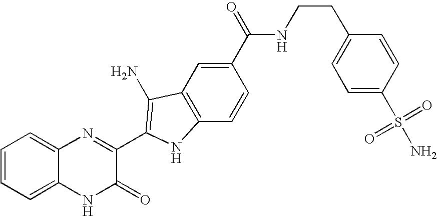 | 2.24 | 503.2 | 56 |
| 63 | 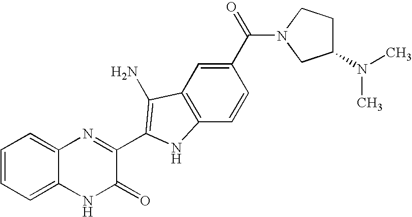 | 1.23 | 417.1 | 56 |
| 64 | 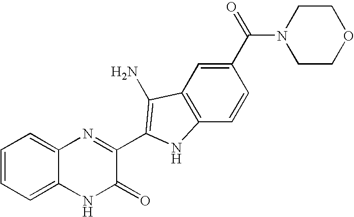 | 2.03 | 390.3 | 56 |
| 65 | 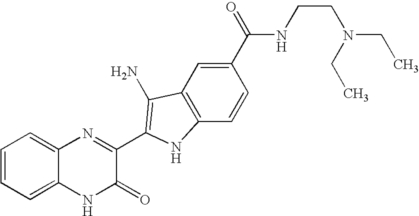 | 1.74 | 419.1 | 56 |
| 66 | 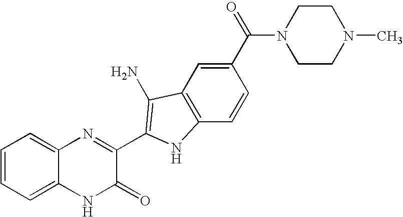 | 1.22 | 403.0 | 56 |
| 67 | 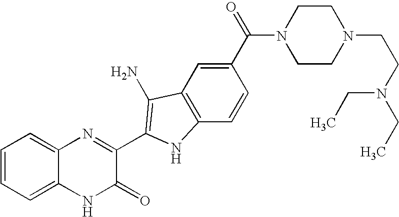 | 1.58 | 488.2 | 56 |
| 68 | 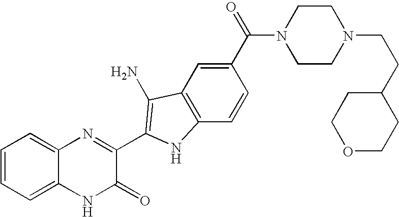 | 1.50 | 502.1 | 56 |
| 69 | 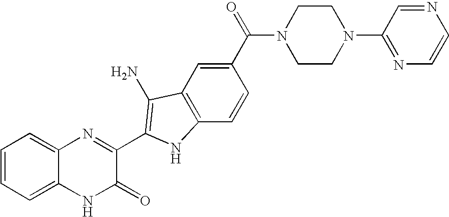 | 2.17 | 466.9 | 56 |
| 70 | 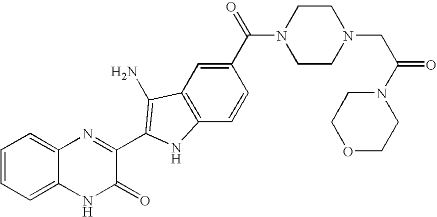 | 1.62 | 516.0 | 56 |
| 71 | 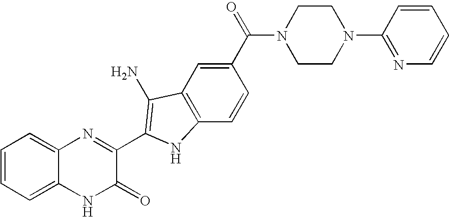 | 1.78 | 466.0 | 56 |
| 72 | 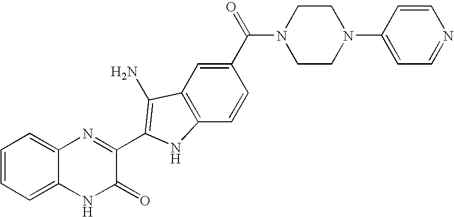 | 1.73 | 466.3 | 56 |
| 73 | 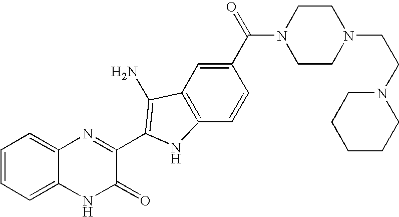 | 1.58 | 500.2 | 56 |
| 74 | 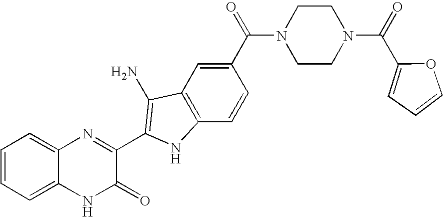 | 2.16 | 258.2 (major ion) | 56 |
| 75 | 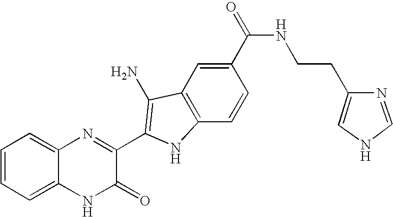 | 1.68 | 414.1 | 56 |
| 76 | 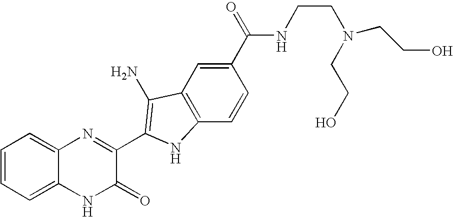 | 1.54 | 451.2 | 56 |
| 77 | 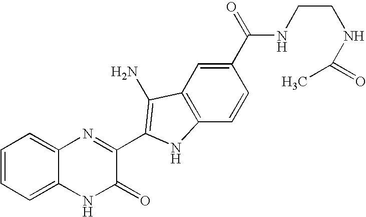 | 1.87 | 405.1 | 56 |
| 78 | 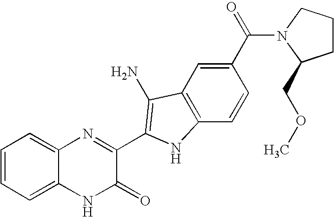 | 2.28 | 418.1 | 56 |
| 79 | 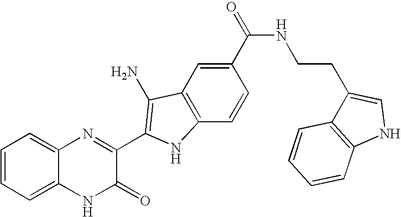 | 2.65 | 463.2 | 56 |
| 80 | 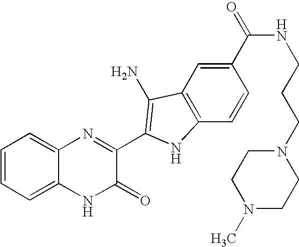 | 1.31 | 460.2 | 56 |
| 81 | 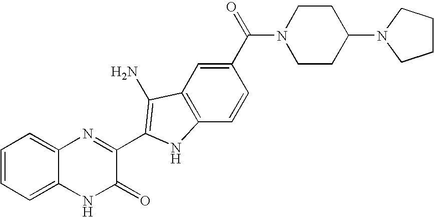 | 1.57 | 457.2 | 56 |
| 82 | 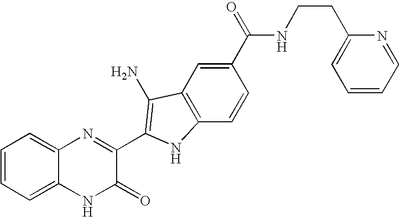 | 1.66 | 425.1 | 56 |
| 83 | 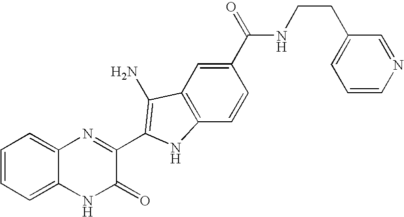 | 1.66 | 425.2 | 56 |
| 84 | 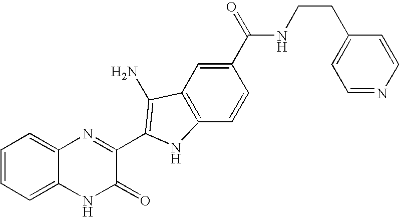 | 1.63 | 425.0 | 56 |
| 85 | 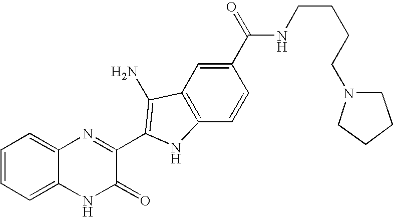 | 1.71 | 445.2 | 56 |
| 86 | 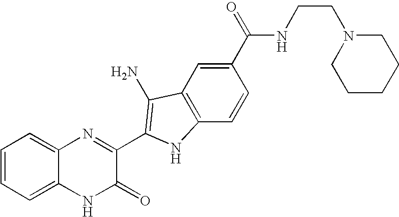 | 1.75 | 431.1 | 56 |
| 87 | 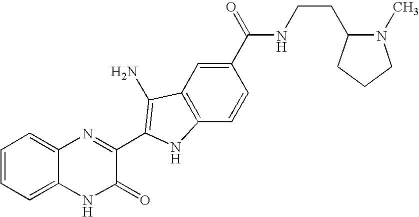 | 1.68 | 431.1 | 56 |
| 88 | 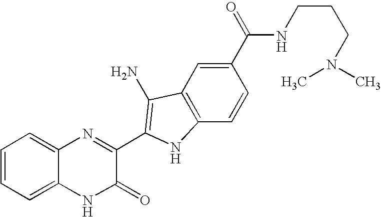 | 1.58 | 405.1 | 56 |
| 89 | 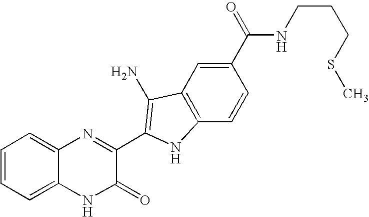 | 2.30 | 408.1 | 56 |
| 90 | 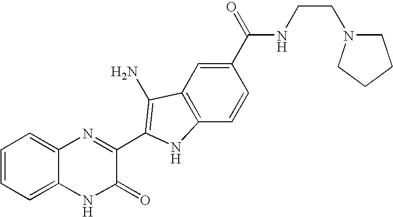 | 1.65 | 417.1 | 56 |
| 91 | 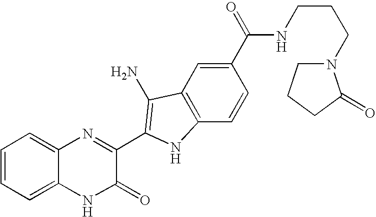 | 1.99 | 445.2 | 56 |
| 92 | 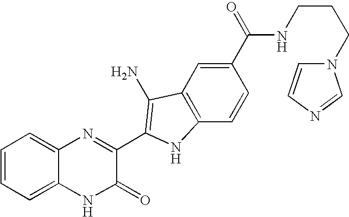 | 1.64 | 428.1 | 56 |
| 93 | 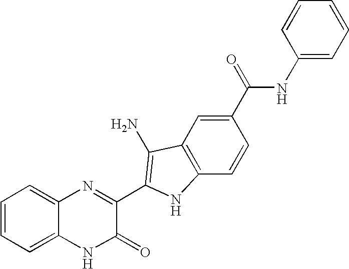 | 2.92 | 396.1 | 56 |
| 94 | 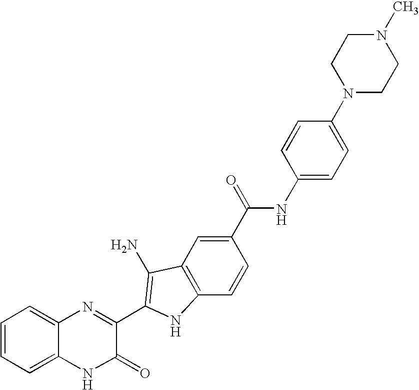 | 2.30 | 494.1 | 56 |
| 95 | 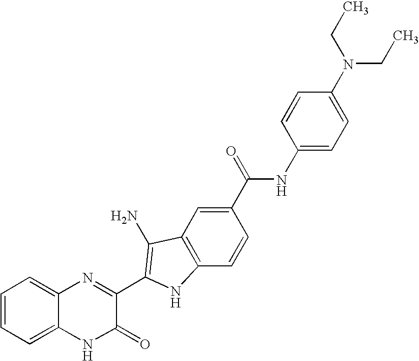 | 2.38 | 467.1 | 56 |
| 96 | 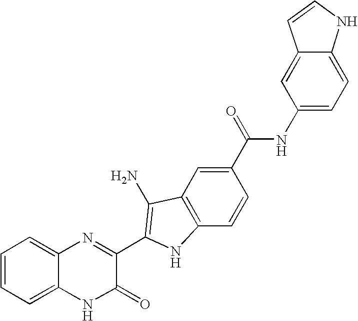 | 2.71 | 435.1 | 56 |
| 97 | 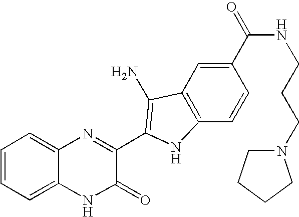 | 1.67 | 431.1 | 56 |
| 98 | 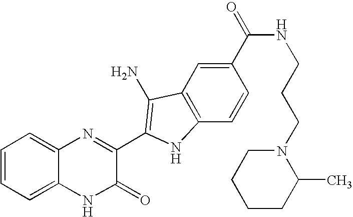 | 1.80 | 459.2 | 56 |
| 99 | 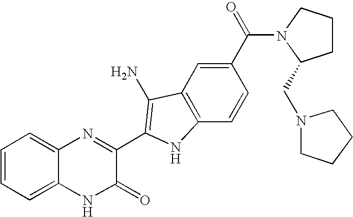 | 1.83 | 457.1 | 56 |
| 100 | 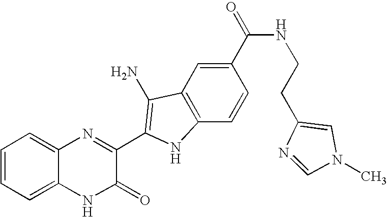 | 1.64 | 428.1 | 56 |
| 101 | 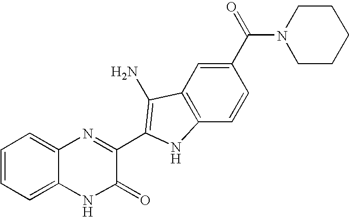 | 2.29 | 388.2 | 56 |
| 102 | 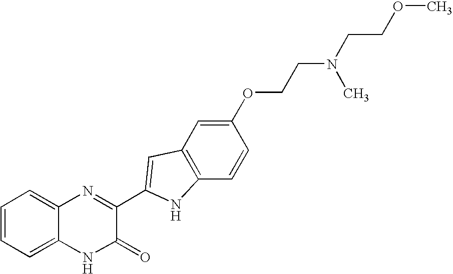 | 1.96 | 393.2 | 18 |
| 103 | 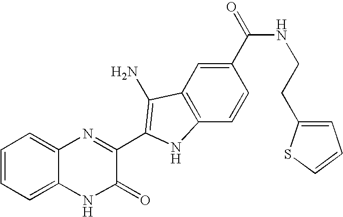 | 2.51 | 430.2 | 56 |
| 104 | 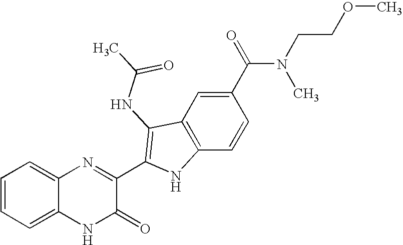 | 2.63 | 434.1 | 104 |
| 105 | 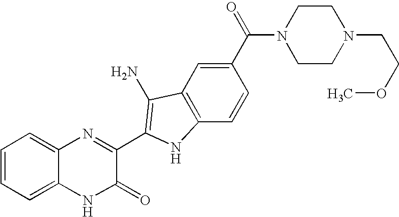 | 1.45 | 447.0 | 56 |
| 106 | 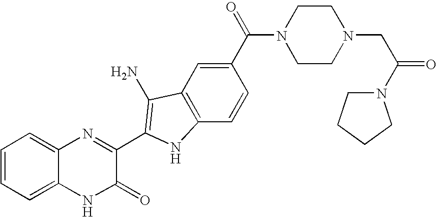 | 1.64 | 500.1 | 56 |
| 107 | 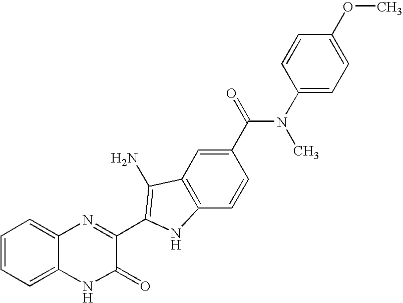 | 2.47 | 440.1 | 56 |
| 108 | 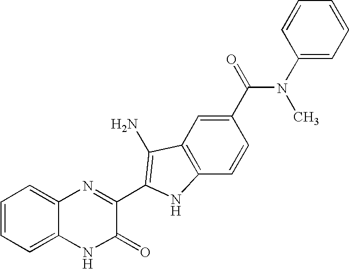 | 2.48 | 410.1 | 56 |
| 109 | 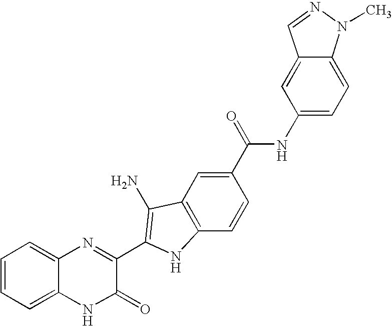 | 2.41 | 450.1 | 56 |
| 110 | 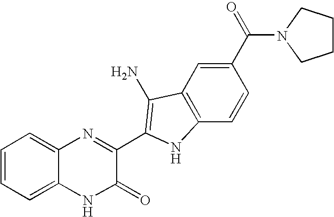 | 2.11 | 374.3 | 56 |
| 111 | 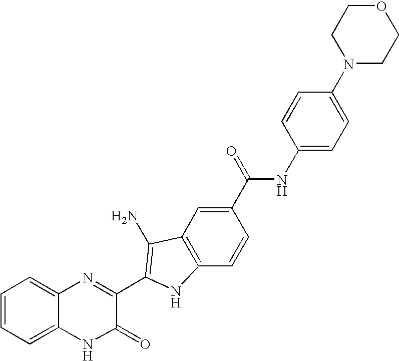 | 2.46 | 481.1 | 56 |
| 112 | 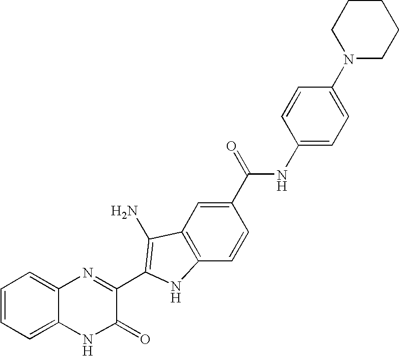 | 2.19 | 479.3 | 56 |
| 113 | 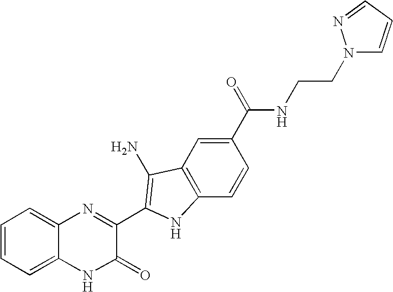 | 2.03 | 413.9 | 56 |
| 114 | 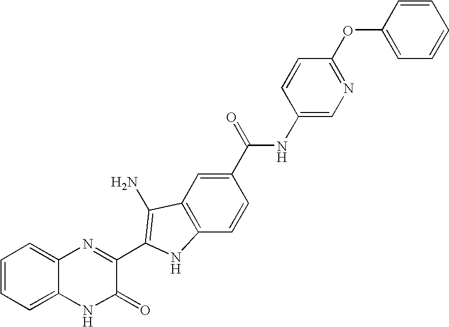 | 2.86 | 489.3 | 56 |
| 115 | 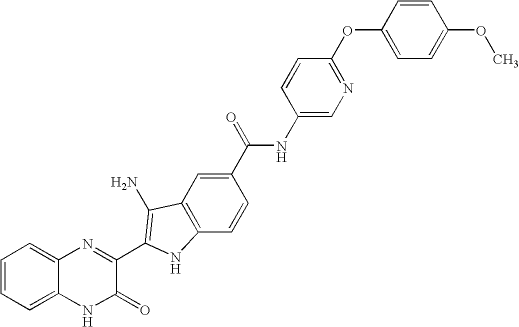 | 3.01 | 519.2 | 56 |
| 116 | 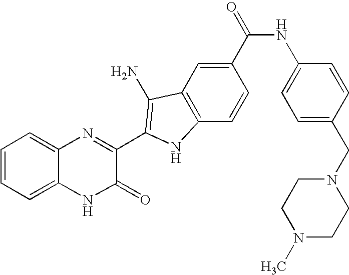 | 1.73 | 508.1 | 56 |
| 117 | 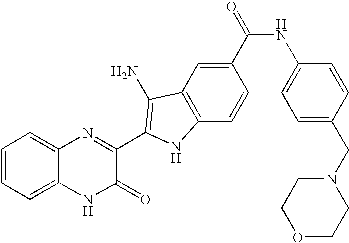 | 1.90 | 494.9 | 56 |
| 118 | 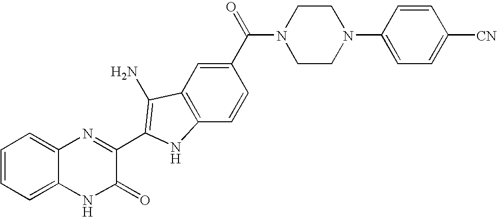 | 2.75 | 490.3 | 56 |
| 119 | 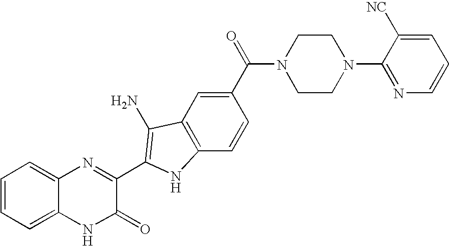 | 2.61 | 491.0 | 56 |
| 120 | 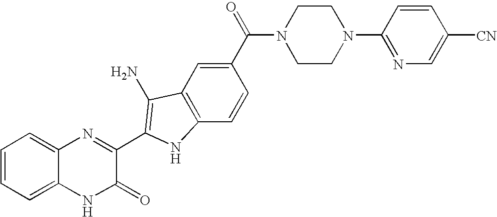 | 2.63 | 491.0 | 56 |
| 121 | 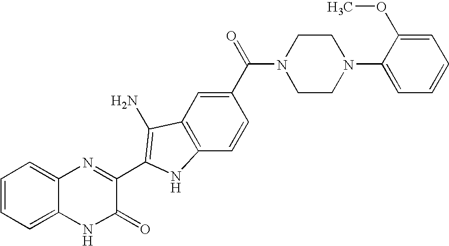 | 1.37 | 495.1 | 56 |
| 122 | 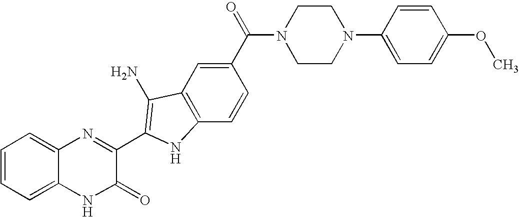 | 2.34 | 495.1 | 56 |
| 123 | 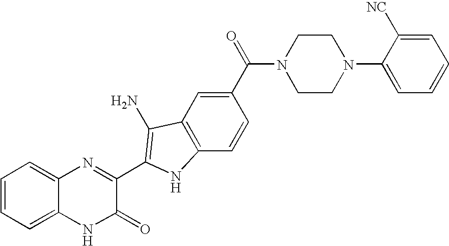 | 2.63 | 490.1 | 56 |
| 124 | 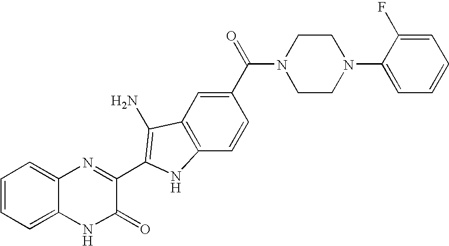 | 2.71 | 483.1 | 56 |
| 125 | 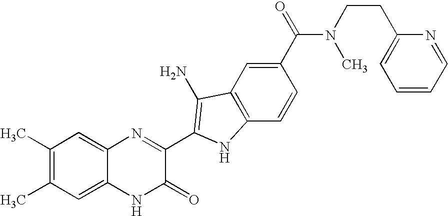 | 1.93 | 467.0 | 12, 2, 22, 56 |
| 126 | 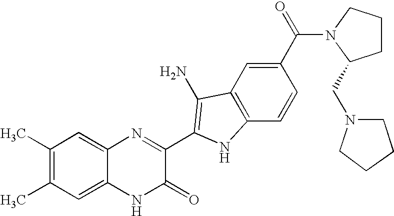 | 2.07 | 485.2 | 12, 2, 22, 56 |
| 127 | 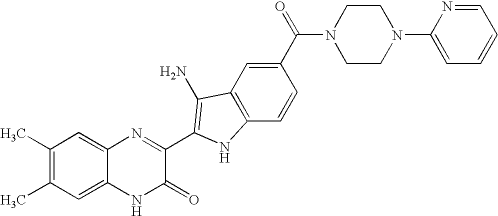 | 1.98 | 494.0 | 12, 2, 22, 56 |
| 128 | 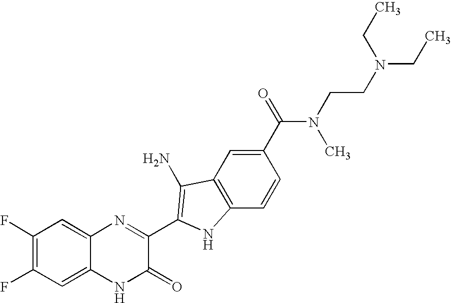 | 2.12 | 469.1 | 12, 2, 22, 56 |
| 129 | 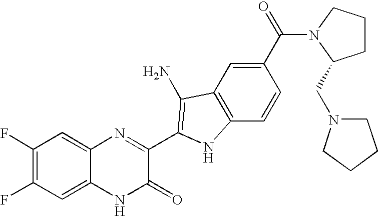 | 2.26 | 493.1 | 12, 2, 22, 56 |
| 130 | 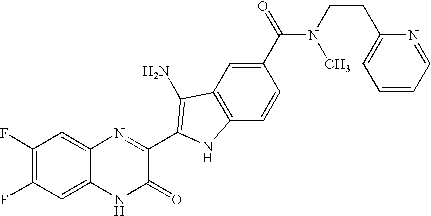 | 2.12 | 475.0 | 12, 2, 22, 56 |
| 131 | 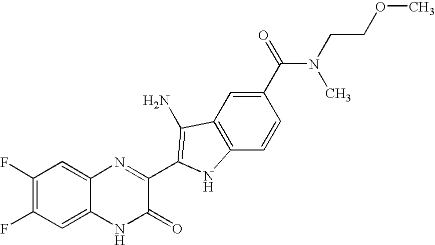 | 2.53 | 428.1 | 12, 2, 22, 56 |
| 132 | 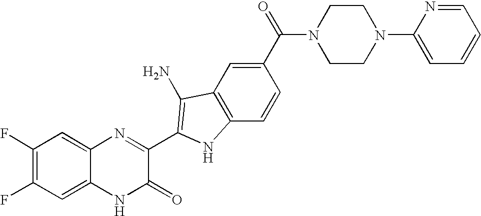 | 2.16 | 502.0 | 12, 2, 22, 56 |
| 133 | 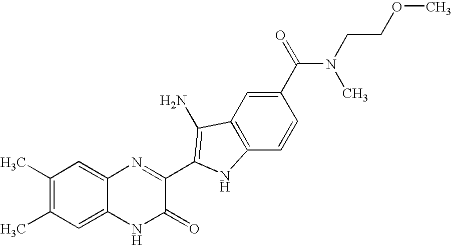 | 2.24 | 420.4 | 12, 2, 22, 56 |
| 134 | 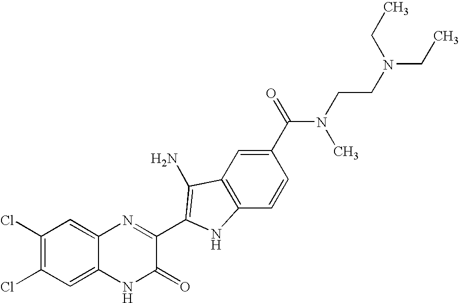 | 2.47 | 501.1 | 134 |
| 135 | 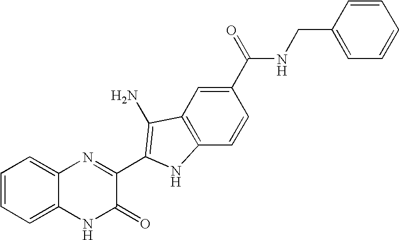 | 2.87 | 410.2 | 56 |
| 136 | 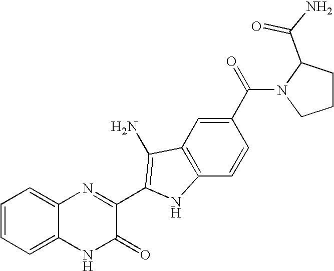 | 1.82 | 416.9 | 56 |
| 137 | 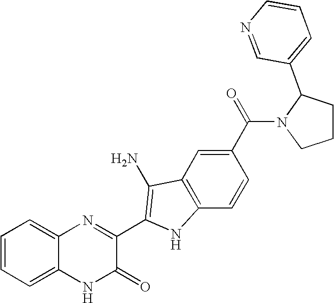 | 1.84 | 451.1 | 56 |
| 138 | 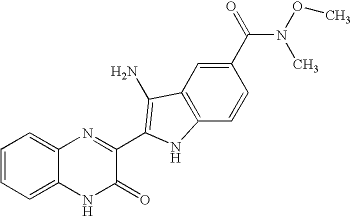 | 2.58 | 364.2 | 56 |
| 139 | 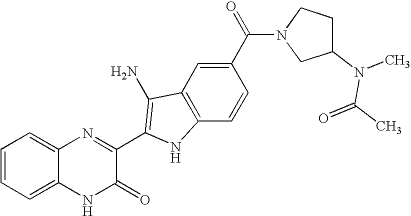 | 2.09 | 445.0 | 56 |
| 140 | 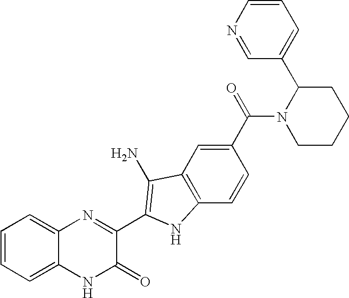 | 2.09 | 465.2 | 56 |
| 141 | 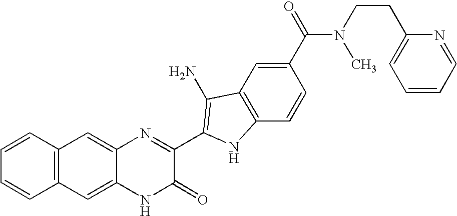 | 2.18 | 489.1 | 12, 2, 22, 56 |
| 142 | 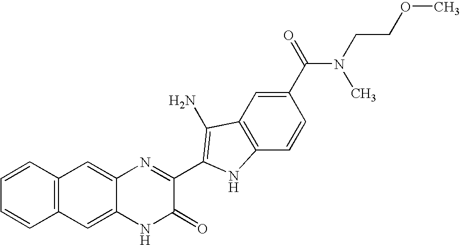 | 2.77 | 442.2 | 12, 2, 22, 56 |
| 143 | 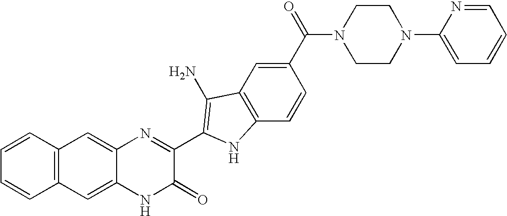 | 2.37 | 516.0 | 12, 2, 22, 56 |
| 144 | 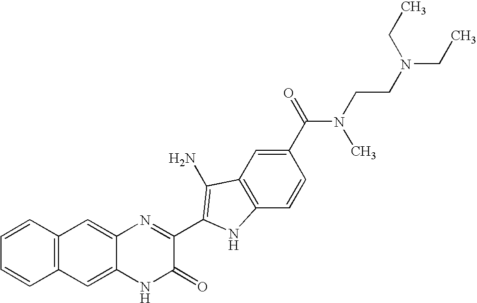 | 2.42 | 483.1 | 12, 2, 22, 56 |
| 145 | 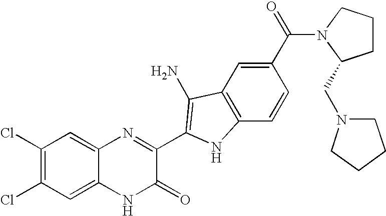 | 2.65 | 525.1 | 12, 2, 22, 134 |
| 146 | 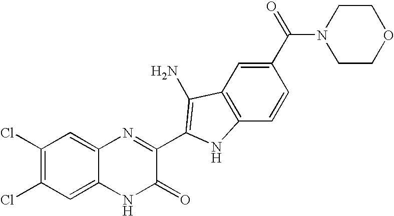 | 2.71 | 458.1 | 12, 2, 22, 134 |
| 147 | 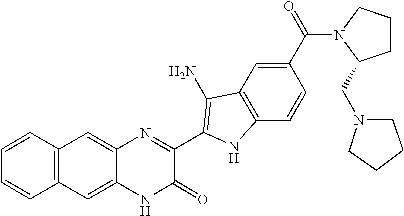 | 2.31 | 507.36 | 12, 2, 22, 56 |
| 148 | 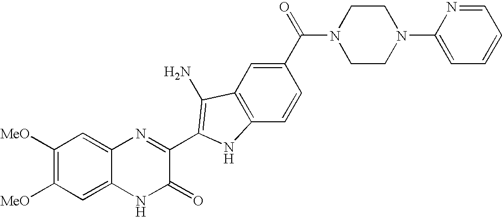 | 1.83 | 526.0 | 12, 2, 22, 56 |
| 149 | 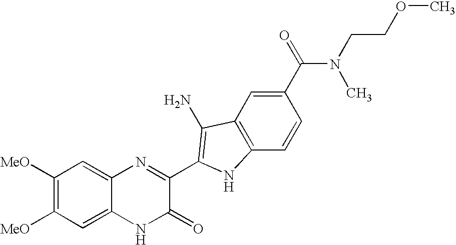 | 2.11 | 452.2 | 12, 2, 22, 56 |
| 150 | 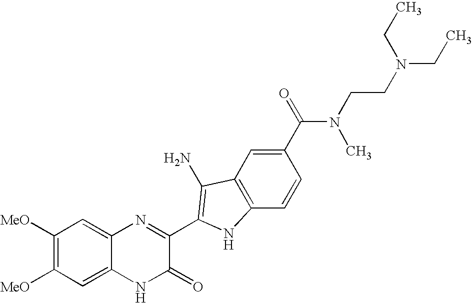 | 1.64 | 493.1 | 12, 2, 22, 56 |
| 151 | 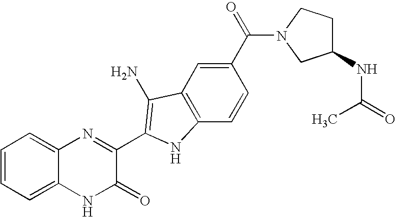 | 1.97 | 431.1 | 151 |
| 152 | 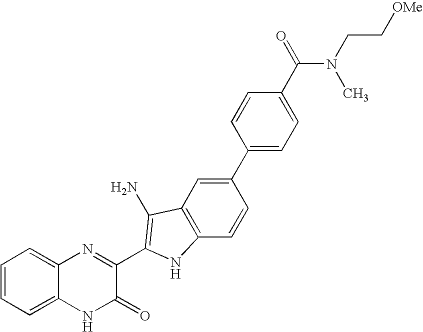 | 2.50 | 468.2 | 152, 12, 2, 22, 56 |
| 153 | 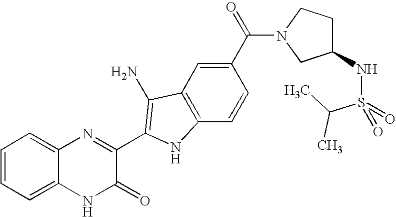 | 2.29 | 495.2 | 151 |
| 154 | 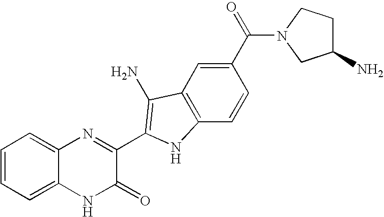 | 1.29 | 389.0 | 151, 3 |
| 155 | 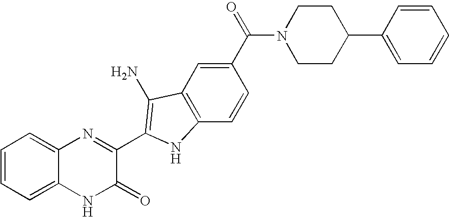 | 2.87 | 464.2 | 155 |
| 156 | 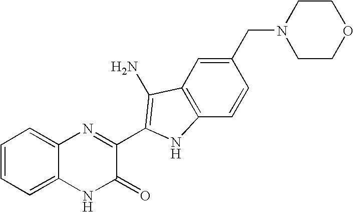 | 1.51 | 375.8 | 156 |
| 157 | 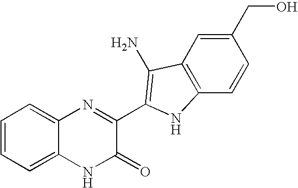 | 1.72 | 307.1 | 156 |
| 158 | 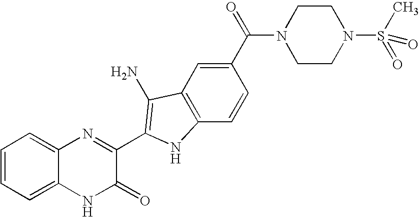 | 2.09 | 467.1 | 158, 56 |
| 159 | 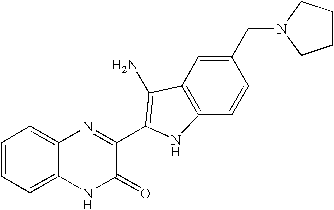 | 1.58 | 359.9 | 156 |
| 160 | 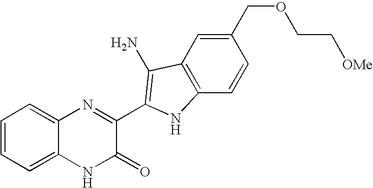 | 2.14 | 365.1 | 160 |
| 161 | 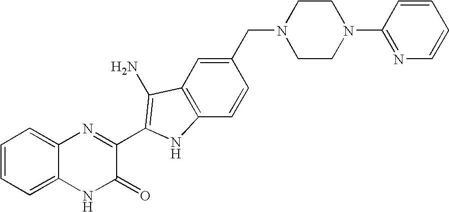 | 1.63 | 451.9 | 156 |
| 162 | 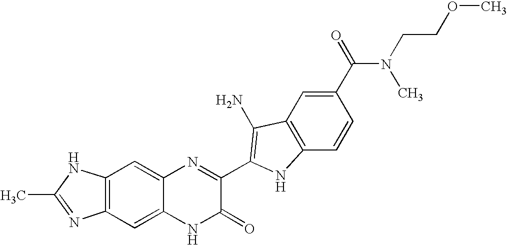 | 1.70 | 446.2 | 12, 2, 22, 56 |
| 163 | 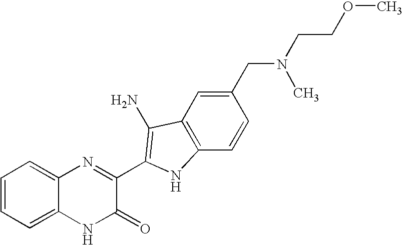 | 1.70 | 377.9 | 156 |
| 164 | 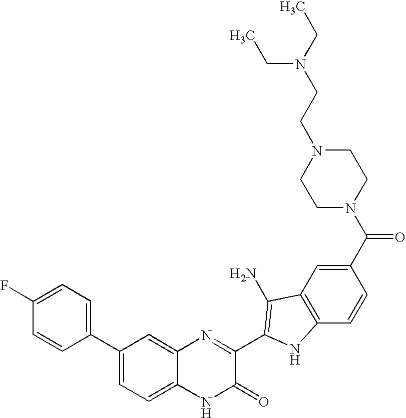 | 2.16 | 582.2 | 336, 2, 22, 56 |
| 165 | 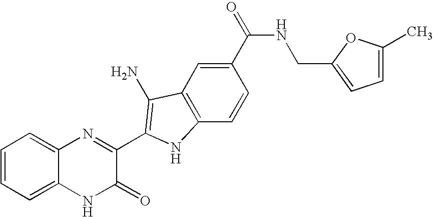 | 2.98 | 414.2 | 56 |
| 166 | 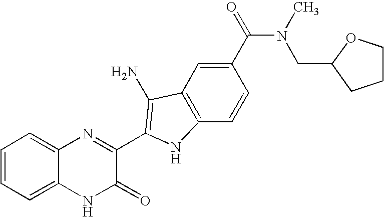 | 2.12 | 418.0 | 56 |
| 167 | 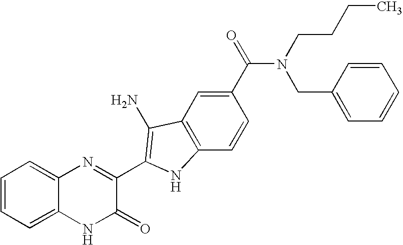 | 3.40 | 467 | 56 |
| 168 | 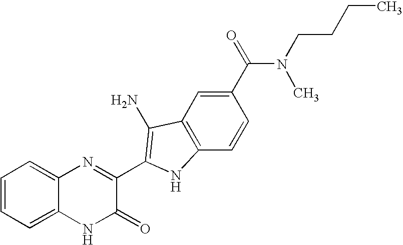 | 2.94 | 390 | 56 |
| 169 | 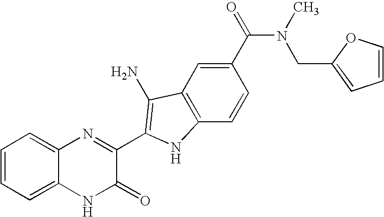 | 2.96 | 414.2 | 56 |
| 170 | 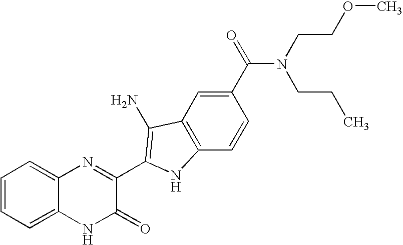 | 2.71 | 420 | 56 |
| 171 | 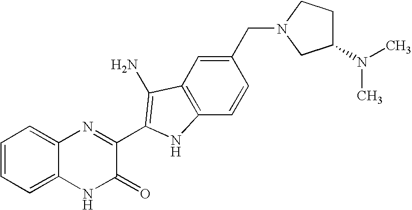 | 1.02 | 402.9 | 156 |
| 172 | 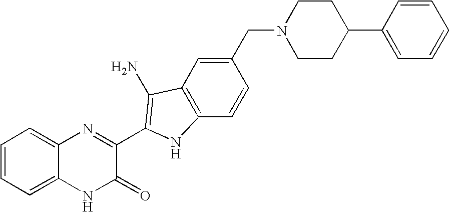 | 2.21 | 450.0 | 156 |
| 173 | 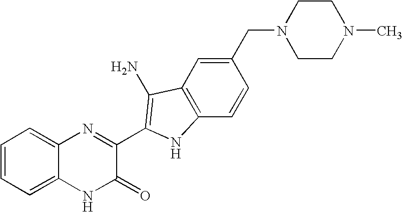 | 1.07 | 388.9 | 156 |
| 174 | 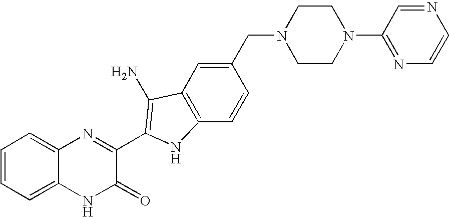 | 1.97 | 452.9 | 156 |
| 175 | 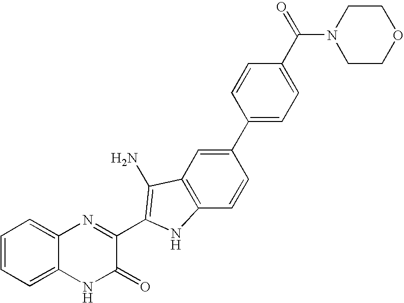 | 2.43 | 466.3 | 152, 12, 2, 22, 56 |
| 176 | 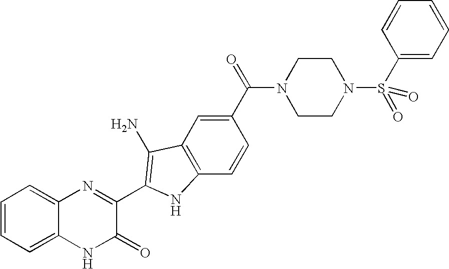 | 2.61 | 529.1 | 158, 56 |
| 177 | 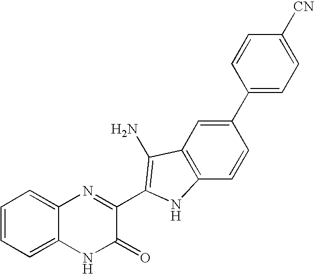 | 2.93 | 378.3 | 152 |
| 178 | 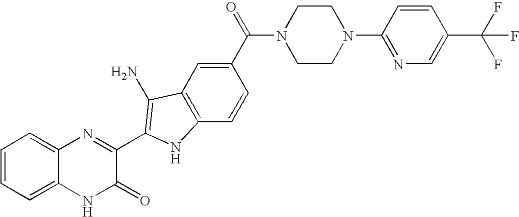 | 2.96 | 534.0 | 56 |
| 179 | 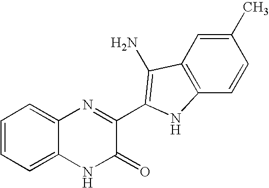 | 2.23 | 291.2 | 12, 2, 3 |
| 180 | 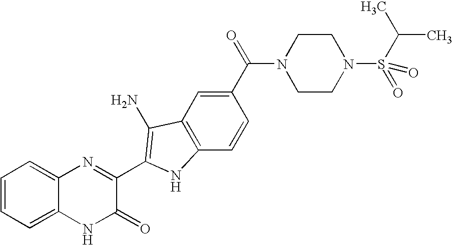 | 2.32 | 495.1 | 158, 56 |
| 181 | 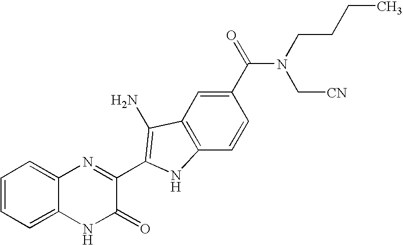 | 2.99 | 415 | 56 |
| 182 | 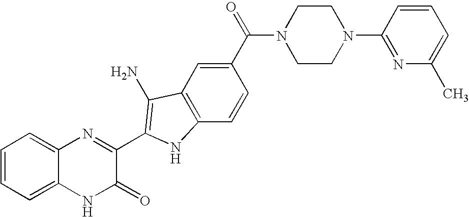 | 1.84 | 480.0 | 56 |
| 183 | 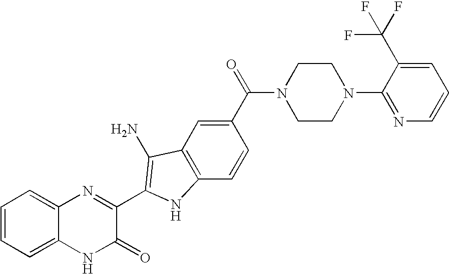 | 3.04 | 534.0 | 56 |
| 184 | 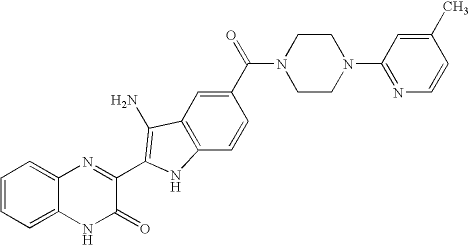 | 1.88 | 480.1 | 56 |
| 185 | 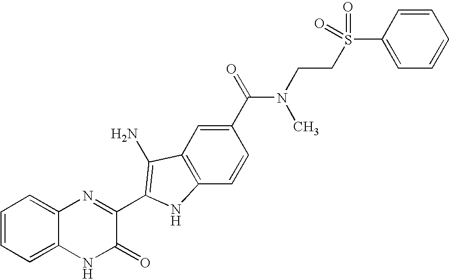 | 2.37 | 502.2 | 56 |
| 186 | 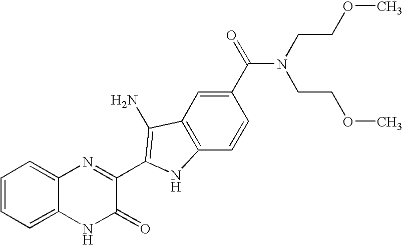 | 2.16 | 436.3 | 56 |
| 187 | 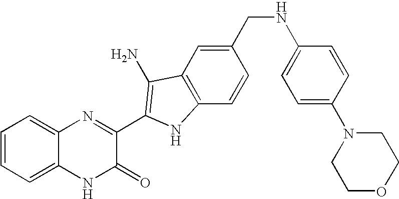 | 2.14 | 467.2 | 156 |
| 188 | 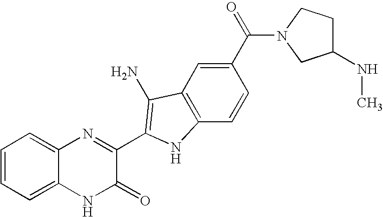 | 1.69 | 403.0 | 56 |
| 189 | 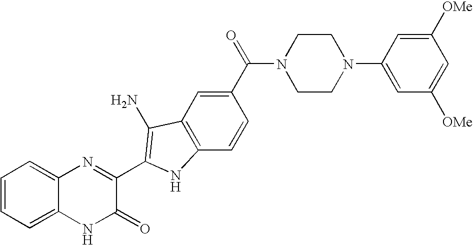 | 2.71 | 525.1 | 56 |
| 190 | 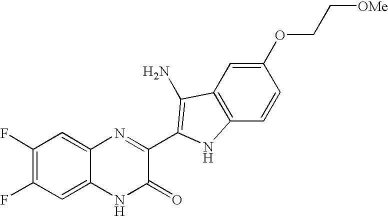 | 2.26 | 387.2 | 18, 2, 3 |
| 191 | 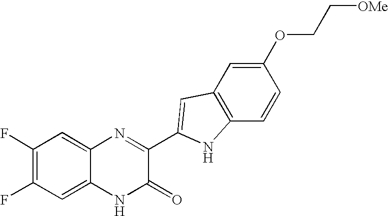 | 2.30 | 372.2 | 18 |
| 192 | 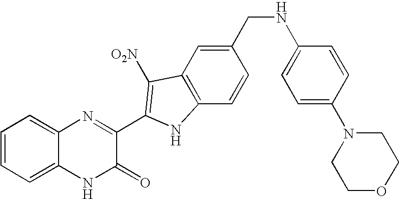 | 2.14 | 497.1 | 156 |
| 193 | 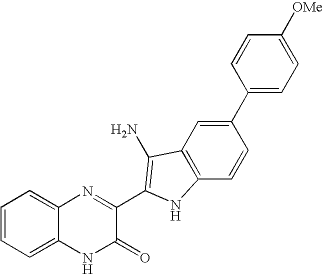 | 3.03 | 383.3 | 152, 1, 2, 3 |
| 194 | 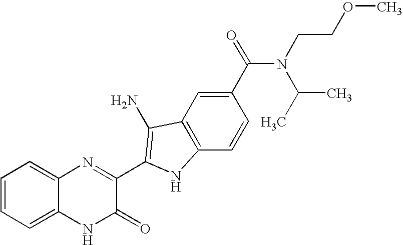 | 2.37 | 420.4 | 56 |
| 195 | 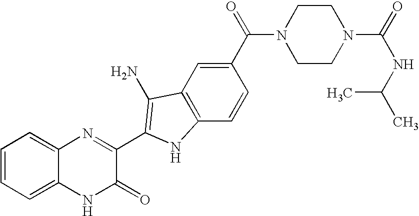 | 2.12 | 474.3 | 214, 3 |
| 196 | 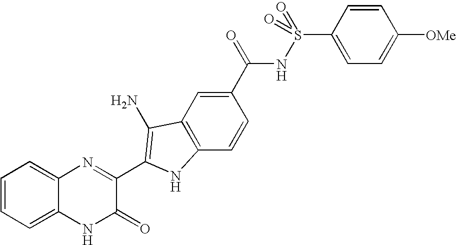 | 2.77 | 490.1 | 196 |
| 197 | 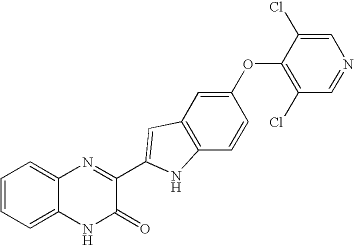 | 3.77 | 423.2 | 217 |
| 198 | 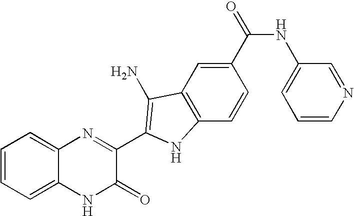 | 2.45 | 397.2 | 56 |
| 199 | 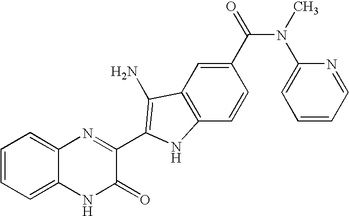 | 2.74 | 411.1 | 56 |
| 200 | 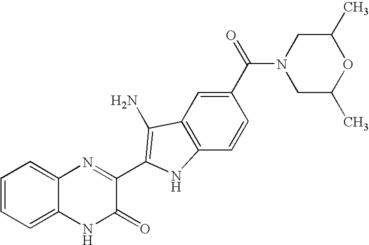 | 2.81 | 418.2 | 56 |
| 201 | 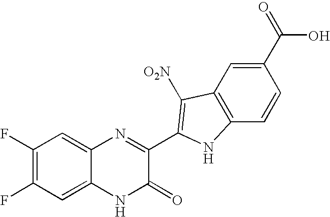 | 2.42 | 386.9 | 12, 2, 22 |
| 202 | 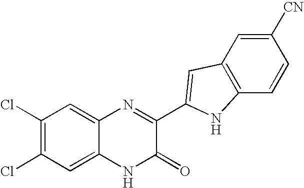 | No data | No data | 12 |
| 203 | 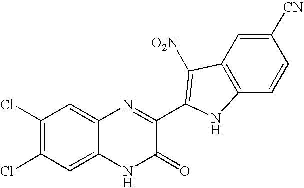 | No data | No data | 12, 2 |
| 204 | 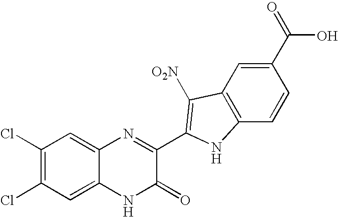 | 2.68 | No data | 12, 2, 3 |
| 205 | 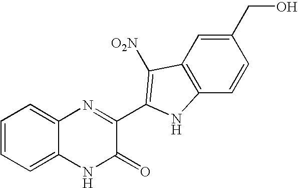 | 2.28 | 337.0 | 156 |
| 206 | 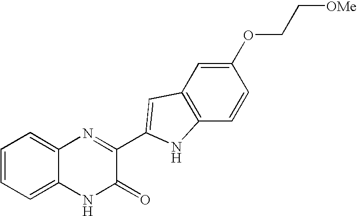 | 3.00 | 336 | 18 |
| 207 | 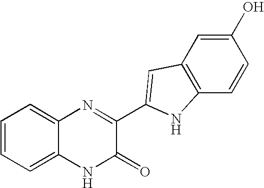 | 2.27 | 278.2 | 217 |
| 208 | 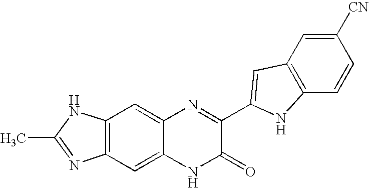 | 2.09 | 341.3 | 12 |
| 209 | 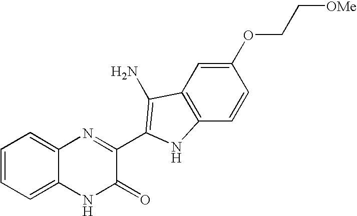 | 2.41 | 351.2 | 18, 2, 3 |
| 210 | 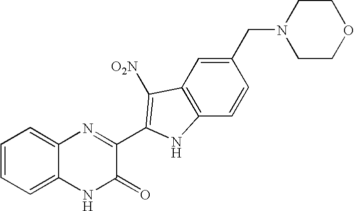 | 1.73 | 406.0 | 156 |
| 211 | 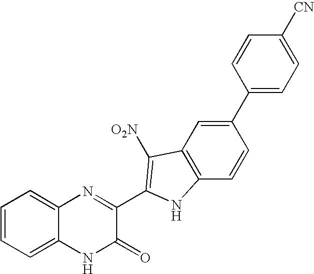 | 2.96 | 408.1 | 152, 1, 2 |
| 212 | 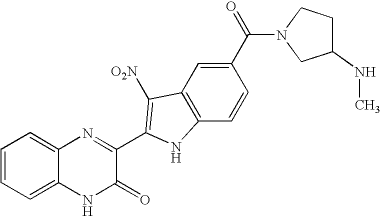 | 1.54 | 433.0 | 151 |
| 213 | 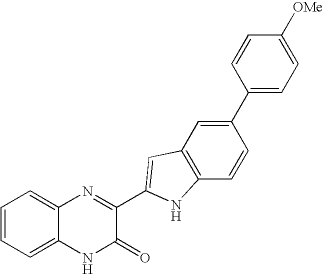 | 3.34 | 368.2 | 152, 1 |
| 214 | 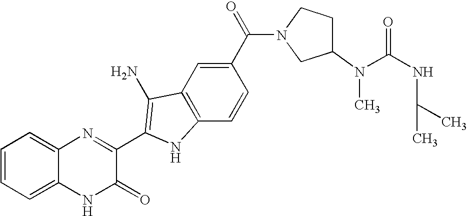 | 2.17 | 488.1 | 214, 3 |
| 215 | 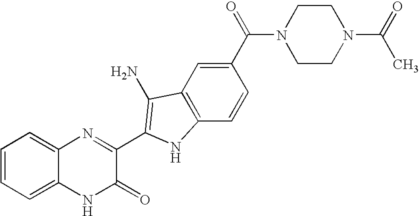 | 1.90 | 431.1 | 214, 3 |
| 216 | 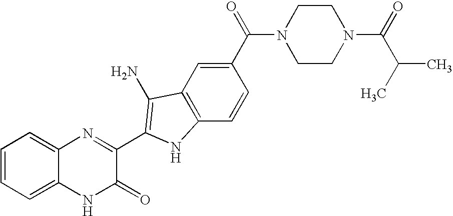 | 2.18 | 459.1 | 214, 3 |
| 217 | 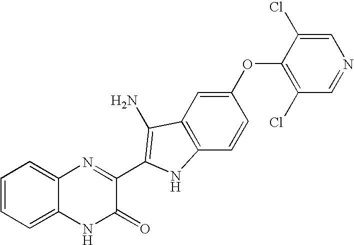 | 2.99 | 438.2 | 217 |
| 218 | 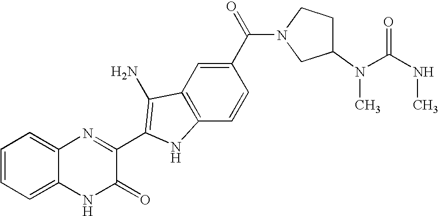 | 2.05 | 460.1 | 214, 3 |
| 219 | 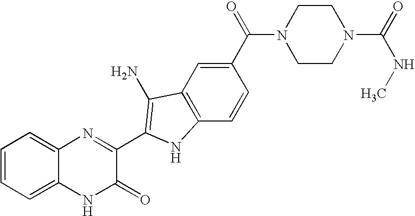 | 1.98 | 446.0 | 214, 3 |
| 220 | 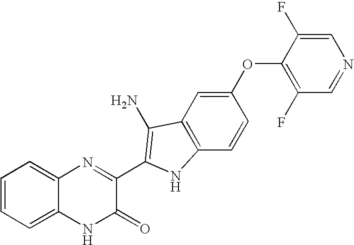 | 3.14 | 406.2 | 217 |
| 221 | 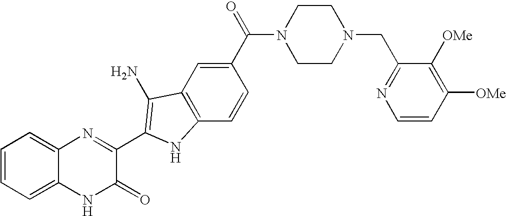 | 1.92 | 540.1 | 214, 3 |
| 222 | 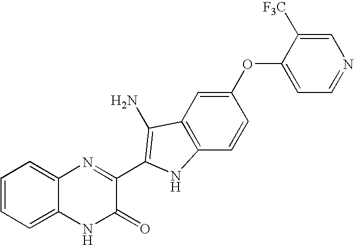 | 3.21 | 438.2 | 217 |
| 223 | 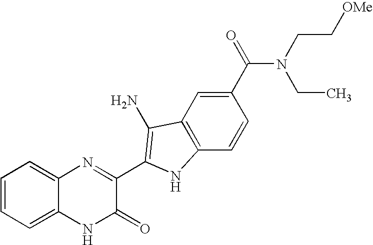 | 2.19 | 406.0 | 56 |
| 224 | 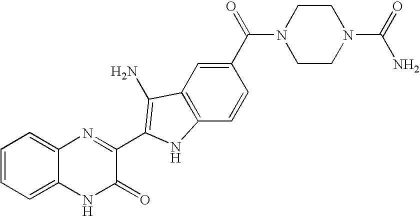 | 1.76 | 431.6 | 214, 3 |
| 225 | 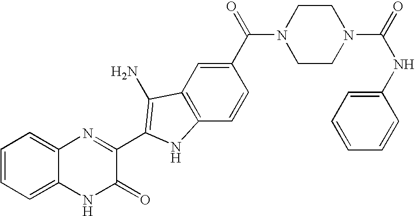 | 2.49 | 508.1 | 158, 56 |
| 226 | 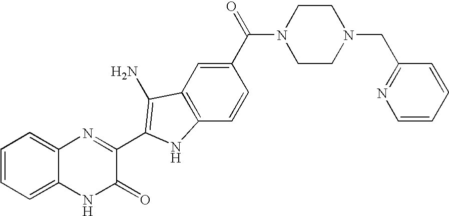 | 1.86 | 480.1 | 214, 3 |
| 227 | 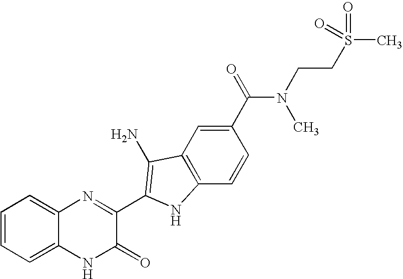 | 2.22 | 440.3 | 56 |
| 228 | 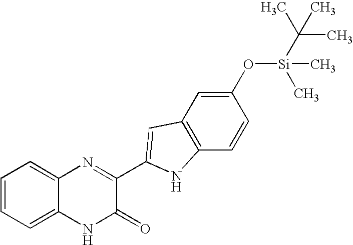 | 3.98 | 392.3 | 228 |
| 229 | 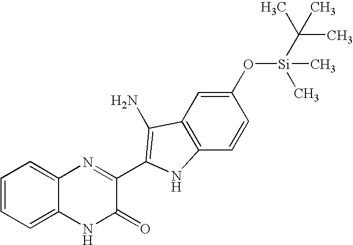 | 3.38 | 407.3 | 228, 2, 3 |
| 230 | 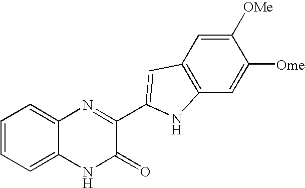 | 2.70 | 322.3 | 1 |
| 231 | 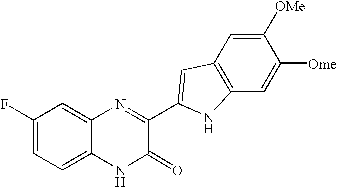 | 2.67 | 340.3 | 12 |
| 232 | 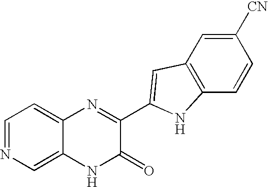 | 1.97 | 288.3 | 12 |
| 233 | 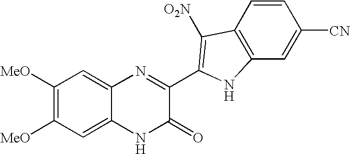 | 2.64 | No data | 12, 2 |
| 234 | 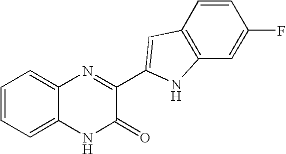 | 2.91 | 280.2 | 1 |
| 235 | 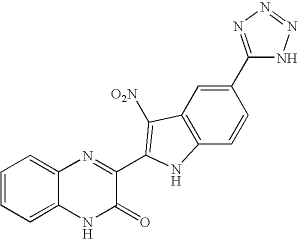 | 2.22 | 375.0 | 12, 2, 236 |
| 236 | 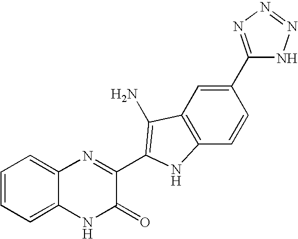 | 2.09 | 345.0 | 12, 2, 236 |
| 237 | 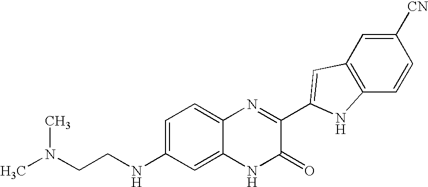 | 2.00 | 373.1 | 13 |
| 238 | 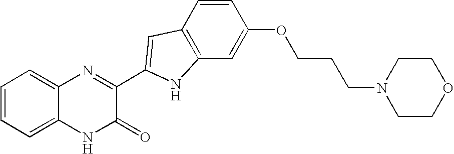 | 2.00 | 405.2 | 18 |
| 239 | 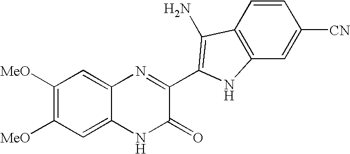 | 2.20 | 362.2 | 12, 2, 3 |
| 240 | 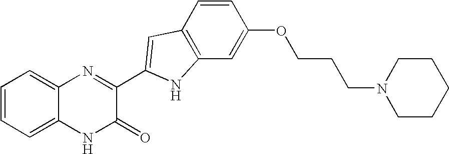 | 2.12 | 403.2 | 18 |
| 241 | 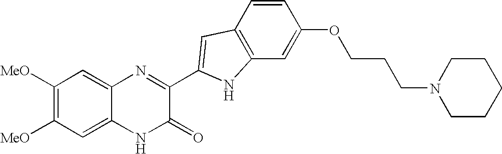 | 2.10 | 463.3 | 18 |
| 242 | 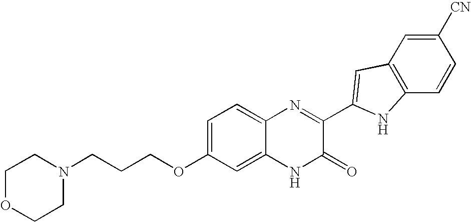 | 2.27 | 428.3 | 49 |
| 243 | 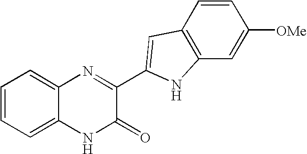 | 2.84 | 292.3 | 1 |
| 244 | 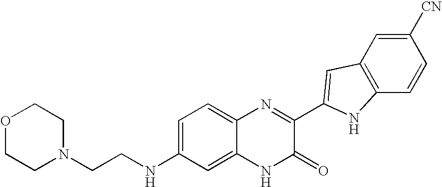 | 2.03 | 415.1 | 13, 49 |
| 245 | 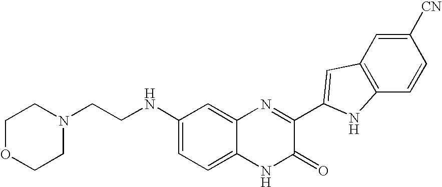 | 1.89 | 415.1 | 13, 49 |
| 246 | 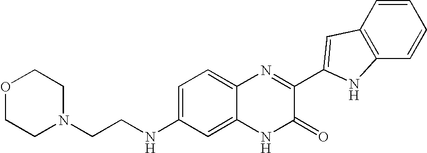 | 2.07 | 390.2 | 13, 49 |
| 247 | 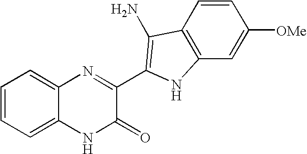 | 2.22 | 307.3 | 1, 2, 3 |
| 248 | 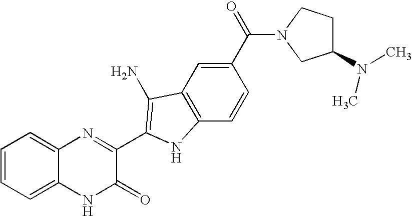 | 1.20 | 417.1 | 56 |
| 249 | 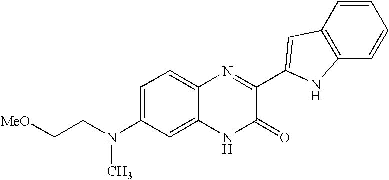 | 2.94 | 349.2 | 13 |
| 250 | 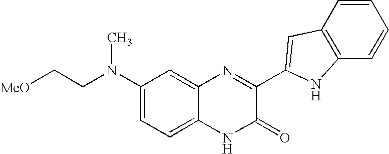 | 2.43 | 349.2 | 13 |
| 251 | 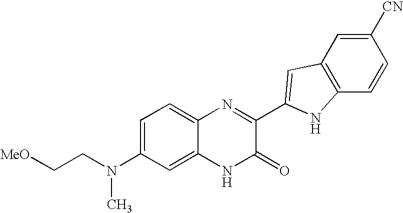 | 2.88 | 374.2 | 13 |
| 252 | 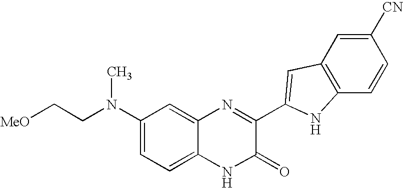 | 2.48 | 374.2 | 13 |
| 253 | 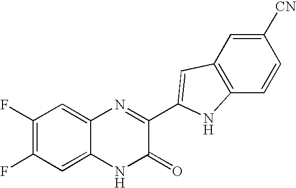 | 2.82 | 323.1 | 12 |
| 254 | 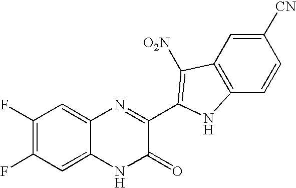 | 3.07 | 368.1 | 12, 2 |
| 255 | 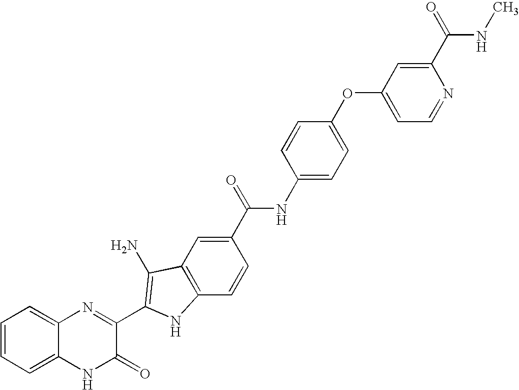 | 2.82 | 546.0 | 56 |
| 256 | 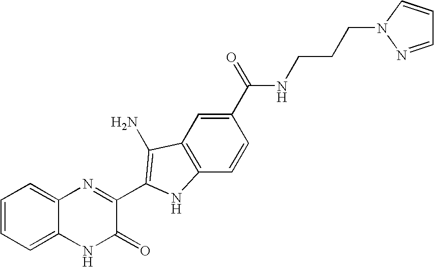 | 2.12 | 428.1 | 56 |
| 257 | 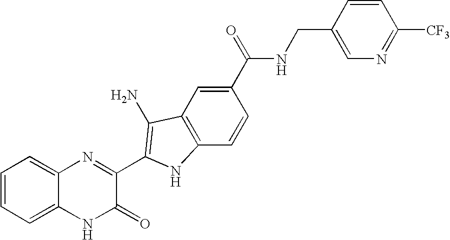 | 2.49 | 479.2 | 56 |
| 258 | 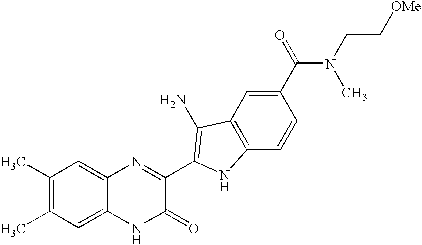 | 2.24 | 2.24 | 12, 2, 22. 56 |
| 259 | 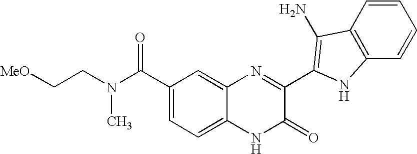 | 2.19 | 392.3 | 12, 2, 22, 56 |
| 260 | 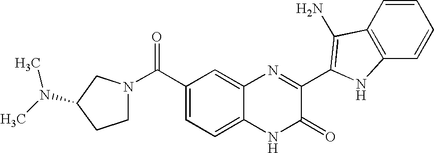 | 1.16 | 417.1 | 12, 2, 22, 56 |
| 261 | 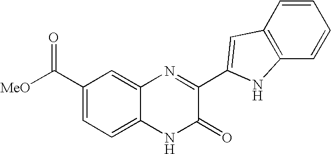 | 3.23 | 320.1 | 1 |
| 262 | 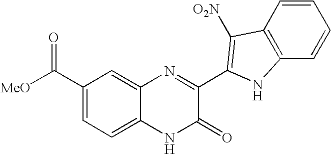 | 2.50 | 351.3 | 1, 2 |
| 263 | 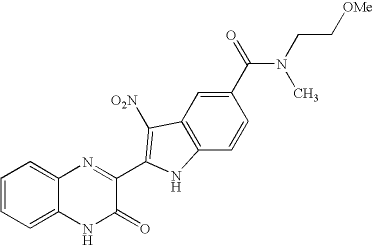 | 2.32 | 422.4 | 56 |
| 264 | 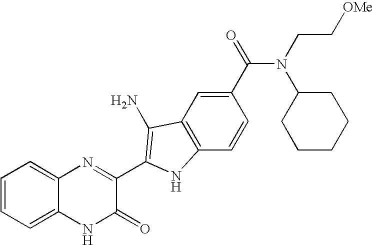 | 2.71 | 460.1 | 56 |
| 265 | 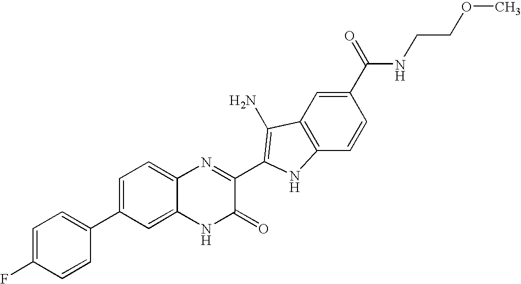 | 2.69 | 472.4 | 336, 2, 22, 56 |
| 266 | 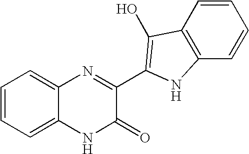 | 2.94 | 278.7 | 266 |
| 267 | 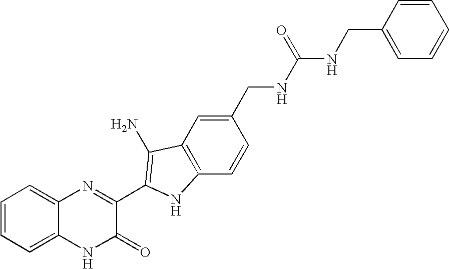 | | | 156, 214 |
| 268 | 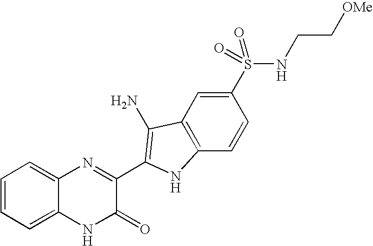 | | | 354 |
| 269 | 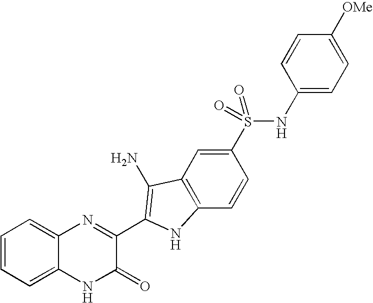 | | | 354 |
| 270 | 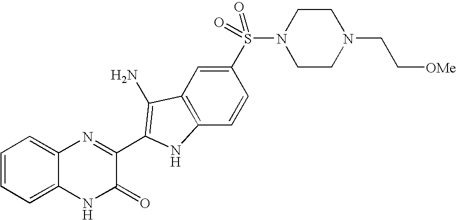 | | | 354 |
| 271 | 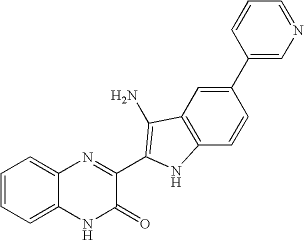 | | | 152, 1, 2, 3, |
| 272 | 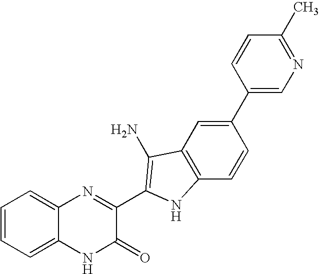 | | | 152, 1, 2, 3 |
| 273 | 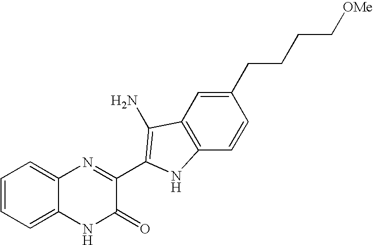 | | | 319, 2, 3 |
| 274 | 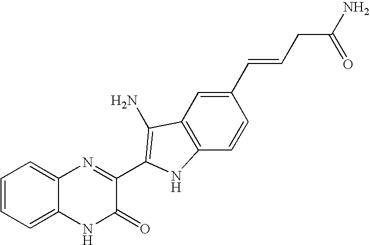 | | | 319, 2, 3 |
| 275 | 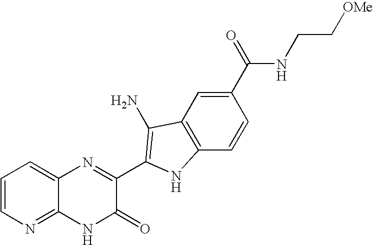 | | | 56 |
| 276 | 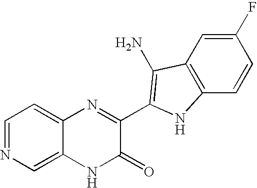 | | | 3 |
| 277 | 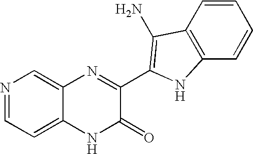 | | | 3 |
| 278 | 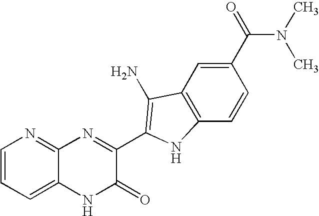 | | | 56 |
| 279 | 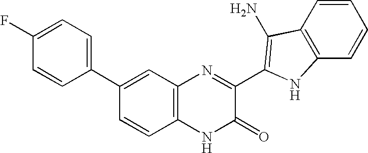 | | | 336, 2, 3 |
| 280 | 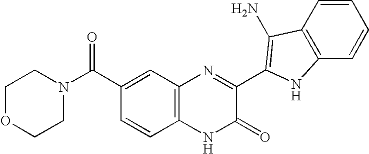 | | | 1, 2, 22, 3 |
| 281 | 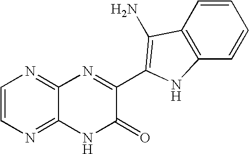 | | | 1, 2, 3 |
| 282 | 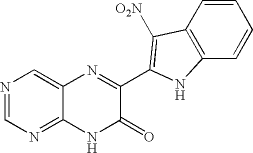 | | | 1, 2 |
| 283 | 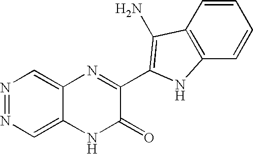 | | | 1, 2, 3 |
| 284 | 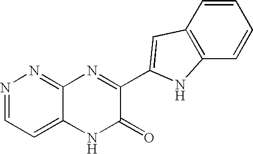 | | | 1 |
| 285 | 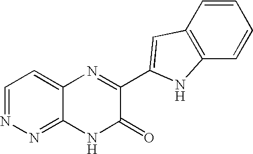 | | | 1 |
| 286 | 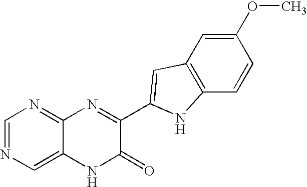 | | | 1 |
| 287 | 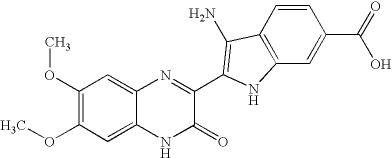 | | | 1, 2, 22, 3 |
| 288 | 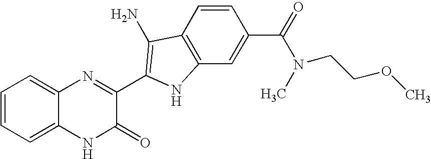 | | | 56 |
| 289 | 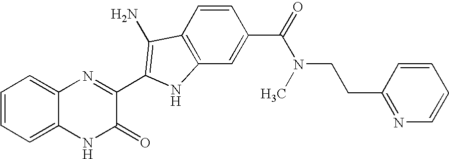 | | | 56 |
| 290 | 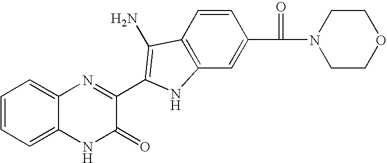 | | | 56 |
| 291 | 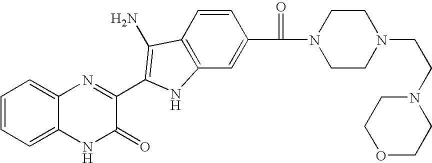 | | | 56 |
| 292 | 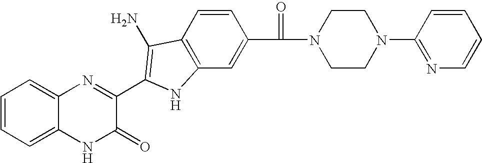 | | | 56 |
| 293 | 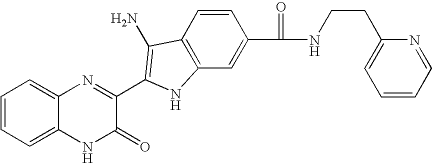 | | | 56 |
| 294 | 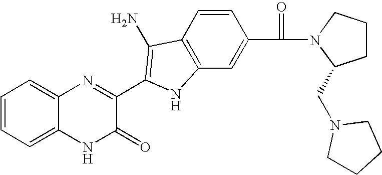 | | | 56 |
| 295 | 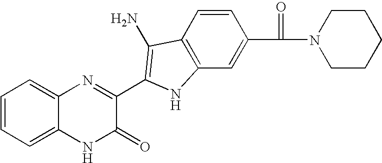 | | | 56 |
| 296 | 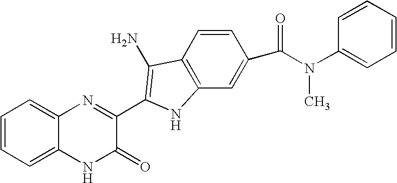 | | | 56 |
| 297 | 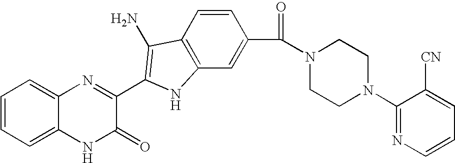 | | | 56 |
| 298 | 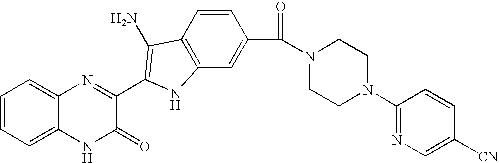 | | | 56 |
| 299 | 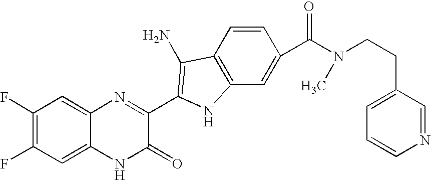 | | | 56 |
| 300 | 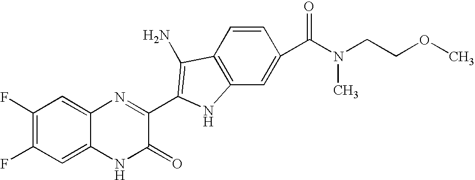 | | | 56 |
| 301 | 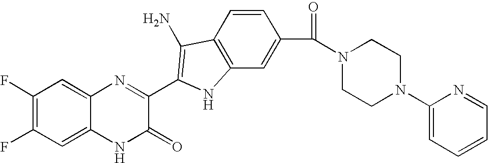 | | | 56 |
| 302 | 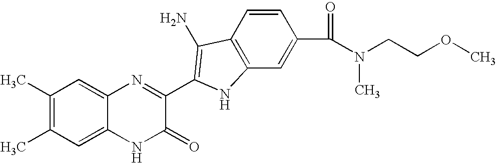 | | | 56 |
| 303 | 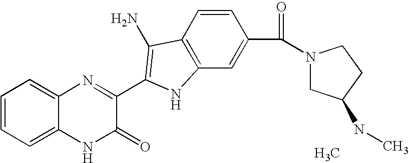 | | | 56 |
| 304 | 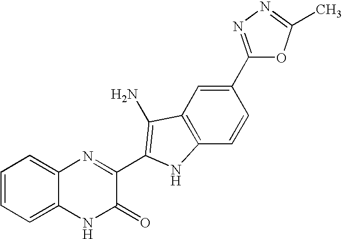 | 2.32 | 359.3 | 304 |
| 305 | 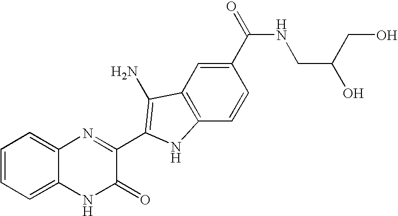 | 1.89 | 394.2 | 56 |
| 306 | 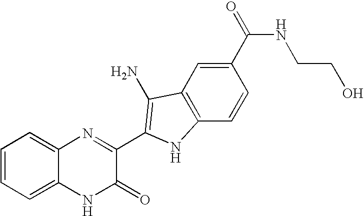 | 1.94 | 364.2 | 56 |
| 307 | 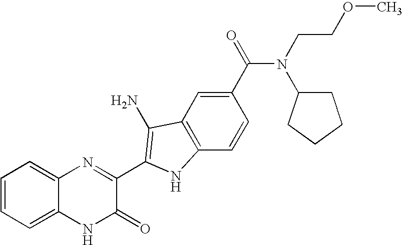 | 2.74 | 446.2 | 307 |
| 308 | 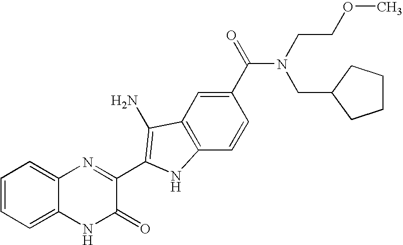 | 2.86 | 460.2 | 307 |
| 309 | 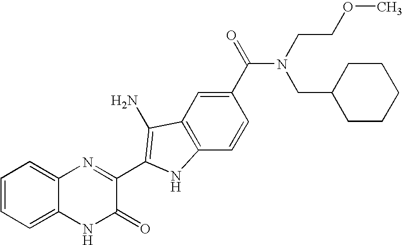 | 3.01 | 474.2 | 307 |
| 310 | 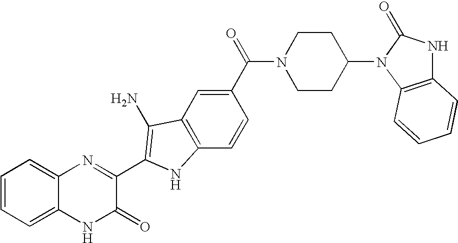 | 2.04 | 520.3 | 56 |
| 311 | 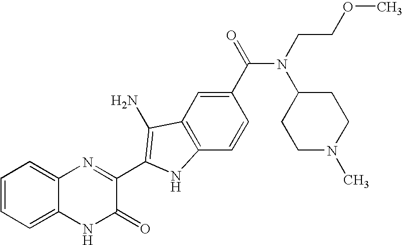 | 1.02 | 475.3 | 56 |
| 312 | 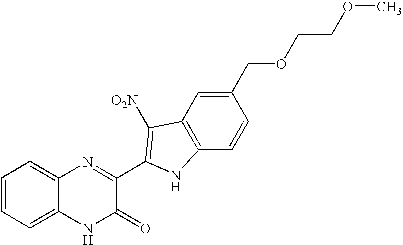 | 2.63 | 395.0 | 160 |
| 313 | 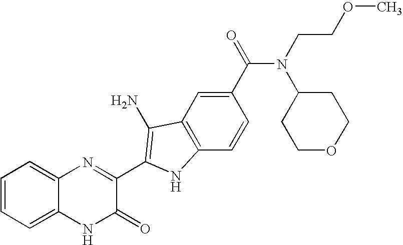 | 2.30 | 462.2 | 307 |
| 314 | 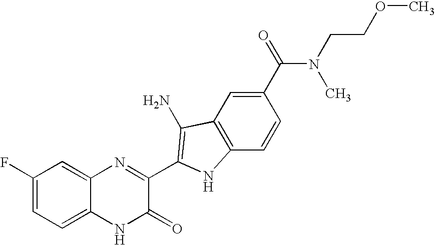 | 2.42 | 410.1 | 56 |
| 315 | 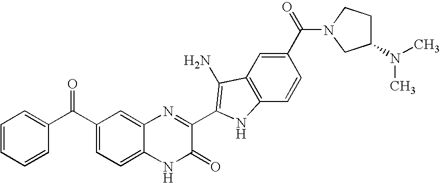 | 2.14 | 521.0 | 12, 2, 22, 56 |
| 316 | 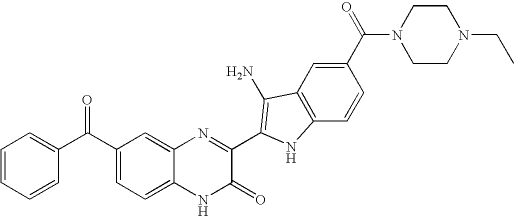 | 2.18 | 551.0 | 12, 2, 22, 56 |
| 317 | 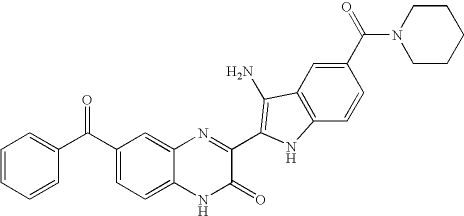 | 2.88 | 492.2 | 12, 2, 22, 56 |
| 318 | 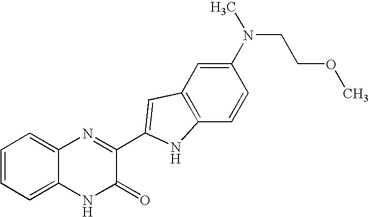 | 1.93 | 349.45 | 319 |
| 319 | 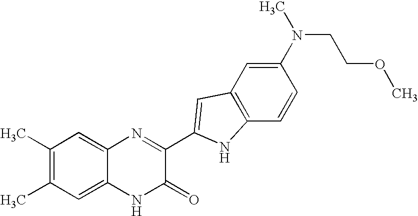 | 1.86 | 377.47 | 319 |
| 320 | 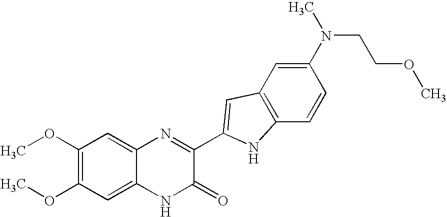 | 1.75 | 409.3 | 319 |
| 321 | 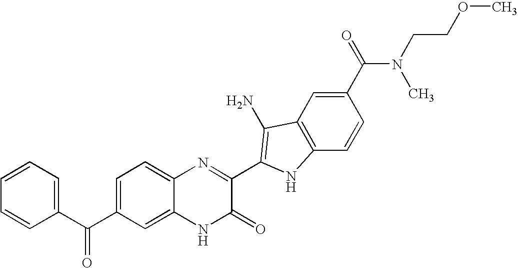 | 3.18 | 496.1 | 12, 2, 22, 56 |
| 322 | 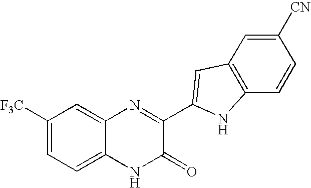 | 3.16 | 395.5 | 12 |
| 323 | 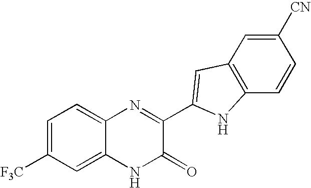 | 3.16 | 395.5 | 12 |
| 324 | 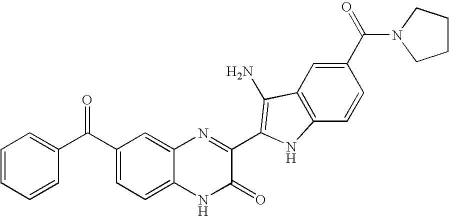 | 2.71 | 478.2 | 12, 2, 22, 56 |
| 325 | 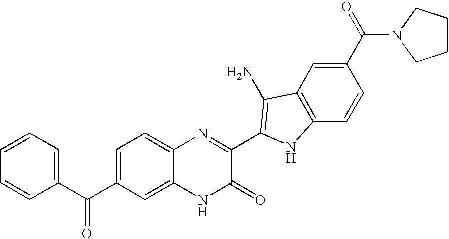 | 2.85 | 478.2 | 12, 2, 22, 56 |
| 326 | 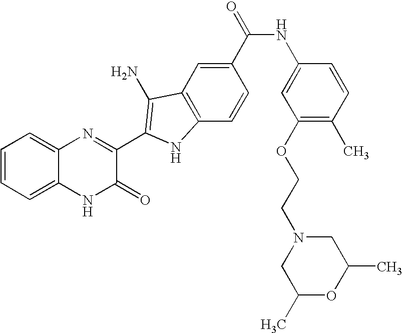 | 2.39 | 568.0 | 12, 2, 22, 21 |
| 327 | 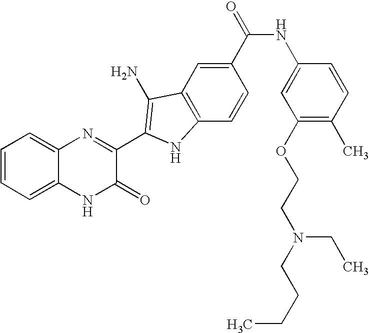 | 2.95 | 553.3 | 12, 2, 22, 21 |
| 328 | 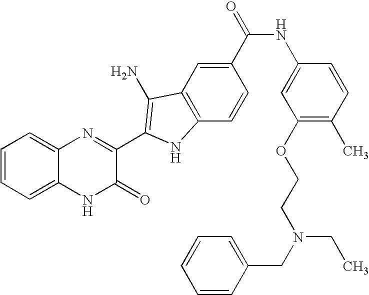 | 3.01 | 587.3 | 12, 2, 22, 21 |
| 329 | 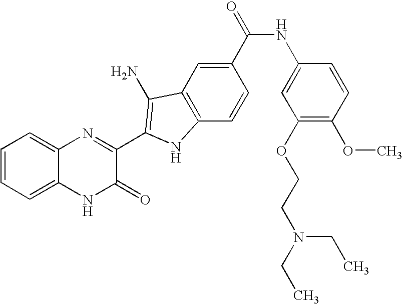 | 2.67 | 541.3 | 12, 2, 22, 21 |
| 330 | 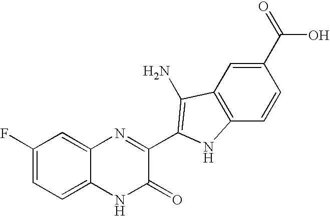 | 2.23 | 339.1 | 12, 2, 22, 3 |
| 331 | 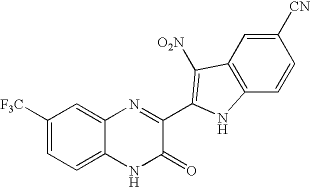 | 3.01 | 400.0 | 12, 2 |
| 332 | 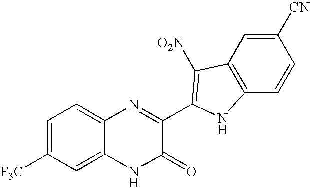 | 3.01 | 400.0 | 12, 2 |
| 333 | 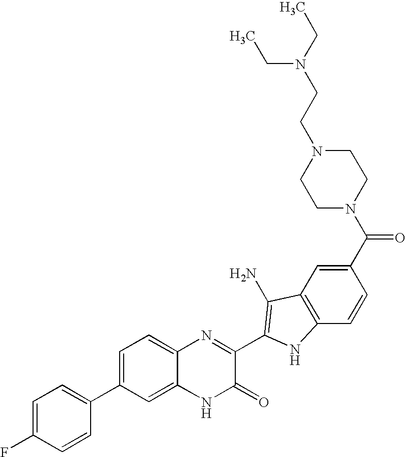 | 2.16 | 582.2 | 336, 2, 22, 56 |
| 334 | 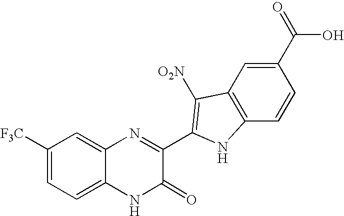 | 2.72 | 419.0 | 12, 2, 22 |
| 335 | 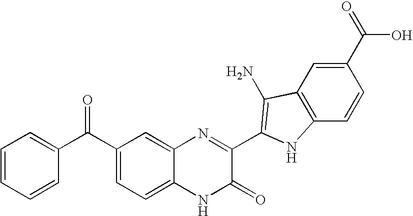 | 2.82 | 425.2 | 12, 2, 22, 3 |
| 336 | 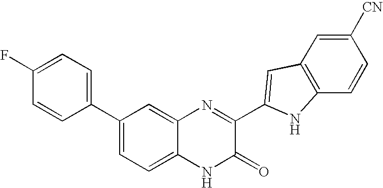 | 3.49 | 381.3 | 336 |
| 337 | 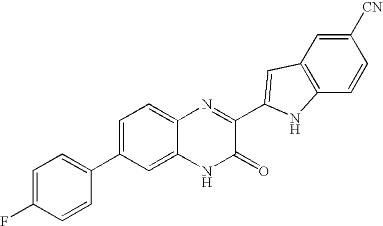 | 3.49 | 381.3 | 337 |
| 338 | 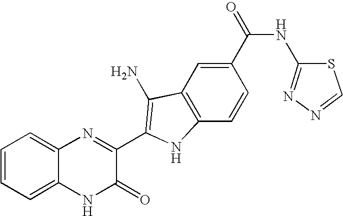 | 2.88 | 404.2 | 12, 2, 22, 21 |
| 339 | 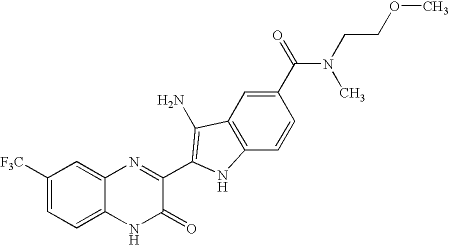 | 2.71 | 460.1 | 12, 2, 22, 56 |
| 340 | 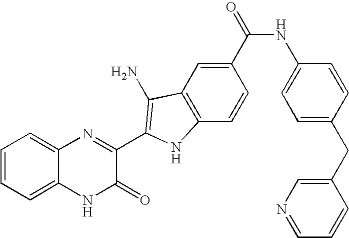 | 2.21 | 487.3 | 56 |
| 341 | 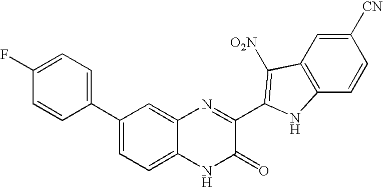 | 3.16 | 426.0 | 336, 2 |
| 342 | 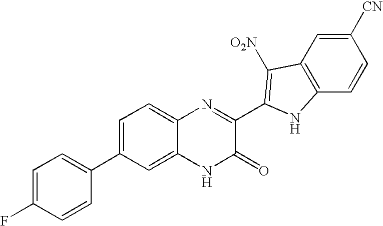 | 3.16 | 426.0 | 336, 2 |
| 343 | 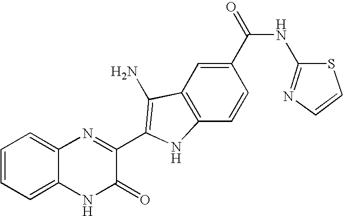 | 3.06 | 403.1 | 12, 2, 22, 21 |
| 344 | 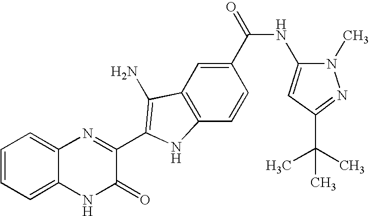 | 3.17 | 456.2 | 12, 2, 22, 21 |
| 345 | 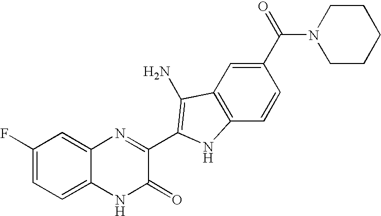 | 2.50 | 406.5 | 12, 2, 22, 56 |
| 346 | 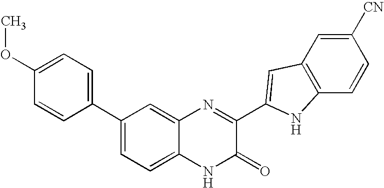 | 3.32 | 393.2 | 336 |
| 347 | 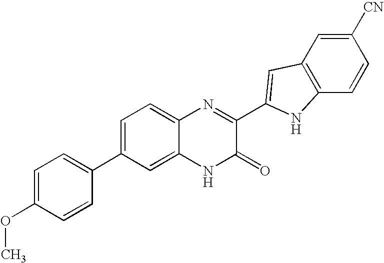 | 3.32 | 393.2 | 336 |
| 348 | 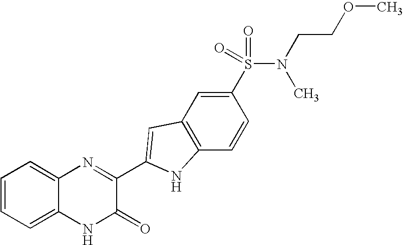 | | 413.6 | 354 |
| 349 | 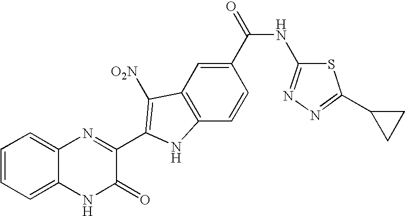 | 2.63 | 474.0 | 12, 2, 22, 21 |
| 350 | 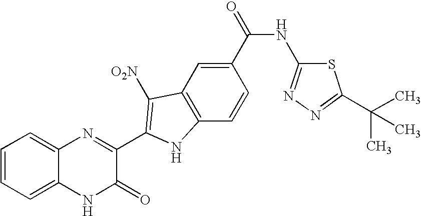 | 2.88 | 490.0 | 12, 2, 22, 21 |
| 351 | 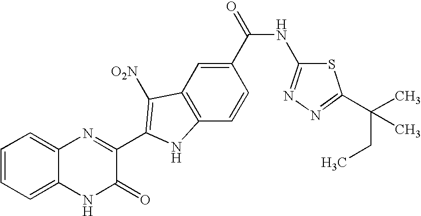 | 3.02 | 504.1 | 12, 2, 22, 21 |
| 352 | 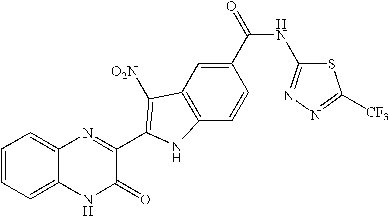 | 3.03 | 501.9 | 12, 2, 22, 21 |
| 353 | 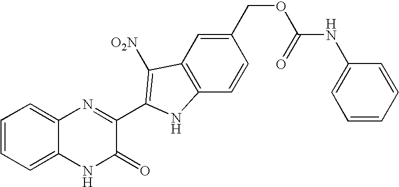 | 3.48 | 478.1 | 375 |
| 354 | 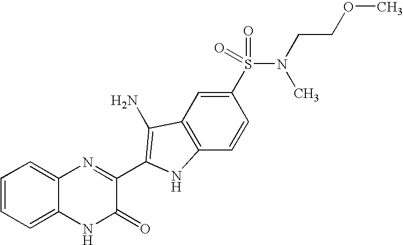 | 2.59 | 428.5 | 354 |
| 355 | 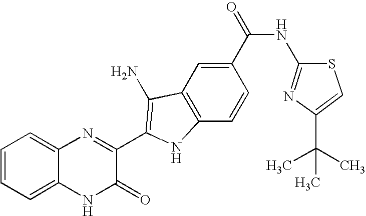 | 3.76 | 459.1 | 12, 2, 22, 21 |
| 356 | 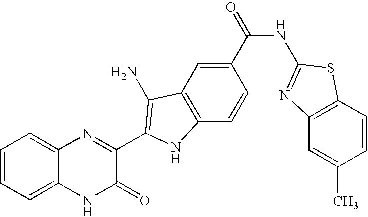 | 3.41 | 467.0 | 12, 2, 22, 21 |
| 357 | 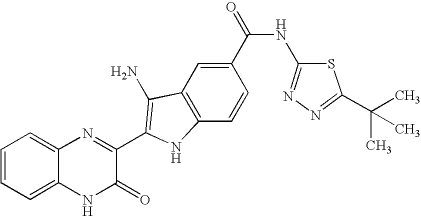 | 3.04 | 460.1 | 12, 2, 22, 21 |
| 358 | 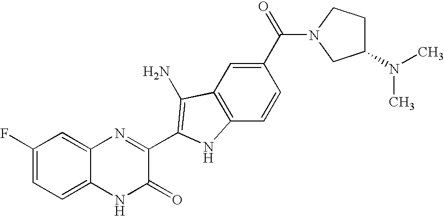 | 1.66 | 435.1 | 12, 2, 22, 56 |
| 359 | 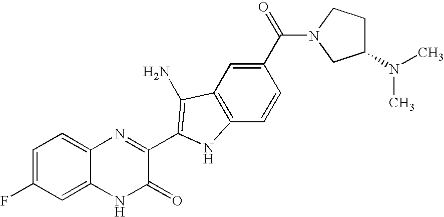 | 1.66 | 435.1 | 12, 2, 22, 56 |
| 360 | 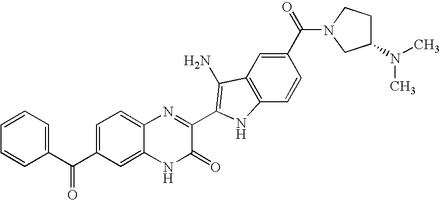 | 2.14 | 521.0 | 12, 2, 22, 56 |
| 361 | 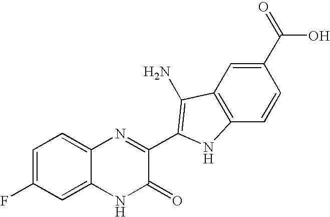 | 2.23 | 339.1 | 12, 2, 22 |
| 362 | 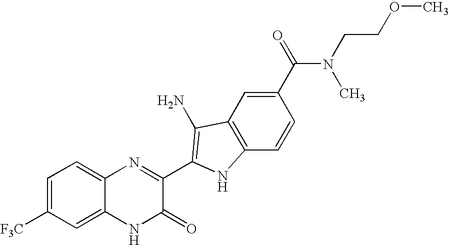 | 2.71 | 460.1 | 12, 2, 22, 56 |
| 363 | 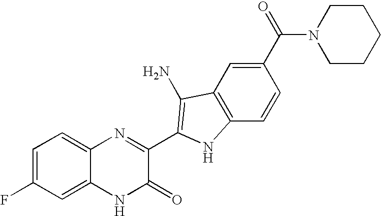 | 2.50 | 406.5 | 12, 2, 22, 56 |
| 364 | 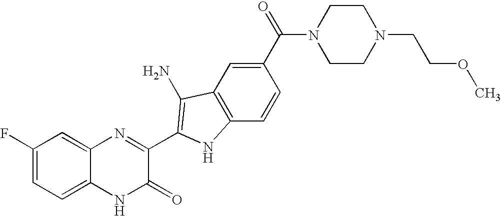 | 1.71 | 465.3 | 12, 2, 22, 56 |
| 365 | 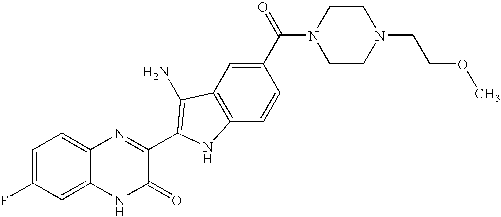 | 1.71 | 465.3 | 12, 2, 22, 56 |
| 366 | 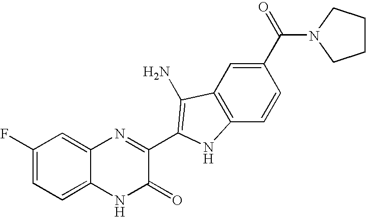 | 2.38 | 392.2 | 12, 2, 22, 56 |
| 367 | 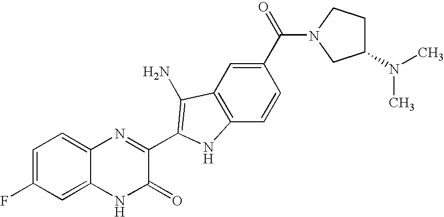 | 2.38 | 392.2 | 12, 2, 22, 56 |
| 368 | 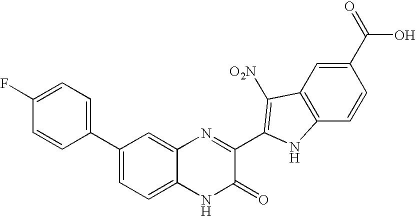 | 2.98 | 446.0 | 336, 2, 22 |
| 369 | 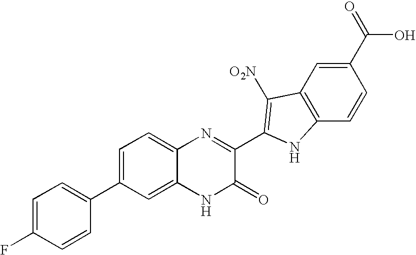 | 2.98 | 446.0 | 336, 2, 22 |
| 370 | 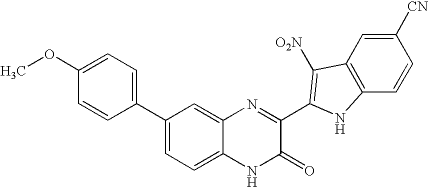 | 3.24 | 438.0 | 336, 2 |
| 371 | 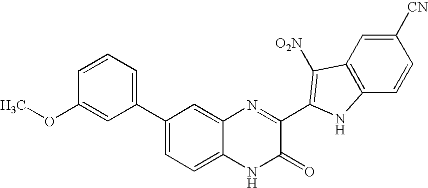 | 3.24 | 438.0 | 336, 2 |
| 372 | 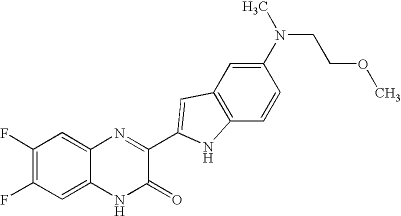 | 1.90 | 385.3 | 319 |
| 373 | 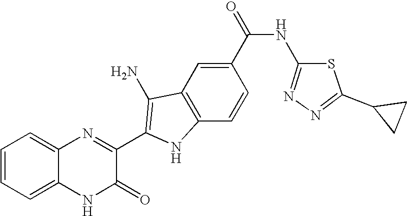 | 2.75 | 444.0 | 12, 2, 22, 21 |
| 374 | 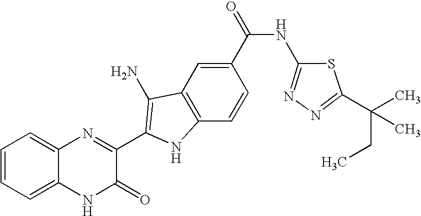 | 3.15 | 474.1 | 12, 2, 22, 21 |
| 375 | 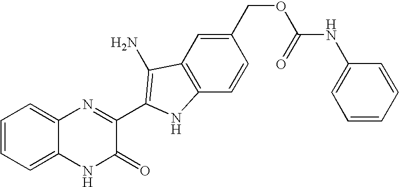 | 2.87 | 426.2 | 375 |
| 376 | 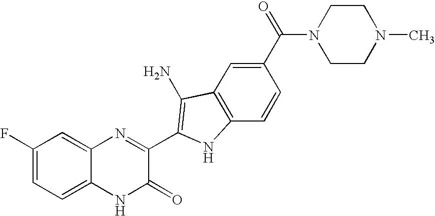 | 1.69 | 421.0 | 12, 2, 22, 56 |
| 377 | 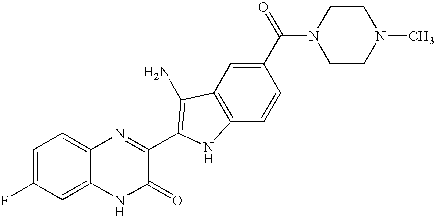 | 1.69 | 421.0 | 12, 2, 22, 56 |
| 378 | 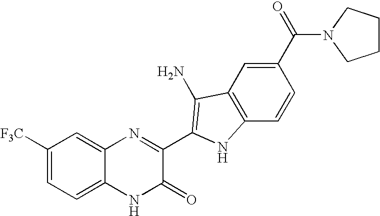 | 2.79 | 442.4 | 12, 2, 22, 56 |
| 379 | 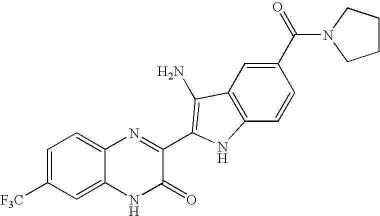 | 2.79 | 442.4 | 12, 2, 22, 56 |
| 380 | 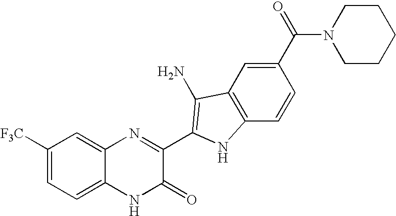 | 2.97 | 456.2 | 12, 2, 22, 56 |
| 381 | 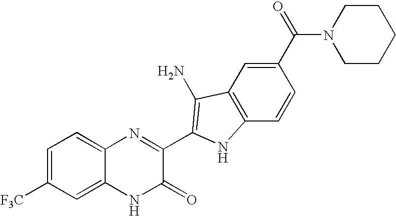 | 2.97 | 456.2 | 12, 2, 22, 56 |
| 382 | 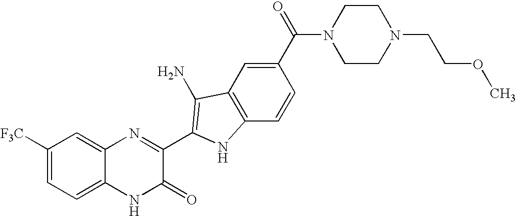 | 2.14 | 515.3 | 12, 2, 22, 56 |
| 383 | 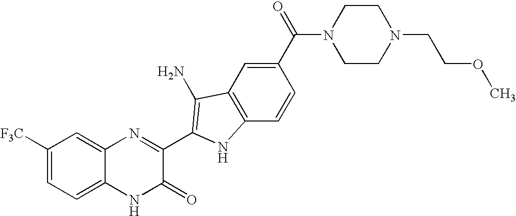 | 2.14 | 515.3 | 12, 2, 22, 56 |
| 384 | 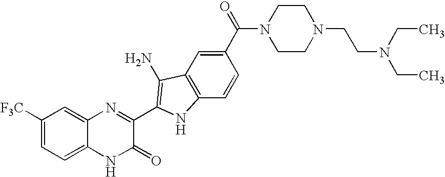 | 2.06 | 556.4 | 12, 2, 22, 56 |
| 385 | 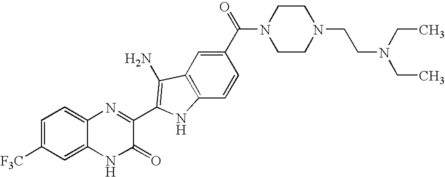 | 2.06 | 556.4 | 12, 2, 22, 56 |
| 386 | 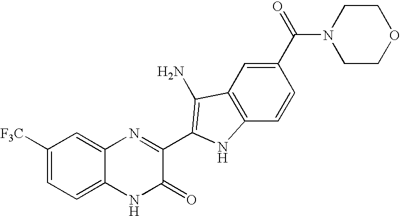 | 2.64 | 458.3 | 12, 2, 22, 56 |
| 387 | 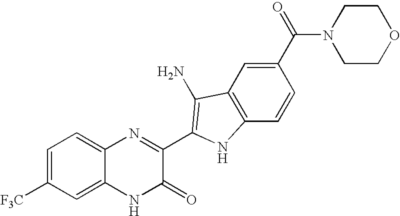 | 2.64 | 458.3 | 12, 2, 22, 56 |
| 388 | 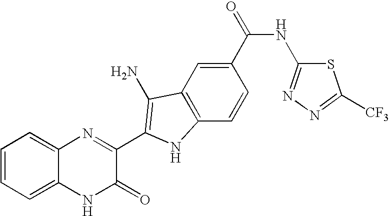 | 3.61 | 472.1 | 12, 2, 22, 21 |
| 389 | 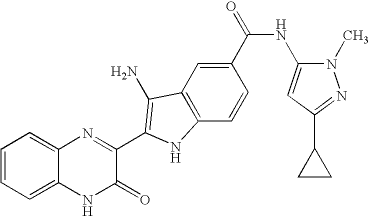 | 2.94 | 440.2 | 12, 2, 22, 21 |
| 390 | 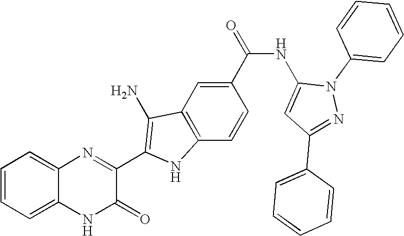 | 3.65 | 538.2 | 12, 2, 22, 21 |
| 391 | 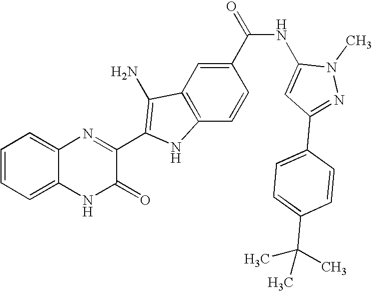 | 3.74 | 532.3 | 12, 2, 22, 21 |
| 392 | 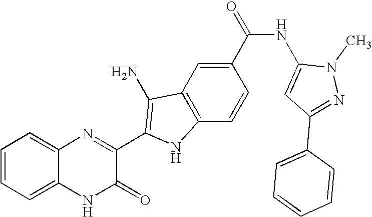 | 3.26 | 476.2 | 12, 2, 22, 21 |
| 393 | 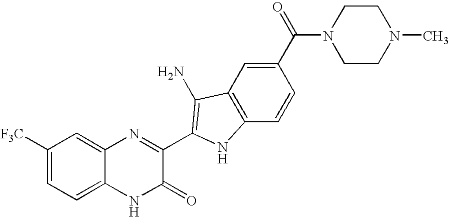 | 2.16 | 471.0 | 12, 2, 22, 56 |
| 394 | 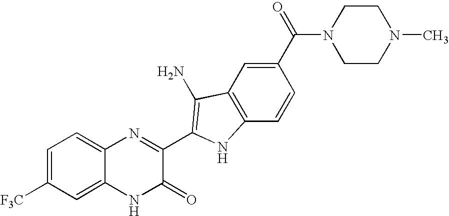 | 2.16 | 471.0 | 12, 2, 22, 56 |
| 395 | 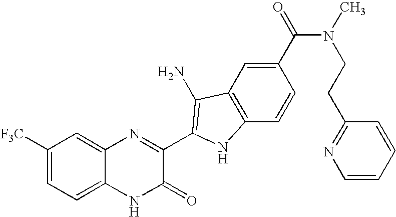 | 2.26 | 507.1 | 12, 2, 22, 56 |
| 396 | 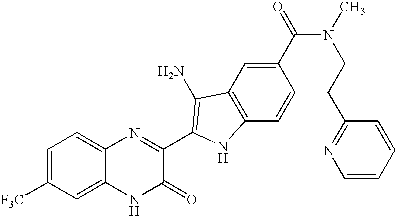 | 2.26 | 507.1 | 12, 2, 22, 56 |
| 397 | 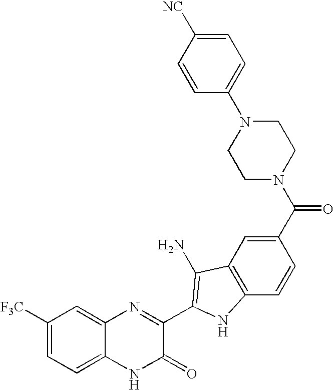 | 3.15 | 558.2 | 12, 2, 22, 56 |
| 398 | 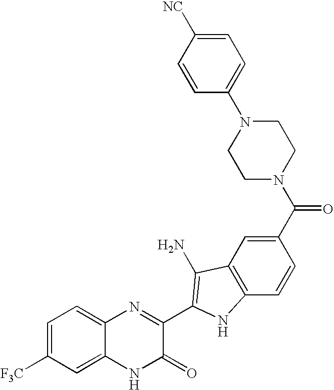 | 3.15 | 558.2 | 12, 2, 22, 56 |
| 399 | 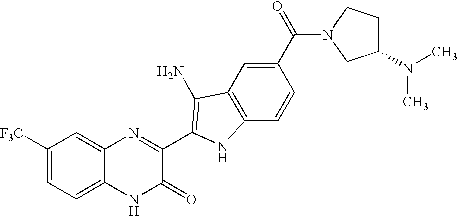 | 2.15 | 485.2 | 12, 2, 22, 56 |
| 400 | 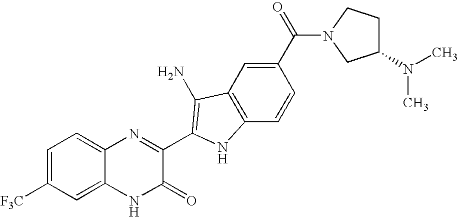 | 2.15 | 485.2 | 12, 2, 22, 56 |
| 401 | 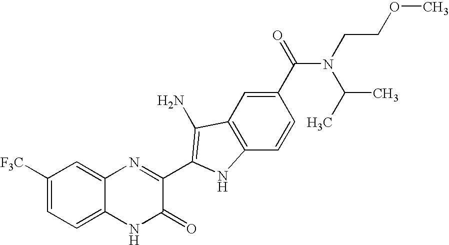 | 2.99 | 488.4 | 12, 2, 22, 56 |
| 402 | 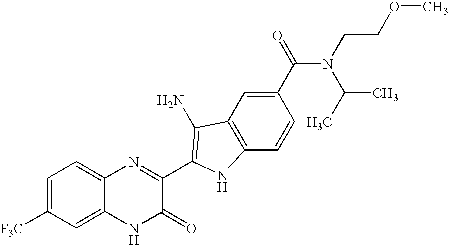 | 2.99 | 488.4 | 12, 2, 22, 56 |
| 403 | 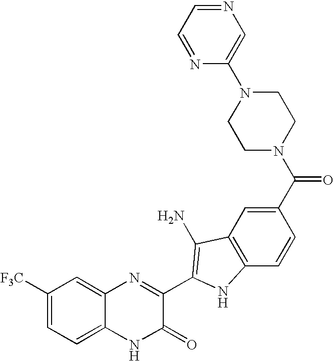 | 2.75 | 535.1 | 12, 2, 22, 56 |
| 404 | 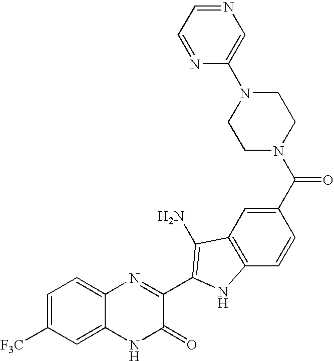 | 2.75 | 535.1 | 12, 2, 22, 56 |
| 405 | 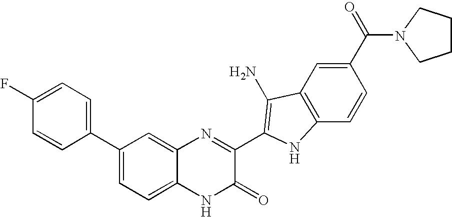 | 2.97 | 468.3 | 336, 2, 22, 56 |
| 406 | 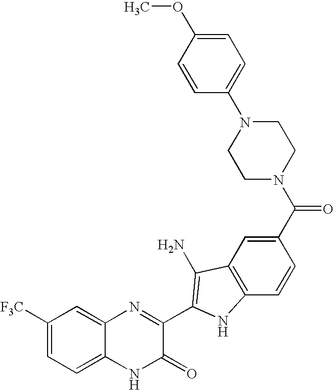 | 2.87 | 563.2 | 336, 2, 22, 56 |
| 407 | 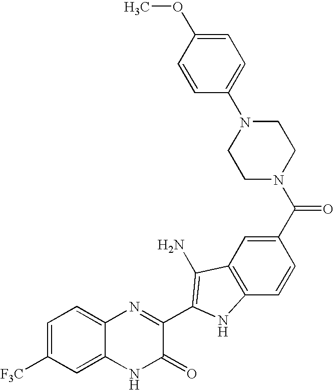 | 2.87 | 563.2 | 12, 2, 22, 56 |
| 408 | 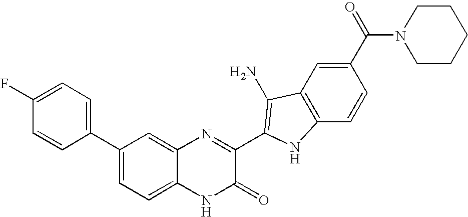 | 3.01 | 482.3 | 336, 2, 22, 56 |
| 409 | 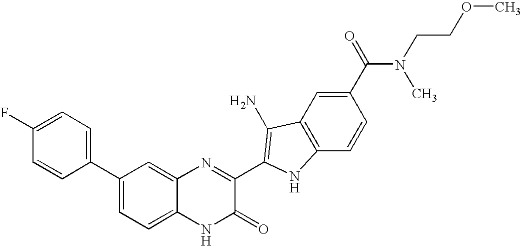 | 2.75 | 486.9 | 336, 2, 22, 56 |
| 410 | 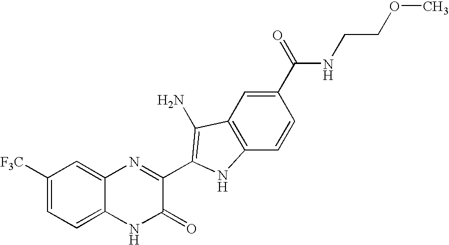 | 2.64 | 446.2 | 12, 2, 22, 56 |
| 411 | 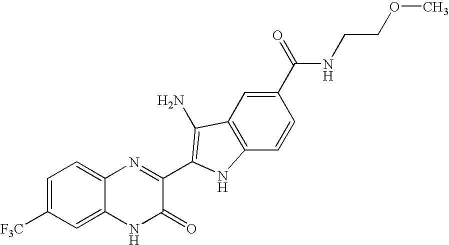 | 2.64 | 446.2 | 12, 2, 22, 56 |
| 412 | 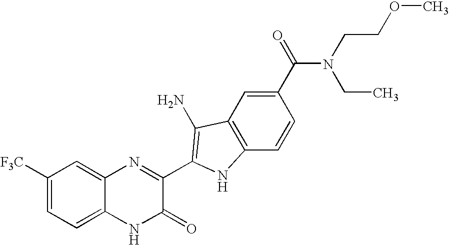 | 2.82 | 474.2 | 12, 2, 22, 56 |
| 413 | 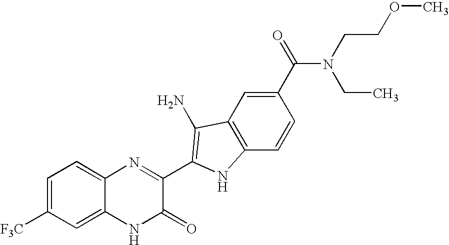 | 2.82 | 474.2 | 12, 2, 22, 56 |
| 414 | 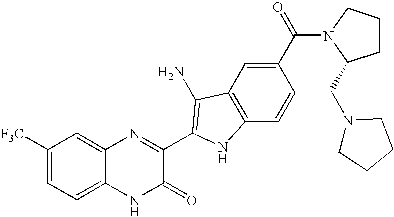 | 2.35 | 525.2 | 12, 2, 22, 56 |
| 415 | 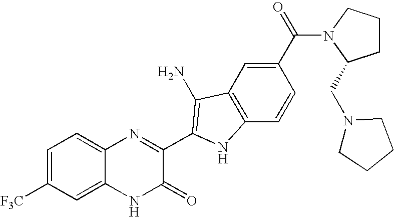 | 2.35 | 525.2 | 12, 2, 22, 56 |
| 416 | 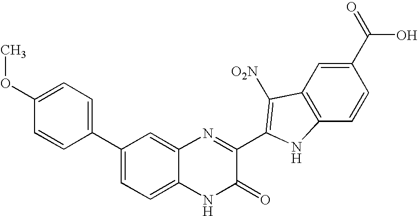 | 2.79 | 457.0 | 336, 2, 22 |
| 417 | 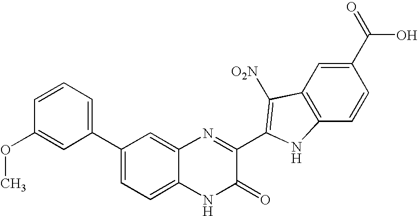 | 2.79 | 457.0 | 336, 2, 22 |
| 418 | 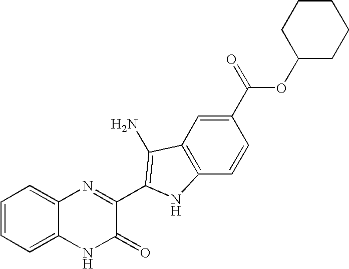 | 3.38 | 403.3 | 418 |
| 419 | 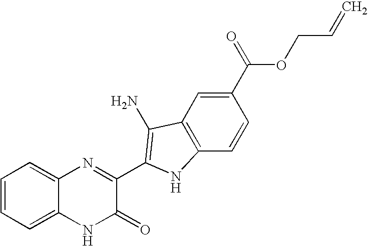 | 2.92 | 361.4 | 418 |
| 420 | 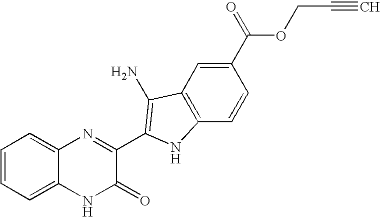 | 2.79 | 359.3 | 418 |
| 421 | 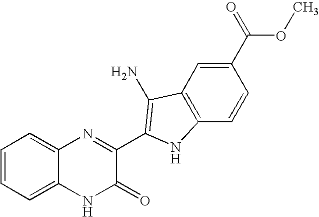 | 2.57 | 335.3 | 418 |
| 422 | 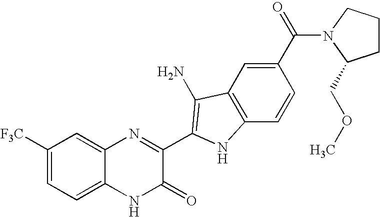 | 2.87 | 486.2 | 12, 2, 22, 56 |
| 423 | 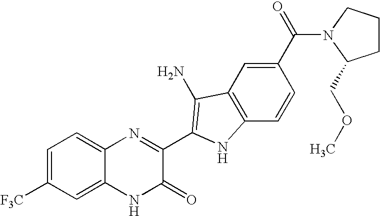 | 2.87 | 486.2 | 12, 2, 22, 56 |
| 424 | 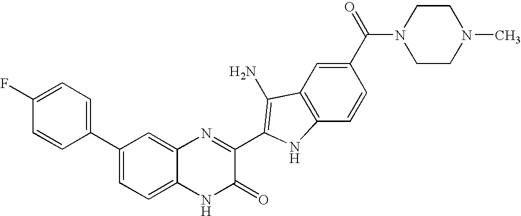 | 497.3 | 2.20 | 336, 2, 22, 56 |
| 425 | 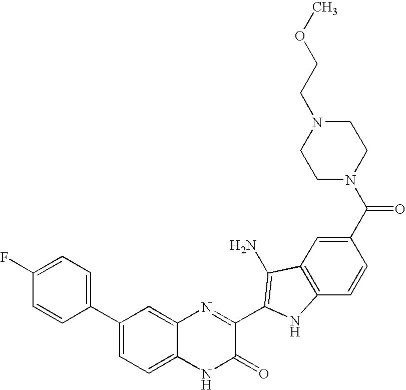 | 2.33 | 541.5 | 336, 2, 22, 56 |
| 426 | 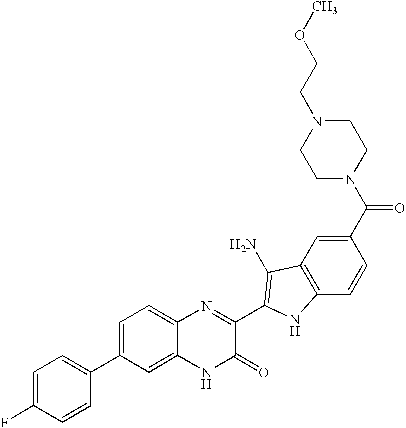 | 2.33 | 541.5 | 336, 2, 22, 56 |
| 427 | 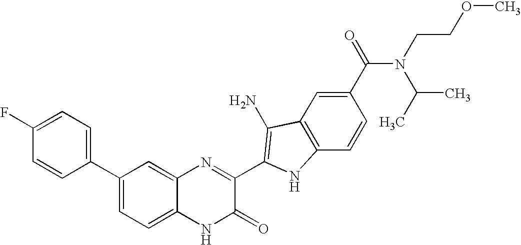 | 2.99 | 514.9 | 336, 2, 22, 56 |
| 428 | 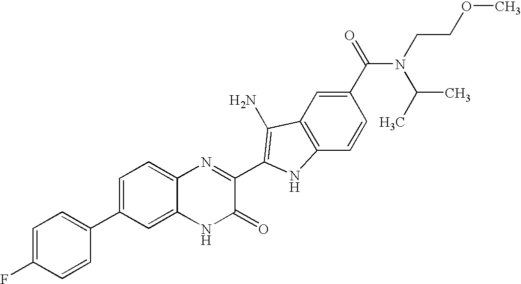 | 2.99 | 514.9 | 336, 2, 22, 56 |
| 429 | 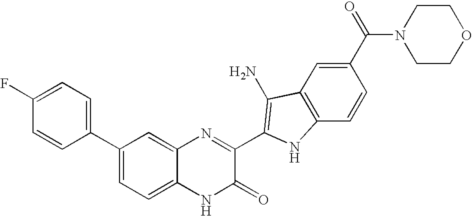 | 2.67 | 484.3 | 336, 2, 22, 56 |
| 430 | 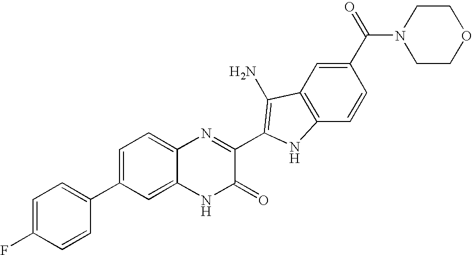 | 2.67 | 484.3 | 336, 2, 22, 56 |
| 431 | 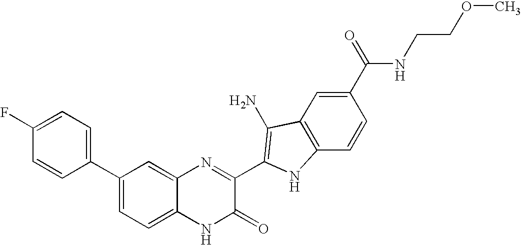 | 2.69 | 472.4 | 336, 2, 22, 56 |
| 432 | 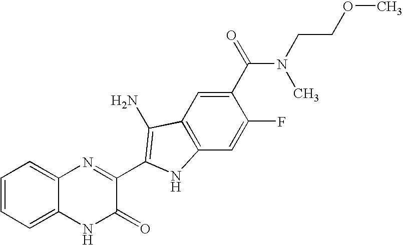 | | | 56 |
| 433 | 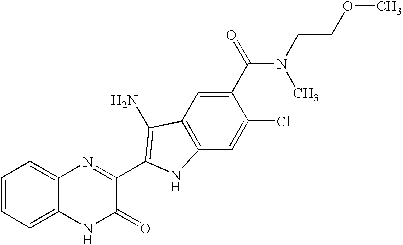 | | | 134 |
| 434 | 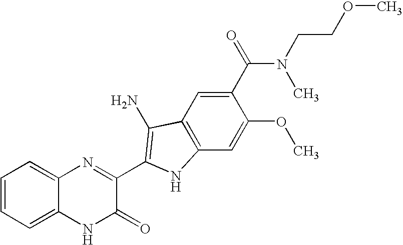 | | | 56 |
| 435 | 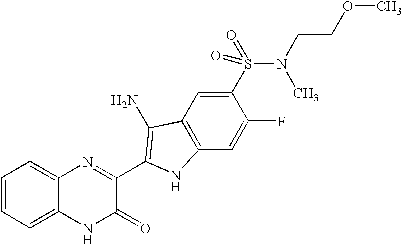 | | | 354 |
| 436 | 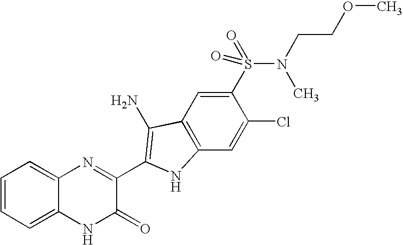 | | | 354, 134 |
| 437 | 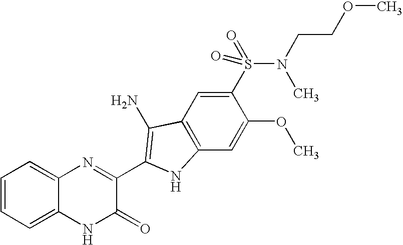 | | | 354 |
| 438 | 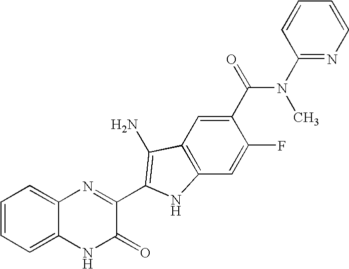 | | | 56 |
| 439 | 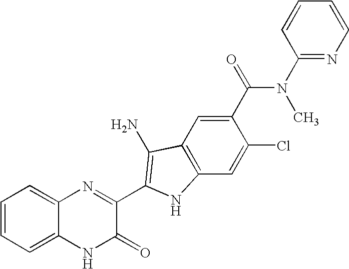 | | | 134 |
| 440 | 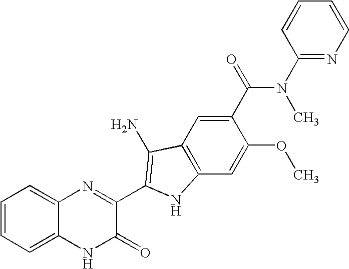 | | | 56 |
| 441 | 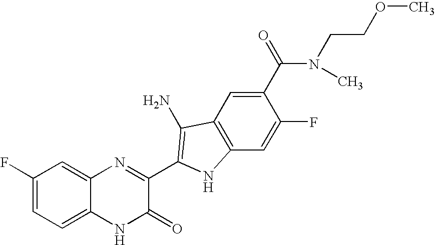 | | | 56 |
| 442 | 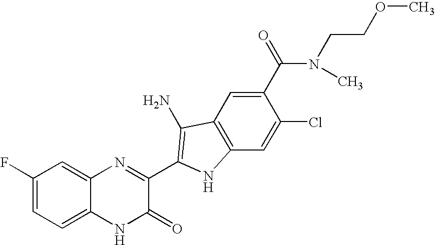 | | | 134 |
| 443 | 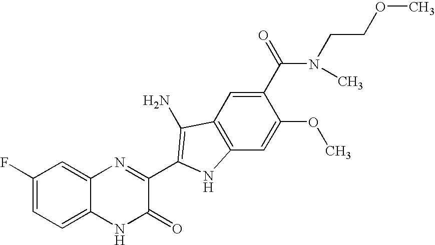 | | | 56 |
| 444 | 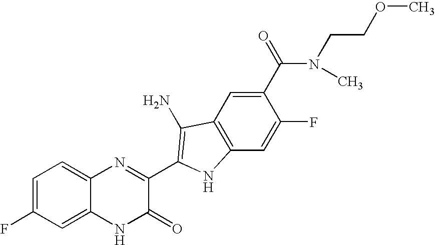 | | | 56 |
| 445 | 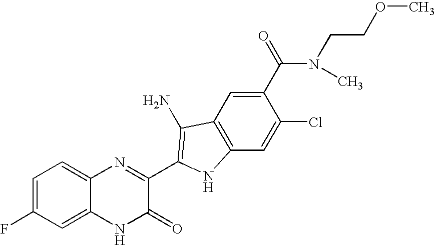 | | | 134 |
| 446 | 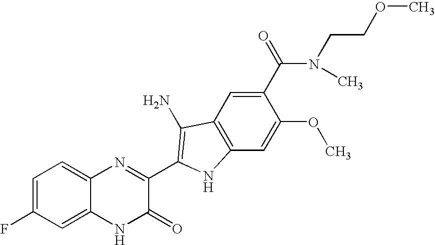 | | | 56 |
| 447 | 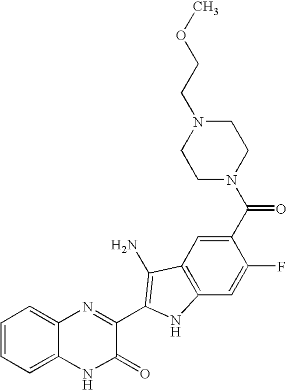 | | | 56 |
| 448 | 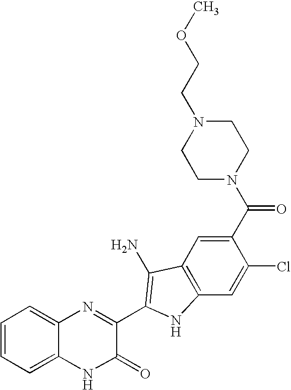 | | | 134 |
| 449 | 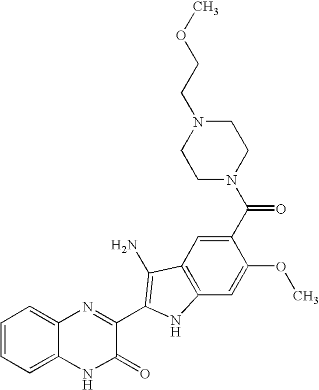 | | | 56 |
| 450 | 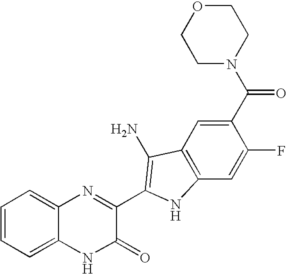 | | | 56 |
| 451 | 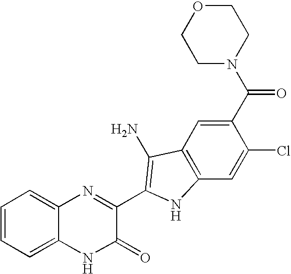 | | | 134 |
| 452 | 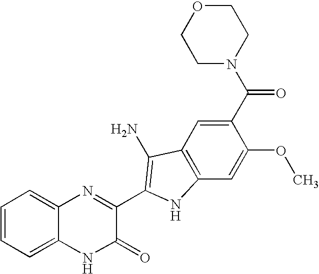 | | | 56 |
| 453 | 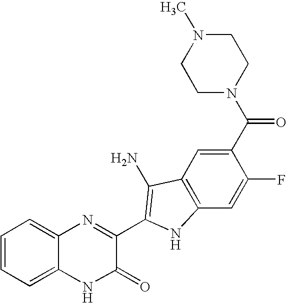 | | | 56 |
| 454 | 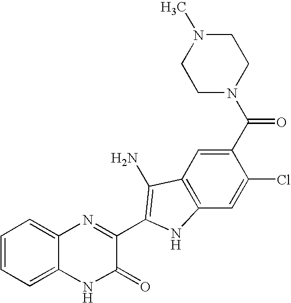 | | | 134 |
| 455 | 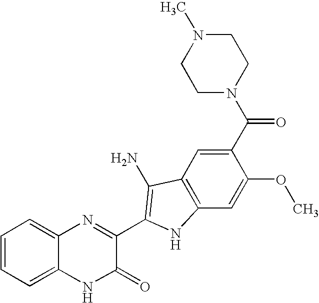 | | | 56 |
| 456 | 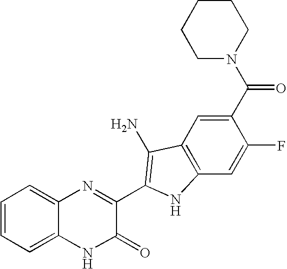 | | | 56 |
| 457 | 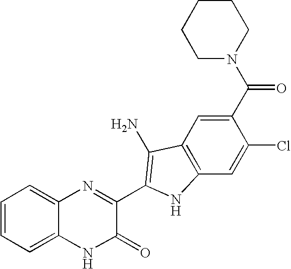 | | | 134 |
| 458 | 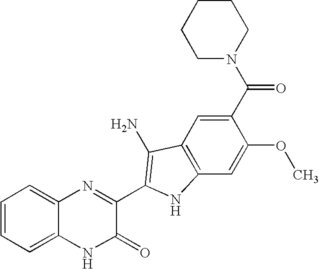 | | | 56 |
| 459 | 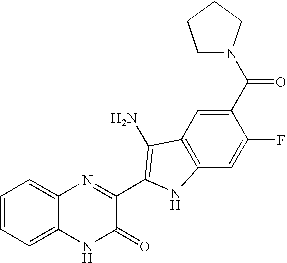 | | | 56 |
| 460 | 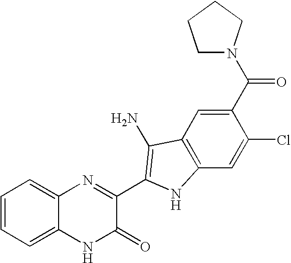 | | | 134 |
| 461 | 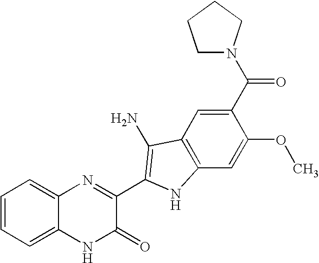 | | | 56 |
| 462 | 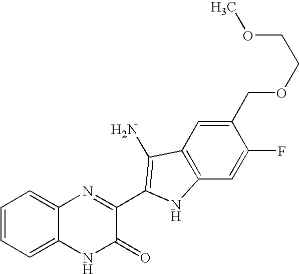 | | | 160 |
| 463 | 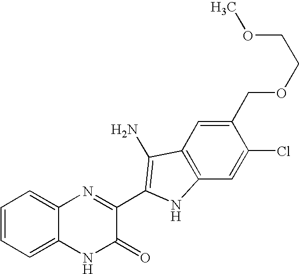 | | | 160, 134 |
| 464 | 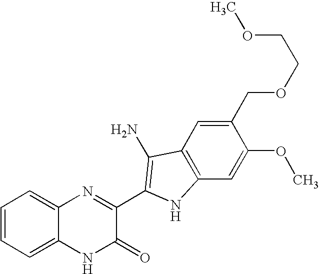 | | | 160 |
| 465 | 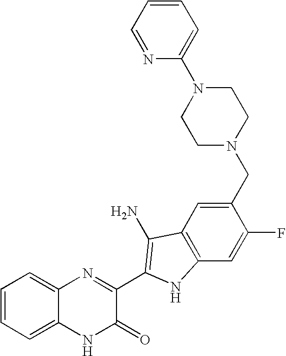 | | | 156 |
| 466 | 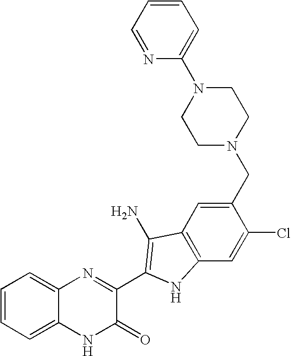 | | | 156, 134 |
| 467 | 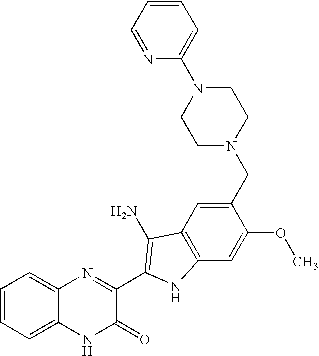 | | | 156 |
| 468 | 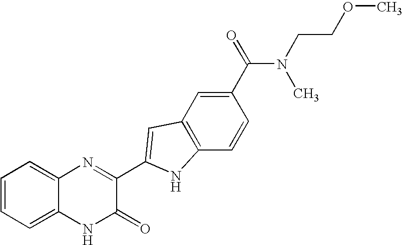 | | | 21 |
| 469 | 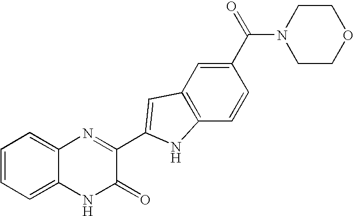 | | | 21 |
| 470 | 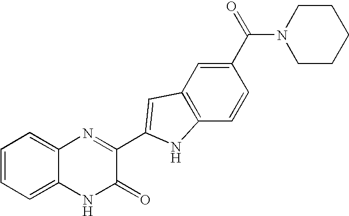 | | | 21 |
| 471 | 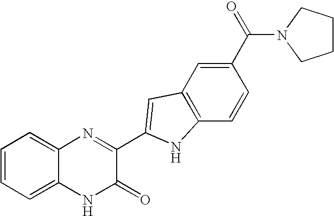 | | | 21 |
| 472 | 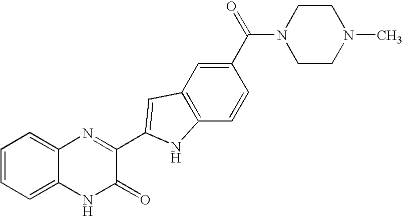 | | | 21 |
| 473 | 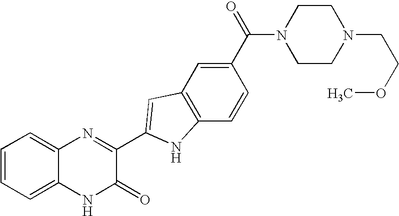 | | | 21 |
| 474 | 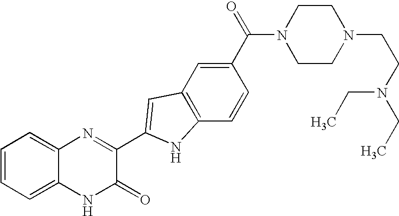 | | | 21 |
| 475 | 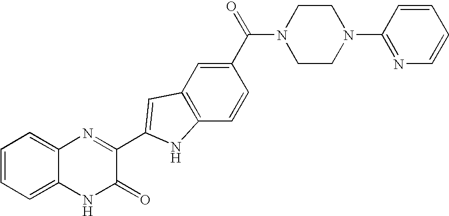 | | | 21 |
| 476 | 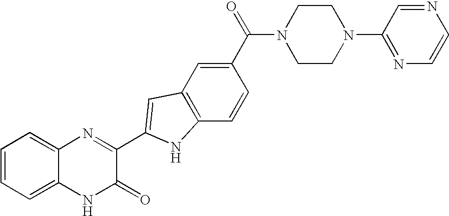 | | | 21 |
| 477 | 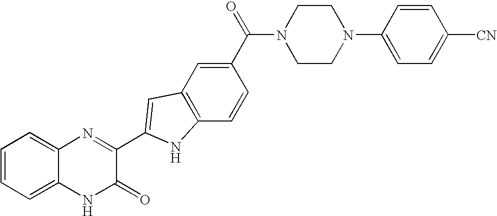 | | | 21 |
| 478 | 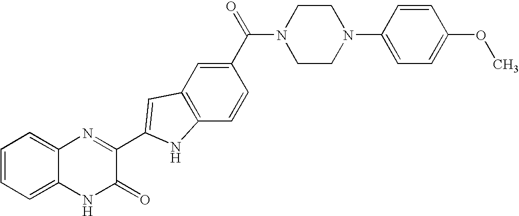 | | | 21 |
| 479 | 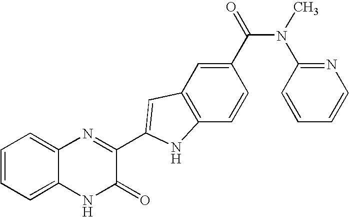 | | | 21 |
| 480 | 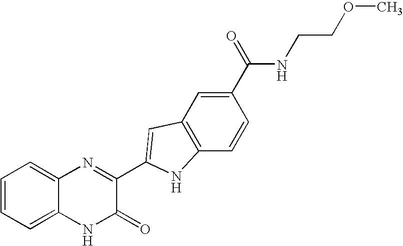 | | | 21 |
| 481 | 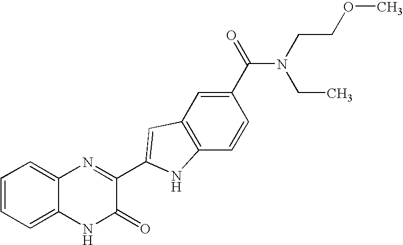 | | | 21 |
| 482 | 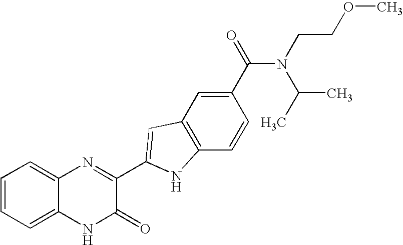 | | | 21 |
| 483 | 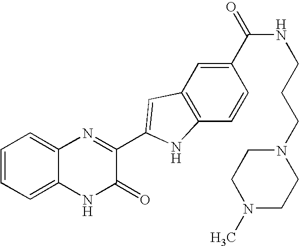 | | | 21 |
| 484 | 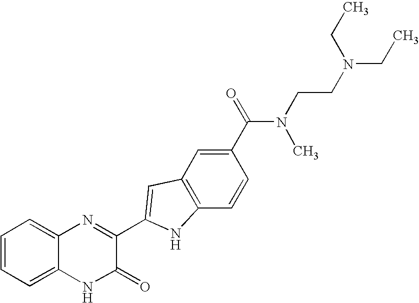 | | | 21 |
| 485 | 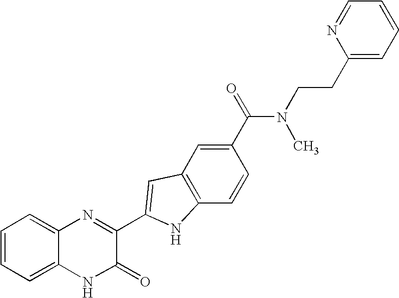 | | | 21 |
| 486 | 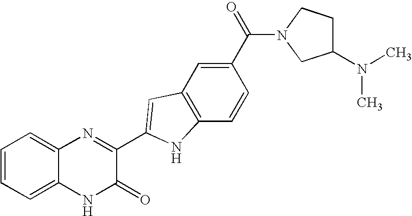 | | | 21 |
| 487 | 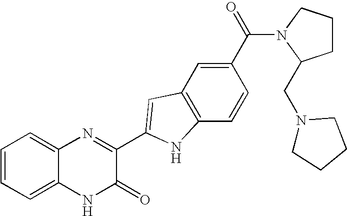 | | | 21 |
| 488 | 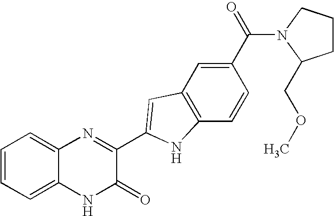 | | | 21 |
| 489 | 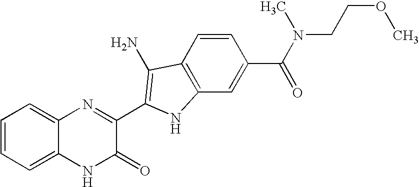 | | | 56 |
| 490 | 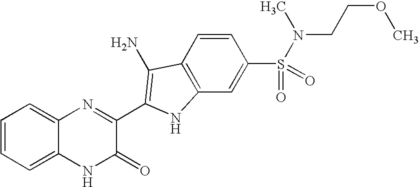 | | | 354 |
| 491 | 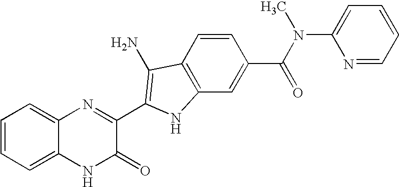 | | | 56 |
| 492 | 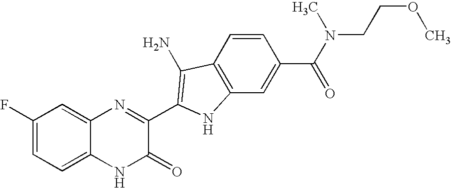 | | | 56 |
| 493 | 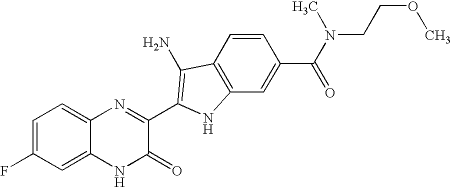 | | | 56 |
| 494 | 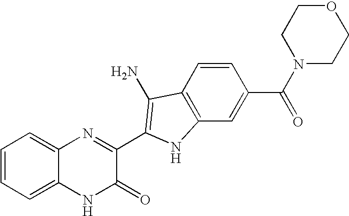 | | | 56 |
| 495 | 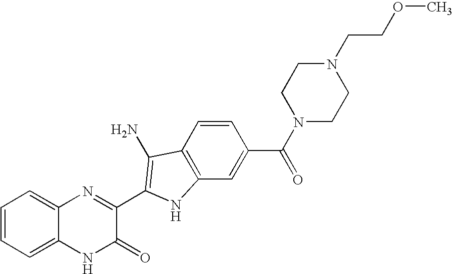 | | | 56 |
| 496 | 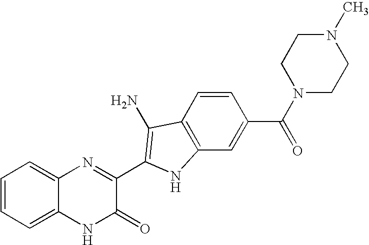 | | | 56 |
| 497 | 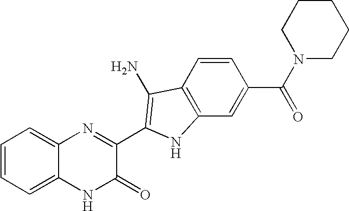 | | | 56 |
| 498 | 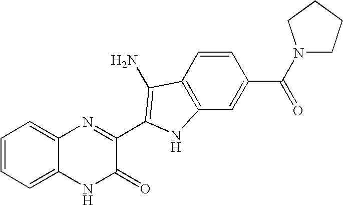 | | | 56 |
| 499 | 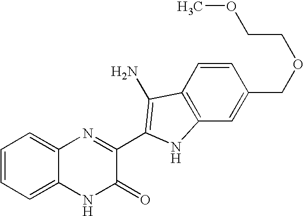 | | | 160 |
| 500 | 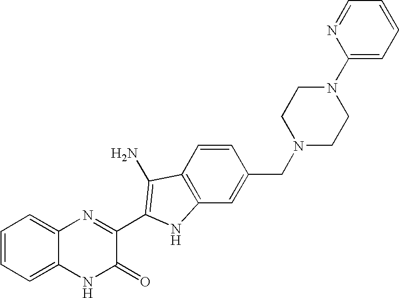 | | | 156 |
| 501 | 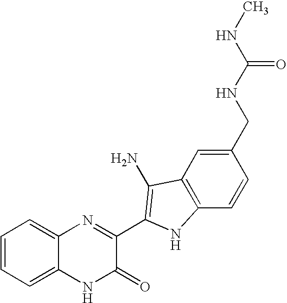 | | | 156, 214 |
| 502 | 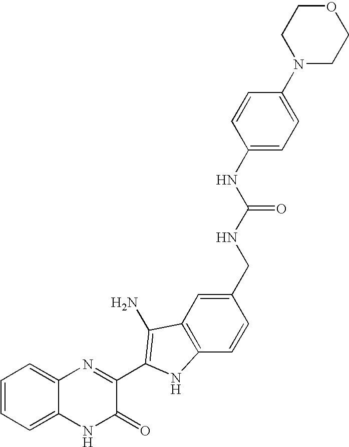 | | | 156, 214 |
| 503 | 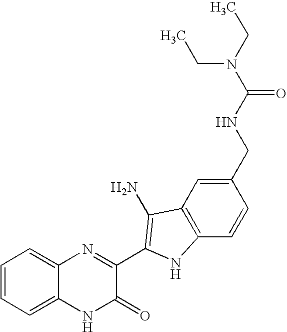 | | | 156, 214 |
| 504 | 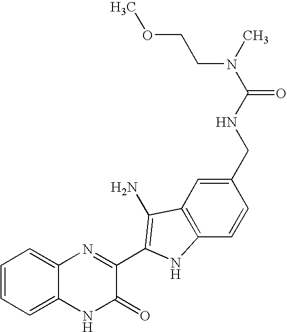 | | | 156, 214 |
| 505 | 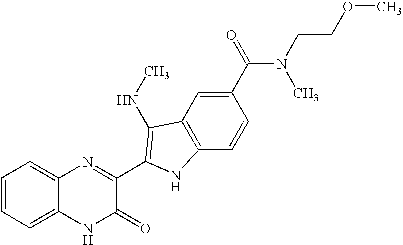 | | | 104 |
| 506 | 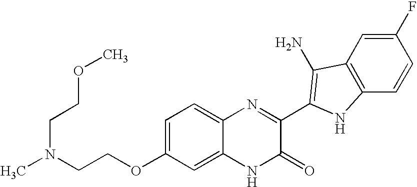 | | | 49, 2, 3 |
| 507 | 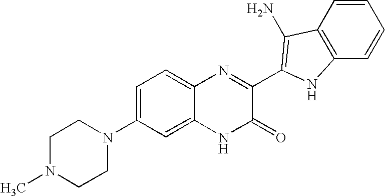 | | | 13, 2, 3 |
| 508 | 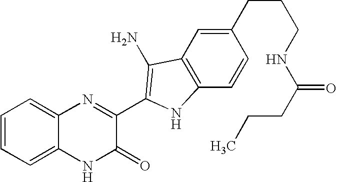 | | | 319, 2, 151, 3 |
| 509 | 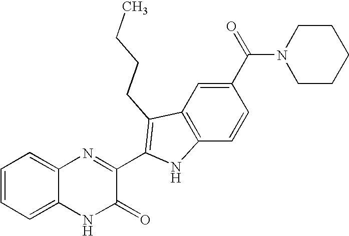 | | | 21 |
| 510 | 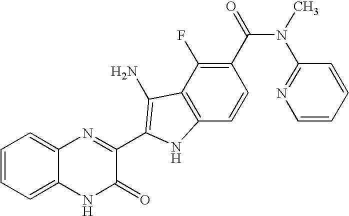 | | | 56 |
| 511 | 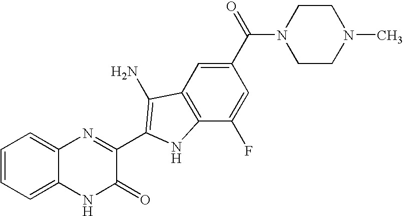 | | | 56 |
| 512 | 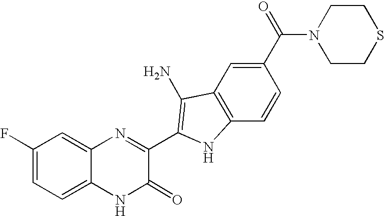 | | | 56 |
| 513 | 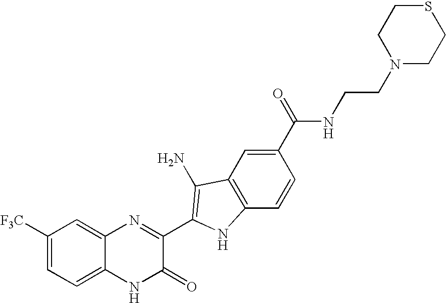 | | | 56 |

*Preparative methods: the numbers in this column indicate the order in which the processes analogous to the numbered specific examples (described below) would be followed, to make the specific compound identified in the row.

Asymmetry, i.e., where a compound's mirror image cannot be super-imposed on the compound, may be present in a compound of Formula (I) due to the inherent structure of the molecule. Examples of such asymmetric molecules include certain allenyl compounds. The compounds of this invention may also contain one or more asymmetric centers depending upon the location and nature of the various substituents selected. A molecule with a single asymmetric center may be a mixture of enantiomers (R,S), or may be a single (R) or (S) enantiomer. A molecule with more than one asymmetric center may be a mixture of diastereomers, or may be a single diastereomer. Additionally, a compound may exhibit asymmetry due to restricted rotation about a given bond, for example, the central bond adjoining two substituted aromatic rings of the specified compound. It is intended that all such configurations and conformations (including enantiomers, diastereomers, and other optical isomers) are included within the scope of the present invention. Separated, pure or partially purified stereo isomers of the compounds of Formula (I) are each included within the scope of the present invention. Preferred compounds are those with the absolute configuration or conformation which produces the more desirable biological activity.

The use of pharmaceutically acceptable salts of the compounds of this invention are also within the scope of this invention. The term “pharmaceutically acceptable salt” refers to either inorganic or organic salts of a compound of the present invention that have properties acceptable for the therapeutic use intended. For example, see S. M. Berge, et al. “Pharmaceutical Salts,” J. Pharm. Sci. 1977, 66, 1-19.

Representative salts of the compounds of this invention include the conventional non-toxic salts and the quaternary ammonium salts that are formed, for example, from inorganic or organic acids or bases by means well known in the art. For example, such acid addition salts include acetate, adipate, alginate, ascorbate, aspartate, benzoate, benzenesulfonate, bisulfate, butyrate, citrate, camphorate, camphorsulfonate, cinnamate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, itaconate, lactate, maleate, mandelate, methanesulfonate, 2-naphthalenesulfonate, nicotinate, nitrate, oxalate, pamoate, pectinate, persulfate, 3-phenylpropionate, picrate, pivalate, propionate, succinate, sulfonate, tartrate, thiocyanate, tosylate, and undecanoate. The term acid addition salts also comprises the hydrates and the solvent addition forms which the compounds of this invention are able to form. Examples of such forms are, for example, hydrates, alcoholates and the like.

Base salts include alkali metal salts such as potassium and sodium salts, alkaline earth metal salts such as calcium and magnesium salts, and ammonium salts with organic bases such as dicyclohexylamine and N-methyl-D-glucamine. Additionally, basic nitrogen containing groups may be quaternized with such agents as lower alkyl halides such as methyl, ethyl, propyl, and butyl chlorides, bromides and iodides; dialkyl sulfates including dimethyl, diethyl, and dibutyl sulfate; and diamyl sulfates, long chain halides such as decyl, lauryl, myristyl and strearyl chlorides, bromides and iodides, aralkyl halides including benzyl and phenethyl bromides, and others.

The esters of appropriate compounds of this invention are pharmaceutically acceptable esters such as alkyl esters, including methyl, ethyl, propyl, isopropyl, butyl, isobutyl or pentyl esters, and the like. Additional esters such as phenyl-(C₁-C₅) alkyl may be used, although methyl ester is preferred.

Unless the context clearly indicates to the contrary, whenever the term “compounds of this invention,” “compounds of the present invention”, and the like, are used herein, they are intended to include the chemically feasible pharmaceutically acceptable salts and/or esters as well as all stereoisomeric forms of the referenced compounds.

### METHOD OF THE MAKING THE COMPOUNDS OF THE PRESENT INVENTION

In general, the compounds used in this invention may be prepared by standard techniques known in the art, by known processes analogous thereto, and/or by the processes described herein, using starting materials which are commercially available, producible according to routine, conventional chemical methods or the synthesis of which is described herein.

Generally, compounds of the Formula (I) where R³ is H, (C₁-C₄)alkyl, or OH, Formula (Ia) [where R³ is H], (Ib) [where R³is NO₂], and (Ic) [where R³ is NH₂], can be synthesized as shown in Scheme 1. Compounds of Formula (I) where R³ is OH require the protection of the OH group prior to the first step; deprotection can occur during the third step. Compounds of Formula (I) where R³ is (C₁-C₄)alkyl are prepared from (Ia) where R³ is H by a three step procedure analogous to that of Zeitschrift fuer Naturforschung, Teil B: Anorganische Chemie, Organische Chemie, 30B(11-12), 954-8; 1975, that is incorporated herein by reference. Except for compounds where R¹ or R² is an optionally substituted amine or pyrrolidinyl (see Scheme 4), or where R³ is H and R⁴ is S(O)₂R⁷ (see Scheme 11), treatment of a substituted indole of Formula (II) with a protecting group produces an N-protected indole of Formula (III). The compound of Formula (III) can then be deprotonated and quenched with an electrophile to furnish a dicarbonyl indole compound of Formula (IV). The Formula (IV) compound can be condensed with an aryl 1,2-diamine of Formula (V) to generate a compound of Formula (Ia). Nitration of the compound of Formula (Ia, where R³ is H) can provide the 3-nitroindole compound of Formula (Ib) [where R³ is NO₂]. Reduction of the nitro functionality of the compound of Formula (Ib) can furnish a Formula (Ic) compound [where R³ is NH₂].

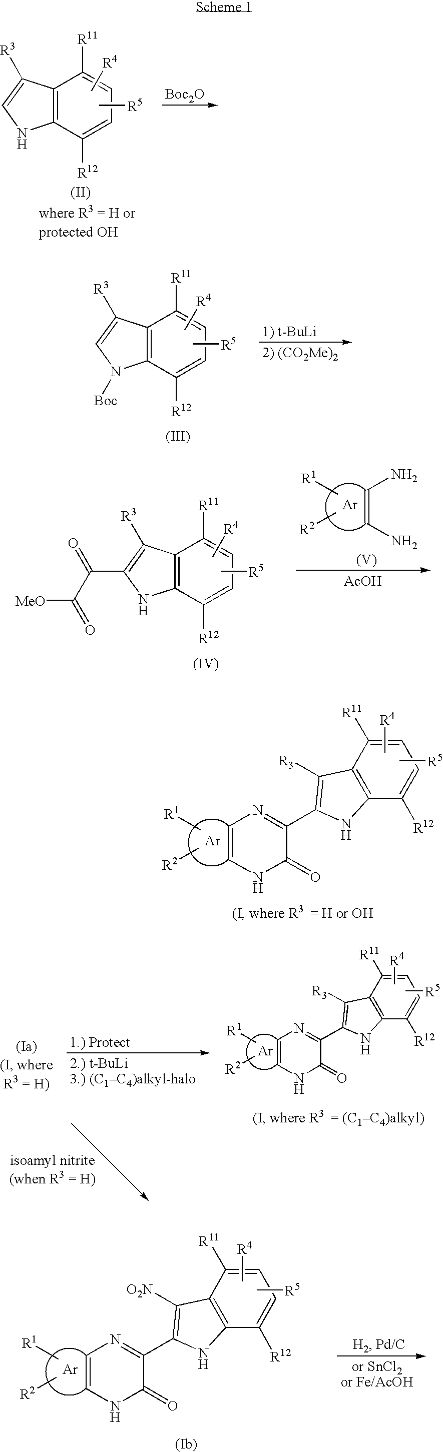

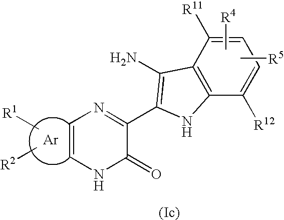

Formula (II) is readily available, or see Scheme 15 (for synthesis of Formula II where R⁴ is optionally substituted phenyl or optionally substituted pyridyl), Scheme 16 (for synthesis of Boc protected Formula (II) [that is, Formula III] where R⁴ is (C₁-C₆)alkoxy optionally substitued with 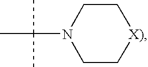
Scheme 19 (for synthesis of Formula (II) where R⁴ is N[(C₁-C₃)alkyl]₂,) and Scheme 21 (for synthesis of Formula (II) where R³ is H).

Formula (V) is readily available or see Scheme 14, where substituted 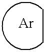
is readily available as a di-nitro compound, or see Scheme 20 where substituted 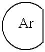
is readily available as a nitroaniline compound.

Compounds where R³ is NH(C₁-C₄)alkyl, NHC(O)(C₁-C₄)alkyl, or NHC(O)phenyl are synthesized starting with Formula (Ic), according to Scheme 5.

Reaction Schemes 2, 3, 5 through 10, 12 and 13 each describe how to make compounds with certain R⁴ sub-groups where the starting material is an R⁴-sub-group compound of Formula (Ia) [Scheme 8, which can be applied when R³ is H, as in Formula (Ia), or when R³ is alkyl], Formula (Ib) [Schemes 2, 3, 6, 7, 9,10, 12 and 13, which can be applied when R³ is NO₂, or is H, or alkyl]. As stated previously, Formula (1c) from Reaction Scheme 1 (where R³ is NH₂ and R⁴ is as described without limitation) can be converted to a compound where R³ is NH(C₁-C₄)alkyl, NHC(O)(C₁-C₄)alkyl, or NHC(O)phenyl according to Scheme 5.

Scheme 2 shows how compounds of Formula (I), where R³ is NO₂ and R⁴ is CN, can be converted to compounds of Formula (I) where R⁴ is C(O)R⁶ by standard functional group manipulation. For example, a cyanoindole (Id) can be hydrolyzed under basic conditions to an indole carboxylic acid (Ie). Coupling of acid (Ie) with an amine provides a variety of amides of general Formula (If).

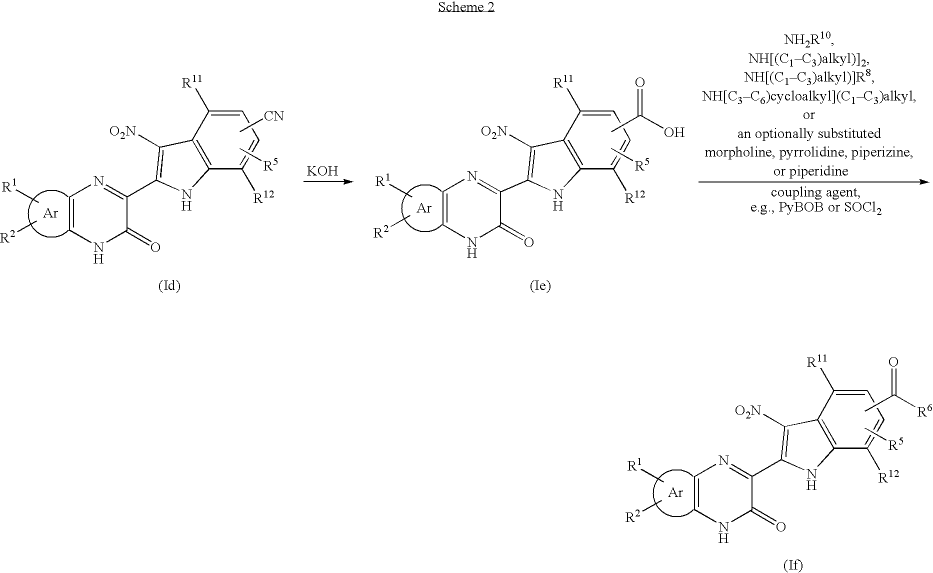

Other Formula (I) compounds where R³ is NO₂, and R¹¹ and R¹² are both H, can be prepared by conversion of the acid (Ie) to the alcohol of Formula (Ig) by a two step procedure employing an imidazolyl carbonyl intermediate followed by reduction as shown in Scheme 3. Treatment of alcohol (Ig) with a halogenating agent such as SOBr₂ produces a compound of Formula (Ih). Reaction of the halide (Ih) with either an alcohol or amine furnishes the ether (Ii) or the amine (Ij), respectively.

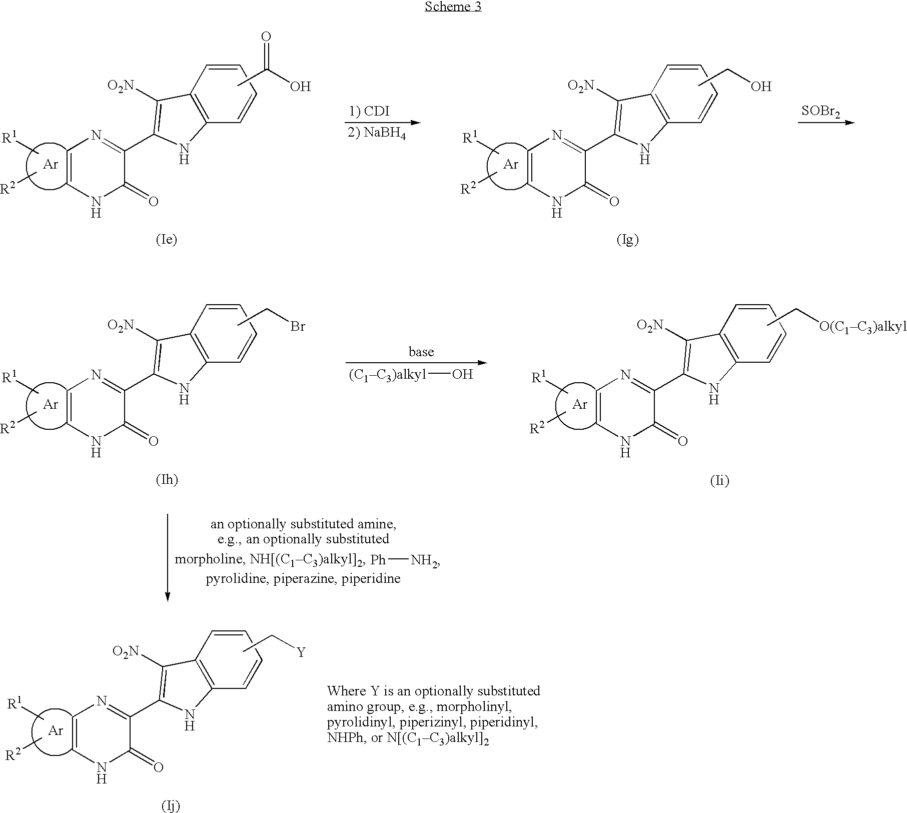

Compounds of Formula (Ik) in which the Ar ring is benzo, R¹ or R² is an amino substituent, and R³ is H can be prepared as shown in Scheme 4. Conversion of a difluoronitrobenzene of Formula (VI) to an aniline of Formula (VII) is accomplished by ammonia displacement of a fluoronitrobenzene. Displacement of a second fluoro group from the compound of Formula (VII) by an amine of Formula (VII) provides a phenylenediamine intermediate of Formula (IX). Reduction of the nitro group in (IX), followed by intramolecular condensation with ketoester (IV) provides a compound of Formula (Ik).

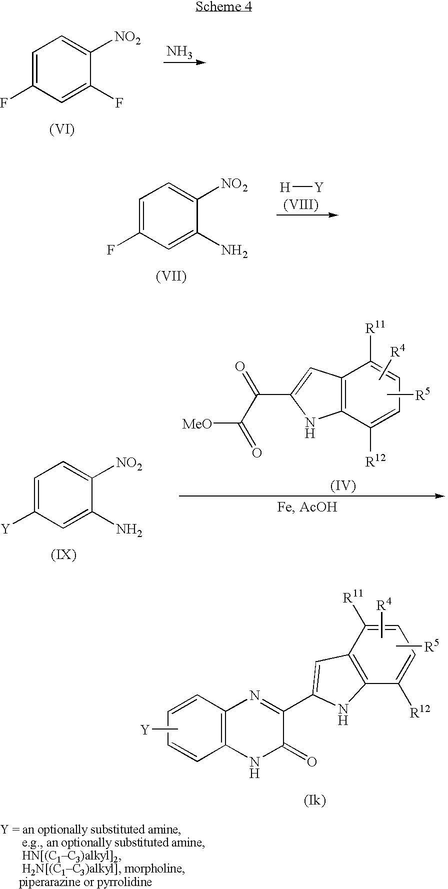

A compound of Formula (Ic) where R³ is NH₂ can be alkylated or acylated to produce compounds of Formula (Im) as shown in Scheme 5.

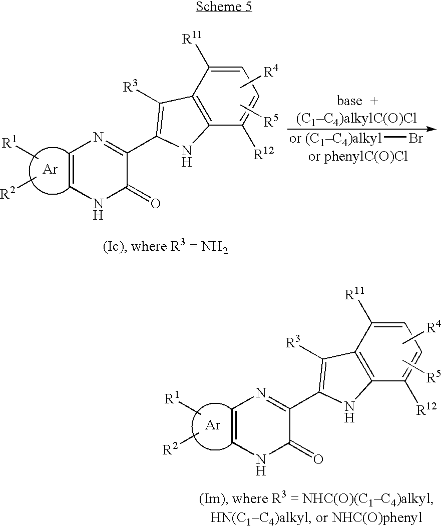

Preparation of compounds of Formula (In), prepared using the method described in Scheme 2, where R³ is NO₂ and R⁴ is an amido substituted pyrrolidine amide is shown in Scheme 6. A pyrrolidine amide (In) can be converted to the primary amine derivative (Io), which can be acylated to provide the amide (Ip).

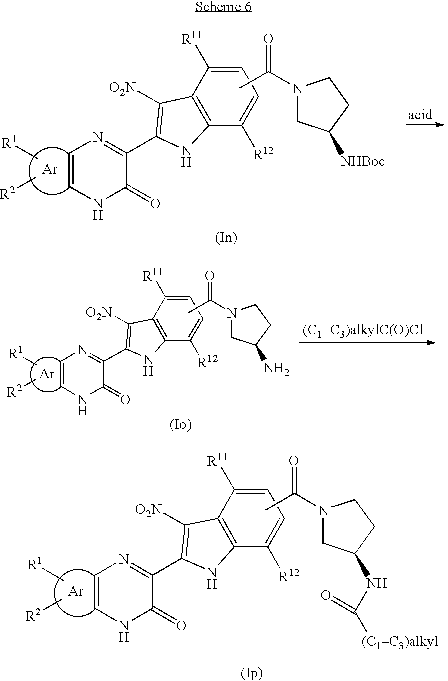

Compounds of Formula (Iq) where R³ is NO₂ and R⁴ is an acylsulfonamide are prepared as shown in Scheme 7. The indole carboxylic acid (Ie) is reacted with a sulfonamide to produce a sulfonyl carboxamide (Iq).

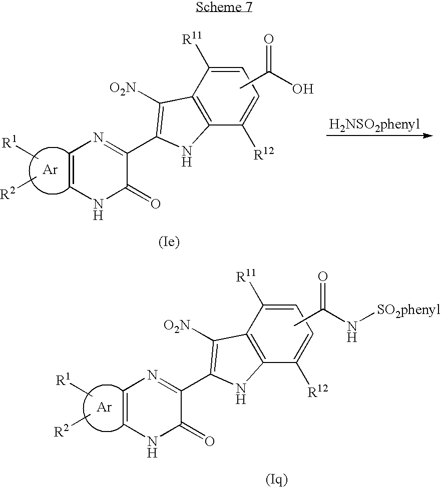

Preparation of compounds of Formula (It) where R³ is H and R⁴ is a pyridyloxy group are shown in Scheme 8. A methoxyindole of Formula (Ir) can be transformed into a hydroxyindole (Is), then coupled with a halopyridine to provide a biaryl ether (It) as shown.

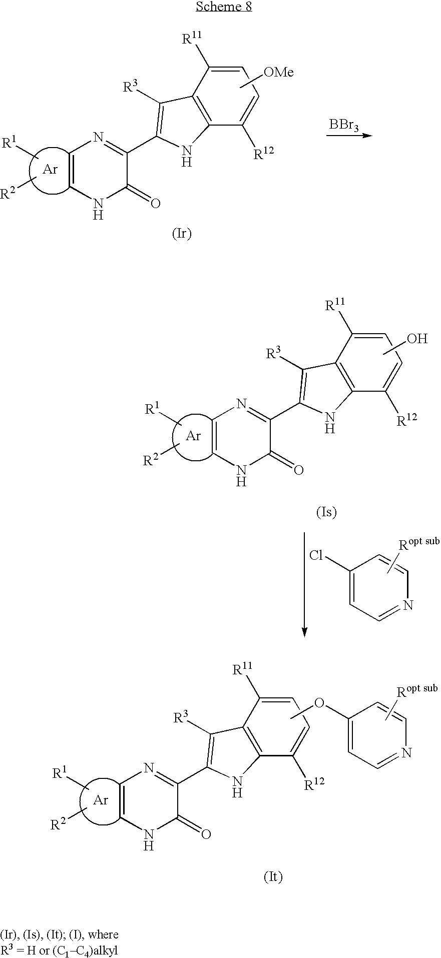

Other Formula (Iw) compounds where R³ is NO₂ and R⁴ is a urea substituted pyrrolidine amide can be prepared as shown in Scheme 9. A protected amine (Iu, prepared using the method described in Scheme 2) can be reacted with TFA to produce an N-methylamine of Formula (Iv). Secondary amine (Iv) can be converted to urea (1w).

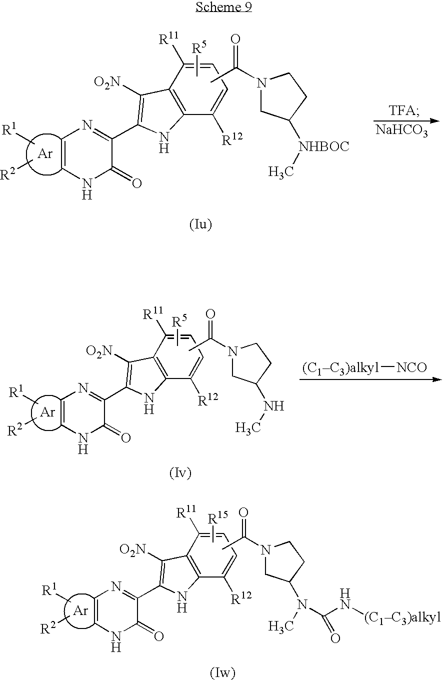

Other Formula (I) compounds where R³ is NO₂ and R⁴ is oxadiazole can be prepared by conversion of an amide of Formula (If) to the dehydrated heterocycle of Formula (Ix) as shown in Scheme 10.

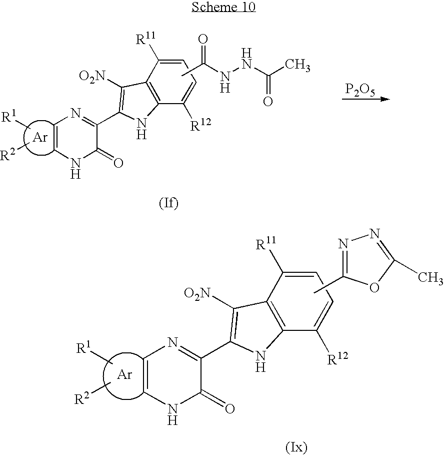

Compounds of Formula (Iy) can be prepared as shown in Scheme 11. For example, boronic acid indoles of Formula (X) can be united with an aryl chloride to provide indoles of Formula (XI). Acidic hydrolysis of aryl chlorides of Formula (XI) could produce quinoxalinones of Formula (Iy).

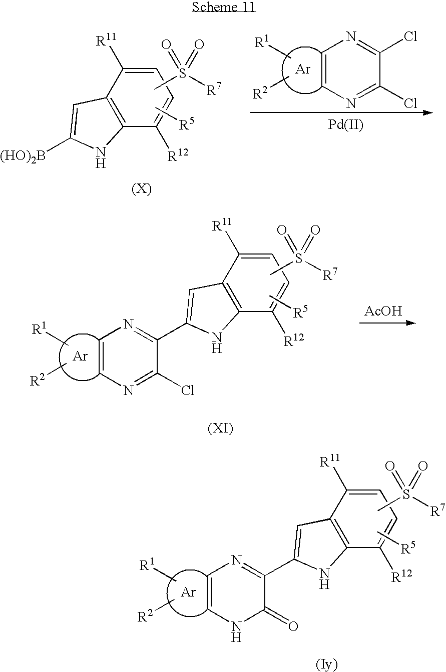

Hydroxymethyl indoles of Formula (Ig) where R³ is NO₂ and R⁴ is hydroxymethyl can be reacted with an isocyanate to furnish carbamates of Formula (Iz) as shown in Scheme 12.

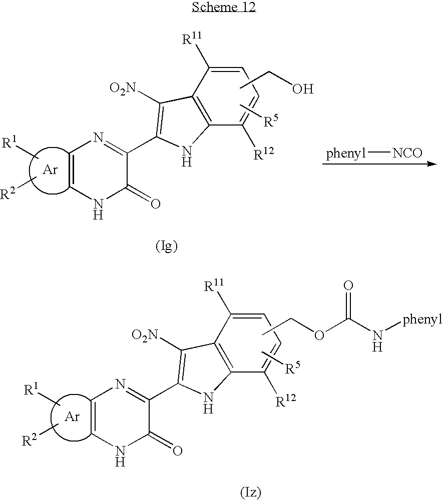

Compounds of Formula (Ie) can be converted to acid chlorides of Formula (XII) and reacted with an alcohol to produce ester derivatives of Formula (Iaa) as shown in Scheme 13. 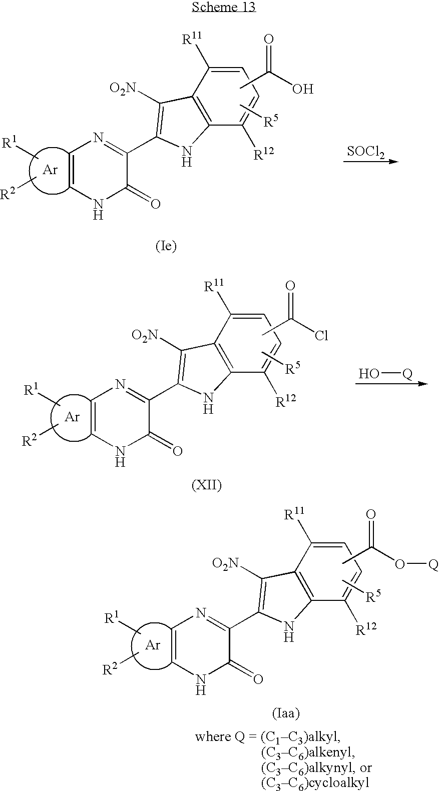
Preparation of Intermediates

Compounds of Formula (V), used in Scheme 1 above are either commercially available or can be prepared by reducing the appropriate 1,2-dinitroaryl precursor (XIII) as shown in Scheme 14.

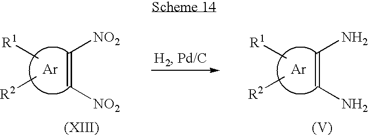

Biaryl indole compounds of Formula (IIIb) where R⁴ is phenyl or pyridyl can be prepared as shown in Scheme 15. Performing a palladium catalyzed cross coupling between an indole boronic acid of Formula (IIb) and an optionally substituted phenyl or pyridyl bromide to provide the indole of Formula (IIc). Protection (IIc) at the indole nitrogen provides the biaryl intermediate of Formula (IIIb).

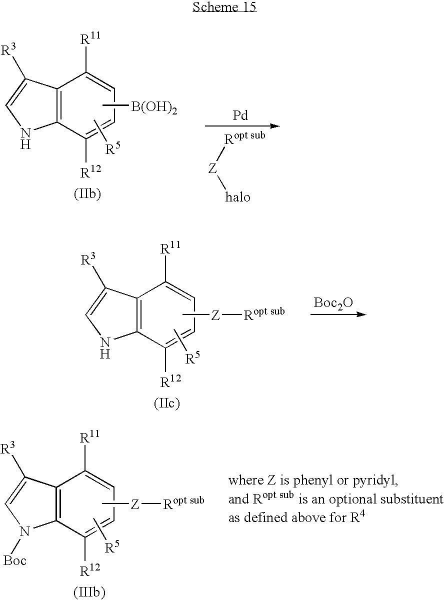

Intermediate indoles, used to prepare compounds of Formula (I), in which R⁴ is an morpholinyl-substituted alkoxy group, can be prepared from a hydroxyindole (IIIc) as shown in Scheme 16. Conversion of (IIIc) to an amine of Formula (IIIe) is accomplished in two steps via an intermediate haloether (IIId). The Formula (IIIe) indole is carried on to final product of Formula (I) in the Schemes described above.

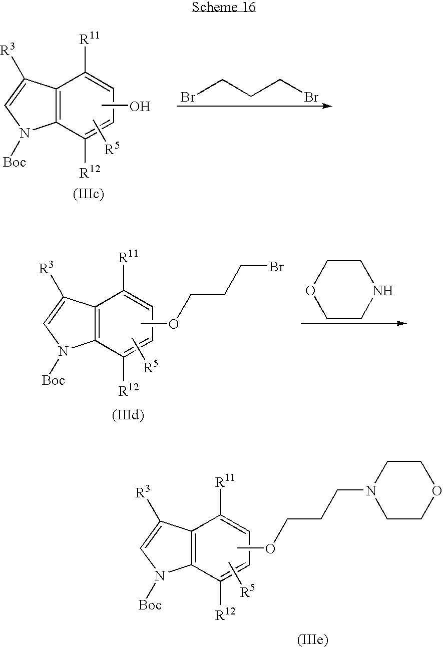

When substituted piperazine is used in the preparation of Formula (I) compounds in which R⁴ is an alkyl or acyl group substituted by piperazine, the substituted piperazine can be prepared by conversion of a compound of Formula (XIV) to a sulfonamide (XV) upon treatment with methylsulfonyl chloride. The product, a N-Boc protected piperazine (XV) can be converted to a monosubstituted piperazine of Formula (XVI) by subjecting (XV) to an acid such as TFA as shown in Scheme 17. The resulting Formula (XVI) can be used, for example, in the last step in Scheme 2.

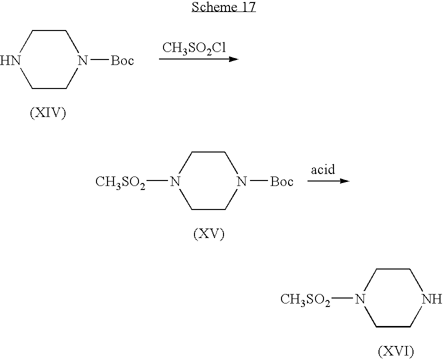

Amine derivatives of Formula (XVIII) can be prepared by conversion of a ketone of Formula (XVII) via reductive amination as shown in Scheme 18. This Scheme includes synthesis of the amine compounds that convert to N[(C₃-C₆)cycloalkyl][(C₁-C₃)alkyl] and to substituted N[C₁-C₄)alkyl]₂, and can be inserted into, for example, the last step of Scheme 2, the last step to make Formula (Ij) in Scheme 3, and as compound (VII) in Scheme 4.

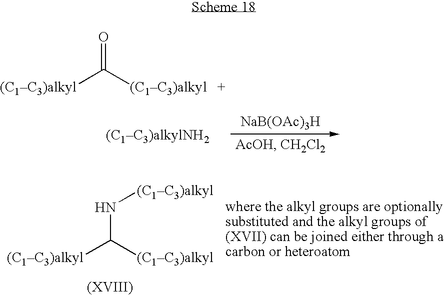

Compounds of Formula (IIIf) can be prepared as shown in Scheme 19. Conversion of a fluoronitrobenzene of Formula (XIX) to an aniline of Formula (XX) can be accomplished by displacement of the fluorine of (XIX). Nitroaniline (XX) can be converted to aminoindole (IIIf).

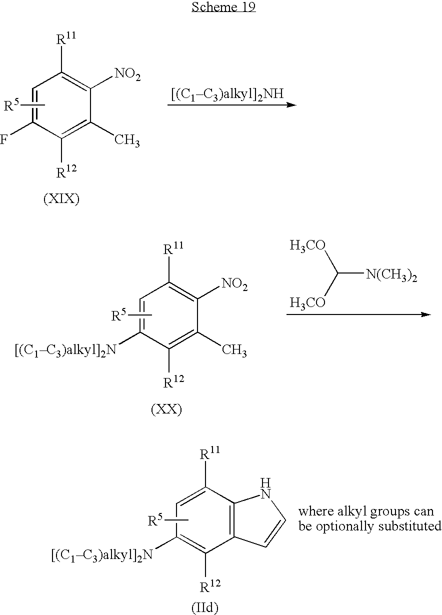

Compounds of Formula (Vb) can be prepared as shown in Scheme 20. Palladium assisted coupling of a bromonitroaniline of Formula (XXI) with aryl boronic acids could provide arylnitroanilines of Formula (XII). Reduction of the nitro group could provide diamines of Formula (Vb).

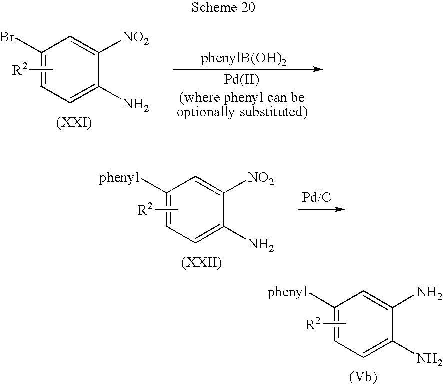

Compounds of Formula (IIIg) can be synthesized from anilines of Formula (XXIII) as shown in Scheme 21. The anilines could be converted to diazonium salts of Formula (XXIV) followed by reduction to substituted phenyl hydrazines of Formula (XXV). The hydrazines can be converted to phenyl hydrazones of Formula (XXVI) which can undergo an acid assisted cyclization to yield substituted indoles of Formula (IIIg).

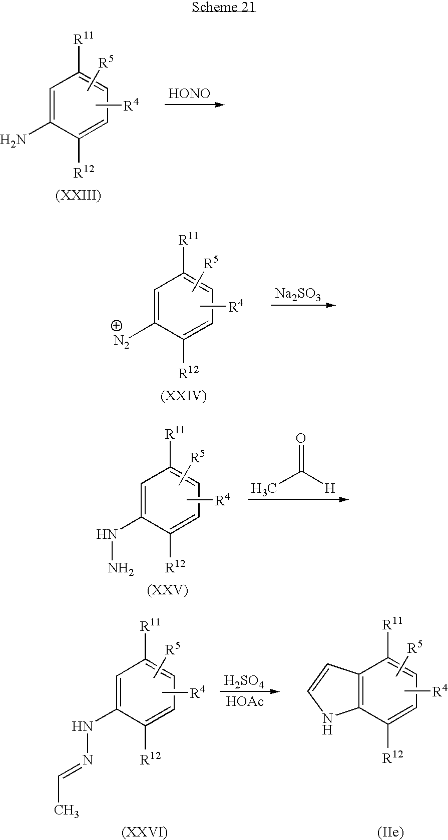

It is to be understood that sensitive or reactive substituents attached to intermediates or to compounds of Formula (I) may need to be protected and deprotected during the preparations described above. Protecting groups in general may be added and removed by conventional methods well known in the art [see, e.g., T. W. Greene and P. G. M. Wuts, Protective Groups in Organic Synthesis; Wiley: New York, (1999)].

In addition, it is to be understood that reaction conditions for N— or O-acylation, alkylation, or sulfonylation of the intermediates and of Formula (I) compounds using acyl halides, alkyl halides and sulfonyl halides, respectively, and a suitable base, are generally interchangeable, as is well known in the art. For example, conditions to effect N-acylation as described in any of the specific examples below can also be used to effect N-sulfonylation by substituting the appropriate sulfonyl halide for the acyl halide.

The following specific examples are presented to illustrate the invention described herein, but should not be construed as limiting the scope of the invention in any way.

### Abbreviations and Acronyms

When the following abbreviations are used throughout the disclosure, they have the following meaning:
AcCl acetyl chloride
AcOH acetic acid
Boc t-butoxycarbonyl
CDI carbonyl diimidazole
Celite® registered trademark of Celite Corp. brand of diatomaceous earth
DMAP 4-(N,N-dimethyl)amino pyridine
DME dimethoxyethane
DMF N,N-dimethyl formamide
DMSO-d₆ dimethylsulfoxide-d₆
ESI electrospray ionization
EtOAc ethyl acetate
EtOH ethanol
¹H NMR proton nuclear magnetic resonance
Hex hexanes
HPLC high performance liquid chromatography
LCMS liquid chromatography/mass spectroscopy
MeOH methanol
MS mass spectrometry
Pd/C palladium on carbon
R_{f} TLC retention factor
rt room temperature
RT retention time (HPLC)
TBDMS tert-butyldimethylsilyl
TBDMSCl tert-butyldimethylsilyl chloride
TFA trifluoroacetic acid
THF tetrahydrofuran
TLC thin layer chromatography
TMS tetramethylsilane
   General Experimental Procedures

Electron impact mass spectra (El-MS) were obtained with a Hewlett Packard 5989A mass spectrometer equipped with a Hewlett Packard 5890 Gas Chromatograph with a J & W DB-5 column (0.25 uM coating; 30 m×0.25 mm). The ion source was maintained at 250° C. and spectra were scanned from 50-800 amu at 2 sec per scan.

High pressure liquid chromatography-electrospray mass spectra (LC-MS) were obtained using either a:
(A) Hewlett-Packard 1100 HPLC equipped with a quaternary pump, a variable wavelength detector set at 254 nm, a YMC pro C-18 column (2×23 mm, 120 A), and a Finnigan LCQ ion trap mass spectrometer with electrospray ionization. Spectra were scanned from 120-1200 amu using a variable ion time according to the number of ions in the source. The eluents were A: 2% acetonitrile in water with 0.02% TFA and B: 2% water in acetonitrile with 0.018% TFA. Gradient elution from 10% B to 95% over 3.5 minutes at a flowrate of 1.0 mL/min was used with an initial hold of 0.5 minutes and a final hold at 95% B of 0.5 minutes. Total run time was 6.5 minutes.
   or

(B) Gilson HPLC system equipped with two Gilson 306 pumps, a Gilson 215 Autosampler, a Gilson diode array detector, a YMC Pro C-18 column (2×23mm, 120 A), and a Micromass LCZ single quadrupole mass spectrometer with z-spray electrospray ionization. Spectra were scanned from 120-800 amu over 1.5 seconds. ELSD (Evaporative Light Scattering Detector) data was also acquired as an analog channel. The eluents were A: 2% acetonitrile in water with 0.02% TFA and B: 2% water in acetonitrile with 0.018% TFA. Gradient elution from 10% B to 90% over 3.5 minutes at a flowrate of 1.5 mL/min was used with an initial hold of 0.5 minutes and a final hold at 90% B of 0.5 minutes. Total run time was 4.8 minutes. An extra switching valve was used for column switching and regeneration.

Routine one-dimensional NMR spectroscopy was performed on 300 MHz Varian Mercury-plus spectrometers. The samples were dissolved in deuterated solvents obtained from Cambridge Isotope Labs, and transferred to 5 mm ID Wilmad NMR tubes. The spectra were acquired at 293 K. The chemical shifts were recorded on the ppm scale and were referenced to the appropriate solvent signals, such as 2.49 ppm for DMSO-d₆, 1.93 ppm for CD₃CN, 3.30 ppm for CD₃OD, 5.32 ppm for CD₂Cl₂ and 7.26 ppm for CDCl₃ for ¹H spectra, and 39.5 ppm for DMSO-d₆, 1.3 ppm for CD₃CN, 49.0 ppm for CD₃OD, 53.8 ppm for CD₂Cl₂ and 77.0 ppm for CDCl₃ for ¹³C spectra.

### EXAMPLE 1

### Preparation of 3-(1H-indol-2-yl)-2(1H)-quinoxalinone

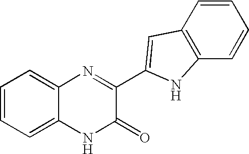

### Step 1. Preparation of tert-butyl 2-[methoxy(oxo)acetyl]-1H-indole-1-carboxylate

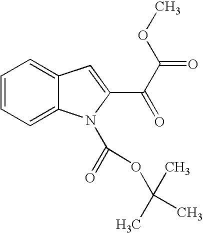

In a 250 mL round-bottom flask was placed 2.0 g (9.21 mmol, 1 equiv) of N-Boc indole in 40 mL of THF. The mixture was cooled to −78° C. and 1.1 equiv (6.33 mL, 1.6 M in pentane) of t-BuLi was added dropwise. The mixture was allowed to stir for 30 min and 2.17 g (18.4 mmol, 2 equiv) of dimethyl oxalate in 20 mL THF was added quickly in one portion. The reaction was then allowed to warm to rt. After 30 min the reaction appeared to be complete by TLC. The mixture was diluted with 50 mL of water and transferred to a separatory funnel where it was extracted with EtOAc (3×200 mL). The combined organics were dried (Na₂SO₄), filtered, and evaporated. The residue was then purified via flash chromatography (15% EtOAc/Hex) to provide 2.02 g (72%) of the desired product as a yellow oil. ¹H-NMR (CD₃CN) δ 8.06 (d, 1H), 7.75 (d, 1H), 7.55 (d, 1H), 7.37 (d, 1H), 7.32 9S, 1H), 3.87 (s, 3H), 1.67 (s, 9H).

### Step 2. Preparation of 3-(1H-indol-2-yl)-2(1H)-quinoxalinone

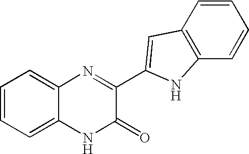

In a 25 mL round-bottom flask was placed 300 mg (0.99 mmol, 1 equiv) of tert-butyl 2-[methoxy(oxo)acetyl]-1H-indole-1-carboxylate and 118 mg (1.09 mmol, 1.1 equiv) 1,2-phenylenediamine in 10 mL of acetic acid. The flask was equipped with a reflux condenser and heated at 130° C. for 2 h. At this point, 1 mL of TFA was added to ensure complete removal of the Boc group. The mixture was then allowed to cool to room temperature and diluted with 10 mL of water. The resulting precipitate was filtered and rinsed with an additional 20 mL of water to provide 199 mg (77%) of the desired product as an orange solid. ¹H-NMR (DMSO-d₆) δ 12.62 (s, 1H), 11.61 (s, 1H), 7.86 (s, 1H), 7.83 (d, 1H), 7.65 (d, 1H), 7.54 (d, 1H), 7.50 (d,₁ H), 7.36 (t, 1H), 7.35 (t, 1H), 7.20 (t, 1H), 7.02 (t, 1H); LCMS RT=2.96 min; [M+H⁺=262.23.

### EXAMPLE 2

### Preparation of 3-(3-nitro-1H-indol-2-yl)-2(1H)-quinoxalinone

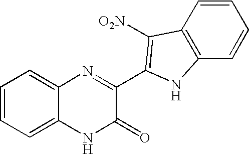

In a 100 mL round-bottom flask equipped with a reflux condenser was placed 3-(1H-indol-2-yl)-2(1H)-quinoxalinone (Example 1, 400 mg, 1.53 mmol) in 30 mL of benzene and 6 mL of DMF. The mixture was heated to 100° C. and 538 mg (4.59 mmol, 3 equiv) of isoamyl nitrite was added. After 2 h, the reaction appeared complete and was allowed to cool to rt. The solvents were removed in vacuo and the residue suspended in CH₃CN and sonicated. The remaining solids were filtered to provide 404 mg (86%) of the desired product as yellow solid. ¹H-NMR (DMSO-d₆) δ13.15 (s, 1H), 12.84 (s, 1H), 8.12-8.08 (m, 1H), 7.89 (d, 1H), 7.69-7.59 (m, 2H), 7.45-7.37 (m, 4H); LCMS RT=2.86 min; [M+H]⁺=307.22.

### EXAMPLE 3

### Preparation of 3-(3-amino-1H-indol-2-yl)-2(1H)-quinoxalinone

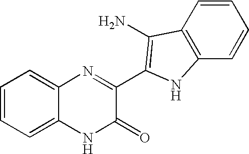

In a 25 mL round-bottom flask was placed 10 mg of 10% Pd/C under argon. To this was added 5 mL of THF. To this mixture was added 100 mg (0.33 mmol) of 3-(3-nitro-1H-indol-2-yl)-2(1H)-quinoxalinone (Example 2) as a solution in 3 mL of DMF and 5 mL of THF. The atmosphere was converted to one of H₂ with a balloon and the reaction allowed to stir at rt for 1 h. The H₂ was then removed and the mixture filtered through Celite® under a blanket of argon. The solvents were then removed to provide 71 mg (78%) of the desired product as a red solid. ¹H-NMR (DMSO-d₆) δ 12.39 (s, 1H), 10.57 (s, 1H), 7.78 (d, 1H), 7.72 (d, 1H), 7.44 (d, 1H), 7.28 (d, 1H), 7.23 (t, 2H), 7.16 (t, 1H), 7.02 (br s, 2H), 6.88 (t, 1H); LCMS RT=2.33 min; [M+H]⁺=277.28.

### EXAMPLE 12

### Preparation of 6,7-dimethoxy-3-(5-cyano-1H-indol-2-yl)-2(1H)-quinoxalinone

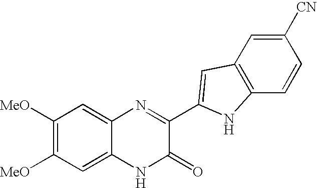

### Step 1. Preparation of tert-butyl 5-cyano-1H-indole-1-carboxylate

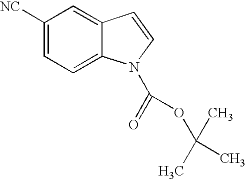

In a 100 mL round-bottom flask was placed 1H-indole-5-carbonitrile (2.0 g, 14.07 mmol) in 20 mL of anhydrous THF. To this solution was added DMAP (0.86 g, 7.03 mmol) and the mixture was allowed to stir for 0.5 h at rt. At this point, Boc₂O (3.07 g, 14.07 mmol) was added and the reaction stirred for an additional 2 h. The reaction was then quenched with water and extracted twice with ethyl ether. The combined organic layers were washed successively with 1N HCl, water, and brine, then dried over MgSO₄ and concentrated to provide 3.26 g (96%) of the desired product as a white solid. ¹H-NMR (DMSO-d₆) δ 8.20-8.14 (m, 2H), 7.83 (d, 1H), 7.70 (d, 1H), 6.80 (d, 1H), 1.63 (s, 9H).

### Step 2. Preparation of methyl (5-cyano-1H-indol-2-yl)(oxo)acetate

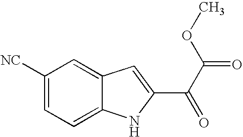

In a 100 mL round-bottom flask was placed 2.0 g (8.26 mmol, 1 equiv) of tert-butyl 5-cyano-1H-indole-1-carboxylate (step 1) in 25 mL of THF. The mixture was cooled to −78° C. and 1.1 equiv (5.34 mL, 1.7 M in pentane) of t-BuLi was added dropwise. The mixture was allowed to stir for 1 h and 2.14 g (18.16 mmol, 2.2 equiv) of dimethyl oxalate in 5 mL of THF was added quickly in one portion. The reaction was then allowed to warm to 0° C. and stirred until complete, as monitored by TLC (about 2 h). The mixture as diluted with 30 mL of water and transferred to a separatory funnel where it was extracted with EtOAc (3×100 mL). The combined organic extract was washed with brine, dried (Na₂SO₄), filtered, and evaporated to give a brown residue. To the residue was added MeOH (10 mL) to give an insoluble yellow solid, which was filtered, washed with MeOH, dried, and purified to provide 444.3 mg (23.6%) of the desired product as a yellow solid. ¹H-NMR (DMSO-d₆) δ 12.63 (s,₁ H), 8.40 (s, 1H), 7.77 (s, 1H), 7.65 (d, 1H), 7.60 (d, 1H), 3.93 (s, 3H).

### Step 3. Preparation: 6,7-dimethoxy-3-(5-cyano-1H-indol-2-yl)-2(1H)-quinoxalinone

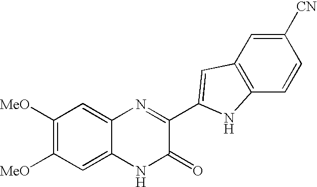

In a 25 mL round-bottom flask was placed 114.1 mg (0.50 mmol, 1 equiv) of 5-cyano-2-[methoxy(oxo)acetyl]-1H-indole (step 2) and 132.6 mg of 1,2-diamino-4,5-dimethoxybenzene hydrochloride (0.55 mmol, 1.1 equiv) in 5 mL of acetic acid. The flask was equipped with a reflux condenser and heated at 130° C. for 3 h. The mixture was then allowed to cool to room temperature and diluted with 5 mL of water. The resulting precipitate was filtered and rinsed with an additional 10 mL of water, 5 mL of MeCN, dried in an oven to provide 127.1 mg (73.4%) of the desired product as a yellow solid. ¹H-NMR (DMSO-d₆) δ 12.67 (s, 1H), 12.08 (s, 1H), 8.20 (s, 1H), 7.80 (s, 1H), 7.65 (d, 1H), 7.49 (d, 1H), 7.27 (s, 1H), 6.85 (s, 1H), 3.87 (s, 6H); LCMS RT=2.68 min; [M+H]⁺=347.2.

### EXAMPLE 13

### Preparation of 6-[(3S)-3-(dimethylamino)-1-pyrrolidinyl]-3-(1H-indol-2-yl)-2(1H)-quinoxalinone

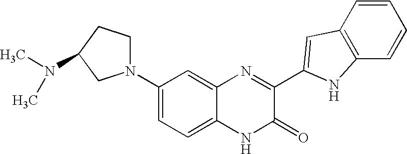

### Step 1. Preparation of 5-fluoro-2-nitroaniline

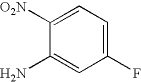

The compound was prepared as described in WO 02/22598. To a round bottom flask equipped with a dry ice condenser (acetone/dry ice) was added 2,4-difluoronitrobenzene (15 g, 94 mmol) and THF (20 mL). Ammonia was bubbled into the solution for 10 min at −78° C. The reaction was allowed to warm to room temperature and the reaction refluxed for 7 h. Stirring was continued overnight allowing the ammonia to evaporate after the condenser was removed. The reaction was diluted with dichloromethane and washed with water (3×100 mL). the organic layer was dried over MgSO₄, filtered, and concentrated under reduced pressure to yield a solid. The solid was purified by chromatography to afford 10.5 g (72%) of 5-fluoro-2-nitroaniline. ¹H NMR (DMSO-d₆): δ 6.38-6.52 (m, 1H), 6.66-6.72 (d, 1H), 7.79 (s, 2H), 7.98-8.09 (dd, 1H). LRMS RT=2.48; [M+H]=157.

### Step 2. Preparation of (3S)-1-(3-amino4-nitrophenyl)-N,N-dimethyl-3-pyrrolidinamine

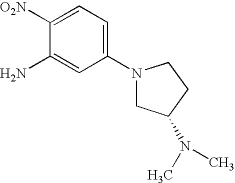

The compound was prepared as described in WO 02/22598. To a round bottom flask equipped with a reflux condenser was added 5-fluoro-2-nitroaniline (4 g., 26.0 mmol) in 1-methyl-2-pyrrolidine (40 mL). (3S)-N,N-dimethyl-3-pyrrolidinamine (5.85 g., 51.2 mmol) was added to the stirring solution and the reaction was heated to 80° C. for 3 h. After cooling to room temperature, the reaction was poured over ice water. The product was diluted with dichloromethane and washed with saturated sodium bicarbonate (3×100 mL). The organic layer was dried with MgSO₄, filtered, and concentrated under reduced pressure to afford a solid. The product was purified by chromatography to yield 4.7 g (74%) of (3S)-1-(3-amino-4-nitrophenyl)-N,N-dimethyl-3-pyrrolidinamine. ¹H NMR (DMSO-d₆): δ 1.81-1.92 (m, 1H), 2.02-2.13 (m, 7H), 2.87-2.91 (m, 1H), 3.06-3.16 (t, 1H), 3.22-3.33 (m, 1H), 3.41-3.8 (dt, 2H), 5.80 (s, 1H), 6.00-6.14 (dd, 1H), 7.25 (s, 2H), 7.75-7.83 (dd, 1H). LRMS RT=0.25; [M+H]=251.

### Step 3. Preparation of 6-[(3S)-3-(dimethylamino)-1-pyrrolidinyl]-3-(1H-indol-2-yl)-2(1H)-quinoxalinone

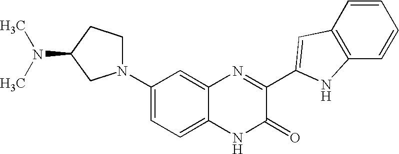

The compound was prepared by reaction of the product prepared in Example 13, step 2, with the product of Example 1, step 1, using the method described for Example 49-50 step 2. ¹H-NMR (DMSO-d₆) δ 12.25 (s, 1H), 11.41 (s, 1H), 7.65-7.51 (m, 3H), 7.58-7.49 (d, 1H), 7.22-7.18 (m, 1H), 7.16-7.01 (m, 1H), 6.80-6.72 (d, 1H), 6.38 (s, 1H), 3.67-3.52 (m, 2H), 3.24-3.14 (t, 1H), 2.97-2.81 (m, 1H), 2.35 (s, 6H), 1.99-1.83 (m, 1H). LCMS RT=2.06 min; [M+H]=374.

### EXAMPLE 18

### Preparation of 3-{5-[3-(1-piperazinyl)propoxy]-1H-indol-2-yl}-2(1H)-quinoxalinone

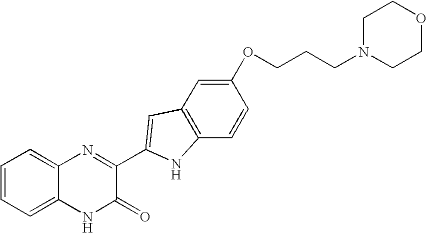

### Step 1. Preparation of tert-butyl 5-hydroxy-1H-indole-1-carboxylate

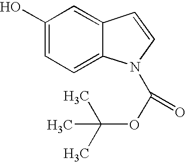

Tert-butyl 5-(benzyloxy)-1H-indole-1-carboxylate (5.75 g, 17.8 mmol), prepared according to the procedure described for Example 12, step 1, was added to a mixture of 10% Pd/C in EtOH. Ammonium formate was added and the reaction stirred for 6 h. The mixture was filtered through Celite® under a blanket of argon and the solvents were then removed. The residue was purified by flash chromatography to yield 3.5 g of tert-butyl 5-hydroxy-1H-indole-1-carboxylate (74%). ¹H-NMR (DMSO-d₆) δ 9.19 (s, 1H), 7.84-7.78 (d, 1H), 7.58-7.52 (d, 1H), 6.91 (s, 1H), 7.78-7.69 (m, 1H), 6.65-6.42 (m, 1H), 1.68-1.59 (s, 9H).

### Step 2. Preparation of tert-butyl 5-(3-bromopropoxy)-1H-indole-1-carboxylate

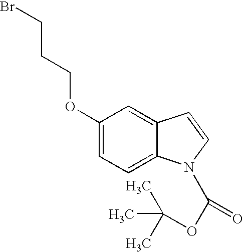

In a 250 mL flask was placed tert-butyl 5-(benzyloxy)-1H-indole-1-carboxylate (3.3 g, 14 mmol) in 100 mL of acetone. 1,3-Dibromopropane (5.74 mL, 56.6 mmol) was added, followed by cesium carbonate (5.5 g, 17 mmol). The reaction was heated to reflux for 5 h. The reaction was cooled to room temperature and diluted with water (200 mL). The mixture was transferred to a separatory funnel and extracted with ethyl acetate (2×150 mL). The combined organics were dried (MgSO₄), filtered, and evaporated. The residue was then purified via flash chromatography to provide 4.7 g of tert-butyl 5-(3-bromopropoxy)-1H-indole-1-carboxylate (94%). ¹H-NMR (DMSO-d₆) δ 7.99-7.89 (d, 1H), 7.61 (s, 1H), 7.17 (s, 1H), 6.98-6.91 (d, 1H), 6.62 (s, 1H), 4.16-4.05 (t, 2H), 3.64 (t, 2H), 2.37-2.20 (m, 2H). LCMS RT=3.55 min; [M]⁺=254.1.

### Step 3. Preparation of tert-butyl 5-[3-(4-morpholinyl)propoxy]-1H-indole-1-carboxylate

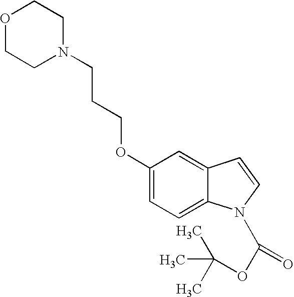

In a 250 mL flask was placed tert-butyl 5-(3-bromopropoxy)-1H-indole-1-carboxylate (1.5 g, 4.2 mmol) in 50 mL of tetrahydrofuran. Morpholine (0.41 mL, 4.66 mmol) was added, followed by pyridine (0.38 mL, 4.66 mmol). The reaction was heated to reflux for 5 h. The reaction was cooled to room temperature and diluted with water (200 mL). The mixture was transferred to a separatory funnel and extracted with ethyl acetate (2×100 mL). The combined organics were dried (MgSO₄), filtered, and evaporated. The residue was then purified via flash chromatography to provide 1.1 g of tert-butyl 5-[3-(morpholinyl)propoxy)-1H-indole-1-carboxylate (72%). ¹H-NMR (DMSO-d₆) δ 7.93-7.85 (d, 1H), 7.59 (s, 1H), 7.09 (s, 1H), 6.93-6.85 (m, 1H), 6.59 (s, 1H), 4.06-3.97 (t, 2H), 3.57 (s, 4H), 2.46-2.23 (m, 6H), 1.92-1.83 (m, 2H), 1.62 (s, 9H). LCMS RT=0.61 min; [M+H]⁺=361.3.

### Step 4. Preparation of tert-butyl 2-[methoxy(oxo)acetyl]-5-[3-(4-morpholinyl)propoxy]-1H-indole-1-carboxylate

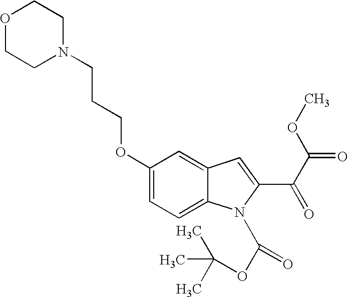

The compound was prepared by the method described for Example 1, step 1, using the product of Example 18, step 3 and dimethyl oxalate as starting materials. ¹H-NMR (DMSO-d₆) δ 7.86-7.80 (d, 1H), 7.38 (s, 1H), 7.20-7.29 (m, 1H), 7.18-7.10 (d, 1H), 4.06-3.99 (t, 2H), 3.80 (s, 3H), 3.57 (s, 4H), 2.47-2.24 (m, 6H), 1.96-1.83 (m, 2H), 1.59 (s, 9H).

### Step 5. Preparation of 3-{5-[3-(1-piperazinyl)propoxy]-1H-indol-2-yl}-2(1H)-quinoxalinone

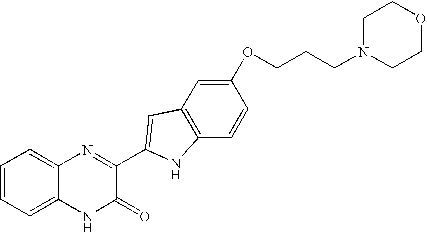

The compound was prepared by the method described for Example 1, step 2, using the product of Example 18, step 4 and 1,2-phenylenediamine as starting materials. ¹H-NMR (DMSO-d₆) δ 12.59 (s, 1H), 11.43 (s, 1H), 7.82-7.78 (d, 1H), 7.72 (s, 1H), 7.55-7.47 (m, 1H), 7.42-7.39 (m, 1H), 7.37-7.29 (m, 2H), 7.13 (s, 1H), 6.87-6.81 (m, 1H), 4.08-3.98 (t, 2H), 3.57 (s, 4H), 2.46-2.23 (m, 6H), 1.97-1.81 (m, 2H). LCMS RT=2.45 min; [M+H]=405.

### EXAMPLE 21

### Preparation of N-[3-(4-morpholinyl)propyl]-2-(3-oxo-3,4-dihydro-2-quinoxalinyl)-1H-indole-5-carboxamide

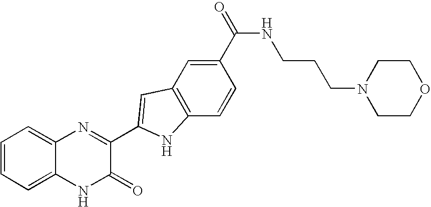

In a 20 mL amber vial was placed 2-(3-oxo-3,4-dihydro-2-quinoxalinyl)-1H-indole-5-carboxylic acid (Example 17, 75.0 mg, 0.25 mmol, and 0.10 mL (0.74 mmol) of TEA in 3 mL of THF and 3 mL of DMF. To this was added PyBOP (39.0 mg, 0.27 mmol) and 3-(4-morpholinyl)propylamine (0.04 mL, 0.27 mmol) and the reaction allowed to stir at rt. After 45 min, the volatiles were removed and the residue purified via preparative HPLC (CH₃CN/H₂O 0.1% TFA). The desired fractions were combined and the CH₃CN removed in vacuo. The remaining aqueous solution was basified with saturated NaHCO₃ and extracted with EtOAc (3×150 mL). The combined organics were dried (Na₂SO₄), filtered, and evaporated. The residue was suspended in CH₃CN, sonicated, and the solids filtered to provide 23 mg (21%) of the desired product as a yellow solid. ¹H-NMR (DMSO-d₆) δ 12.64 (s, 1H), 11.84 (s, 1H), 8.38 (t, 1H), 8.17 (s, 1H), 7.90 (s, 1H), 7.83 (d, 1H), 7.70 (d, 1H), 7.52 (dd, 2H), 7.37-7.32 (m, 2H), 3.57 (t, 4H), 3.30 (m, 2H), 2.40-2.31 (m, 6H), 1.70 (quint, 2H); LCMS RT=2.30 min; [M+H]⁺=432.29.

### EXAMPLE 22

### Preparation of 3-nitro-2-(3-oxo-3,4-dihydro-2-quinoxalinyl)-1H-indole-5-carboxylic acid

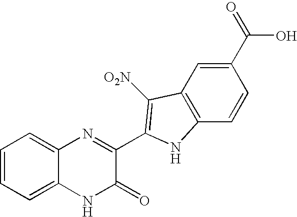

In a 15 mL round-bottom flask with condenser was placed 3-nitro-2-(3-oxo-3,4-dihydro-2-quinoxalinyl)-1H-indole-5-carbonitrile (Example 19, 52.0 mg, 0.16 mmol) in 6 mL of 4 M KOH. The mixture was heated at 120° C. for 3 h. At this point, the reaction was allowed to cool to rt and acidified with conc. HCl. The solids were filtered and dried in vacuo at 60° C. to provide 52 mg (95%) of the desired product as a yellow solid. ¹H-NMR (DMSO-d₆) δ 13.43 (s, 1H), 12.99 (br s, 1H), 12.90 (s, 1H), 8.73 (s, 1H), 7.98 (d, 1H), 7.90 (d, 1H), 7.70 (d, 1H), 7.66 (d, 1H), 7.40 (dd, 2H); LCMS RT=2.58 min; [M+H]⁺=351.26.

### EXAMPLES 49-50

### Preparation of 3-(1H-indol-2-yl)-6-[3-(4-morphlinyl)propoxy]-2(1H)-quinoxalinone and 3-(1H-indol-2-yl)-7-[3-(4-morphlinyl)propoxy]-2(1H)-quinoxalinone

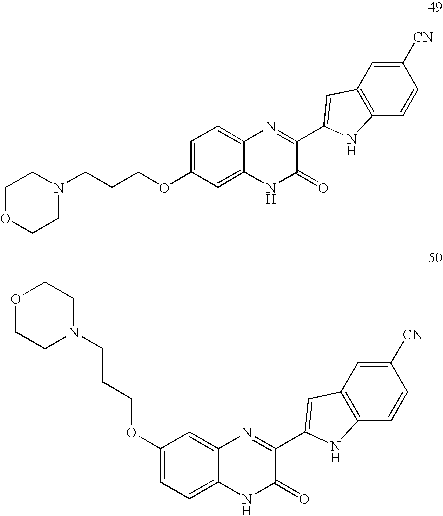

### Step 1. Preparation of 5-[3-(4-morpholinyl)propoxy]-2-nitroaniline

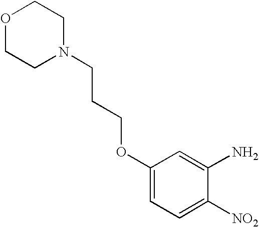

5-[3-(4-morpholinyl)propoxy]-2-nitroaniline (706 mg g, 69%) was obtained in two steps by O-alkylation of 3-amino-4-nitrophenol (1.0 g, 3.6 mmol) with 1,3-dibromopropane, catalyzed by Cs₂CO₃, followed by N-alkylation of morpholine catalyzed by pyridine: ¹H-NMR (DMSO-d6) δ 7.36 (s, 1H), 7.25 (s, 2H), 7.17-7.09 (m, 1H), 6.97-6.88 (d, 1H), 3.98-3.84 (t, 2H), 3.56 (s, 4H), 2.50-2.22 (m, 6H), 1.85-1.78 (m, 2H). LCMS RT=0.25 min; (M+H]⁺=282.3.

### Step 2. Preparation of 3-(1H-indol-2-yl)-6-[3-(4-morphlinyl)propoxy]-2(1H)-quinoxalinone and 3-(1H-indol-2-yl)-7-[3-(4-morphlinyl)propoxy]-2(1H)-quinoxalinone

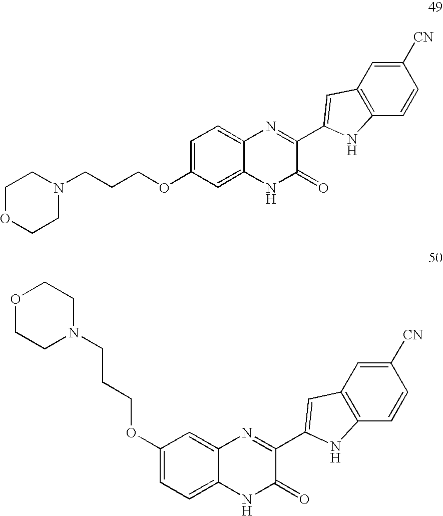

In a 25 mL round-bottom flask was placed methyl (5-cyano-1H-indol-2-yl)(oxo)acetate (255 mg, 0.79 mmol, Example 12, step 2) and 219 mg (0.79 mmol) of 5-[3-(4-morpholinyl)propoxy]-2-nitroaniline (from step 1) in 10 mL of acetic acid, followed by iron powder (219 mg). The flask was equipped with a reflux condenser and heated at 130° C. for 2 h. The mixture was then allowed to cool to room temperature and diluted with 80 mL of diethyl ether. The resulting precipitate was filtered and dissolved in water (100 mL) and EtOAc/MeOH (100 mL, 10 mL). The organic layer separated and the aqueous layer was extracted two times with EtOAc/MeOH (100 mL, 10 mL). The organic extracts were combined and dried with MgSO₄. Filtration and concentrated under reduced pressure afforded a residue. The two regioisomers were separated by flash chromatography (30% EtOAc/5% MeOH/Hex) yielding 45 mg of 3-(1H-indol-2-yl)-7-[3-(4-morphlinyl)propoxy]-2(1H)-quinoxalinone (Example 49, 17%) and 15 mg of 3-(1H-indol-2-yl)-6-[3-(4-morphlinyl)propoxy]-2(1H-quinoxalinone (Example 50, 5%).

### EXAMPLE 49

### 3-(1H-Indol-2-yl)-7-[3-(4-morphlinyl)propoxy]-2(1H)-quinoxalinone

¹H-NMR (DMSO-d₆) δ 12.61 (s, 1H), 12.09 (s, 1H), 8.21 (s, 1H), 7.88 (s, 1H), 7.65-7.60 (d, 1H), 7.57-7.47 (d, 1H), 7.37-7.22 (m, 2H), 7.20-7.16 (m, 1H), 4.14-4.01 (t, 2H), 3.55 (s, 1H), 2.58-2.20 (m, 6H), 1.97-1.81 (t, 2H); LCMS RT=2.11 min; [M+H]⁺=430.

### EXAMPLE 50

### 3-(1H-Indol-2-yl)-7-[3-(4-morphlinyl)propoxy]-2(1H)-quinoxalinone

¹H-NMR (DMSO-d₆) δ 12.61 (s, 1H), 12.09 (s, 1H), 8.21 (s, 1H), 7.82 (s, 1H), 7.78-7.73 (d, 1H), 7.64-7.58 (d, 1H), 7.52-7.43 (d, 1H), 6.98-6.91 (d, 1H), 6.79 (s, 1H), 4.14-4.01 (t, 2H), 3.55 (s, 1H), 2.58-2.20 (m, 6H), 1.97-1.81 (m, 2H); LCMS RT=2.21 min; [M+H]³⁰ =430.2.

### EXAMPLE 56

### 3-amino-2-(3-oxo-3,4-dihydro-quinoxalin-2-yl)-1H-indole-5-carboxylic acid (2-methoxy-ethyl)-methyl-amide

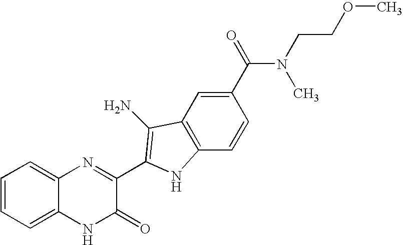

In a 500 mL round bottomed flask was placed 3-nitro-2-(3-oxo-3,4-dihydro-2-quinoxalinyl)-1H-indole-5-carboxylic acid (3.77 g, 10.8 mmol, 1 equiv, Example 22) in 250 mL of DMF To this was added 1.65 mL of triethylamine (11.8 mmol, 1.1 equiv). Upon dissolution of all solids, 6.16 g (11.8 mmol, 1.1 equiv) of PyBOP® was added. After stirring for 5 minutes at room temperature, (2-methoxy-ethyl)-methyl-amine (1.06 g, 11.8 mmol, 1.1 equiv) was added and the mixture allowed to stir overnight (17 h). At this point, the mixture was placed under low vacuum (˜10 min) and back filled with dry argon. To this was added 377 mg of 10% Pd/C (dry), the atmosphere removed under vacuum and converted to one of hydrogen. The reduction was followed via HPLC, where after consumption of the starting material, the Pd was removed by filtration under a blanket of argon. The filtrate was evaporated to dryness and the residue purified via HPLC (5-85% 0.1% TFA CH₃CN/0.1% TFA water). The desired fractions were combined and the CH₃CN removed in vacuo. The remaining aqueous solution was then basified with saturated NaHCO₃ and extracted with EtOAc (1×350 mL). The organic was separated, rinsed with water (100 mL) and then brine (100 mL), dried over Na₂SO₄, filtered, and evaporated. To the red solid was added 75 mL of hot water and the solids sonicated and then filtered to provide 2.77 g (66%) of the desired product as a red solid. ¹H-NMR (DMSO-d₆) δ 12.43 (br s, 1H), 10.79 (br s, 1H), 7.94 (s, 1H), 7.73 (d, 1H), 7.46 (d, 1H), 7.33-7.28 (m, 1H), 7.26-7.18 (m, 3H), 7.10 (br s, 1H); LCMS RT=2.11 min; [M+H]=392.2; EA Calcd C 64.44; H 5.41; N 17.89, Found C 64.18, H 5.19, N 17.70.

### EXAMPLE 104

### Preparation of 3-acetylamino-2-(3-oxo-3,4-dihydro-quinoxalin-2-yl)-1H-indole-5-carboxylic acid (2-methoxy-ethyl)-methyl-amide

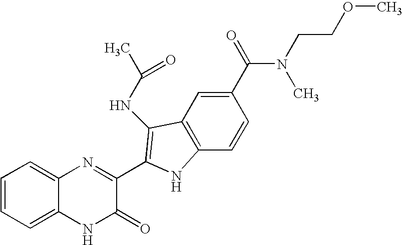

In a 50 mL round bottom flask was placed 52.0 mg (0.13 mmol, 1 equiv) of 3-amino-2-(3-oxo-3,4-dihydro-quinoxalin-2-yl)-1H-indole-5-carboxylic acid (2-methoxy-ethyl)methyl-amide (Example 56) in 5 mL of THF. To this was added 12.6 mg (0.16 mmol, 0.013 ml, 1.2 equiv) of pyridine and 11.5 mg (0.15 mmol, 0.010 mL, 1.1 equiv) of acetyl chloride. This was allowed to stir at room temperature for 72 h. The mixture was then diluted with 40 mL of water and 50 mL of brine and transferred to a separatory funnel. This mixture was then extracted with EtOAc (3×75 mL). The combined organics were dried (Na₂SO₄), filtered, and evaporated to provide 45 mg (78%) of the pure desired product as an orange solid. ¹H-NMR (DMSO-d₆) δ 12.82 (s, 1H), 11.72 (s, 1H), 10.73 (s, 1H), 7.93 (s, 1H), 7.87 (d, 1H), 7.62 (d, 1H), 7.53 (dt, 1H), 7.37 (dt, 2H), 7.23 (d, 1H), 3.70-3.11 (br m, 7H), 3.01 (s, 3H), 2.22 (s, 3H); LCMS RT=2.63 min; [M+H]=434.14.

### EXAMPLE 134

### Preparation of 3-amino-2-(6,7-dichloro-3-oxo-3,4-dihydro-2-quinoxalinyl)-N-[2-(diethylamino)ethyl]-N-methyl-1H-indole-5-carboxamide

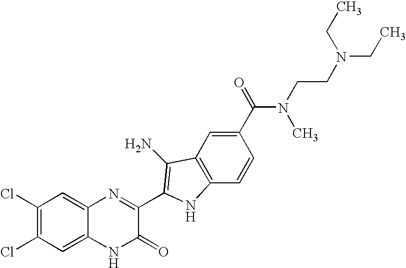

In a 25 mL flask was placed 2-(6,7-dichloro-3-oxo-3,4-dihydro-2-quinoxalinyl)-3-nitro-1H-indole-5-carboxylic acid (0.100 g, 0.239 mmol), DMF (5 mL), and Et₃N (0.037 mL, 0.262 mmol). To this solution was added PyBOP (0.137 g, 0.262 mmol) and then N-[2-(diethylamino)ethyl]-N-methylamine (0.034 g, 0.262 mmol). The mixture was allowed to stir at rt overnight. SnCl₂ (0.226 g, 1.913 mmol) was added and the mixture was stirred at 80° C. for 4 h. The mixture was filtered and concentrated. The residue was taken up in 30 mL of water and extracted with EtOAc (3×20 mL). The organics were concentrated and the residue was purified by preparative HPLC (CH₃CN/H₂O 0.1% TFA). ¹H NMR (400 MHz, DMSO) δ 12.49 (s, 1H), 10.71 (s, 1H), 8.10 (s, 1H), 7.98 (s, 1H), 7.44 (d, J=8.8 Hz, 2H), 7.34 (s, 1H), 7.30 (s, 1H), 7.21 (d, J=9.6 Hz, 2H), 3.44 (bs, 2H), 2.99 (s, 3H), 2.33 (bs, 2H), 0.93 (br d, 6H); LCMS RT=2.47 min; [M+H]=501.1.

### EXAMPLE 151

### Preparation of N-((3R)-1-{[3-amino-2-(3-oxo-3,4-dihydroquinoxalin-2-yl)-1H-indol-5-yl]carbonyl}pyrrolidin-3-yl)acetamide

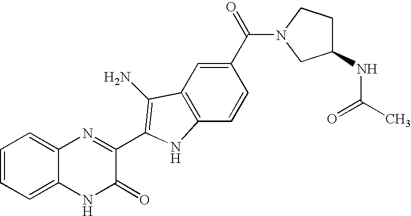

### Step 1. Preparation of 3-(5-{[(3R)-3-aminopyrrolidin-1-yl]carbonyl}-3-nitro-1H-indol-2-yl)quinoxalin-2(1H)-one

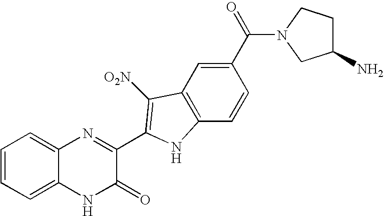

To a solution of tert-butyl ((3R)-1-{[3-nitro-2-(3-oxo-3,4dihydroquinoxalin-2-yl)-1H-indol-5-yl]carbonyl}pyrrolidin-3-yl)carbamate (Prepared using the experimental method described to produce Example 56, 0.40 g, 0.77 mmol) in CH₂Cl₂ (5 mL was added TFA (1 mL). The resulting red solution was stirred at rt for 3 h before the volatiles were removed and Et₂O was added. The volatiles were removed to provide a yellow crude residue. To this residue was added Et₂O and the mixture was sonicated. The precipitated yellow solid was filtered and washed with Et₂O before being dried in an oven to provide 360 mg of a yellow solid (88%). This material was used in next step reaction without purification.

### Step 2. Preparation of N-((3R)-1-{[3-nitro-2-(3-oxo-3,4-dihydroquinoxalin-2-yl)-1H-indol-5-yl]carbonyl}pyrrolidin-3-yl)acetamide

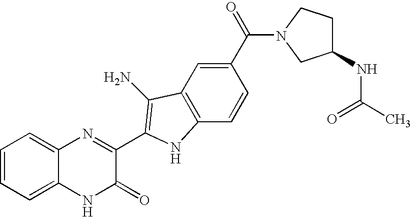

In a 50 mL rb flask was placed 3-(5-{[(3R)-3-aminopyrrolidin-1-yl]carbonyl}-3-nitro-1H-indol-2-yl)quinoxalin-2(1H)-one (0.10 g, 0.19 mmol) in DMF (5 mL). To this solution was added AcCl (0.015 g, 0.19 mmol) and the mixture was allowed to stir for 3 h at rt. Pd/C was added and the atmosphere was converted to H₂ before the reaction was stirred for 3 h. The resulting red solution was filtered and concentrated providing a residue that was purified via HPLC (CH₃CN/water=15-80%) to yield 14.6 mg of a red solid (18%). ¹H-NMR (DMSO-d₆) δ 12.44 (s, 1H), 10.82 (s, 1H), 8.17 (s, 1H), 8.06 (s, 1H), 7.75-7.71 (d, 1H), 7.47-7.43 (d, 1H), 7.39-7.35 (d, 1H), 7.32-7.28 (m, 1H), 7.25-7.21 (m, 2H), 7.13 (s, 2H), 4.13 (s, 1H), 3.80 (s, 1H), 3.70-3.49 (m, 3H), 2.11-2.01 (m, 1H), 1.85-1.74 (m, 4H). LCMS RT=1.97 min; [M+H]⁺=431.0.

### EXAMPLE 152

### Preparation of tert-butyl 5-(4-cyanophenyl)-1H-indole-1-carboxylate

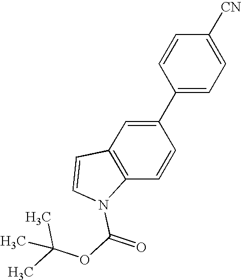

### Step 1. Preparation of 4-(1H-indol-5-yl)benzonitrile

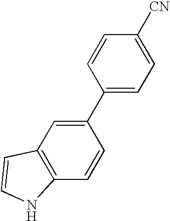

N₂ was bubbled through a solution of 5-indolylboronic acid (1.50 g, 9.32 mmol) in DME (55 mL) for 10 min. To this solution was added 1,1′-bis-(diphenylphosphine-ferrocene) dichloropalladium (II) complex with CH₂Cl₂ (1:1) (0.382 g, 0.440 mmol), 1.0M solution of Na₂CO₃ (22 ml, 22 mmol) and 4-bromobenzonitrile (1.60 g, 8.87 mmol). N₂ was then bubbled through the reaction mixture for 10 min before the mixture was heated at 60° C. for 1 h. The reaction was quenched with H₂O and extracted with EtOAc (3×). The combined organic layers were washed with H₂O, brine, dried (MgSO₄), and concentrated to provide 2.24 g of crude brownish solid residue which was used in next step reaction without purification. ¹H-NMR (DMSO-d₆) δ 11.24 (s 1H), 7.91 (s, 1H), 7.85 (s, 4H), 7.47-7.45 (m, 2H), 7.39 (d, 1H), 6.49 (d, 1H).

### Step 2. Preparation of tert-butyl 5-(4-cyanophenyl)-1H-indole-1-carboxylate

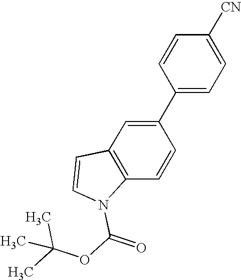

In a 100 mL rb flask was placed 4(1H-indol-5-yl)benzonitrile (2.24 g, 10.3 mmol) in 100 mL of anhydrous THF. To this solution was added DMAP (0.630 g, 5.13 mmol) and the mixture allowed to stir for 0.5 h at rt. Boc₂O (2.24 g, 10.3 mmol) was added and the reaction stirred for 2 h. The reaction was then quenched with H₂O and extracted with Et₂O (2×). The combined organic layers were washed with 1N HCl, H₂O (2×), brine, dried (MgSO₄), and concentrated to provide 2.20 g (67%) of an off-white solid. ¹H-NMR (DMSO-d₆) δ 8.13-8.11 (d, 1H), 8.00 (s, 1H), 7.90 (s, 4H), 7.72-7.68 (m, 2H), 6.77 (d, 1H), 1.63 (s, 9H).

### EXAMPLE 155

### Preparation of 343-amino-5-[(4-phenylpiperidin-1-yl)carbonyl]-1H-indol-2-yl]quinoxalin-2(1H)-one

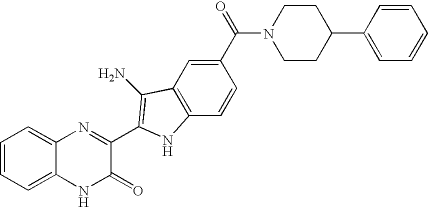

To a solution of SOCl₂ (20.0 mL, 272 mmol) was added 3-nitro-2-(3-oxo-3,4-dihydroquinoxalin-2-yl)-1H-indole-5-carboxylic acid (Example 22, 250 mg, 0.710 mmol) at rt and the resulting brown suspension was heated at 85° C. for 4 h. The suspension was concentrated under reduced pressure and the residue dried for 24 h in vacuo to give 262 mg of light yellow solid. The crude acid chloride was used without further purification. The solid was suspended in anhydrous CH₂Cl₂ (30 mL) and 4-phenylpiperidine (128 mg, 0.780 mmol) was added at rt followed by Et₃N (0.110 mL, 0.780 mmol). The reaction becomes a clear solution after a few minutes and it was stirred at rt for 24 h. To the solution was added 10% Pd/C (50 mg) and the reaction hydrogenated at 1 atm and rt for 2 h. The reaction was diluted with DMF (100 mL) to dissolve the red precipitate (product) then quenched by addition of sat. NH₄Cl (200 mL). The mixture was extracted with EtOAc (2×200 mL) and the organics dried (Na₂SO₄). The solution was filtered and concentrated in vacuo to give a red residue. The crude product was dissolved in DMF and purified by reverse-phase prep-HPLC. Desired fractions were diluted in EtOAc (150 mL) and washed with sat. NaHCO₃ (100 mL). The organics were dried (Na₂SO₄), filtered, and concentrated in vacuo to give a red solid. This was suspended in CH₂Cl₂ and hexane, sonicated, and filtered washing with hexane to give the product as a brick red solid powder in 9% yield (30 mg, 0.065 mmol) after drying. TLC: R_{f}=0.40 (66% EtOAc/hexane; LC-MS (ESI): [M+H]⁺=464.2 @ RT=2.87 min.; ¹H NMR (DMSO-d₆) δ 12.43 (1H, s), 10.81 (1H, s), 7.98 (1H, s), 7.73 (1H, d, J=9.2 Hz), 7.48 (1H, d, J=8.8 Hz), 7.15-7.35 (9H, m), 4.25 (2H, v bs), 3.00 (2H, bs), 2.83 (1H, m), 1.80 (2H, m), 1.64 (2H, m).

### EXAMPLE 156

### Preparation of 3-[3-amino-5-(morpholin-4-ylmethyl)-1H-indol-2-yl]quinoxalin-2(1H)-one

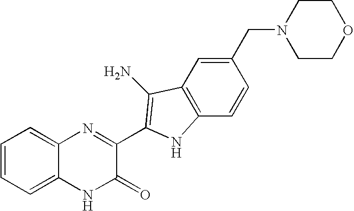

### Step 1. Preparation of 3-[3-amino-5-(hydroxymethyl)-1H-indol-2-yl]quinoxalin-2(1H)-one

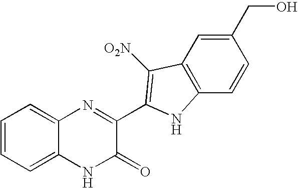

To a solution of 3-nitro-2-(3-oxo-3,4dihydroquinoxalin-2-yl)-1H-indole-5-carboxylic acid (Example 22, 4.36 g, 12.3 mmol) in anhydrous DMF (500 mL) at rt was added CDI (3.03 g, 18.5 mmol) and the dark amber solution stirred at rt for 48 h. The reaction was concentrated under reduced pressure at 30° C. to a volume of 200 mL, then diluted with anhydrous THF (100 mL). The reaction was cooled to 0° C. in an ice bath and vigorously stirred as a rt solution of NaBH₄ (980 mg, 24.64 mmol) in H₂O (100 ml) was added. The reaction, which evolves gas for the first minute, was stirred at 0° C. for 40 min, then quenched with conc. HCl (50 mL) added over 2 min. The mixture was stirred in the ice bath for 5 min, then added portionwise to a stirring solution of sat. NaHCO₃ (1 L) at rt over 10 min. This was extracted with EtOAc (3×1 L). A yellow precipitate was filtered from the biphase and washed with water then EtOAc. The organics were dried (Na₂SO₄), then filtered and concentrated to a volume of approx. 50 mL (DMF). This was diluted with 1:1 MeOH/EtOAc (300 mL), sonicated for 30 min, then let sit for 24 h. The yellow precipitate was filtered washing with EtOAc then hexane. The two precipitates were combined and dried in vacuo under P₂O₅ to give the product as a yellow solid in 64% yield (2.74 g, 8.16 mmol). TLC: R_{f}=0.69 (EtOAc); LC-MS (ESI): [M+H]⁺=337.0 @ RT=2.13 min.; ¹H NMR (DMSO-d₆) δ 12.80 (2H, v bs), 8.04 (1H, s), 7.85 (1H, d, J=8.0 Hz), 7.63 (1H, m), 7.52 (1H, d, J=8.4 Hz), 7.38 (2H, m), 7.29 (1H, d, J=7.2 Hz), 5.29 (1H, t, J=5.6 Hz), 4.64 (2H, d, J=5.2 Hz).

### Step 2. Preparation of 3-[3-nitro-5-(bromomethyl)-1H-indol-2-yl]quinoxalin-2(1H)-one

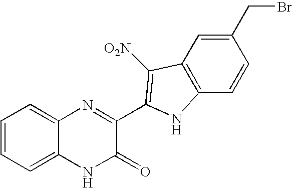

To a solution of 3-[3-nitro-5-(hydroxymethyl)-1H-indol-2-yl]quinoxalin-2(1H)-one (3.04 g, 8.94 mmol) in anhydrous DMF (15.0 mL, 195 mmol) was added anhydrous CH₂Cl₂ (30 mL), followed by SOBr₂ (17.5 mL, 223 mmol) at ambient temp. over 1 min. The reaction becomes hot and bubbles vigorously for several minutes. The mixture was stirred at ambient temp. for 1 h during which time the reaction becomes a dark gray solution. The reaction was poured into CH₂Cl₂ (1.5 L) and carefully quenched with sat. NaHCO₃ (1.7 L). The reaction temp. was kept below 25° C. and the final pH is 7.5. The yellow precipitate which forms in the biphase aqueous layer during the quench was filtered off, washed with H₂O (3×50 mL). The aqueous was extracted with CH₂Cl₂ (1 L), and the combined organics dried (Na₂SO₄), filtered, and concentrated in vacuo to a yellow semi-suspension in the remaining DMF (5 mL). This was diluted with CH₂Cl₂ (10 mL) and copious amounts of hexane was added (200 mL) to give a yellow precipitate. The solid was filtered, washed with hexane, added to the precipitate obtained above, and the combined solids dried in vacuo under P₂O₅ to give the product as a yellow solid (2.81 g, 75%). The crude bromide was used without further purification. TLC: R_{f}=0.35 (66% EtOAc/hexane); LC-MS (ESI): [M+H]⁺=398.9/400.8 @ RT=2.85 min.

### Step 3. Preparation 3-[5-(morpholin-4-ylmethyl)-3-nitro-1H-indol-2-yl]quinoxalin-2(1H)-one

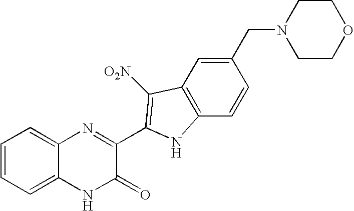

To a solution of the crude 3-[3-amino-5-(bromomethyl)-1H-indol-2-yl]quinoxalin-2(1H)one (100 mg, 0.250 mmol) in anhydrous DMF (1.5 mL) at rt was added morpholine (1.00 mL, 11.5 mmol) and the amber solution stirred at rt for 5 h. The reaction was quenched with sat. NH₄Cl (200 mL) and extracted with EtOAc (3×250 mL). The combined organics were dried (Na₂SO₄), filtered, then concentrated in vacuo to give a yellow oil. This was purified by silica gel chromatography (10% MeOH/EtOAc) to give the product as a yellow solid in 99% yield (105 mg, 0.250 mmol). TLC: R_{f}=0.33 (5% MeOH/EtOAc); LC-MS (ESI): [M+H]⁺=406.0 @ RT=1.73 min.; ¹H NMR (DMSO-d₆) δ 13.11 (1H, bs), 12.82 (1H, bs), 8.02 (1H, s), 7.87 (1H, d, J=8.0 Hz), 7.65 (1H, t, J=7.2 Hz), 7.56 (1H, d, J=8.4 Hz), 7.38 (3H, m), 3.62 (2H, s), 3.57 (4H, m), 2.34 (4H, m).

### Step 4. Preparation of 3-[3-amino-5-(morpholin-4-ylmethyl)-1H-indol-2-yl quinoxalin-2(1H)-one

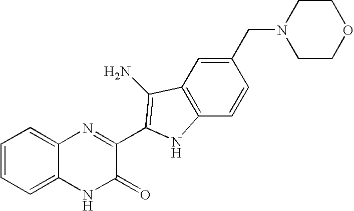

To a solution of 3-[5-(morpholin-4-ylmethyl)-3-nitro-1H-indol-2-yl]quinoxalin-2(1H)-one (100 mg, 0.240 mmol) in anhydrous DMF (5 mL) at rt was added 10% Pd/C (10 mg). The reaction was hydrogenated at 1 atm for 1 h. The mixture was purified directly by silica gel chromatography (10% MeOH/EtOAc) to give the product as a brick red solid in 28% yield (78 mg, 0.21 mmol). TLC: R_{f}=0.55 (EtOAc); LC-MS (ESI): [M+H]⁺=375.8@ RT=1.51 min.; ¹H NMR (DMSO-d₆) δ 12.38 (1H, s), 10.53 (1H, s), 7.70 (2H, m), 7.37 (1H, d, J=8.4 Hz), 7.28 (1H, m), 7.22 (2H, m), 7.11 (1H, d, J=8.4 Hz), 7.01 (2H, s), 3.56 (4H, m), 3.47 (2H, s), 2.35 (4H, bs).

### EXAMPLE 158

### Preparation of 1-(methylsulfonyl)piperazine

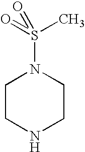

### Step 1. Preparation of tert-butyl 4-(methylsulfonyl)piperazine-1-carboxylate

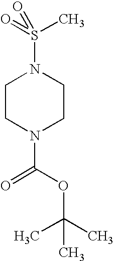

To a solution of tert-butyl piperazine-1-carboxylate (0.60 g, 3.2 mmol) in CH₂Cl₂ (10 mL) was added Et₃N (0.65 g, 6.4 mmol). The mixture was stirred for 10 min before methanesulfonyl chloride (0.40 g, 3.5 mmol) was added and the mixture allowed to stir overnight at rt. The reaction was quenched with H₂O and extracted with CH₂Cl₂ (2×). The combined organic layers were washed with H₂O, brine, dried (MgSO₄), filtered and concentrated to provide 0.80 g of an off-white solid (93%). ¹H-NMR (DMSO-d₆) δ 3.41-3.38 (t, 4H), 3.08-3.04 (t, 4H), 2.85 (s, 3H), 1.40 (s, 9H).

### Step 2. Preparation of 1-(methylsulfonyl)piperazine

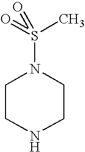

To a solution of tert-butyl 4-(methylsulfonyl)piperazine-1-carboxylate (0.80 g, 3.0 mmol) in CH₂Cl₂ (10 mL) was added TFA (1 mL). The mixture was stirred at rt for 3 h before the volatiles were removed. Et₂O was added to the residue then removed in vacuo to provide a yellow residue. Et₂O was added and the mixture was sonicated. The white solid precipitate was filtered, washed with Et₂O, and dried in an oven to provide 530 mg of an off-white solid (64%). ¹H-NMR (DMSO-d₆) δ 9.06 (s, 2H), 3.34-3.31 (m, 4H), 3.21-3.18 (m, 4H), 2.98 (s, 3H). LCMS [M+H]⁺=165.1.

### EXAMPLE 160

### Preparation of 3-{3-amino-5-[(2-methoxyethoxy)methyl]-1H-indol-2-yl}quinoxalin-2(1H)-one

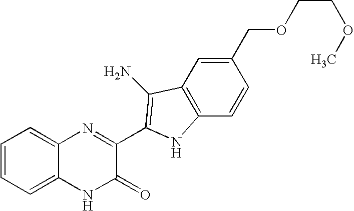

To a suspension of the crude 3-[3-amino-5-(bromomethyl)-1H-indol-2-yl]quinoxalin-2(1H)-one (see steps 1-2, example 156; 1.50 g, 3.76 mmol) in 2-methoxyethanol (29.4 mL, 372 mmol) at rt was added the minimal amount of anhydrous DMF to give a clear solution (35 mL). To this was added K₂CO₃ powder (1.56 g, 11.3 mmol) and additional DMF (10 mL). The mixture was stirred at rt for 18 h. The reaction was filtered to remove K₂CO₃ and concentrated to an oily residue. This was redissolved in DMF (5 mL) and refiltered to remove more K₂CO₃ solids. The solvent was again removed in vacuo and the gum dried in vacuo for 3 h to remove all solvents. The ether-coupled intermediate was dissolved in anhydrous DMF (60 mL) and 10% Pd/C added (3.0 g). This was hydrogenated at 1 atm for 45 min. The red reaction solution was filtered to remove Pd/C, washing with MeOH (200 mL), then concentrated to a volume of 50 mL (DMF). This was refiltered to remove traces of Pd/C and more precipitated K₂CO₃. The filtrate was concentrated further to a volume of 30 mL DMF and purified by reverse-phase prep-HPLC. Desired fractions were diluted in EtOAc (1 L) and washed with sat. NaHCO₃ (500 mL). The organic layer was dried (Na₂SO₄), filtered, and concentrated in vacuo to give a red solid. This was suspended in CH₂Cl₂ (150 mL) and diluted with hexane (200 mL). The brick-red solid was filtered, washed with hexane, and dried in vacuo under P₂O₅ to give the product in 29% yield (400 mg, 1.10 mmol). TLC: R_{f}=0.70 (EtOAc); LC-MS (ESI): [M+H]⁺=365.1 @ RT=2.14 min.; ¹H NMR (DMSO-d₆) δ 12.40 (1H, s), 10.60 (1H, s), 7.73 (2H, m), 7.41 (1H, d, J=8.4 Hz), 7.29 (1H, m), 7.24 (2H, m), 7.13 (1H, dd, J=1.2, 8.4 Hz), 7.04 (2H, s), 4.49 (2H, s), 3.55 (2H, m), 3.49 (2H, m), 3.25 (3H, s).

### EXAMPLE 196

### Preparation of 3-amino-N-[(4methoxyphenyl)sulfonyl]-2-(3-oxo-3,4-dihydroquinoxalin-2-yl)-1H-indole-5-carboxamide

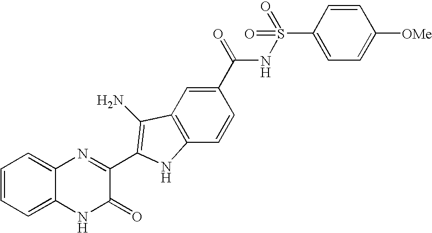

To a 25 mL rb flask was added 3-nitro-2-(3-oxo-3,4-dihydroquinoxalin-2-yl)-1H-indole-5-carboxylic acid (Example 22, 0.100 g, 0.285 mmol), DMF (5 mL), 4-methoxybenzenesulfonamide (0.059 g, 0.314 mmol), DMAP (0.038 g, 0.314 mmol) followed by EDCI (0.060 g, 0.314 mmol). The reaction mixture was allowed to stir at ambient temperature for 4 h before the flask was purged with argon. Pd/C (0.250 g) was added to the flask and a balloon was fitted with hydrogen and the flask was purged (3×). The hydrogenation was allowed to stir at rt for 12 h. The reaction mixture was then filtered and purified by HPLC. The desired fractions were then combined, sat. NaHCO₃ was added (5 mL), and the mixture was extracted with EtOAc (3×50 mL). The combined organics were dried over Na₂SO₄ and then concentrated to provide a red solid. ¹H NMR (400 MHz, DMSO) δ 12.43 (s, 1H), 10.90 (s, 1H) 8.5 (s, 1H), 7.93 (d, J=8.8 Hz, 2H), 7.70 (d, J=8.8 Hz), 7.45 (d, J=9.2), 7.35 (m, 3H), 7.29 (m, 3H),7.25 (bs, 2H), 7.09 (d, J=8.4), 3.85 (s, 3H). LCMS RT=2.77 min; [M+H]⁺=490.1.

### EXAMPLE 214

### Preparation of N-(1-{[3-amino-2-(3-oxo-3,4-dihydroquinoxalin-2-yl)-1H-indol-5-yl]carbonyl}pyrrolidin-3-yl)-N′-isopropyl-N-methylurea

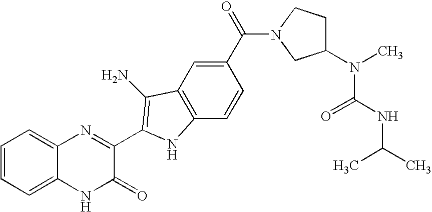

### Step 1. Preparation of 3-(5-{[3-(methylamino)pyrrolidin-1-yl]carbonyl}-3-nitro-1H-indol-2-yl)quinoxalin-2(1 H)-one

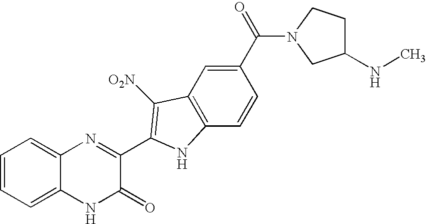

To a solution of tert-butyl methyl(1-{[3-nitro-2-(3-oxo-3,4-dihydroquinoxalin-2-yl)-1H-indol-5-yl]carbonyl}pyrrolidin-3-yl)carbamate (Prepared using the experimental method described to produce Example 56, 0.70 9, 0.77 mmol) in CH₂Cl₂ (10 mL) was added TFA (10 mL), and the resulting red solution was stirred at rt overnight. The volatiles were evaporated and ethyl ether was added. The volatiles were evaporated again to provide a crude yellow residue. This residue was basified with saturated NaHCO₃ to pH 9. The precipitated yellow solid was filtered, washed with water, and dried in an oven to provide 471 mg of a yellow solid (83%). This material was used without further purification.

### Step 2. Preparation of N-(1-{[3-amino-2-(3-oxo-3.4-dihydroquinoxalin-2-yl)-1H-indol-5-yl]carbonyl}pyrrolidin-3-yl)-N′-isopropyl-N-methylurea

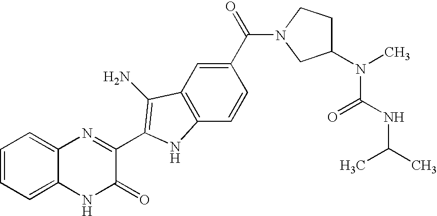

In a 25 mL rb flask was placed 3-(5-{[3-(methylamino)pyrrolidin-1-yl]carbonyl}-3-nitro-1H-indol-2-yl)quinoxalin-2(1H)-one (0.10 g, 0.23 mmol) in toluene (10 mL). To this suspension was added isopropyl isocyanate (0.020 g, 0.23 mmol) and the mixture was allowed to stir overnight at reflux. The solvent was evaporated and to the residue was added ether followed by sonication. The precipitated solid was filtered, washed with ether, and dried in an oven to provide desired a yellow solid. This crude yellow solid was dissolved in DMF (5 mL) and to this solution was added Pd/C. The atmosphere was converted to hydrogen and the reaction was stirred for 3 h. The resulting red solution was filtered and the Pd residue was washed with DMF. The red solution was concentrated and residue was purified via HPLC (MeCN/water=15-80%). The fractions were combined and evaporated to remove acetonitrile. The red solution was basified (saturated NaHCO₃) and the red precipitate was filtered, washed with water (5×), and dried in the oven to provide 66 mg of a red solid (59%). ¹H-NMR (DMSO-d₆) δ 12.43 (s, 1H), 10.81 (s, 1H), 8.12 (s, 1H), 7.76-7.73 (d, 1H), 7.49-7.43 (d, 1H), 7.40-7.35 (m, 1H), 7.33-7.29 (m, 1H), 7.28-7.21 (m, 2H), 7.15 (s, 2H), 6.02 (s, 1H), 3.81-3.39 (m, 5H), 2.73 (s, 3H), 1.99-1.87 (m, 2H), 1.09-0.95 (m, 6H). LCMS RT =2.17 min; [M+H]⁺=488.1.

### EXAMPLE 217

### Preparation of 3-[3-amino-5-(3,5-dichloro-pyridin-4-yloxy)-1H-indol-2-yl]-1H-quinoxalin-2-one

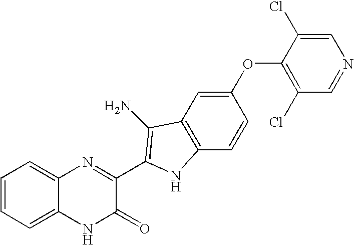

### Step 1. Preparation of 3-(5-Hydroxy-1H-indol-2-yl)-1H-quinoxalin-2-one

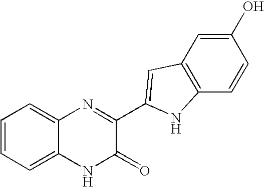

A solution of 3-(5-methoxy-1H-indol-2-yl-1H-quinoxalin-2-one (Example 6, 1.80 g, 6.18 mmol) in CH₂Cl₂ (150 mL) was cooled to 0° C. BBr₃ (5.84 mL, 61.8 mmol) was added to the solution dropwise. The mixture was stirred at rt for 24 h. The reaction was poured onto ice (200 g). The resulting mixture was extracted with EtOAc (3×300 mL). The organic layers were washed with brine (500 mL), dried (MgSO₄) and concentrated under reduced pressure. The residue was crystallized in MeOH and water (1:6) to afford 1.70 g (99%) of product. 1H NMR (400 MHz, DMSO) δ 12.56 (s, 1H), 11.31 (s, 1H), 8.79 (s, 1H), 7.78 (d, J=8.4 Hz, 1H), 7.64 (s, 1H), 7.46 (t, J=7.2 Hz, 1H), 7.30-7.32 (m, 3H), 6.90 (s, 1H), 6.73 (d J=8.4 Hz, 1H); LCMS (ESI-MS) RT=2.27; 276.2 (M+H)⁺.

### Step 2. Preparation of 3-[5-(3,5-Dichloro-pyridin-4-yloxy)-1H-indol-2-yl]-1-quinoxalin-2-one

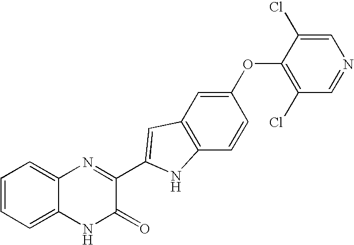

A solution of 3-(5-hydroxy-1H-indol-2-yl)-1H-quinoxalin-2-one (100 mg, 0.36 mmol) and potassium tert-butoxide (44.5 mg, 0.40 mmol) in DMF (2 mL) was stirred at rt for 2 h. To the solution, was added 3, 4, 5-trichloropyridine (65.8 mg, 0.36 mmol) and K₂CO₃ (29.9 mg, 0.22 mmol). The mixture was heated to 100° C. overnight. The reaction was allowed to cool to rt and poured into water (20 mL). The crude product was precipitated as a yellow solid. The solid was filtered, washed with water and dried to give 120 mg (79%) of product. 1H NMR (400 MHz, DMSO) δ 12.60 (b, 1H), 11.69 (s, 1H), 8.75 (s, 2H), 7.81 (d, J=8.8 Hz, 1H), 7.73 (s, 1H), 7.51 (m, 2H), 7.34-7.32 (m, 2H), 7.05 (s, 1H), 6.95 (d J=8.8 Hz, 1H); LCMS (ESI-MS) RT=3.77; 423.2 (M+H)⁺.

### Step 3. Preparation of 3-[5-(3,5-dichloro-pryrindine-4-yloxy)-3-nitro-1H-indol-2-yl]-1H-quinoxalin-2-one

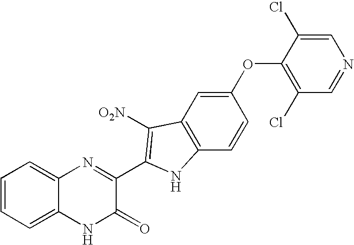

To a solution of 3-[5-(3,5-dichloro-pyridin-4-yloxy)-1H-indol-2-yl]-1H-quinoxalin-2-one (110 mg, 0.26 mmol) in DMF (3 mL) was added isoamyl nitrite (80 uL, 0.57 mmol). The reaction was heated at 90° C. for 2 h, then allowed to cool to rt. The mixture was poured into water (25 mL). The product was precipitated as a yellow solid. The solid was filtered, washed with water and dried to afford 95 mg (62%) of crude product which was used without further purification.

### Step 4. Preparation of 3-[3-Amino-5-(3,5-dichloro-pyridin-4-yloxy)-1H-indol-2-yl]-1H-quinoxalin-2-one

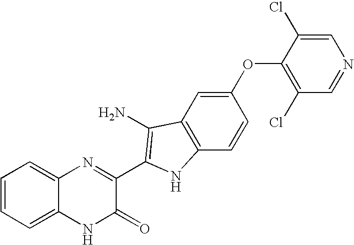

A solution of 3-[5-(3,5-dichloro-pryrindine-4-yloxy)-3-nitro-1H-indol-2-yl]-1H-quinoxalin-2-one (95 mg, 0.16 mmol) in AcOH (2 mL) and water (20 uL) was degassed with nitrogen for 5 min. Activated iron powder (325 mesh, 95 mg, 1.70 mmol) was added and the mixture was stirred at rt overnight. The reaction was neutralized by NaHCO₃ solution (50 mL) and extracted with EtOAc (3×30 mL). The organic layers were washed with brine (50 mL), dried and concentrated. The residue was purified by a silica gel column chromatography (EtOAc: Hexanes=1:1) to afford 35 mg (49%) of desired product. 1H NMR (400 MHz, DMSO) δ 12.45 (s, 1H), 10.66 (s, 1H), 8.78 (s, 2H), 7.73 (d, J=8.8 Hz, 1H), 7.48 (d, J=8.8 Hz, 1H), 7.24-7.00 (m, 5H), 6.85 (s, 2H); LCMS (ESI-MS) RT=2.99; 438.2 (M+H)⁺.

### EXAMPLE 228

### Preparation of 3-(5-{[tert-butyl(dimethyl)silyl]oxy}-1H-indol-2-yl)quinoxalin-2(1H)-one

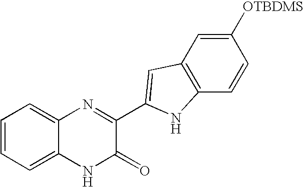

### Step 1. Preparation of 5-{[tert-butyl(dimethyl)silyl]oxy}-1H-indole

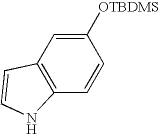

In a 250 mL rb flask was placed 5-hydroxyindole (5.00 g, 37.6 mmol, 1 equiv.) in 75 mL of DMF. To this was added imidazole (2.7 g, 1.05 equiv.) and TBDMSCl (5.90 g, 1.05 equiv.) and the reaction was allowed to stir at room temperature for 2 h. The DMF was removed in vacuo and the residue was partitioned between water (150 mL) and EtOAc (150 mL). The EtOAc was removed and the aqueous extracted (2×100 mL) with EtOAc. The combined organics were dried (Na₂SO₄), filtered, and evaporated to provide 9.2 g of a white solid which was used without further purification.

### Step 2. Preparation of tert-butyl-5-{[tert-butyl(dimethyl)silyl]oxy}-1H-indole-1-carboxylate

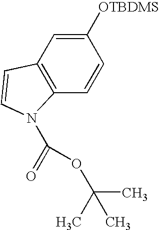

In a 250 mL rb flask was placed 9.3 g (37.6 mmol, 1 equiv) of 5-t-butyldimethylsiloxyindole in 75 mL of THF. To this was added 4-DMAP (4.8 g, 1.05 equiv) and di-t-butyl dicarbonate (8.6 g, 1.05 equiv) after which gas evolution was evident. After gas evolution ceased (5 min.) the reaction appeared complete via TLC. The THF was then removed and the residue partitioned between water (150 mL) and EtOAc (150 mL). The organics were separated, dried (Na₂SO₄), filtered, and evaporated. The residue was then filtered through a silica plug to remove any remaining 4-DMAP. The desired fractions were combined and evaporated to provide 12.2 g of a white solid which was used without further purification.

### Step 3. Preparation of tert-butyl 5-{[tert-butyl(dimethyl)silyl]oxy}-2-[methoxy(oxo)acetyl]-1H-indole-1-carboxylate

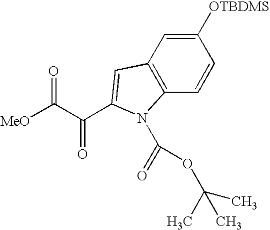

In a 500 mL rb flask was placed N-Boc-5-t-butyldimethylsiloxyindole (12.2 g, 35.1 mmol, 1 equiv) in 100 mL of THF. This was cooled to −78° C. where 24.1 mL of t-BuLi (1.7 M in pentane, 38.6 mmol, 1.1 equiv) was added dropwise. This was allowed to stir for 1 h, where dimethyl oxalate (9.1 g, 2.2 equiv) was added as a solution in 40 mL of THF quickly in one portion. The reaction was then allowed to warm to room temp. and stir for an additional 2 h. At this point, the reaction was diluted with water (200 mL) and extracted with EtOAc (3×150 mL). The combined organics were dried (Na₂SO₄), filtered, and evaporated. The residue was then purified by silica gel chromatography (10% EtOAc/Hex) to provide 8.9 g (58%) as a white solid. ¹H NMR (CD₂Cl₂) δ 7.72 (d, 1H), 7.34 (d, 1H), 6.77 (s, 1H), 6.61 (d, 1H), 6.25 (d, 1H), 1.44 (s, 9H), 0.80 (s,9H), 0.80 (s, 9H), 0.00 (s, 6H); TLC R_{f}=0.60 (25% EtOAc/Hex).

### Step 4. Preparation of 3-(5-{[tert-butyl(dimethyl)silyl[oxy}1H-indol-2-yl)quinoxalin-2(1H)-one

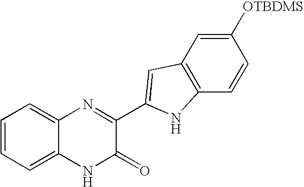

In a 500 mL rb flask was placed N-Boc-5-t-butyldimethylsiloxy-2-methyloxalylindole (8.70 g, 20.0 mmol, 1 equiv) in 250 mL of AcOH. To this was added 1,2-phenylenediamine (2.4 g, 1.1 equiv) and the reaction mixture heated at 130° C. After 1 h, 1.7 mL of TFA (1.1 equiv) was added turning the solution red. After 2 min, the reaction was cooled to room temp. and poured into 60 mL of water resulting in a yellow solid. The solid was filtered and dried in vacuo at 60° C. to provide 7.8 g (99%) of the desired product as a yellow solid. ¹H NMR (DMSO-d₆) δ 12.39 (s, 1H), 11.26 (s, 1H), 7.61 (d, 1H), 7.52 (s, 1H), 7.30 (dt, 1H), 7.20 (d, 1H), 7.14 (m, 2H), 6.85 (d, 1H), 6.57 (dd, 1H), 0.78 (s, 9H), 0.00 (s, 6H); LCMS RT=3.98 min; [M+H]=392.3.

### EXAMPLE 236

### Preparation of 3-[3-amino-5-(2,3-dihydro-1H-tetrazol-5-yl)-1H-indol-2-yl]quinoxalin-2(1H)-one

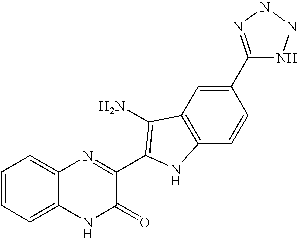

### Step 1. Preparation of 3-[5-(2,3-dihydro-1H-tetrazol-5-yl)-3-nitro-1H-indol-2-yl]quinoxalin-2(1H)-one

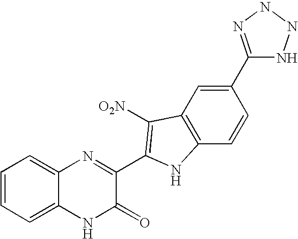

In a 25 ml rb flask was placed 3-[3-nitro-5-cyano-1H-indol-2-yl]-1H-quinoxalin-2-one (Example 19, 150 mg, 0.45 mmol) in 5 ml of DMF. To this was added NaN₃ (58.9 mg, 0.90 mmol) and NH₄Cl (48.4 mg, 0.90mmol) before the reaction was heated at 120° C. After 1 h, only minor product was seen and 1 ml of water was added. Stirring another 1 h produced only a minor change. Additional NaN₃ (176 mg) and NH₄Cl (145 mg) were added and the reaction was allowed to stir over the weekend. At this point, no starting material remained. The solids were filtered off and the majority of volatiles (˜2 ml) removed. The mixture was then diluted with water and filtered to provide 117 mg (69%) of yellow solid that was used without further purification. LCMS Rt=2.22 min; [M+H]=375.0.

### Step 2. Preparation of 3-[3-amino-5-(2,3-dihydro-1H-tetrazol-5-yl)-1H-indol-2-yl]quinoxalin-2(1H)-one

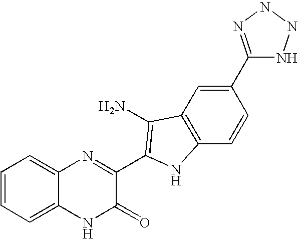

In a 25 ml rb flask was placed 3-[3-nitro-5-(1H-tetrazol-5-yl)-1H-indol-2-yl]-1H-quinoxalin-2-one (117 mg, 0.31 mmol) in 6 ml of DMF. To this was added catalytic 10% Pd/C and the dissolved gases removed under vacuum. The atmosphere was converted to one of H₂ and the reaction was allowed to stir at rt until complete. The Pd/C was then filtered off and the volatiles removed in vacuo. The solids were suspended in CH₃CN, sonicated for 2 minutes, and refiltered to remove any remaining DMF. The desired product was isolated a red solid (84 mg, 82%). ¹H-NMR (DMSO-d₆; tetrazole N—H undescribed) δ 12.47 (s, 1H), 10.93 (s, 1H), 8.59 (s, 1H), 7.76 (d, 2H), 7.60 (s, 1H), 7.32 (t, 1H), 7.25 (t, 2H), 7.19 (br s, 2H). LCMS RT=2.09 min; [M+H]=345.0.

### EXAMPLE 266

### Preparation of 3-(3-hydroxy-1H-indol-2-yl)quinoxalin-2(1H)-one

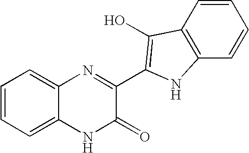

### Step 1. Preparation of 3-{[tert-butyl(dimethyl)silyl[oxy}1H-indole

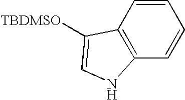

To a 100 mL rb flask was added 3-hydroxyindole (1.00 g, 7.51 mmol) followed by TBDMSCl (11.3 mL, 1.0 M in THF). Imidazole (0.767 g, 11.3 mmol) was added followed by DBU (0.057 g, 0.38 mmol). The mixture was allowed to stir at rt for 18 h before it was concentrated and used without further purification.

### Step 2. Preparation of tert-butyl 3-{[tert-butyl(dimethylsilyl]oxy}-1H-indole-1-carboxylate

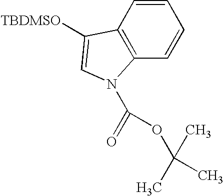

To a 100 mL rb flask was added 3-{[tert-butyl(dimethyl)silyl]oxy}-1H-indole (0.600 g, 2.42 mmol) and by 100 mL of THF followed by DMAP (0.311 g, 2.55 mmol) and di-tert-butyl di-carbonate (0.556 g, 2.55 mmol). The reaction was allowed to stir at rt for 3 h before it was concentrated and used without further purification.

### Step 3. Preparation of tert-butyl 3-{[tert-butyl(dimethyl)silyl]oxy}-2-[methoxy(oxo)acetyl]-1H-indole-1-carboxylate

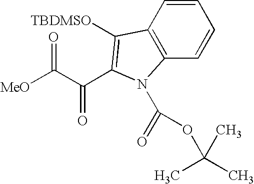

To a 100 mL rb flask was added tert-butyl 3-{[tert-butyl(dimethyl)silyl]oxy}1H-indole-1-carboxylate (0.500 g, 1.44 mol) and 100 ml of THF. The reaction was cooled to −78° C. before t-BuLi (1.7 M in pentane, 0.93 mL, 1.6 mmol) was added slowly over 30 min. The solution was then allowed to stir for 2 h at −78° C. before dimethyl oxalate (0.425 g, 3.60 mmol) was added in one portion. The mixture was allowed to stir at −78° C. for 10 min before it was warmed to 0° C. for 1.5 h. The mixture was quenched with water (100 mL) and concentrated. The residue was purified by column chromatography with Hex/EtOAC (1:1) to provide material that was 80% pure. This material was taken on to the next reaction without further purification.

### Step 4. Preparation of 3-(3-hydroxy-1H-indol-2-yl)quinoxalin-2(1H)-one

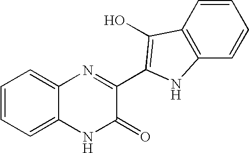

To a 100 mL rb flask was added tert-butyl 3-{[tert-butyl(dimethyl)silyl]oxy}2-[methoxy(oxo)acetyl]-1H-indole-1-carboxylate (0.030 g, 0.090 mmol) followed by 1,2-phenylenediamine (0.010 g, 0.094 mmol) and AcOH (3 mL). The mixture was then heated at 100° C. for 18. The mixture was then concentrated and purified by HPLC. LCMS: RT=2.94 min., [M+H]+=278.7.

### EXAMPLE 304

### Preparation of 3-[3-amino-5-(5-methyl-1,3,4-oxadiazol-2-yl)-1H-indol-2-yl]quinoxalin-2(1H)-one

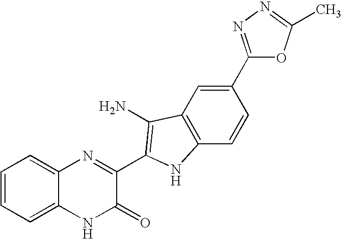

To a 30 mL rb flask was added N′-acetyl-3-nitro-2-(3-oxo-3,4dihydroquinoxalin-2-yl)-1H-indole-5-carbohydrazide carbamate (Prepared using the experimental method described to produce Example 56, 0.200 g, 0.492 mmol) followed by 5 mL of DMF/Benzene (1/1). P₂O₅ (0.284 g, 2.46 mmol) was added and the reaction was heated at 100° C. for 18 h. The mixture was cooled to rt before 10% Pd/C (100 mg) was added and the atmosphere was converted to H₂. The mixture was stirred overnight before it was diluted with water (30 mL) and extracted with EtOAc (3×30 mL). The residue was purified by HPLC to provide 0.009 g of a red solid (5%). ¹H NMR (DMSO-d₆) δ 12.49 (s, 1H), 11.06 (s, 1H), 8.55 (s, 1H), 7.70 (m, 1H), 7.62 (d, 1H), 7.33 (s, 1H), 7.25 (m, 1H), 2.58 (s, 3H); LCMS RT=2.32 min; [M+H]=359.3.

### EXAMPLE 307

### Preparation of 3-amino-N-cyclopentyl-N-2-methoxyethyl)-2-(3-oxo-3,4-dihydroquinoxalin-2-yl)-1H-indole-5-carboxamide

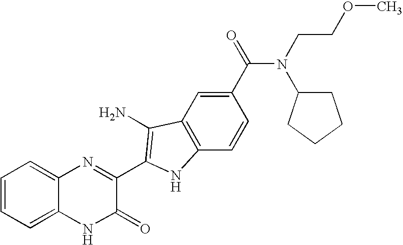

### Step 1. Preparation of N-(2-methoxyethyl)cyclopentylamine

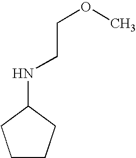

To a solution of cyclopentanone (2.00 g, 23.8 mmol) and 2-methoxyethylamine (1.78 g, 23.8 mmol) in CH₂Cl₂ (10 mL) was added sodium triacetoxyborohydride (7.05 g, 33.3 mmol) followed by AcOH (1.36 mL, 23.8 mmol). The reaction mixture was stirred at rt overnight. The reaction was quenched by adding sat'd NaHCO₃ and extracted with CH₂Cl₂(2×). The combined organic layers were washed with brine, dried (MgSO₄), and concentrated to provide 0.70 g (20.5%) of crude free base as an yellowish oil which was used in next step reaction without further purification. ¹H-NMR (DMSO-d₆) δ 3.37-3.33 (m, 2H), 3.21 (s, 3H), 2.98-2.95 (m, 1H), 2.63-2.58 (t, 2H), 1.71-1.62 (m, 2H), 1.59-1.52 (m, 2H), 1.43-1.38 (m, 2H), 1.27-1.17 (m, 2H). LCMS RT=0.76 min; [M+H]⁺=144.2

### Step 2. Preparation of 3-amino-N-cyclopentyl-N-(2-methoxyethyl)-2-(3-oxo-3,4-dihydroquinoxalin-2-yl)-1H-indole-5-carboxamide

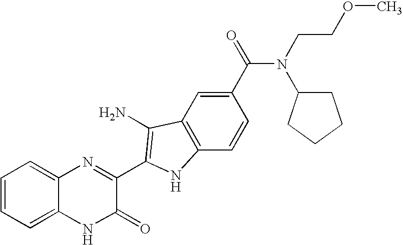

Using the method from Example 56, 3-amino-N-cyclopentyl-N-(2-methoxyethyl)-2-(3-oxo-3,4-dihydroquinoxalin-2-yl)-1H-indole-5-carboxamide was obtained as a red solid (50 mg, 39%) from the product of Step 1, Example 307 and 3-nitro-2-(3-oxo-3,4-dihydro-2-quinoxalinyl)-1H-indole-5-carboxylic acid (Example 22). ¹H-NMR (DMSO-d₆) δ 12.45 (s, 1H), 10.81 (s, 1H), 7.94 (s, 1H), 7.79-7.74 (d, 1H), 7.46-7.41 (d, 1H), 7.38-6.98 (m, 6H), 4.24-4.12 (m, 1H), 3.58-3.20 (m, 7H), 1.82-1.58 (m, 6H), 1.45-1.36 (m, 2H). LCMS RT=2.74 min; [M+H]⁺=446.2.

### EXAMPLE 319

### Preparation of tert-butyl 5-[(2-methoxyethyl)(methyl)amino]-2-[methoxy(oxo)acetyl]-1H-indole-1-carboxylate

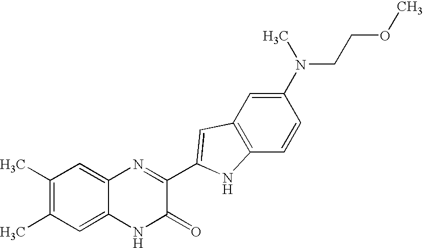

### Step 1. Preparation of N-(2-methoxyethyl)-N-3-dimethyl-4-nitroaniline

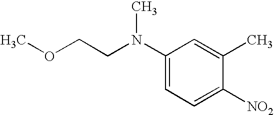

To a round bottom flask equipped with a reflux condenser was added 5-fluoro-2-nitrotoluene (10 g, 65.0 mmol) in 1-methyl-2-pyrrolidine (150 mL). N-(2-methoxyethyl)methylamine (21 mL, 200 mmol) was added to the stirring solution and the reaction was heated at 80° C. for 3 h. After cooling to rt, the product was purified by chromatography to yield 10.5 g (72%) of a yellow solid. ¹H NMR (DMSO-d₆): δ 2.62 (s, 3H), 3.01 (s, 3H), 3.24 (s, 3H), 3.40-3.45 (m, 2H), 3.57-3.61 (m, 2H), 6.58-6.62 (m, 2H).

### Step 2. N-(2-methoxyethyl)-N-methyl-1H-indol-5-amine

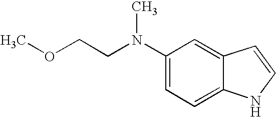

To a round bottom flask equipped with a reflux condenser was charged with N-(2-methoxyethyl)-N-3-dimethyl-4-nitroaniline (9.5 g, 42 mmol) and DMF (200 mL). N,N-dimethylformamide dimethylacetal (6.0 g. 50 mmol) and pyrrolidine (3.6 g, 50 mmol) were added and the reaction was heated at reflux for 3 h. After cooling to rt, the volatile components were removed in vacuo and the oily residue was dissloved in DMF (100 mL). The solution was added to 10% Pd/C (950 mg) under argon. The atmosphere was converted to H₂ with a balloon and the reaction allowed to stir at rt for 17 h. The H₂ was then removed and the mixture filtered through Celite@ under a blanket of argon. The solvents were then removed and the product was purified by chromatography. The desired product was a red oil (7.9 g, 92%). ¹H-NMR (DMSO-d₆) δ 2.80-2.83 (m, 4H), 3.22 (s, 3H), 2.39-2.43 (m, 2H), 2.46-2.52 (m, 2H), 6.22-6.24 (m, 1H), 6.72-6.76 (d, 1H), 6.81 (s, 1H), 7.18-7.23 (m, 2H), 10.61 (brs, 1H); LCMS RT=0.27 min; [M+H]⁺=205.09.

### Step 3. Preparation of tert-butyl 5-[(2-methoxyethyl)(methyl)amino]-1H-indole-1-carboxylate

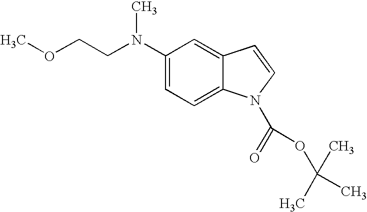

Using the method described in Example 12 Step 1, tert-butyl 5-[(2-methoxyethyl)(methyl)amino]-1H-indole-1-carboxylate was obtained as a coloress solid (7.2 g, 61%) from N-(2-methoxyethyl)-N-methyl-1H-indol-5-amine (7.8 g, 38 mmol). ¹H-NMR (DMSO-d₆) δ 1.63 (s, 9H), 2.90(s, 3H), 3.21 (s, 3H), 3.40-3.45 (m, 4H), 6.43-6.46 (m, 1H), 6.75-6.84 (m, 2H), 7.46-7.50 (d, 1H), 7.78-7.83 (d, 1H).

### Step 4. Preparation of tert-butyl 5-](2-methoxyethyl)(methyl)amino]-2-[methoxy(oxo)acetyl-1H-indole-1-carboxylate

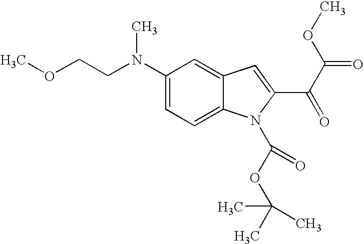

Using the method described in Example 18 Step 2, tert-butyl 5-[(2-methoxyethyl)(methyl)amino]-2-[methoxy(oxo)acetyl]-1H-indole-1-carboxylate was obtained as an oil (3.6 g, 80%) from tert-butyl 5-[(2-methoxyethyl)(methyl)amino]-1H-indole-1-carboxylate (3.5 g, 12 mmol). ¹H-NMR (DMSO-d₆) δ 1.59 (s, 9H), 3.93 (s, 3H), 3.21 (s, 3H), 3.41-3.58 (m, 4H), 6.85 (m, 1H), 7.05-7.13 (d, 1H), 7.25 (s, 1H), 7.75-7.79 (d, 1H).

### Step 5. Preparation of 3-{(2-methoxyethyl)(methyl)amino]-1H-indol-2-yl}-6,7-dimethylquinoxalin-2(1H)-one

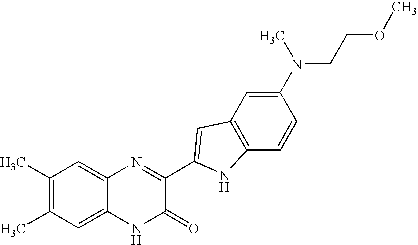

Using the method described in Example 12 Step 3, 3-{5-[(2-methoxyethyl)(methyl)amino]-1H-indol-2-yl}-6,7-dimethylquinoxalin-2(1H)-one was obtained as a red powder (204 mg, 88%) from tert-butyl 5-[(2-methoxyethyl)(methyl)amino]-2-[methoxy(oxo)acetyl]-1H-indole-1-carboxylate (240 mg, 0.62 mmol) and 1,2-diamino-4,5-dimethylbenzene (69 mg, 0.64 mmol). ¹H-NMR (DMSO-d₆) δ2.35 (s, 6H), 2.83 (s, 3H), 3.20 (s, 3H), 3.38-3.51 (m, 4H), 6.80-6.85 (m, 1H), 7.09 (s, 1H), 7.36-7.40 (d, 1H), 7.55-7.61 (1H), 11.19 (s, 1H), 12.41 (s, 1H); LCMS RT=1.86 min; [M+H]⁺=377.46.

### EXAMPLE 336-337

### Preparation of 2-[7-(4-fluorophenyl)-3-oxo-3,4-dihydroquinoxalin-2-yl]-1H-indole-5-carbonitrile and 2-[6-(4-fluorophenyl)-3-oxo-3,4-dihydroquinoxalin-2-yl]-1H-indole-5-carbonitrile

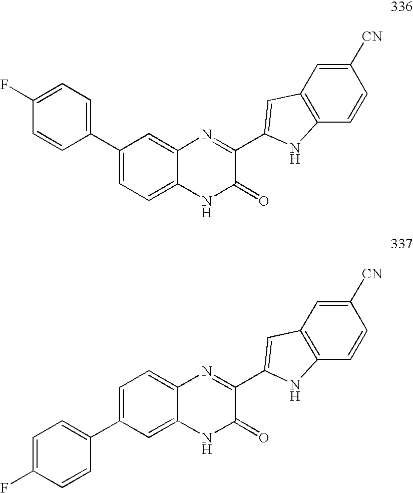

### Step 1. Preparation of (4′-fluoro-3-nitrobiphenyl-4-yl)amine

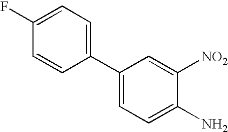

N₂ was bubbled through a solution of 4-bromo-6-nitroaniline (3.0 g, 14 mmol) in DME (25 mL) for 10 min before 1,1′-bis(diphenylphosphino-ferrocene)dichloropalladium (II), complex with CH₂Cl₂ (1:1) (0.60 g, 0.69 mmol), 1.0M solution of Na₂CO₃ (35 mL, 35 mmol), and 4-fluorophenylboronic acid (2.0 g, 15 mmol) were added. The reaction mixture was bubbled with nitrogen for an additional 10 min and then heated at 60° C. for 1 h. The reaction was quenched with water, extracted with EtOAc (3×). The combined organic layers were washed with water, brine, dried (MgSO₄), and concentrated to obtain a crude residue which was chromatograghed with hexane/EtOAc=3/1 to provide 2.8 g (88%) of the product as an orange solid. ¹H-NMR (DMSO-d6) δ 8.16 (s 1H), 7.75-7.73 (d, 1H), 7.68-7.54 (m, 2H), 7.53 (s, 2H), 7.27-7.11 (m, 2H), 7.09-7.08 (d, 1H).

### Step 2. Preparation of 4′-fluorobiphenyl-3,4-diamine

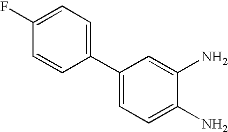

To a dry flask was added 10% Pd/C (0.013 g) under argon. MeOH (100 mL) and (4′-fluoro-3-nitrobiphenyl-4-yl)amine (2.71 g, 11.7 mmol) were added before the atmosphere was converted to hydrogen and the mixture stirred at rt overnight. The reaction mixture was filtered through Celite, washed with MeOH, and concentrated to provide 2.26 g (96%) of a purplish solid. ¹H-NMR (DMSO-d₆) δ 7.47-7.44 (m, 2H), 7.17-7.13 (m, 2H), 6.78 (s, 1H), 6.67-6.64 (d, 1H), 6.55-6.53 (d, 1H), 4.59-4.54 (d, 4H). LCMS RT=1.74 min; [M+H]⁺=203.2.

### Step 3. Preparation of 2-[7-(4-fluorophenyl)-3-oxo-3,4-dihydroquinoxalin-2-yl]-1H-indole-5-carbonitrile and 2-[6-(4-fluorophenyl)-3-oxo-3,4-dihydroquinoxalin-2-yl]-1H-indole-5-carbonitrile

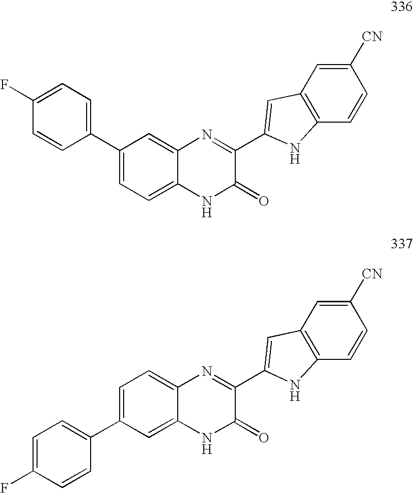

A solution of 4′-fluorobiphenyl-3,4-diamine (2.28 g, 11.3 mmol) and 2-[methoxy(oxo)acetyl]-1H-indole (5) (2.33 g, 10.3 mmol) in AcOH (10 mL) was heated at 100° C. overnight. The reaction mixture was cooled to rt and diluted with water. The precipitant yellow solid was filtered, washed with water (5×), and dried in an oven to provide 3.11 g (80%) of a yellow solid. ¹H-NMR (DMSO-dr) δ 12.83-12.79 (d, 1H), 12.21-12.19 (d, 1H), 8.24 (d, 1H), 8.05-7.64 (m, 6H), 7.55-7.32 (m, 4H). LCMS RT=3.4 [M+H]⁺=381.3.

### EXAMPLE 354

### Preparation of 3-amino-N-(2-methoxyethyl)-N-methyl-2-(3-oxo-3,4-dihydroquinoxalin-2-l)-1H-indole-5-sulfonamide

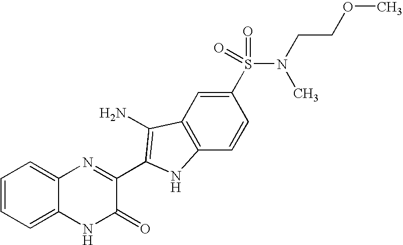

### Step 1. Preparation of 2-(3-chloroquinoxalin-2-yl)-N-(2-methoxyethyl)-N-methyl-1H-indole-5-sulfonamide

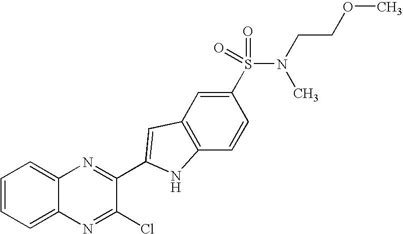

A vial charged with bis(diphenylphosphino)ferrocenepalladium(II)chloride (0.164 g, 0.220 mmol) was added 2,3-dichloroquinoxaline (0.669 g, 3.36 mmol), (5-(2-methoxyethyl)(methyl)amino]sulfonyl}-1 H-indol-2-yl)boronic acid (0.700 g, 2.24 mmol), and NaHCO₃ (0.951 g, 8.97 mmol), followed by DME (5 mL). Water (0.5 mL, bubbled with nitrogen for 10 minutes) was added to the reaction and the mixture was heated to 75 ° C. for 1 h. An additonal 10 mol % of Pd was added and the mixture was stirred 1.5 h. Upon consumption of the boronic acid the reaction was concentrated under vacuum and the residue was purified by HPLC to provide 0.300 g of a brown solid (31%). ¹H NMR (DMSO-d₆) δ 12.39 (s, 1H), 8.22 (d, 1H), 8.20 (dd, 1H), 8.08 (dd, 1H), 7.98-7.89 (m, 2H), 7.74 (dd, 1H), 7.61 (dd, 1H), 3.46 (t, 2H), 3.23 (s, 3H), 3.13 (t, 2H), 2.72 (s, 3H); LCMS RT=3.34 min; [M+H]=431.1.

### Step 2. Preparation of N-(2-methoxyethyl)-N-methyl-2-(3-oxo-3,4-dihydroquinoxalin-2-yl)-1H-indole-5-sulfonamide

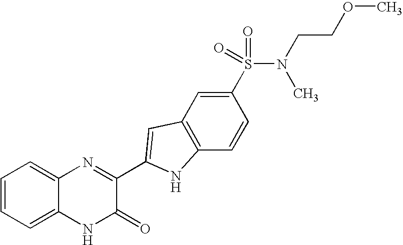

2-(3-chloroquinoxalin-2-yl)-N-(2-methoxyethyl)-N-methyl-1H-indole-5-sulfonamide (0.280 g, 0.650 mmol) was dissolved in AcOH (20 mL) and was heated at reflux (130° C.) for 18 h. The solvent was removed under the vacuum and the residue was taken up in EtOAc. The organic layer was washed with sat. NaHCO₃ and concentrated to afford the product as a red solid (0.250 g, 93%). ¹H NMR (DMSO-d₆) δ 8.15 (s, 1H), 7.98 (s, 1H), 7.83 (dd, 1H), 7.71 (s, 1H), 7.69 (d, 1H), 7.56-7.52 (m, 2H), 7.34 (d, 1H), 3.44 (t, 2H), 3.21 (s, 3H), 3.10 (t, 2H), 2.68 (s, 3H); LCMS RT=2.71 min; [M+H]=413.6.

### Step 3. Preparation of N-(2-methoxyethyl)-N-methyl-3-nitro-2-(3-oxo-3,4- dihydroquinoxalin-2-yl)-1H-indole-5-sulfonamide

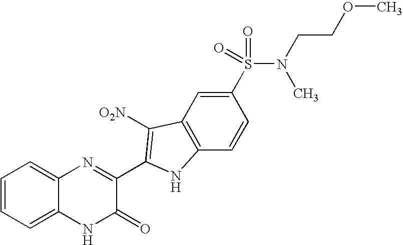

Using the method described in Example 2, N-(2-methoxyethyl)-N-methyl-3-nitro-2-(3-oxo-3,4-dihydroquinoxalin-2-yl)-1H-indole-5-sulfonamide was obtained as a dark yellow solid (0.085 g, 76%) from N-(2-methoxyethyl)-N-methyl-2-(3-oxo-3,4-dihydroquinoxalin-2-yl)-1H-indole-5-sulfonamide (0.100 g, 0.240 mmol). This material was taken on without purification or characterization.

### Step 4. Preparation of 3-amino-N-(2-methoxyethyl)-N-methyl-2-(3-oxo-3,4-dihydroquinoxalin-2-yl)-1H-indole-5-sulfonamide

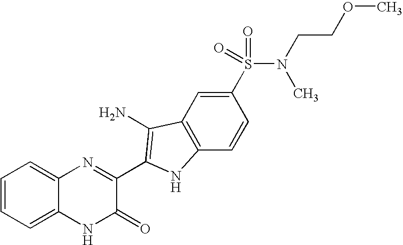

Using the method described in Example 3, N-(2-methoxyethyl)-N-methyl-2-(3-oxo- 3,4-dihydroquinoxalin-2-yl)-1H-indole-5-sulfonamide was obtained as a red solid (0.012 g, 17%) from N-(2-methoxyethyl)-N-methyl-3-nitro-2-(3-oxo-3,4-dihydroquinoxalin-2-yl)-1H-indole-5-sulfonamide (0.085 g, 0.160 mmol). ¹H NMR (DMSO-d₆) δ 12.50 (s,1H), 11.16 (s, 1H), 8.40 (s, 1H), 7.79 (dd, 1H), 7.63 (d, 1H), 7.47 (dd, 1H), 7.35-7.31 (m, 1H), 7.26-7.23 (m, 2H), 3.44 (t, 2H), 3.12 (s, 3H), 3.09 (t, 2H), 2.71 (s, 3H); LCMS RT=2.59 min; [M+H]=428.5.

### EXAMPLE 375

### Preparation of [3-amino-2-(3-oxo-3,4-dihydroquinoxalin-2-yl)-1H-indol-5-yl]methyl phenylcarbamate

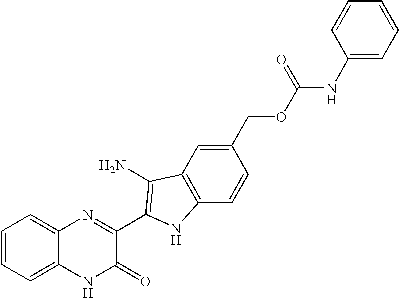

### Step 1. Preparation of [3-nitro-2-(3-oxo-3,4-dihydroquinoxalin-2-yl)-1H-indol-5-ylmethyl phenylcarbamate

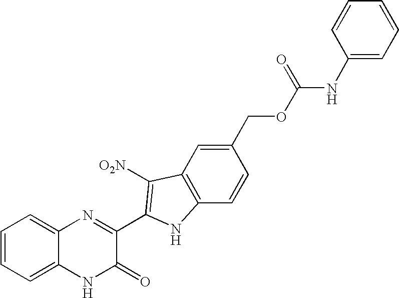

To a solution of 3-[5-(hydroxymethyl)-3-nitro-1H-indol-2-yl]quinoxalin-2(1H)-one (Example 156, 0.100 g, 0.290 mmol) in anhydrous DMF (5 mL) at rt was added phenylisocyanate (0.193 g, 1.62 mmol) and the amber solution was stirred at 80° C. for 24 h. The reaction was diluted with CH₂Cl₂ (20 mL) and purified by silica gel chromatography (hexane/EtOAc) to give 0.099 g of a yellow solid (72%). TLC: R_{f}=0.80 (50% hexane/EtOAc); LC-MS (ESI): [M+H]⁺=456.2 and [M+Na]⁺=478.1 @ RT=3.48 min.

### Step 2. Preparation of [3-amino-2-(3-oxo-3,4-dihydroquinoxalin-2-yl)-1H-indol-5-yl]methyl phenylcarbamate

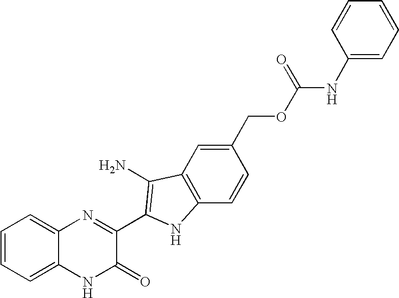

A suspension of the [3-nitro-2-(3-oxo-3,4-dihydroquinoxalin-2-yl)-1H-indol-5-yl]methyl phenylcarbamate (0.040 9, 0.09 mmol) in glacial AcOH (12 mL) was sonicated for 1 h before iron powder (325 mesh, 0.100 g, 1.79 mmol) added to the very fine yellow suspension. The mixture was stirred at rt for 2 h under nitrogen. The red suspension was quenched by adding it slowly to sat. NaHCO₃ (300 mL). The mixture was extracted with EtOAc (2×300 mL). The combined organic layers were dried (Na₂SO₄), filtered, and concentrated in vacuo to give 0.026 g of a red solid (70%). TLC: R_{f}=0.61 (50% hexane/EtOAc); LC-MS (ESI): [M+H]⁺=426.2 @ RT=2.87 min.

### EXAMPLE 418

### Preparation of cyclohexyl 3-amino-2-(3-oxo-3,4-dihydroquinoxalin-2-yl)-1H-indole-5-carboxylate

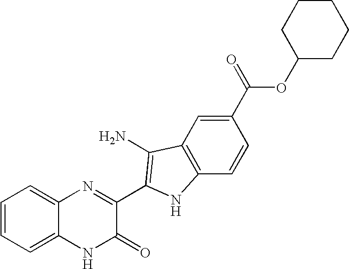

To a solution of SOCl₂ (20.0 mL, 272 mmol) was added 3-nitro-2-(3-oxo-3,4-dihydroquinoxalin-2-yl)-1H-indole-5-carboxylic acid (Example 22, 250 mg, 0.710 mmol) at rt and the resulting brown suspension was heated at 85° C. for 4 h. The suspension was concentrated under reduced pressure and the residue dried for 24 h in vacuo to give 262 mg of 3-nitro-2-(3-oxo-3,4-dihydroquinoxalin-2-yl)-1H-indole-5-carbonyl chloride as a light yellow solid. To a 250 mL rb flask was placed 3-nitro-2-(3-oxo-3,4-dihydroquinoxalin-2-yl)-1H-indole-5-carbonyl chloride (0.07 g, 0.19 mmol) and CH₂Cl₂ (3 mL). To this was added cyclohexanol (0.03 mL, 0.38 mmol) and Et₃N (0.03 mL, 0.21) and the reaction was allowed to stir at 60° C. for 18 h. SnCl₂ (0.43 g, 1.9 mmol) was added and the reaction was allowed to stir at 60° C. for 12 h. The reaction was diluted with water (30 mL) and DMF (100 mL). The mixture was extracted with EtOAc (3×100 mL) and the combined organics were dried (Na₂SO₄) and purified by HPLC. The combined fractions were concentrated to provide 0.026 g of a red solid (33%). ¹H NMR (DMSO-d₆) δ12.55 (s, 1H), 11.19 (s, 1H), 8.56 (s, 1H), 7.78 (m, 1H), 7.54 (d, 1H), 7.36 (m, 1H), 7.27 (m, 1H), 4.93 (m, 1H), 1.98-1.55 (m, 10H); LCMS RT=3.38 min; [M+H]=403.3.

Variations of the compounds of the invention can be readily prepared using the processes described above, or by other standard chemical processes known in the art, by employing appropriate starting materials that are readily available and/or are already described herein.

Generally, a desired salt of a compound of this invention can be prepared in situ during the final isolation and purification of a compound by means well known in the art. For example, a desired salt can be prepared by separately reacting the purified compound in its free base or free acid form with a suitable organic or inorganic acid, or suitable organic or inorganic base, respectively, and isolating the salt thus formed. In the case of basic compounds, for example, the free base is treated with anhydrous HCl in a suitable solvent such as THF, and the salt isolated as a hydrochloride salt. In the case of acidic compounds, the salts may be obtained, for example, by treatment of the free acid with anhydrous ammonia in a suitable solvent such as ether and subsequent isolation of the ammonium salt. These methods are conventional and would be readily apparent to one skilled in the art.

Esters of the compounds identified herein can be obtained by conventional means, for example, by reaction of a carboxylic acid compound with an alcohol facilitated by an acid catalyst, or by reaction of the carboxylic acid compound and alcohol under Mitsunobu conditions. These methods are conventional and would be readily apparent to one skilled in the art.

The purification of isomers of a compound of this invention, and the separation of said isomeric mixtures can be accomplished by standard techniques known in the art.

Compositions of the Compounds of this Invention

The compounds of this invention can be utilized to achieve the desired pharmacological effect by administration to a patient in need thereof in an appropriately formulated pharmaceutical composition. A patient, for the purpose of this invention, is a mammal, including a human, in need of treatment (including prophylactic treatment) for the particular condition or disease. Therefore, the present invention includes pharmaceutical compositions that are comprised of a pharmaceutically acceptable carrier and a pharmaceutically effective amount of a compound, or salt or ester thereof, of the present invention. A pharmaceutically acceptable carrier is any carrier that is relatively non-toxic and innocuous to a patient at concentrations consistent with effective activity of the active ingredient so that any side effects ascribable to the carrier do not vitiate the beneficial effects of the active ingredient. A pharmaceutically effective amount of compound is that amount which produces a result or exerts an influence on the particular condition being treated. The compounds of the present invention can be administered with pharmaceutically-acceptable carriers well known in the art using any effective conventional dosage unit forms, including immediate, slow and timed release preparations, orally, parenterally, topically, nasally, ophthalmically, otically, sublingually, rectally, vaginally, and the like.

For oral administration, the compounds can be formulated into solid or liquid preparations such as capsules, pills, tablets, troches, lozenges, melts, powders, solutions, suspensions, or emulsions, and may be prepared according to methods known to the art for the manufacture of pharmaceutical compositions. The solid unit dosage forms can be a capsule which can be of the ordinary hard- or soft-shelled gelatin type containing, for example, surfactants, lubricants, and inert fillers such as lactose, sucrose, calcium phosphate, and corn starch.

In another embodiment, the compounds of this invention may be tableted with conventional tablet bases such as lactose, sucrose and cornstarch in combination with binders such as acacia, corn starch or gelatin, disintegrating agents intended to assist the break-up and dissolution of the tablet following administration such as potato starch, alginic acid, corn starch, and guar gum, gum tragacanth, acacia, lubricants intended to improve the flow of tablet granulation and to prevent the adhesion of tablet material to the surfaces of the tablet dies and punches, for example talc, stearic acid, or magnesium, calcium or zinc stearate, dyes, coloring agents, and flavoring agents such as peppermint, oil of wintergreen, or cherry flavoring, intended to enhance the aesthetic qualities of the tablets and make them more acceptable to the patient. Suitable excipients for use in oral liquid dosage forms include dicalcium phosphate and diluents such as water and alcohols, for example, ethanol, benzyl alcohol, and polyethylene alcohols, either with or without the addition of a pharmaceutically acceptable surfactant, suspending agent or emulsifying agent. Various other materials may be present as coatings or to otherwise modify the physical form of the dosage unit. For instance tablets, pills or capsules may be coated with shellac, sugar or both.

Dispersible powders and granules are suitable for the preparation of an aqueous suspension. They provide the active ingredient in admixture with a dispersing or wetting agent, a suspending agent and one or more preservatives. Suitable dispersing or wetting agents and suspending agents are exemplified by those already mentioned above. Additional excipients, for example those sweetening, flavoring and coloring agents described above, may also be present.

The pharmaceutical compositions of this invention may also be in the form of oil-in-water emulsions. The oily phase may be a vegetable oil such as liquid paraffin or a mixture of vegetable oils. Suitable emulsifying agents may be (1) naturally occurring gums such as gum acacia and gum tragacanth, (2) naturally occurring phosphatides such as soy bean and lecithin, (3) esters or partial esters derived form fatty acids and hexitol anhydrides, for example, sorbitan monooleate, (4) condensation products of said partial esters with ethylene oxide, for example, polyoxyethylene sorbitan monooleate. The emulsions may also contain sweetening and flavoring agents.

Oily suspensions may be formulated by suspending the active ingredient in a vegetable oil such as, for example, arachis oil, olive oil, sesame oil or coconut oil, or in a mineral oil such as liquid paraffin. The oily suspensions may contain a thickening agent such as, for example, beeswax, hard paraffin, or cetyl alcohol. The suspensions may also contain one or more preservatives, for example, ethyl or n-propyl p-hydroxybenzoate; one or more coloring agents; one or more flavoring agents; and one or more sweetening agents such as sucrose or saccharin.

Syrups and elixirs may be formulated with sweetening agents such as, for example, glycerol, propylene glycol, sorbitol or sucrose. Such formulations may also contain a demulcent, and preservative, such as methyl and propyl parabens and flavoring and coloring agents.

The compounds of this invention may also be administered parenterally, that is, subcutaneously, intravenously, intraocularly, intrasynovially, intramuscularly, or interperitoneally, as injectable dosages of the compound in a physiologically acceptable diluent with a pharmaceutical carrier which can be a sterile liquid or mixture of liquids such as water, saline, aqueous dextrose and related sugar solutions, an alcohol such as ethanol, isopropanol, or hexadecyl alcohol, glycols such as propylene glycol or polyethylene glycol, glycerol ketals such as 2,2-dimethyl-1,1-dioxolane4-methanol, ethers such as poly(ethylene glycol) 400, an oil, a fatty acid, a fatty acid ester or, a fatty acid glyceride, or an acetylated fatty acid glyceride, with or without the addition of a pharmaceutically acceptable surfactant such as a soap or a detergent, suspending agent such as pectin, carbomers, methycellulose, hydroxypropylmethylcellulose, or carboxymethylcellulose, or emulsifying agent and other pharmaceutical adjuvants.

Illustrative of oils which can be used in the parenteral formulations of this invention are those of petroleum, animal, vegetable, or synthetic origin, for example, peanut oil, soybean oil, sesame oil, cottonseed oil, corn oil, olive oil, petrolatum and mineral oil. Suitable fatty acids include oleic acid, stearic acid, isostearic acid and myristic acid. Suitable fatty acid esters are, for example, ethyl oleate and isopropyl myristate. Suitable soaps include fatty acid alkali metal, ammonium, and triethanolamine salts and suitable detergents include cationic detergents, for example dimethyl dialkyl ammonium halides, alkyl pyridinium halides, and alkylamine acetates; anionic detergents, for example, alkyl, aryl, and olefin sulfonates, alkyl, olefin, ether, and monoglyceride sulfates, and sulfosuccinates; non-ionic detergents, for example, fatty amine oxides, fatty acid alkanolamides, and poly(oxyethylene-oxypropylene)s or ethylene oxide or propylene oxide copolymers; and amphoteric detergents, for example, alkyl-beta-aminopropionates, and 2-alkylimidazoline quarternary ammonium salts, as well as mixtures.

The parenteral compositions of this invention will typically contain from about 0.5% to about 25% by weight of the active ingredient in solution. Preservatives and buffers may also be used advantageously. In order to minimize or eliminate irritation at the site of injection, such compositions may contain a non-ionic surfactant having a hydrophile-lipophile balance (HLB) of from about 12 to about 17. The quantity of surfactant in such formulation ranges from about 5% to about 15% by weight. The surfactant can be a single component having the above HLB or can be a mixture of two or more components having the desired HLB.

Illustrative of surfactants used in parenteral formulations are the class of polyethylene sorbitan fatty acid esters, for example, sorbitan monooleate and the high molecular weight adducts of ethylene oxide with a hydrophobic base, formed by the condensation of propylene oxide with propylene glycol.

The pharmaceutical compositions may be in the form of sterile injectable aqueous suspensions. Such suspensions may be formulated according to known methods using suitable dispersing or wetting agents and suspending agents such as, for example, sodium carboxymethylcellulose, methylcellulose, hydroxypropylmethyl-cellulose, sodium alginate, polyvinylpyrrolidone, gum tragacanth and gum acacia; dispersing or wetting agents which may be a naturally occurring phosphatide such as lecithin, a condensation product of an alkylene oxide with a fatty acid, for example, polyoxyethylene stearate, a condensation product of ethylene oxide with a long chain aliphatic alcohol, for example, heptadeca-ethyleneoxycetanol, a condensation product of ethylene oxide with a partial ester derived form a fatty acid and a hexitol such as polyoxyethylene sorbitol monooleate, or a condensation product of an ethylene oxide with a partial ester derived from a fatty acid and a hexitol anhydride, for example polyoxyethylene sorbitan monooleate.

The sterile injectable preparation may also be a sterile injectable solution or suspension in a non-toxic parenterally acceptable diluent or solvent. Diluents and solvents that may be employed are, for example, water, Ringer's solution, isotonic sodium chloride solutions and isotonic glucose solutions. In addition, sterile fixed oils are conventionally employed as solvents or suspending media. For this purpose, any bland, fixed oil may be employed including synthetic mono- or diglycerides. In addition, fatty acids such as oleic acid can be used in the preparation of injectables.

A composition of the invention may also be administered in the form of suppositories for rectal administration of the drug. These compositions can be prepared by mixing the drug with a suitable non-irritation excipient which is solid at ordinary temperatures but liquid at the rectal temperature and will therefore melt in the rectum to release the drug. Such material are, for example, cocoa butter and polyethylene glycol.

Another formulation employed in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art (see, e.g., U.S. Pat. No. 5,023,252, issued Jun. 11, 1991, incorporated herein by reference). Such patches may be constructed for continuous, pulsatile, or on demand delivery of pharmaceutical agents.

Controlled release formulations for parenteral administration include liposomal, polymeric microsphere and polymeric gel formulations which are known in the art.

It may be desirable or necessary to introduce the pharmaceutical composition to the patient via a mechanical delivery device. The construction and use of mechanical delivery devices for the delivery of pharmaceutical agents is well known in the art. Direct techniques for, for example, administering a drug directly to the brain usually involve placement of a drug delivery catheter into the patient's ventricular system to bypass the blood-brain barrier. One such implantable delivery system, used for the transport of agents to specific anatomical regions of the body, is described in U.S. Pat. No. 5,011,472, issued Apr. 30, 1991.

The compositions of the invention can also contain other conventional pharmaceutically acceptable compounding ingredients, generally referred to as carriers or diluents, as necessary or desired. Conventional procedures for preparing such compositions in appropriate dosage forms can be utilized. Such ingredients and procedures include those described in the following references, each of which is incorporated herein by reference: Powell, M. F. et al, “Compendium of Excipients for Parenteral Formulations” PDA Journal of Pharmaceutical Science & Technology 1998, 52(5), 238-311; Strickley, R. G “Parenteral Formulations of Small Molecule Therapeutics Marketed in the United States (1999)-Part-1” PDA Journal of Pharmaceutical Science & Technology 1999, 53(6), 324-349; and Nema, S. et al, “Excipients and Their Use in Injectable Products” PDA Journal of Pharmaceutical Science & Technology 1997, 51(4), 166-171.

Commonly used pharmaceutical ingredients which can be used as appropriate to formulate the composition for its intended route of administration include:

acidifying agents (examples include but are not limited to acetic acid, citric acid, fumaric acid, hydrochloric acid, nitric acid);

alkalinizing agents (examples include but are not limited to ammonia solution, ammonium carbonate, diethanolamine, monoethanolamine, potassium hydroxide, sodium borate, sodium carbonate, sodium hydroxide, triethanolamine, trolamine);

adsorbents (examples include but are not limited to powdered cellulose and activated charcoal);

aerosol propellants (examples include but are not limited to carbon dioxide, CCl₂F₂, F₂ClC—CClF₂ and CClF₃);

air displacement agents (examples include but are not limited to nitrogen and argon);

antifungal preservatives (examples include but are not limited to benzoic acid, butylparaben, ethylparaben, methylparaben, propylparaben, sodium benzoate);

antimicrobial preservatives (examples include but are not limited to benzalkonium chloride, benzethonium chloride, benzyl alcohol, cetylpyridinium chloride, chlorobutanol, phenol, phenylethyl alcohol, phenylmercuric nitrate and thimerosal);

antioxidants (examples include but are not limited to ascorbic acid, ascorbyl palmitate, butylated hydroxyanisole, butylated hydroxytoluene, hypophosphorus acid, monothioglycerol, propyl gallate, sodium ascorbate, sodium bisulfite, sodium formaldehyde sulfoxylate, sodium metabisulfite);

binding materials (examples include but are not limited to block polymers, natural and synthetic rubber, polyacrylates, polyurethanes, silicones, polysiloxanes and styrene-butadiene copolymers);

buffering agents (examples include but are not limited to potassium metaphosphate, dipotassium phosphate, sodium acetate, sodium citrate anhydrous and sodium citrate dihydrate);

carrying agents (examples include but are not limited to acacia syrup, aromatic syrup, aromatic elixir, cherry syrup, cocoa syrup, orange syrup, syrup, corn oil, mineral oil, peanut oil, sesame oil, bacteriostatic sodium chloride injection and bacteriostatic water for injection);

chelating agents (examples include but are not limited to edetate disodium and edetic acid);

colorants (examples include but are not limited to FD&C Red No. 3, FD&C Red No. 20, FD&C Yellow No. 6, FD&C Blue No. 2, D&C Green No. 5, D&C Orange No. 5, D&C Red No. 8, caramel and ferric oxide red);

clarifying agents (examples include but are not limited to bentonite);

emulsifying agents (examples include but are not limited to acacia, cetomacrogol, cetyl alcohol, glyceryl monostearate, lecithin, sorbitan monooleate, polyoxyethylene 50 monostearate);

encapsulating agents (examples include but are not limited to gelatin and cellulose acetate phthalate);

flavorants (examples include but are not limited to anise oil, cinnamon oil, cocoa, menthol, orange oil, peppermint oil and vanillin);

humectants (examples include but are not limited to glycerol, propylene glycol and sorbitol);

levigating agents (examples include but are not limited to mineral oil and glycerin);

oils (examples include but are not limited to arachis oil, mineral oil, olive oil, peanut oil, sesame oil and vegetable oil);

ointment bases (examples include but are not limited to lanolin, hydrophilic ointment, polyethylene glycol ointment, petrolatum, hydrophilic petrolatum, white ointment, yellow ointment, and rose water ointment);

penetration enhancers (transdermal delivery) (examples include but are not limited to monohydroxy or polyhydroxy alcohols, mono-or polyvalent alcohols, saturated or unsaturated fatty alcohols, saturated or unsaturated fatty esters, saturated or unsaturated dicarboxylic acids, essential oils, phosphatidyl derivatives, cephalin, terpenes, amides, ethers, ketones and ureas);

plasticizers (examples include but are not limited to diethyl phthalate and glycerol);

solvents (examples include but are not limited to ethanol, corn oil, cottonseed oil, glycerol, isopropanol, mineral oil, oleic acid, peanut oil, purified water, water for injection, sterile water for injection and sterile water for irrigation);

stiffening agents (examples include but are not limited to cetyl alcohol, cetyl esters wax, microcrystalline wax, paraffin, stearyl alcohol, white wax and yellow wax);

suppository bases (examples include but are not limited to cocoa butter and polyethylene glycols (mixtures);

surfactants (examples include but are not limited to benzalkonium chloride, nonoxynol 10, oxtoxynol 9, polysorbate 80, sodium lauryl sulfate and sorbitan mono-palmitate);

suspending agents (examples include but are not limited to agar, bentonite, carbomers, carboxymethylcellulose sodium, hydroxyethyl cellulose, hydroxypropyl cellulose, hydroxypropyl methylcellulose, kaolin, methylcellulose, tragacanth and veegum);

sweetening agents (examples include but are not limited to aspartame, dextrose, glycerol, mannitol, propylene glycol, saccharin sodium, sorbitol and sucrose);

tablet anti-adherents (examples include but are not limited to magnesium stearate and talc);

tablet binders (examples include but are not limited to acacia, alginic acid, carboxymethylcellulose sodium, compressible sugar, ethylcellulose, gelatin, liquid glucose, methylcellulose, non-crosslinked polyvinyl pyrrolidone, and pregelatinized starch);

tablet and capsule diluents (examples include but are not limited to dibasic calcium phosphate, kaolin, lactose, mannitol, microcrystalline cellulose, powdered cellulose, precipitated calcium carbonate, sodium carbonate, sodium phosphate, sorbitol and starch);

tablet coating agents (examples include but are not limited to liquid glucose, hydroxyethyl cellulose, hydroxypropyl cellulose, hydroxypropyl methylcellulose, methylcellulose, ethylcellulose, cellulose acetate phthalate and shellac);

tablet direct compression excipients (examples include but are not limited to dibasic calcium phosphate);

tablet disintegrants (examples include but are not limited to alginic acid, carboxymethylcellulose calcium, microcrystalline cellulose, polacrillin potassium, cross-linked polyvinylpyrrolidone, sodium alginate, sodium starch glycollate and starch);

tablet glidants (examples include but are not limited to colloidal silica, corn starch and talc);

tablet lubricants (examples include but are not limited to calcium stearate, magnesium stearate, mineral oil, stearic acid and zinc stearate);

tablet/capsule opaquants (examples include but are not limited to titanium dioxide);

tablet polishing agents (examples include but are not limited to carnuba wax and white wax);

thickening agents (examples include but are not limited to beeswax, cetyl alcohol and paraffin);

tonicity agents (examples include but are not limited to dextrose and sodium chloride);

viscosity increasing agents (examples include but are not limited to alginic acid, bentonite, carbomers, carboxymethylcellulose sodium, methylcellulose, polyvinyl pyrrolidone, sodium alginate and tragacanth); and

wetting agents (examples include but are not limited to heptadecaethylene oxycetanol, lecithins, sorbitol monooleate, polyoxyethylene sorbitol monooleate, and polyoxyethylene stearate).

It is believed that one skilled in the art, utilizing the preceding information, can utilize the present invention to its fullest extent. Nevertheless, the following are examples of pharmaceutical formulations that can be used in the method of the present invention. They are for illustrative purposes only, and are not to be construed as limiting the invention in any way.

Pharmaceutical compositions according to the present invention can be illustrated as follows:

Sterile IV Solution: A 5 mg/mL solution of the desired compound of this invention is made using sterile, injectable water, and the pH is adjusted if necessary. The solution is diluted for administration to 1-2 mg/mL with sterile 5% dextrose and is administered as an IV infusion over 60 min.

Lyophilized powder for IV administration: A sterile preparation can be prepared with (i) 100-1000 mg of the desired compound of this invention as a lypholized powder, (ii) 32-327 mg/mL sodium citrate, and (iii) 300-3000 mg Dextran 40. The formulation is reconstituted with sterile, injectable saline or dextrose 5% to a concentration of 10 to 20 mg/mL, which is further diluted with saline or dextrose 5% to 0.2-0.4 mg/mL, and is administered either IV bolus or by IV infusion over 15-60 min.

Intramuscular suspension: The following solution or suspension can be prepared, for intramuscular injection:
50 mg/mL of the desired, water-insoluble compound of this invention
5 mg/mL sodium carboxymethylcellulose
4 mg/mL TWEEN 80
9 mg/mL sodium chloride
9 mg/mL benzyl alcohol

Hard Shell Capsules: A large number of unit capsules are prepared by filling standard two-piece hard galantine capsules each with 100 mg of powdered active ingredient, 150 mg of lactose, 50 mg of cellulose and 6 mg of magnesium stearate.

Soft Gelatin Capsules: A mixture of active ingredient in a digestible oil such as soybean oil, cottonseed oil or olive oil is prepared and injected by means of a positive displacement pump into molten gelatin to form soft gelatin capsules containing 100 mg of the active ingredient. The capsules are washed and dried. The active ingredient can be dissolved in a mixture of polyethylene glycol, glycerin and sorbitol to prepare a water miscible medicine mix.

Tablets: A large number of tablets are prepared by conventional procedures so that the dosage unit was 100 mg of active ingredient, 0.2 mg. of colloidal silicon dioxide, 5 mg of magnesium stearate, 275 mg of microcrystalline cellulose, 11 mg. of starch, and 98.8 mg of lactose. Appropriate aqueous and non-aqueous coatings may be applied to increase palatability, improve elegance and stability or delay absorption.

Immediate Release Tablets/Capsules: These are solid oral dosage forms made by conventional and novel processes. These units are taken orally without water for immediate dissolution and delivery of the medication. The active ingredient is mixed in a liquid containing ingredient such as sugar, gelatin, pectin and sweeteners. These liquids are solidified into solid tablets or caplets by freeze drying and solid state extraction techniques. The drug compounds may be compressed with viscoelastic and thermoelastic sugars and polymers or effervescent components to produce porous matrices intended for immediate release, without the need of water.

Method of Treating Pharmacological Disorders

The present invention also relates to a method of using the compounds or compositions described herein for the treatment or prevention of, or in the manufacture of a medicament for treating or preventing, mammalian hyper-proliferative disorders. This method comprises administering to a patient (or a mammal) in need thereof, including a human, an amount of a compound, a pharmaceutically acceptable salt or ester thereof, or a composition of this invention which is effective to treat or prevent the disorder.

Hyper-proliferative disorders include but are not limited to solid tumors, such as cancers of the breast, respiratory tract, brain, reproductive organs, digestive tract, urinary tract, eye, liver, skin, head and neck, thyroid, parathyroid and their distant metastases. Those disorders also include lymphomas, sarcomas, and leukemias.

The present invention also relates to a method for using the compounds of this invention as prophylactic or chemopreventive agents for prevention of the mammalian hyper-proliferative disorders described herein. This method comprises administering to a mammal in need thereof, including a human, an amount of a compound of this invention, or a pharmaceutically acceptable salt or ester thereof, which is effective to delay or diminish the onset of the disorder.

Examples of breast cancer include, but are not limited to invasive ductal carcinoma, invasive lobular carcinoma, ductal carcinoma in situ, and lobular carcinoma in situ.

Examples of hyper-proliferative disorders of the cardiovacular system include, but are not limited to, restenosis.

Examples of cancers of the respiratory tract include, but are not limited to small-cell and non-small-cell lung carcinoma, as well as bronchial adenoma and pleuropulmonary blastoma.

Examples of brain cancers include, but are not limited to brain stem and hypophtalmic glioma, cerebellar and cerebral astrocytoma, medulloblastoma, ependymoma, as well as neuroectodermal and pineal tumor.

Tumors of nervous system include, but not limited to glioblastoma.

Tumors of the male reproductive organs include, but are not limited to prostate and testicular cancer. Tumors of the female reproductive organs include, but are not limited to endometrial, cervical, ovarian, vaginal, and vulvar cancer, as well as sarcoma of the uterus.

Tumors of the digestive tract include, but are not limited to anal, colon, colorectal, esophageal, gallbladder, gastric, pancreatic, rectal, small-intestine, and salivary gland cancers.

Tumors of the urinary tract include, but are not limited to bladder, penile, kidney, renal pelvis, ureter, and urethral cancers.

Eye cancers include, but are not limited to intraocular melanoma and retinoblastoma.

Examples of liver cancers include, but are not limited to hepatocellular carcinoma (liver cell carcinomas with or without fibrolamellar variant), cholangiocarcinoma (intrahepatic bile duct carcinoma), and mixed hepatocellular cholangiocarcinoma.

Skin cancers include, but are not limited to squamous cell carcinoma, Kaposi's sarcoma, malignant melanoma, Merkel cell skin cancer, and non-melanoma skin cancer.

Head-and-neck cancers include, but are not limited to laryngeal/hypopharyngeal/nasopharyngeal/oropharyngeal cancer, and lip and oral cavity cancer.

Lymphomas include, but are not limited to AIDS-related lymphoma, non-Hodgkin's lymphoma, cutaneous T-cell lymphoma, Hodgkin's disease, and lymphoma of the central nervous system.

Sarcomas include, but are not limited to sarcoma of the soft tissue, osteosarcoma, malignant fibrous histiocytoma, lymphosarcoma, and rhabdomyosarcoma.

Leukemias include, but are not limited to acute myeloid leukemia, acute lymphoblastic leukemia, chronic lymphocytic leukemia, chronic myelogenous leukemia, and hairy cell leukemia.

These disorders have been well characterized in humans, and also exist with a similar etiology in other mammals which can also be treated by the administration of the compounds and/or pharmaceutical compositions of the present invention.

The utility of the compounds of the present invention can be illustrated, for example, by their activity in vitro in the in vitro tumor cell proliferation assay described below. The link between activity in tumor cell proliferation assays in vitro and anti-tumor activity in the clinical setting has been very well established in the art. For example, the therapeutic utility of taxol (Silvestrini et al. Stem Cells 1993, 11(6), 528-35), taxotere (Bissery et al. Anti Cancer Drugs 1995, 6(3), 339), and topoisomerase inhibitors (Edelman et al. Cancer Chemother. Pharmacol. 1996, 37(5), 385-93) were demonstrated with the use of in vitro tumor proliferation assays.

The present compounds and compositions exhibit anti-proliferative activity and are thus useful to treat the indications listed above, e.g. indications mediated by hyperproliferative disorders. Indications mediated by hyperproliferative disorders means diseases or conditions whose progression proceeds, at least in part, via proliferation. The following assay is one of the methods by which compound activity relating to treatment of the disorders identified herein can be determined.

In Vitro Tumor Model Assay

Measurement of anti-proliferative activity can be evaluated as follows. A human tumor cell line such as HCT-116, was cultured under conditions recommended by the supplier (CCL-247, American Type Culture Collection, Manassas, Va., USA). To prepare the assay plates cells were removed from the culture dishes as a single cell suspension and plated at 5000 cell/well in a 96-well plate. Test compounds exemplified by Formula 1 above were dissolved in 100% dimethylsulfoxide at a concentration of 10 mmoles/L and diluted to the appropriated concentration such that the final dimethylsulfoxide concentration in the culture media did not exceed 0.25%. The day after cell plating, the test compounds were added to the culture medium at the appropriate dilutions, and the cells with the test compound were allowed to remain in contact under normal cell culture conditions for 72 hours. The inhibitory activity was measured using a CellTiter-Glo assay kit, using the instructions provided by the manufacture (Promega, Madison, Wis., USA). The % growth inhibition was calculated using the formula % inhibition=(value with test compound/value without test compound)×100.

Representative compounds of the invention were tested in the above assay and were found to be active.

Additionally, the compounds of this invention are useful in the prevention and/or treatment of, or in the manufacture of a medicament for treating, angiogenesis dependent disorders. A number of diseases are known to be associated with angiogenesis such as, for example, ocular neovascular disease, neovascular glaucoma, diabetic retinopathy, retrolental fibroplasia, hemangiomas, angiofibromas, psoriasis, age-related macula degeneration, haemangioblastoma, haemangioma, pain and inflammatory diseases such as rheumatoid or rheumatic inflammatory diseases including rheumatoid arthritis, as well as neoplastic diseases including, for example, so-called solid tumors and liquid tumors such as leukemias. As angiogenesis inhibitors, the compounds of this inveniton are also useful to control solid tumor growth such as breast, prostate, lung, pancreatic, renal, colon, and cervical cancer, melanoma, tumor metastasis, and the like as are well known in the art.

Tumors smaller than about 1-2 mm in diameter may receive oxygen and nutrients through diffusion directly into the tumor cells. However, angiogenesis is regarded as an absolute prerequisite for tumors that grow beyond that diameter. The principal mechanisms that play an important role in inhibition of tumor angiogenesis include inhibition of the growth of blood vessels, especially capillaries, into an avascular resting turmor, resulting in no net tumor growth due to the balance that is achieved between apoptosis and proliferation. Another route to treatment is through decreasing or preventing the migration of tumor cells throughout the body through the blood stream due to the inhibition of angiogenesis in relation to the tumor. Additionally, endothelial cell growth may be inhibited to aviod the paracrine growth-stimulating effect exerted on the surrounding tissue by the endothelial cells which normally line the blood vessels.

Measurement of anti-angiogenic activity can be evaluated as follows:

Xenograph Tumor Model Assay:

Female Ncr nude mice [Taconic Laboratories, NY] were inoculated subcutaneously with 5×106 MDA-MB-231 breast tumor cells (NCI, MD) on day 0. When tumors reached the size about 75 to 150 mm3, tumor-bearing animals were randomly divided into several groups with 10 mice per group and received the treatment with either vehicle or test compounds. All test compounds were formulated in PEG 400: Ethanol: 50 mM methanesulfonic acid (40:10:50, v/v/v) vehicle, and given orally for 14 days. The dosing volumes were 0.1 mL-test article/10 g body weight or 10 mL/kg. During the course of the study, the length and width of each tumor was measured with electronic calipers every 2 or 3 days, and tumor size was calculated at each measuring time-point based on the formula of [length (mm)×width (mm)2]/2. Animal body weights were also recorded at the same time. All animals were observed for clinical signs daily after compound administration. At the end of the treatment period, tumors from both control animals and from animals treated with test compounds were resected and fixed in 10% buffered formalin and imbedded in paraffin. Tissue sections were prepared for immunohistochemistry and stained with anit-CD31 antibodies (sc-1506, Santa Cruz, Calif.) and developed using an ABC kit (Vector, Burlingame, Calif.) according to the manufacturer's instructions. The amount of CD31 staining as a percentage of the total area relative to untreated tumors was determined from images of sections using ImagePro Plus (Media Cybernetics, Silver Spring, Md.) software.

Representative compounds of the invention were tested in the above assay and were found be active in reducing tumor size and in inhibiting angiogenisis.

Based upon the above and other standard laboratory techniques known to evaluate compounds useful for the prevention and/or treatment of the diseases or disorders described above by standard toxicity tests and by standard pharmacological assays for the determination of the prevention and/or treatment of the conditions identified above in mammals, and by comparison of these results with the results of known medicaments that are used to treat these conditions, the effective dosage of the compounds of this invention can readily be determined for prevention and/or treatment of each desired indication. The amount of the active ingredient to be administered in the prevention and/or treatment of one of these conditions can vary widely according to such considerations as the particular compound and dosage unit employed, the mode of administration, the duration of treatment (including prophylactic treatment), the age and sex of the patient treated, and the nature and extent of the condition to be prevented and/or treated.

The total amount of the active ingredient to be administered will generally range from about 0.001 mg/kg to about 300 mg/kg, and preferably from about 0.10 mg/kg to about 150 mg/kg body weight per day. A unit dosage may contain from about 0.5 mg to about 1500 mg of active ingredient, and can be administered one or more times per day. The daily dosage for administration by injection, including intravenous, intramuscular, subcutaneous and parenteral injections, and use of infusion techniques will preferably be from 0.01 to 200 mg/kg of total body weight. The daily rectal dosage regimen will preferably be from 0.01 to 200 mg/kg of total body weight. The daily vaginal dosage regimen will preferably be from 0.01 to 200 mg/kg of total body weight. The daily topical dosage regimen will preferably be from 0.1 to 200 mg administered between one to four times daily. The transdermal concentration will preferably be that required to maintain a daily dose of from 0.01 to 200 mg/kg. The daily inhalation dosage regimen will preferably be from 0.01 to 100 mg/kg of total body weight.

Of course the specific initial and continuing dosage regimen for each patient will vary according to the nature and severity of the condition as determined by the attending diagnostician, the activity of the specific compound employed, the age and general condition of the patient, time of administration, route of administration, rate of excretion of the drug, drug combinations, and the like. The desired mode of administration and number of doses of a compound of the present invention or a pharmaceutically acceptable salt or ester or composition thereof can be ascertained by those skilled in the art using conventional prevention and/or treatment tests.

The compounds of this invention can be administered as the sole pharmaceutical agent or in combination with one or more other pharmaceutical agents where the combination causes no unacceptable adverse effects. For example, the compounds of this invention can be combined with other anti-hyper-proliferative or other indication agents, and the like, as well as with admixtures and combinations thereof.

For example, optional anti-hyper-proliferative agents which can be added to the composition include but are not limited to compounds listed on the cancer chemotherapy drug regimens in the 11^{th} Edition of the Merck Index, (1996), which is hereby incorporated by reference, such as asparaginase, bleomycin, carboplatin, carmustine, chlorambucil, cisplatin, colaspase, cyclophosphamide, cytarabine, dacarbazine, dactinomycin, daunorubicin, doxorubicin (adriamycine), epirubicin, etoposide, 5-fluorouracil, hexamethylmelamine, hydroxyurea, ifosfamide, irinotecan, leucovorin, lomustine, mechlorethamine, 6-mercaptopurine, mesna, methotrexate, mitomycin C, mitoxantrone, prednisolone, prednisone, procarbazine, raloxifen, streptozocin, tamoxifen, thioguanine, topotecan, vinblastine, vincristine, and vindesine.

Other anti-hyper-proliferative agents suitable for use with the composition of the invention include but are not limited to those compounds acknowledged to be used in the treatment and/or prevention of neoplastic diseases in Goodman and Gilman's The Pharmacological Basis of Therapeutics (Ninth Edition), editor Molinoff et al., publ. by McGraw-Hill, pages 1225-1287, (1996), which is hereby incorporated by reference, such as aminoglutethimide, L-asparaginase, azathioprine, 5-azacytidine cladribine, busulfan, diethylstilbestrol, 2′,2′-difluorodeoxycytidine, docetaxel, erythrohydroxynonyladenine, ethinyl estradiol, 5-fluorodeoxyuridine, 5-fluorodeoxyuridine monophosphate, fludarabine phosphate, fluoxymesterone, flutamide, hydroxyprogesterone caproate, idarubicin, interferon, medroxyprogesterone acetate, megestrol acetate, melphalan, mitotane, paclitaxel, pentostatin, N-phosphonoacetyl-L-aspartate (PALA), plicamycin, semustine, teniposide, testosterone propionate, thiotepa, trimethylmelamine, uridine, and vinorelbine.

Other anti-hyper-proliferative agents suitable for use with the composition of this invention include but are not limited to other anti-cancer agents such as epothilone, irinotecan, raloxifen and topotecan.

It is believed that one skilled in the art, using the preceding information and information available in the art, can utilize the present invention to its fullest extent.

It should be apparent to one of ordinary skill in the art that changes and modifications can be made to this invention without departing from the spirit or scope of the invention as it is set forth herein.

What is claimed is:

1. A compound of Formula I 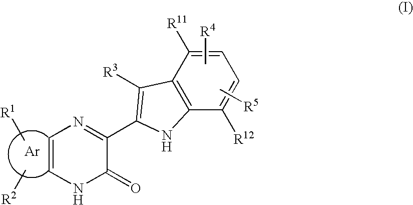
wherein 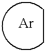
represents a 6 membered aromatic ring containing 0, 1 or 2 N atoms;
R¹ and R² are each independently selected from H, halo, CF₃, C(O)R⁹, 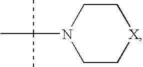
(C₁-C₆)alkyl optionally substituted with up to two substituents selected from OH, (C₁-C₃)alkoxy, F, and phenyl,
(C₁-C₆)alkoxy optionally substituted with one or two substituents each independently selected from 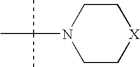
and N[(C₁-C₃)alkyl]₂ where each alkyl is independently optionally substituted up to two times with (C₁-C₃)alkoxy,
NH(C₁-C₃)alkyl where said alkyl is optionally substituted with up to two substitutents each selected independently from OH, F, (C₁-C₃)alkoxy, N[(C₁-C₃)alkyl]₂, NH(C₁-C₃)alkyl, phenyl, pyrrolidinyl, and 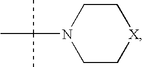
N[(C₁-C₃)alkyl]₂ where each alkyl is independently optionally substituted with up to two substitutents each selected independently from OH, F, phenyl, and (C₁-C₃)alkoxy, said alkoxy being optionally substituted with 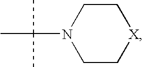
pyrrolidinyl optionally substituted up to two times with N[(C₁-C₃)alkyl]₂,
phenyl optionally substituted with up to two substitutents each selected independently from (C₁-C₃)alkyl, (C₁-C₃)alkoxy, halo, CF₃, and CN,
with the proviso that when 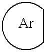
contains 1 or 2 N atoms, R¹ and R² must each be H,
and, R¹ and R² together with the adjacent C atoms to which they are attached form a ring selected from benzo, dioxolo and imidazo,
said imidazo being optionally substituted up to two times with (C₁-C₃)alkyl,
with the proviso that R¹ and R² together with the adjacent C atoms to
which they are attached form a ring only when 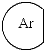
contains no N atoms;
R³ is selected from H, (C₁-C₄)alkyl, OH, NO₂, NH₂, NH(C₁-C₄)alkyl, NHC(O)(C₁-C₄)alkyl and NHC(O)phenyl, said phenyl being optionally substituted with up to two substituents independently selected from (C₁-C₃)alkyl, (C₁-C₃)alkoxy, halo, CF₃, and CN;
R⁴ is selected from H, OH, halo, CN, C(O)R⁶, S(O)₂R⁷, OSi[(C₁-C₄)alkyl]₃, tetrazolyl, thienyl, pyrrolyl, pyrimidinyl, oxazolyl, furanyl,
(C₁-C₆)alkyl, (C₂-C₆)alkenyl or (C₂-C₆)alkynyl, each optionally substituted with OH, F, OC(O)NHphenyl, NHC(O)(C₁-C₃)alkyl, C(O)NH₂, C(O)NH(C₁-C₃)alkyl, C(O)N[(C₁-C₃)alkyl]₂, 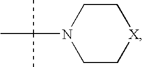
(C₁-C₃)alkoxy optionally substituted up to two times with (C₁-C₃)alkoxy,
NHC(O)NH(C₁-C₃)alkyl where said alkyl is optionally substituted with up to two substituents independently selected from OH, (C₁-C₃)alkoxy, F and phenyl,
NHC(O)NHphenyl where said phenyl is optionally substituted with up to two substituents independently selected from (C₁-C₃)alkyl, (C₁-C₃)alkoxy, halo, CF₃, CN, and 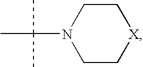
NHC(O)N[(C₁-C₃)alkyl]₂ where each alkyl is independently optionally substituted up to two times with (C₁-C₃)alkoxy,
NH-phenyl, said phenyl being optionally substituted with up to two substituents independently selected from (C₁-C₃)alkyl, (C₁-C₃)alkoxy, halo, CN, and 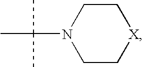
N[(C₁-C₃)alkyl]₂ where each alkyl is independently optionally substituted up to two times with (C₁-C₃)alkoxy,
phenyl optionally substituted with up to two substituents independently selected from (C₁-C₃)alkyl, (C₁-C₃)alkoxy, halo, CN, CF₃, and 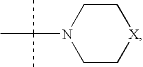
pyrrolidinyl optionally substituted up to two times with N[(C₁-C₃)alkyl]₂,
(C₁-C₆)alkoxy optionally substituted with up to two substituents independently selected from (C₁-C₃)alkoxy, pyrrolidinyl, 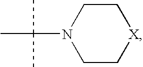
and N[(C₁-C₃)alkyl]₂ where each alkyl is independently optionally substituted with up to two substituents independently selected from OH, F, (C₁-C₃)alkoxy and phenyl,
N[(C₁-C₄)alkyl]₂ where each of said alkyl groups are independently optionally substituted with up to two substituents independently selected from OH, (C₁-C₃)alkyl, F, (C₁-C₃)alkoxy, and phenyl,
oxadiazolyl optionally substituted up to two times with (C₁-C₃)alkyl,
phenyl optionally substituted with up to two substituents independently selected from (C₁-C₃)alkoxy, CN, (C₁-C₃)alkyl, halo, 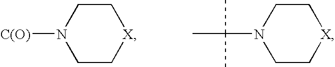
C(O)(C₁-C₃)alkyl optionally substituted with up to two substituents independently selected from (C₁-C₃)alkoxy, OH, (C₁-C₃)alkoxy, F, and phenyl, and
C(O)N((C₁-C₃)alkyl]₂ where each of said alkyl groups are independently optionally substituted up to two times with (C₁-C₃)alkoxy,
pyridyl optionally substituted with up to two substituents independently selected from (C₁-C₃)alkyl,
C(O)N[(C₁-C₃)alkyl]₂ where each of said alkyl groups are independently optionally substituted up to two times with (C₁-C₃)alkoxy, and
O-pyridyl optionally substituted with up to two substituents independently selected from CF₃, halo, and (C₁-C₃)alkyl;
R⁵ is selected from H, halo, CN, (C₁-C₆)alkoxy, and (C₁-C₆)alkyl;
R⁶ is selected from OH, NHR¹⁰, O—(C₃-C₆)cycloakyl, (C₁-C₃)alkoxy, O—(C₂-C₆)alkenyl, O—(C₃-C₆)alkynyl,
(C₁-C₆)alkyl optionally substituted with up to two substituents independently selected from OH, (C₁-C₃)alkoxy, F, and phenyl,
N[(C₁-C₄)alkyl]₂ where each of said alkyl groups are independently optionally substituted with up to two substituents independently selected from OH, CN, N[(C₁-C₄)alkyl]₂, (C₁-C₃)alkoxy, S(O)₂-phenyl, S(O)₂(C₁-C₃)alkyl, phenyl, furyl, tetrahydrofuryl, (C₃-C₆)cycloalkyl, and pyridyl,
N[(C₁-C₃)alkyl]R⁸ where C(C₁-C₃)alkyl] is optionally substituted up to two times with (C₁-C₃)alkoxy,
N[(C₃-C₆)cycloalkyl](C₁-C₃)alkyl where said alkyl is substituted with up to two substituents independently selected from (C₁-C₃)alkoxy, OH, CN, N[(C₁-C₄)alkyl]₂, S(O)₂-phenyl, S(O)₂(C₁-C₃)alkyl, phenyl, furyl, tetrahydrofuryl, (C₅-C₆)cycloalkyl, and pyridyl,
pyrrolidinyl optionally substituted with up to two substituents independently selected from NH₂, NH(C₁-C₃)alkyl, N[(C₁-C₄)alkyl]₂, C(O)NH₂,
NHC(O)(C₁-C₃)alkyl, NHS(O)₂(C₁-C₃)alkyl, pyridyl,
N[(C₁-C₃)alkyl]C(O)NH(C₁-C₃)alkyl, N[(C₁-C₃)alkyl]C(O)(C₁-C₃)alkyl, and
(C₁-C₃)alkyl optionally substituted with up to two substituents independently selected from N[(C₁-C₄)alkyl]₂, (C₁-C₃)alkoxy, and pyrrolidinyl,
morpholinyl optionally substituted up to two times with (C₁-C₃)alkyl,
thiomorpholinyl optionally substituted up to two times with (C₁-C₃)alkyl,
piperazinyl optionally substituted with up to two substituents independently selected from pyrazinyl, C(O)NH₂, C(O)NH-phenyl, C(O)-furanyl, C(O)(C₁-C₃)alkyl, C(O)NH(C₁-C₃)alkyl, C(O)N[(C₁-C₃)alkyl]R⁸, S(O)₂(C₁-C₃)alkyl, S(O)₂-phenyl, 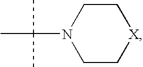
pyridyl optionally substituted with up to two substituents independently selected from (C₁-C₃)alkyl, CN and CF₃,
phenyl optionally substituted with up to two substituents independently selected from (C₁-C₃)alkyl, CN, halo, CF₃, and (C₁-C₃)alkoxy,
(C₁-C₃)alkyl optionally substituted with up to two substituents independently selected from OH, F, phenyl, (C₁-C₃)alkoxy, N[(C₁-C₃)alkyl]₂, pyrrolinidyl, C(O)-pyrrolidinyl, 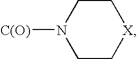 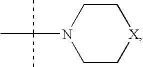
and pyridyl optionally substituted up to two times with (C₁-C₃)alkoxy, and
piperidinyl optionally substituted with up to two substituents independently selected from phenyl, pyridyl, pyrrolidinyl and oxo-dihydrobenzimidazolyl;
R⁷ is selected from NH₂, pyrrolidinyl, 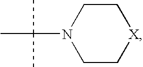
NH(C₁-C₃)alkyl said alkyl being optionally substituted up to two times with (C₁-C₃)alkoxy,
NH-phenyl said phenyl being optionally substituted with up to two substituents independently selected from (C₁-C₃)alkyl, CN, (C₁-C₄)alkoxy, halo and CF₃,
N[(C₁-C₃)alkyl]₂ wherein each alkyl is independently optionally substituted up to two times with (C₁-C₄)alkoxy, and
phenyl optionally substituted with up to two substituents independently selected from (C₁-C₃)alkyl, (C₁-C₃)alkoxy, halo, CF₃ and CN;
R⁸ is selected from (C₁-C₃)alkoxy, pyridyl, piperidinyl, pyranyl and phenyl, where each ring moiety is optionally substituted with up to two substituents independently selected from (C₁-C₃)alkoxy, and (C₁-C₃)alkyl;
R⁹ is selected from (C₁-C₃)alkyl, (C₁-C₃)alkoxy, OH, 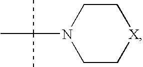
phenyl optionally substituted with (C₁-C₃)alkyl, (C₁-C₃)alkoxy, halo, CF₃, and CN,
N[(C₁-C₄)alkyl]₂ where each of said alkyl groups are independently optionally substituted with OH, CN, N[(C₁-C₄)alkyl]₂, (C₁-C₄)alkoxy, S(O)₂-phenyl, S(O)₂(C₁-C₃)alkyl, phenyl, furyl, tetrahydrofuryl, (C₃-C₆)cycloalkyl, and pyridyl, and
pyrrolidinyl optionally substituted with N[(C₁-C₃)alkyl]₂,
and, only when 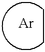
contains no N atoms, R⁹ is also selected from pyridyl, thienyl, and NHR¹⁰;
R¹⁰ is selected from H, indolyl,
(C₁-C₄)alkyl optionally substituted with up to two substituents independently selected from OH, F, phenyl, (C₁-C₄)alkoxy, NHC(O)(C₁-C₃)alkyl, S—(C₁-C₃)alkyl, benzimidazolyl, indolyl, thienyl, pyrazolyl, 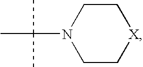
N[(C₁-C₄)alkyl]₂ where each alkyl is independently optionally substituted with up to two substituents independently selected from OH, (C₁-C₃)alkoxy, F, and phenyl,
phenyl optionally substituted with up to two substituents independently selected from (C₁-C₃)alkyl, (C₁-C₃)alkoxy, CN, halo, CF₃, S(O)₂(C₁-C₃)alkyl, S(O)₂phenyl, and S(O)₂NH₂,
pyridyl optionally substituted up to two times with CF₃,
imidazolyl optionally substituted up to two times with (C₁-C₃)alkyl,
furyl optionally substituted up to two times with (C₁-C₄)alkyl, and
pyrrolidinyl optionally substituted with up to two substituents independently selected from (C₁-C₄)alkoxy, (O), and
(C₁-C₄)alkyl optionally substituted with up to two substituents independently selected from OH, (C₁-C₃)alkoxy, F, and phenyl,
S(O)₂-phenyl optionally substituted with up to two substituents independently selected from (C₁-C₄)alkoxy, (C₁-C₃)alkyl, halo, and CN,
pyrazolyl optionally substituted with up to two substituents independently selected from (C₁-C₄)alkyl, (C₃-C₆)cycloalkyl, and
phenyl, said phenyl being optionally substituted with up to two substituents independently selected from (C₁-C₄)alkoxy, (C₁-C₄)alkyl, halo, CF₃, and CN,
benzothiazolyl optionally substituted up to two times with (C₁-C₄)alkyl,
thiazolyl, optionally substituted up to two times with (C₁-C₄)alkyl,
thiadiazolyl, optionally substituted with up to two substituents independently selected from CF₃, (C₃-C₆)cycloalkyl, and (C₁-C₆)alkyl,
phenyl optionally substituted with up to two substituents independently selected
from CN, halo, CF₃, N[(C₁-C₄)alkyl]₂, indolyl, 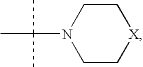
O-pyridyl optionally substituted with C(O)NH(C₁-C₄)alkyl,
(C₁-C₄)alkyl optionally substituted with up to two substituents independently selected from pyridyl, 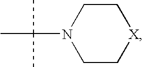
OH,
(C₁-C₃)alkoxy, F, and phenyl, and
(C₁-C₄)alkoxy optionally substituted with N[(C₁-C₄)alkyl]₂ where one alkyl group is optionally substituted with phenyl, or
(C₁-C₄)alkoxy optionally substituted with 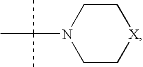
pyridyl optionally substituted with phenoxy where said phenoxy is optionally substituted with up to two substituents independently selected
from (C₁-C₄)alkyl and (C₁-C₄)alkoxy, and
indazolyl optionally substituted up to two times with (C₁-C₄)alkyl;
R¹¹ and R¹² are each selected independently from H, F and Cl with the proviso that when one of R¹¹ and R¹² is F or Cl, the other must be H;
X is selected from O, S, CH₂, and NH, and
when X is NH, the H on NH is optionally replaced with pyridyl, pyrazinyl, phenyl, or (C₁-C₄)alkyl optionally substituted with up to two substituents
independently selected from OH, (C₁-C₃)alkoxy, N[(C₁-C₃)alkyl]₂,
C(O)-pyrrolidinyl, N[(C₁-C₄)alkyl]₂, and phenyl said phenyl being optionally substituted with up to two substituents independently selected from CN and (C₁-C₃)alkoxy,
and when X is O, S, or CH₂, the 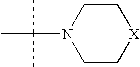
moiety is optionally substituted by replacing any H atom in the 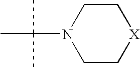
moiety with (C₁-C₄)alkyl;
or a pharmaceutically acceptable salt or ester thereof.

2. A compound of claim 1 wherein 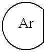
represents a 6 membered ring containing 0 N atoms.

3. A compound of claim 2 wherein R¹ and R² are each independently selected from H, (C₁-C₃)alkoxy, F, and CF₃; R³ is selected from H, NH₂, and NHC(O)(C₁-C₃)alkyl; R⁴ is selected from H, halo, (C₁-C₃)alkoxy, CN, COR⁶, S(O)₂R⁷, N[(C₁-C₃)alkyl]₂, optionally substituted phenyl and optionally substituted (C₁-C₄)alkyl; and R⁵ is selected from H, (C₁-C₃)alkoxy, F and CN.

4. A compound of claim 3 wherein R⁵ is selected from H and F; and R⁴ is selected from H, halo, (C₁-C₃)alkoxy, CN, COR⁶, S(O)₂R⁷, N[(C₁-C₃)alkyl]₂, and optionally substituted (C₁-C₄)alkyl.

5. A compound of claim 4 wherein R¹ and R² are each H; R³ is NH₂; R⁴ is COR⁶, S(O)₂R⁷, and (C₁-C₄)alkyl optionally substituted with N[(C₁-C₃)alkyl]₂ and N[(C₃-C₆)cycloalkyl][(C₁-C₃)alkyl]; R⁵ is H; R⁶ is N[(C₁-C₃)alkyl]₂ and N[(C₃-C₃)alkyl], R⁷ is N[(C₁-C₃)alkyl]₂; and R¹¹ and R¹² are each H.

6. A compound of claim 1 wherein 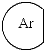
is 6 membered aromatic ring containing 1 or 2 N atoms.

7. A compound of claim 6 wherein R³ is selected from H, NH₂, and NHC(O)(C₁-C₃)alkyl; R⁴ is selected from H, halo, (C₁-C₃)alkoxy, CN, COR⁶, S(O)₂R⁷, N[(C_{1-C}₃)alkyl]₂, optionally substituted phenyl and optionally substituted (C₁-C₄)alkyl; and R⁵ is selected from H, (C₁-C₃)alkoxy, F and CN.

8. A method of treating a hyper-proliferative disorder comprising the administration to a mammal in need thereof of an effective amount of a compound of Formula I 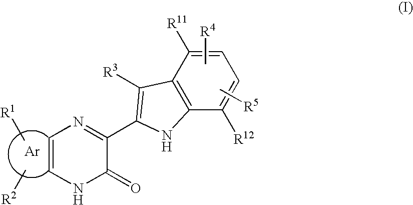
wherein 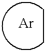
represents a 6 membered aromatic ring containing 0, 1 or 2 N atoms;
R¹ and R² are each independently selected from H, halo, CF₃, C(O)R⁹, 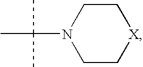
(C₁-C₆)alkyl optionally substituted with up to two substituents selected from OH, (C₁-C₃)alkoxy, F, and phenyl,
(C₁-C₆)alkoxy optionally substituted with one or two substituents each independently selected from 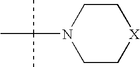
and N[(C₁-C₃)alkyl]₂ where each alkyl is independently optionally substituted up to two times with (C₁-C₃)alkoxy,
NH(C₁-C₃)alkyl where said alkyl is optionally substituted with up to two substitutents each selected independently from OH, F, (C₁-C₃)alkoxy, N[(C₁-C₃)alkyl]₂, NH(C₁-C₃)alkyl, phenyl, pyrrolidinyl, and 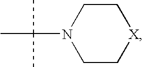
N[(C₁-C₃)alkyl]₂ where each alkyl is independently optionally substituted with up to two substitutents each selected independently from OH, F, phenyl, and (C₁-C₃)alkoxy, said alkoxy being optionally substituted with 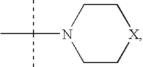
pyrrolidinyl optionally substituted up to two times with N[(C₁-C₃)alkyl]₂,
phenyl optionally substituted with up to two substitutents each selected independently from (C₁-C₃)alkyl, (C₁-C₃)alkoxy, halo, CF₃, and CN,
with the proviso that when 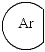
contains 1 or 2 N atoms, R¹ and R² must each be H,
and, R¹ and R² together with the adjacent C atoms to which they are attached form a ring selected from benzo, dioxolo and imidazo,
said imidazo being optionally substituted up to two times with (C₁-C₃)alkyl,
with the proviso that R¹ and R² together with the adjacent C atoms to
which they are attached form a ring only when 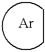
contains no N atoms;
R³ is selected from H, (C₁-C₄)alkyl, OH, NO₂, NH₂, NH(C₁-C₄)alkyl, NHC(O)(C₁-C₄)alkyl and NHC(O)phenyl, said phenyl being optionally substituted with up to two substituents independently selected from (C₁-C₃)alkyl, (C₁-C₃)alkoxy, halo, CF₃, and CN;
R⁴ is selected from H, OH, halo, CN, C(O)R⁶, S(O)₂R⁷, OSi[(C₁-C₄)alkyl]₃, tetrazolyl, thienyl, pyrrolyl, pyrimidinyl, oxazolyl, furanyl,
(C₁-C₆)alkyl, (C₂-C₆)alkenyl or (C₂-C₆)alkynyl, each optionally substituted with OH, F, OC(O)NHphenyl, NHC(O)(C₁-C₃)alkyl, C(O)NH₂, C(O)NH(C₁-C₃)alkyl, C(O)N[(C₁-C₃)alkyl]₂, 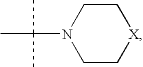
(C₁-C₃)alkoxy optionally substituted up to two times with (C₁-C₃)alkoxy,
NHC(O)NH(C₁-C₃)alkyl where said alkyl is optionally substituted with up to two substituents independently selected from OH, (C₁-C₃)alkoxy, F and phenyl,
NHC(O)NHphenyl where said phenyl is optionally substituted with up to two substituents independently selected from (C₁-C₃)alkyl, (C₁-C₃)alkoxy, halo, CF₃, CN, and 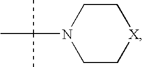
NHC(O)N[(C₁-C₃)alkyl]₂ where each alkyl is independently optionally substituted up to two times with (C₁-C₃)alkoxy,
NH-phenyl, said phenyl being optionally substituted with up to two substituents independently selected from (C₁-C₃)alkyl, (C₁-C₃)alkoxy, halo, CN, and 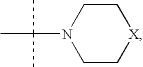
N[(C₁-C₃)alkyl]₂ where each alkyl is independently optionally substituted up to two times with (C₁-C₃)alkoxy,
phenyl optionally substituted with up to two substituents independently selected from (C₁-C₃)alkyl, (C₁-C₃)alkoxy, halo, CN, CF₃, and 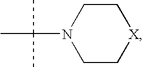
pyrrolidinyl optionally substituted up to two times with N[(C₁-C₃)alkyl]₂,
(C₁-C₆)alkoxy optionally substituted with up to two substituents independently selected from (C₁-C₃)alkoxy, pyrrolidinyl, 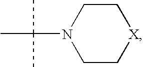
and N[(C₁-C₃)alkyl]₂ where each alkyl is independently optionally substituted with up to two substituents independently selected from OH, F, (C₁-C₃)alkoxy and phenyl,
N[(C₁-C₄)alkyl]₂ where each of said alkyl groups are independently optionally substituted with up to two substituents independently selected from OH, (C₁-C₃)alkyl, F, (C₁-C₃)alkoxy, and phenyl,
oxadiazolyl optionally substituted up to two times with (C₁-C₃)alkyl, phenyl optionally substituted with up to two substituents independently selected from (C₁-C₃)alkoxy, CN, (C₁-C₃)alkyl, halo, 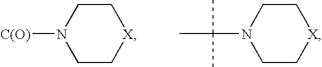
C(O)(C₁-C₃)alkyl optionally substituted with up to two substituents independently selected from (C₁-C₃)alkoxy, OH, (C₁-C₃)alkoxy, F, and phenyl, and
C(O)N[(C₁-C₃)alkyl]₂ where each of said alkyl groups are independently optionally substituted up to two times with (C₁-C₃)alkoxy,
pyridyl optionally substituted with up to two substituents independently selected from (C₁-C₃)alkyl,
C(O)N[(C₁-C₃)alkyl]₂ where each of said alkyl groups are independently optionally substituted up to two times with (C₁-C₃)alkoxy, and
O-pyridyl optionally substituted with up to two substituents independently selected from CF₃, halo, and (C₁-C₃)alkyl;
R⁵ is selected from H, halo, CN, (C₁-C₆)alkoxy, and (C₁-C₆)alkyl;
R⁶ is selected from OH, NHR¹⁰, O—(C₃-C₆)cycloakyl, (C₁-C₃)alkoxy, O—(C₂-C₆)alkenyl,
O—(C₃-C₆)alkynyl,
(C₁-C₆)alkyl optionally substituted with up to two substituents independently selected from OH, (C₁-C₃)alkoxy, F, and phenyl,
N[(C₁-C₄)alkyl]₂ where each of said alkyl groups are independently optionally substituted with up to two substituents independently selected from OH, CN, N[(C₁-C₄)alkyl]₂, (C₁-C₃)alkoxy, S(O)₂-phenyl, S(O)₂(C₁-C₃)alkyl, phenyl, furyl, tetrahydrofuryl, (C₃-C₆)cycloalkyl, and pyridyl,
N[(C₁-C₃)alkyl]R⁸ where [(C₁-C₃)alkyl] is optionally substituted up to two times with (C₁-C₃)alkoxy,
N[(C₃-C₆)cycloalkyl](C₁-C₃)alkyl where said alkyl is substituted with up to two substituents independently selected from (C₁-C₃)alkoxy, OH, CN, N[(C₁-C₄)alkyl]₂, S(O)₂-phenyl, S(O)₂(C₁-C₃)alkyl, phenyl, furyl, tetrahydrofuryl, (C₅-C₆)cycloalkyl, and pyridyl,
pyrrolidinyl optionally substituted with up to two substituents independently selected from NH₂, NH(C₁-C₃)alkyl, N[(C₁-C₄)alkyl]₂, C(O)NH₂, NHC(O)(C₁-C₃)alkyl, NHS(O)₂(C₁-C₃)alkyl, pyridyl, N[(C₁-C₃)alkyl]C(O)NH(C₁-C₃)alkyl, N[(C₁-C₃)alkyl]C(O)(C₁-C₃)alkyl, and (C₁-C₃)alkyl optionally substituted with up to two substituents independently selected from N[(C₁-C₄)alkyl]₂, (C₁-C₃)alkoxy, and pyrrolidinyl,
morpholinyl optionally substituted up to two times with (C₁-C₃)alkyl,
thiomorpholinyl optionally substituted up to two times with (C₁-C₃)alkyl,
piperazinyl optionally substituted with up to two substituents independently selected from pyrazinyl, C(O)NH₂, C(O)NH-phenyl, C(O)-furanyl,
C(O)(C₁-C₃)alkyl, C(O)NH(C₁-C₃)alkyl, C(O)N[(C₁-C₃)alkyl]R⁸,
S(O)₂(C₁-C₃)alkyl, S(O)₂-phenyl, 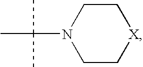
pyridyl optionally substituted with up to two substituents independently selected from (C₁-C₃)alkyl, CN and CF₃,
phenyl optionally substituted with up to two substituents independently selected from (C₁-C₃)alkyl, CN, halo, CF₃, and (C₁-C₃)alkoxy,
(C₁-C₃)alkyl optionally substituted with up to two substituents independently selected from OH, F, phenyl, (C₁-C₃)alkoxy, N[(C₁-C₃)alkyl]₂, pyrrolinidyl, C(O)-pyrrolidinyl, 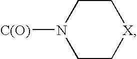 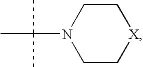
and pyridyl optionally substituted up to two times with (C₁-C₃)alkoxy, and
piperidinyl optionally substituted with up to two substituents independently selected from phenyl, pyridyl, pyrrolidinyl and oxo-dihydrobenzimidazolyl;
R⁷ is selected from NH₂, pyrrolidinyl, 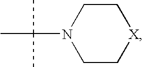
NH(C₁-C₃)alkyl said alkyl being optionally substituted up to two times with (C₁-C₃)alkoxy,
NH-phenyl said phenyl being optionally substituted with up to two substituents independently selected from (C₁-C₃)alkyl, CN, (C₁-C₄)alkoxy, halo and CF₃,
N[(C₁-C₃)alkyl]₂ wherein each alkyl is independently optionally substituted up to two times with (C₁-C₄)alkoxy, and
phenyl optionally substituted with up to two substituents independently selected from (C₁-C₃)alkyl, (C₁-C₃)alkoxy, halo, CF₃ and CN;
R₃ is selected from (C₁-C₃)alkoxy, pyridyl, piperidinyl, pyranyl and phenyl, where each ring moiety is optionally substituted with up to two substituents independently selected from (C₁-C₃)alkoxy, and (C₁-C₃)alkyl;
R⁹ is selected from (C₁-C₃)alkyl, (C₁-C₃)alkoxy, OH, 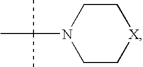
phenyl optionally substituted with (C₁-C₃)alkyl, (C₁-C₃)alkoxy, halo, CF₃, and CN,
N[(C₁-C₄)alkyl]₂ where each of said alkyl groups are independently optionally substituted with OH, CN, N[(C₁-C₄)alkyl]₂, (C₁-C₄)alkoxy, S(O)₂-phenyl, S(O)₂(C₁-C₃)alkyl, phenyl, furyl, tetrahydrofuryl, (C₃-C₆)cycloalkyl, and pyridyl, and
pyrrolidinyl optionally substituted with N[(C₁-C₃)alkyl]₂,
and, only when 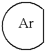
contains no N atoms, R⁹ is also selected from pyridyl, thienyl, and NHR¹⁰;
R¹⁰ is selected from H, indolyl,
(C₁-C₄)alkyl optionally substituted with up to two substituents independently selected from OH, F, phenyl, (C₁-C₄)alkoxy, NHC(O)(C₁-C₃)alkyl, S—(C₁-C₃)alkyl, benzimidazolyl, indolyl, thienyl, pyrazolyl, 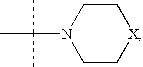
N[(C₁-C₄)alkyl]₂ where each alkyl is independently optionally substituted with up to two substituents independently selected from OH, (C₁-C₃)alkoxy, F, and phenyl,
phenyl optionally substituted with up to two substituents independently selected from (C₁-C₃)alkyl, (C₁-C₃)alkoxy, CN, halo, CF₃, S(O)₂(C₁-C₃)alkyl, S(O)₂phenyl, and S(O)₂NH₂,
pyridyl optionally substituted up to two times with CF₃,
imidazolyl optionally substituted up to two times with (C₁-C₃)alkyl,
furyl optionally substituted up to two times with (C₁-C₄)alkyl, and
pyrrolidinyl optionally substituted with up to two substituents independently selected from (C₁-C₄)alkoxy, (O), and
(C₁-C₄)alkyl optionally substituted with up to two substituents independently selected from OH, (C₁-C₃)alkoxy, F, and phenyl,
S(O)₂-phenyl optionally substituted with up to two substituents independently selected from (C₁-C₄)alkoxy, (C₁-C₃)alkyl, halo, and CN,
pyrazolyl optionally substituted with up to two substituents independently selected from (C₁-C₄)alkyl, (C₃-C₆)cycloalkyl, and
phenyl, said phenyl being optionally substituted with up to two substituents independently selected from (C₁-C₄)alkoxy, (C₁-C₄)alkyl, halo, CF₃, and CN,
benzothiazolyl optionally substituted up to two times with (C₁-C₄)alkyl,
thiazolyl, optionally substituted up to two times with (C₁-C₄)alkyl,
thiadiazolyl, optionally substituted with up to two substituents independently selected from CF₃, (C₃-C₆)cycloalkyl, and (C₁-C₆)alkyl,
phenyl optionally substituted with up to two substituents independently selected from CN, halo, CF₃, N[(C₁-C₄)alkyl]₂, indolyl, 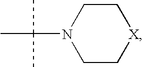
O-pyridyl optionally substituted with C(O)NH(C₁-C₄)alkyl,
(C₁-C₄)alkyl optionally substituted with up to two substituents independently selected from pyridyl, 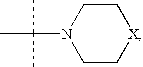
OH,
(C₁-C₃)alkoxy, F, and phenyl, and
(C₁-C₄)alkoxy optionally substituted with N[(C₁-C₄)alkyl]₂ where one alkyl group is optionally substituted with phenyl, or
(C₁-C₄)alkoxy optionally substituted with 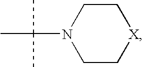
pyridyl optionally substituted with phenoxy where said phenoxy is optionally substituted with up to two substituents independently selected from (C₁-C₄)alkyl and (C₁-C₄)alkoxy, and
indazolyl optionally substituted up to two times with (C₁-C₄)alkyl;
R¹¹ and R¹² are each selected independently from H, F and Cl with the proviso that when one of R¹¹ and R¹² is F or Cl, the other must be H;
X is selected from O, S, CH₂, and NH, and
when X is NH, the H on NH is optionally replaced with pyridyl, pyrazinyl, phenyl, or (C₁-C₄)alkyl optionally substituted with up to two substituents independently selected from OH, (C₁-C₃)alkoxy, N[(C₁-C₃)alkyl]₂, C(O)-pyrrolidinyl, N[(C₁-C₄)alkyl]₂, and phenyl said phenyl being optionally substituted with up to two substituents independently selected from CN and (C₁-C₃)alkoxy,
and when X is O, S, or CH₂, the 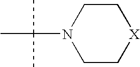
moiety is optionally substituted by replacing any H atom in the 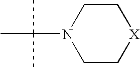
moiety with (C₁-C₄)alkyl;
or a pharmaceutically acceptable salt or ester thereof.

9. A method according to claim 8 wherein the hyperproliferative disorder is selected from breast cancer, lung cancer, colon cancer, pancreatic cancer, prostate cancer, skin cancer, leukemia, lymphoma, glioblastoma and head and neck cancers.

10. A method according to claim 9 wherein the hyperproliferative disorder is selected from breast cancer, lung cancer, colon cancer and pancreatic cancer.

11. A method of treating a angiogenic disorder comprising the administration to a mammal in need thereof of an effective amount of a compound of Formula I 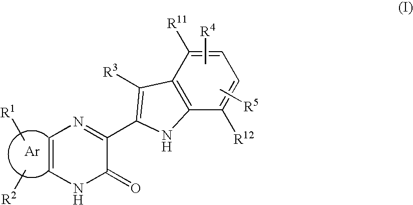
wherein 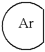
represents a 6 membered aromatic ring containing 0, 1 or 2 N atoms;
R¹ and R² are each independently selected from H, halo, CF₃, C(O)R⁹, 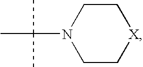
(C₁-C₆)alkyl optionally substituted with up to two substituents selected from OH, (C₁-C₃)alkoxy, F, and phenyl,
(C₁-C₆)alkoxy optionally substituted with one or two substituents each independently selected from 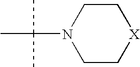
and N[(C₁-C₃)alkyl]₂ where each alkyl is independently optionally substituted up to two times with (C₁-C₃)alkoxy,
NH(C₁-C₃)alkyl where said alkyl is optionally substituted with up to two substitutents each selected independently from OH, F, (C₁-C₃)alkoxy, N[(C₁-C₃)alkyl]₂, NH(C₁-C₃)alkyl, phenyl, pyrrolidinyl, and 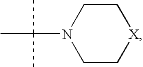
N[(C₁-C₃)alkyl]₂ where each alkyl is independently optionally substituted with up to two substitutents each selected independently from OH, F, phenyl, and (C₁-C₃)alkoxy, said alkoxy being optionally substituted with 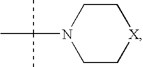
pyrrolidinyl optionally substituted up to two times with N[(C₁-C₃)alkyl]₂,
phenyl optionally substituted with up to two substitutents each selected independently from (C₁-C₃)alkyl, (C₁-C₃)alkoxy, halo, CF₃, and CN,
with the proviso that when 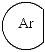
contains 1 or 2 N atoms, R¹ and R² must each be H,
and, R¹ and R² together with the adjacent C atoms to which they are attached form a ring selected from benzo, dioxolo and imidazo,
said imidazo being optionally substituted up to two times with (C₁-C₃)alkyl,
with the proviso that R¹ and R² together with the adjacent C atoms to which they are attached form a ring only when 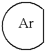
contains no N atoms;
R³ is selected from H, (C₁-C₄)alkyl, OH, NO₂, NH₂, NH(C₁-C₄)alkyl, NHC(O)(C₁-C₄)alkyl and NHC(O)phenyl, said phenyl being optionally substituted with up to two substituents independently selected from (C₁-C₃)alkyl, (C₁-C₃)alkoxy, halo, CF₃, and CN;
R⁴ is selected from H, OH, halo, CN, C(O)R⁶, S(O)₂R⁷, OSi[(C₁-C₄)alkyl]₃, tetrazolyl, thienyl, pyrrolyl, pyrimidinyl, oxazolyl, furanyl,
(C₁-C₆)alkyl, (C₂-C₆)alkenyl or (C₂-C₆)alkynyl, each optionally substituted with OH, F, OC(O)NHphenyl, NHC(O)(C₁-C₃)alkyl, C(O)NH₂, C(O)NH(C₁-C₃)alkyl, C(O)N[(C₁-C₃)alkyl]₂, 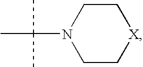
(C₁-C₃)alkoxy optionally substituted up to two times with (C₁-C₃)alkoxy,
NHC(O)NH(C₁-C₃)alkyl where said alkyl is optionally substituted with up to two substituents independently selected from OH, (C₁-C₃)alkoxy, F and phenyl,
NHC(O)NHphenyl where said phenyl is optionally substituted with up to two substituents independently selected from (C₁-C₃)alkyl, (C₁-C₃)alkoxy, halo, CF₃, CN, and 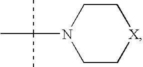
NHC(O)N[(C₁-C₃)alkyl]₂ where each alkyl is independently optionally substituted up to two times with (C₁-C₃)alkoxy,
NH-phenyl, said phenyl being optionally substituted with up to two substituents independently selected from (C₁-C₃)alkyl, (C₁-C₃)alkoxy, halo, CN, and 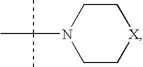
N[(C₁-C₃)alkyl]₂ where each alkyl is independently optionally substituted up to two times with (C₁-C₃)alkoxy, phenyl optionally substituted with up to two substituents independently selected from (C₁-C₃)alkyl, (C₁-C₃)alkoxy, halo, CN, CF₃, and 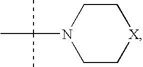
pyrrolidinyl optionally substituted up to two times with N[(C₁-C₃)alkyl]₂,
(C₁-C₆)alkoxy optionally substituted with up to two substituents independently selected from (C₁-C₃)alkoxy, pyrrolidinyl, 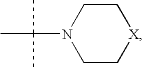
and N[(C₁-C₃)alkyl]₂ where each alkyl is independently optionally substituted with up to two substituents independently selected from OH, F, (C₁-C₃)alkoxy and phenyl,
N[(C₁-C₄)alkyl]₂ where each of said alkyl groups are independently optionally substituted with up to two substituents independently selected from OH, (C₁-C₃)alkyl, F, (C₁-C₃)alkoxy, and phenyl,
oxadiazolyl optionally substituted up to two times with (C₁-C₃)alkyl,
phenyl optionally substituted with up to two substituents independently selected from (C₁-C₃)alkoxy, CN, (C₁-C₃)alkyl, halo, 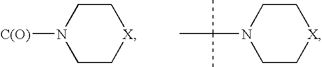
C(O)(C₁-C₃)alkyl optionally substituted with up to two substituents independently selected from (C₁-C₃)alkoxy, OH, (C₁-C₃)alkoxy, F, and phenyl, and
C(O)N[(C₁-C₃)alkyl]₂ where each of said alkyl groups are independently optionally substituted up to two times with (C₁-C₃)alkoxy,
pyridyl optionally substituted with up to two substituents independently selected from (C₁-C₃)alkyl,
C(O)N[(C₁-C₃)alkyl]₂ where each of said alkyl groups are independently optionally substituted up to two times with (C₁-C₃)alkoxy, and
O-pyridyl optionally substituted with up to two substituents independently selected from CF₃, halo, and (C₁-C₃)alkyl;
R⁵ is selected from H, halo, CN, (C₁-C₆)alkoxy, and (C₁-C₆)alkyl;
R⁶ is selected from OH, NHR¹⁰, O—(C₃-C₆)cycloakyl, (C₁-C₃)alkoxy, O—(C₂-C₆)alkenyl,
O—(C₃-C₆)alkynyl,
(C₁-C₆)alkyl optionally substituted with up to two substituents independently selected from OH, (C₁-C₃)alkoxy, F, and phenyl,
N[(C₁-C₄)alkyl]₂ where each of said alkyl groups are independently optionally substituted with up to two substituents independently selected from OH, CN, N[(C₁-C₄)alkyl]₂, (C₁-C₃)alkoxy, S(O)₂-phenyl, S(O)₂(C₁-C₃)alkyl, phenyl, furyl, tetrahydrofuryl, (C₃-C₆)cycloalkyl, and pyridyl,
N[(C₁-C₃)alkyl]R⁸where [(C₁-C₃)alkyl] is optionally substituted up to two times with (C₁-C₃)alkoxy,
N[(C₃-C₆)cycloalkyl](C₁-C₃)alkyl where said alkyl is substituted with up to two substituents independently selected from (C₁-C₃)alkoxy, OH, CN, N[(C₁-C₄)alkyl]₂, S(O)₂-phenyl, S(O)₂(C₁-C₃)alkyl, phenyl, furyl, tetrahydrofuryl, (C₅-C₆)cycloalkyl, and pyridyl,
pyrrolidinyl optionally substituted with up to two substituents independently selected from NH₂, NH(C₁-C₃)alkyl, N[(C₁-C₄)alkyl]₂, C(O)NH₂, NHC(O)(C₁-C₃)alkyl, NHS(O)₂(C₁-C₃)alkyl, pyridyl, N[(C₁-C₃)alkyl]C(O)NH(C₁-C₃)alkyl, N[(C₁-C₃)alkyl]C(O)(C₁-C₃)alkyl, and (C₁-C₃)alkyl optionally substituted with up to two substituents independently selected from N[(C₁-C₄)alkyl]₂, (C₁-C₃)alkoxy, and pyrrolidinyl,
morpholinyl optionally substituted up to two times with (C₁-C₃)alkyl,
thiomorpholinyl optionally substituted up to two times with (C₁-C₃)alkyl,
piperazinyl optionally substituted with up to two substituents independently selected from pyrazinyl, C(O)NH₂, C(O)NH-phenyl, C(O)-furanyl, C(O)(C₁-C₃)alkyl, C(O)NH(C₁-C₃)alkyl, C(O)N[(C₁-C₃)alkyl]R⁸, S(O)₂(C₁-C₃)alkyl, S(O)₂-phenyl, 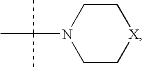
pyridyl optionally substituted with up to two substituents independently selected from (C₁-C₃)alkyl, CN and CF₃,
phenyl optionally substituted with up to two substituents independently selected from (C₁-C₃)alkyl, CN, halo, CF₃, and (C₁-C₃)alkoxy,
(C₁-C₃)alkyl optionally substituted with up to two substituents independently selected from OH, F, phenyl, (C₁-C₃)alkoxy, N[(C₁-C₃)alkyl]₂, pyrrolinidyl, C(O)-pyrrolidinyl, 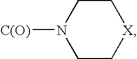 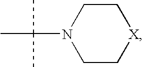
and pyridyl optionally substituted up to two times with (C₁-C₃)alkoxy, and
piperidinyl optionally substituted with up to two substituents independently selected from phenyl, pyridyl, pyrrolidinyl and oxo-dihydrobenzimidazolyl;
R⁷ is selected from NH₂, pyrrolidinyl, 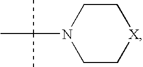
NH(C₁-C₃)alkyl said alkyl being optionally substituted up to two times with (C₁-C₃)alkoxy,
NH-phenyl said phenyl being optionally substituted with up to two substituents independently selected from (C₁-C₃)alkyl, CN, (C₁-C₄)alkoxy, halo and CF₃,
N[(C₁-C₃)alkyl]₂ wherein each alkyl is independently optionally substituted up to
two times with (C₁-C₄)alkoxy, and
phenyl optionally substituted with up to two substituents independently selected from (C₁-C₃)alkyl, (C₁-C₃)alkoxy, halo, CF₃ and CN;
R₈ is selected from (C₁-C₃)alkoxy, pyridyl, piperidinyl, pyranyl and phenyl, where each ring moiety is optionally substituted with up to two substituents independently selected from (C₁-C₃)alkoxy, and (C₁-C₃)alkyl;
R⁹ is selected from (C₁-C₃)alkyl, (C₁-C₃)alkoxy, OH, 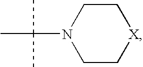
phenyl optionally substituted with (C₁-C₃)alkyl, (C₁-C₃)alkoxy, halo, CF₃, and CN,
N[(C₁-C₄)alkyl]₂ where each of said alkyl groups are independently optionally substituted with OH, CN, N[(C₁-C₄)alkyl]₂, (C₁-C₄)alkoxy, S(O)₂-phenyl, S(O)₂(C₁-C₃)alkyl, phenyl, furyl, tetrahydrofuryl, (C₃-C₆)cycloalkyl, and pyridyl, and
pyrrolidinyl optionally substituted with N[(C₁-C₃)alkyl]₂,
and, only when 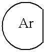
contains no N atoms, R⁹ is also selected from pyridyl, thienyl, and NHR¹⁰;
R¹⁰ is selected from H, indolyl,
(C₁-C₄)alkyl optionally substituted with up to two substituents independently selected from OH, F, phenyl, (C₁-C₄)alkoxy, NHC(O)(C₁-C₃)alkyl,
S—(C₁-C₃)alkyl, benzimidazolyl, indolyl, thienyl, pyrazolyl, 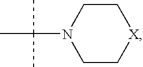
N[(C₁-C₄)alkyl]₂ where each alkyl is independently optionally substituted with up to two substituents independently selected from OH, (C₁-C₃)alkoxy, F, and phenyl,
phenyl optionally substituted with up to two substituents independently selected from (C₁-C₃)alkyl, (C₁-C₃)alkoxy, CN, halo, CF₃, S(O)₂(C₁-C₃)alkyl, S(O)₂phenyl, and S(O)₂NH₂,
pyridyl optionally substituted up to two times with CF₃,
imidazolyl optionally substituted up to two times with (C₁-C₃)alkyl,
furyl optionally substituted up to two times with (C₁-C₄)alkyl, and
pyrrolidinyl optionally substituted with up to two substituents independently selected from (C₁-C₄)alkoxy, (O), and
(C₁-C₄)alkyl optionally substituted with up to two substituents independently selected from OH, (C₁-C₃)alkoxy, F, and phenyl,
S(O)₂-phenyl optionally substituted with up to two substituents independently selected from (C₁-C₄)alkoxy, (C₁-C₃)alkyl, halo, and CN,
pyrazolyl optionally substituted with up to two substituents independently selected from (C₁-C₄)alkyl, (C₃-C₆)cycloalkyl, and
phenyl, said phenyl being optionally substituted with up to two substituents independently selected from (C₁-C₄)alkoxy, (C₁-C₄)alkyl, halo, CF₃, and CN,
benzothiazolyl optionally substituted up to two times with (C₁-C₄)alkyl,
thiazolyl, optionally substituted up to two times with (C₁-C₄)alkyl,
thiadiazolyl, optionally substituted with up to two substituents independently selected from CF₃, (C₃-C₆)cycloalkyl, and (C₁-C₆)alkyl,
phenyl optionally substituted with up to two substituents independently selected from CN, halo, CF₃, N[(C₁-C₄)alkyl]₂, indolyl, 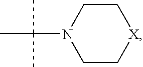
O-pyridyl optionally substituted with C(O)NH(C₁-C₄)alkyl, (C₁-C₄)alkyl optionally substituted with up to two substituents independently selected from pyridyl, 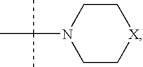
OH,
(C₁-C₃)alkoxy, F, and phenyl, and
(C₁-C₄)alkoxy optionally substituted with N((C₁-C₄)alkyl]₂ where one alkyl group is optionally substituted with phenyl, or
(C₁-C₄)alkoxy optionally substituted with 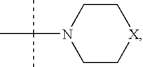
pyridyl optionally substituted with phenoxy where said phenoxy is optionally substituted with up to two substituents independently selected from (C₁-C₄)alkyl and (C₁-C₄)alkoxy, and
indazolyl optionally substituted up to two times with (C₁-C₄)alkyl;
R¹¹ and R¹² are each selected independently from H, F and Cl with the proviso that when one of R¹¹ and R¹² is F or Cl, the other must be H;
X is selected from O, S, CH₂, and NH, and
when X is NH, the H on NH is optionally replaced with pyridyl, pyrazinyl, phenyl,
or (C₁-C₄)alkyl optionally substituted with up to two substituents independently selected from OH, (C₁-C₃)alkoxy, N[(C₁-C₃)alkyl]₂, C(O)-pyrrolidinyl, N[(C₁-C₄)alkyl]₂, and phenyl said phenyl being optionally substituted with up to two substituents independently selected from CN and (C₁-C₃)alkoxy,
and when X is O, S, or CH₂, the 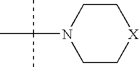
moiety is optionally substituted by replacing any H atom in the 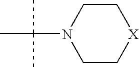
moiety with (C₁-C₄)alkyl;
or a pharmaceutically acceptable salt or ester thereof.

12. A method of claim 11 where the angiogenic disorder is selected from diabetic retinopathy, macular degeneration, angiofibromas, a rheumatic inflammatory disease, a neoplastic disease, and a solid tumor growth.

13. A method of claim 12 where the angiogenic disorder is selected from breast cancer, lung cancer, colon cancer, prostate cancer and pancreatic cancer.

14. A pharmaceutical composition comprising a compound of Formula I 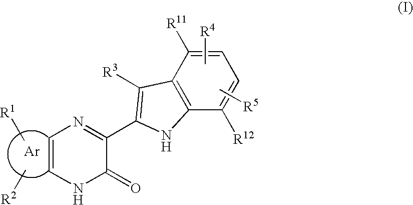
wherein 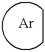
represents a 6 membered aromatic ring containing 0, 1 or 2 N atoms;
R¹ and R² are each independently selected from H, halo, CF₃, C(O)R⁹, 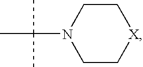
(C₁-C₆)alkyl optionally substituted with up to two substituents selected from OH, (C₁-C₃)alkoxy, F, and phenyl,
(C₁-C₆)alkoxy optionally substituted with one or two substituents each independently selected from 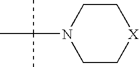
and N[(C₁-C₃)alkyl]₂ where each alkyl is independently optionally substituted up to two times with (C₁-C₃)alkoxy,
NH(C₁-C₃)alkyl where said alkyl is optionally substituted with up to two substitutents each selected independently from OH, F, (C₁-C₃)alkoxy, N[(C₁-C₃)alkyl]₂, NH(C₁-C₃)alkyl, phenyl, pyrrolidinyl, and 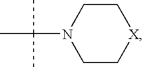
N((C₁-C₃)alkyl]₂ where each alkyl is independently optionally substituted with up to two substitutents each selected independently from OH, F, phenyl, and (C₁-C₃)alkoxy, said alkoxy being optionally substituted with 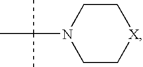
pyrrolidinyl optionally substituted up to two times with N[(C₁-C₃)alkyl]₂,
phenyl optionally substituted with up to two substitutents each selected independently from (C₁-C₃)alkyl, (C₁-C₃)alkoxy, halo, CF₃, and CN,
with the proviso that when 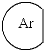
contains 1 or 2 N atoms, R¹ and R² must each be H,
and, R¹ and R² together with the adjacent C atoms to which they are attached form a ring selected from benzo, dioxolo and imidazo,
said imidazo being optionally substituted up to two times with (C₁-C₃)alkyl,
with the proviso that R¹ and R² together with the adjacent C atoms to
which they are attached form a ring only when 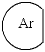
contains no N atoms;
R³ is selected from H, (C₁-C₄)alkyl, OH, NO₂, NH₂, NH(C₁-C₄)alkyl, NHC(O)(C₁-C₄)alkyl and NHC(O)phenyl, said phenyl being optionally substituted with up to two substituents independently selected from (C₁-C₃)alkyl, (C₁-C₃)alkoxy, halo, CF₃, and CN;
R⁴ is selected from H, OH, halo, CN, C(O)R⁶, S(O)₂R⁷, OSi[(C₁-C₄)alkyl]₃, tetrazolyl, thienyl, pyrrolyl, pyrimidinyl, oxazolyl, furanyl,
(C₁-C₆)alkyl, (C₂-C₆)alkenyl or (C₂-C₆)alkynyl, each optionally substituted with OH, F, OC(O)NHphenyl, NHC(O)(C₁-C₃)alkyl, C(O)NH₂, C(O)NH(C₁-C₃)alkyl, C(O)N[(C₁-C₃)alkyl]₂, 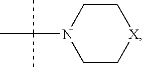
(C₁-C₃)alkoxy optionally substituted up to two times with (C₁-C₃)alkoxy,
NHC(O)NH(C₁-C₃)alkyl where said alkyl is optionally substituted with up to two substituents independently selected from OH, (C₁-C₃)alkoxy, F and phenyl,
NHC(O)NHphenyl where said phenyl is optionally substituted with up to two substituents independently selected from (C₁-C₃)alkyl, (C₁-C₃)alkoxy, halo, CF₃, CN, and 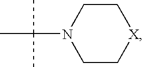
NHC(O)N[(C₁-C₃)alkyl]₂ where each alkyl is independently optionally substituted up to two times with (C₁-C₃)alkoxy,
NH-phenyl, said phenyl being optionally substituted with up to two substituents independently selected from (C₁-C₃)alkyl, (C₁-C₃)alkoxy, halo, CN, and 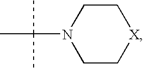
N[(C₁-C₃)alkyl]₂ where each alkyl is independently optionally substituted up to two times with (C₁-C₃)alkoxy,
phenyl optionally substituted with up to two substituents independently selected from (C₁-C₃)alkyl, (C₁-C₃)alkoxy, halo, CN, CF₃, and 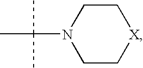
pyrrolidinyl optionally substituted up to two times with N[(C₁-C₃)alkyl]₂,
(C₁-C₆)alkoxy optionally substituted with up to two substituents independently selected from (C₁-C₃)alkoxy, pyrrolidinyl, 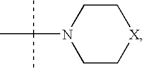
and N[(C₁-C₃)alkyl]₂ where each alkyl is independently optionally substituted with up to two substituents independently selected from OH, F, (C₁-C₃)alkoxy and phenyl,
N[(C₁-C₄)alkyl]₂ where each of said alkyl groups are independently optionally substituted with up to two substituents independently selected from OH, (C₁-C₃)alkyl, F, (C₁-C₃)alkoxy, and phenyl,
oxadiazolyl optionally substituted up to two times with (C₁-C₃)alkyl,
phenyl optionally substituted with up to two substituents independently selected from (C₁-C₃)alkoxy, CN, (C₁-C₃)alkyl, halo, 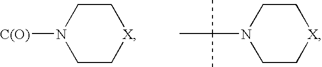
C(O)(C₁-C₃)alkyl optionally substituted with up to two substituents independently selected from (C₁-C₃)alkoxy, OH, (C₁-C₃)alkoxy, F, and phenyl, and
C(O)N[(C₁-C₃)alkyl]₂ where each of said alkyl groups are independently optionally substituted up to two times with (C₁-C₃)alkoxy,
pyridyl optionally substituted with up to two substituents independently selected from (C₁-C₃)alkyl,
C(O)N[(C₁-C₃)alkyl]₂ where each of said alkyl groups are independently optionally substituted up to two times with (C₁-C₃)alkoxy, and
O-pyridyl optionally substituted with up to two substituents independently selected from CF₃, halo, and (C₁-C₃)alkyl;
R⁵ is selected from H, halo, CN, (C₁-C₆)alkoxy, and (C₁-C₆)alkyl;
R⁶ is selected from OH, NHR¹⁰, O—(C₃-C₆)cycloakyl, (C₁-C₃)alkoxy, O—(C₂-C₆)alkenyl,
O—(C₃-C₆)alkynyl,
(C₁-C₆)alkyl optionally substituted with up to two substituents independently selected from OH, (C₁-C₃)alkoxy, F, and phenyl,
N[(C₁-C₄)alkyl]₂ where each of said alkyl groups are independently optionally substituted with up to two substituents independently selected from OH, CN, N[(C₁-C₄)alkyl]₂, (C₁-C₃)alkoxy, S(O)₂-phenyl, S(O)₂(C₁-C₃)alkyl, phenyl, furyl, tetrahydrofuryl, (C₃-C₆)cycloalkyl, and pyridyl,
N[(C₁-C₃)alkyl]R⁸ where [(C₁-C₃)alkyl] is optionally substituted up to two times with (C₁-C₃)alkoxy,
N[(C₃-C₆)cycloalkyl](C₁-C₃)alkyl where said alkyl is substituted with up to two substituents independently selected from (C₁-C₃)alkoxy, OH, CN, N[(C₁-C₄)alkyl]₂, S(O)₂-phenyl, S(O)₂(C₁-C₃)alkyl, phenyl, furyl, tetrahydrofuryl, (C₅-C₆)cycloalkyl, and pyridyl,
pyrrolidinyl optionally substituted with up to two substituents independently selected from NH₂, NH(C₁-C₃)alkyl, N[(C₁-C₄)alkyl]₂, C(O)NH₂, NHC(O)(C₁-C₃)alkyl, NHS(O)₂(C₁-C₃)alkyl, pyridyl, N[(C₁-C₃)alkyl]C(O)NH(C₁-C₃)alkyl, N[(C₁-C₃)alkyl]C(O)(C₁-C₃)alkyl, and (C₁-C₃)alkyl optionally substituted with up to two substituents independently selected from N[(C₁-C₄)alkyl]₂, (C₁-C₃)alkoxy, and pyrrolidinyl,
morpholinyl optionally substituted up to two times with (C₁-C₃)alkyl,
thiomorpholinyl optionally substituted up to two times with (C₁-C₃)alkyl,
piperazinyl optionally substituted with up to two substituents independently selected from pyrazinyl, C(O)NH₂, C(O)NH-phenyl, C(O)-furanyl, C(O)(C₁-C₃)alkyl, C(O)NH(C₁-C₃)alkyl, C(O)N[(C₁-C₃)alkyl]R⁸, S(O)₂(C₁-C₃)alkyl, S(O)₂-phenyl, 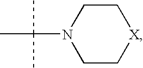
pyridyl optionally substituted with up to two substituents independently selected from (C₁-C₃)alkyl, CN and CF₃,
phenyl optionally substituted with up to two substituents independently selected from (C₁-C₃)alkyl, CN, halo, CF₃, and (C₁-C₃)alkoxy,
(C₁-C₃)alkyl optionally substituted with up to two substituents independently selected from OH, F, phenyl, (C₁-C₃)alkoxy, N[(C₁-C₃)alkyl]₂, pyrrolinidyl, C(O)-pyrrolidinyl, 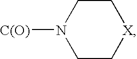 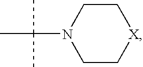
and pyridyl optionally substituted up to two times with (C₁-C₃)alkoxy, and
piperidinyl optionally substituted with up to two substituents independently selected from phenyl, pyridyl, pyrrolidinyl and oxo-dihydrobenzimidazolyl;
R⁷ is selected from NH₂, pyrrolidinyl, 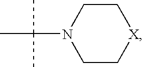
NH(C₁-C₃)alkyl said alkyl being optionally substituted up to two times with (C₁-C₃)alkoxy,
NH-phenyl said phenyl being optionally substituted with up to two substituents independently selected from (C₁-C₃)alkyl, CN, (C₁-C₄)alkoxy, halo and CF₃,
N[(C₁-C₃)alkyl]₂ wherein each alkyl is independently optionally substituted up to two times with (C₁-C₄)alkoxy, and
phenyl optionally substituted with up to two substituents independently selected from (C₁-C₃)alkyl, (C₁-C₃)alkoxy, halo, CF₃ and CN;
R₈ is selected from (C₁-C₃)alkoxy, pyridyl, piperidinyl, pyranyl and phenyl, where each ring moiety is optionally substituted with up to two substituents independently selected from (C₁-C₃)alkoxy, and (C₁-C₃)alkyl;
R⁹ is selected from (C₁-C₃)alkyl, (C₁-C₃)alkoxy, OH, 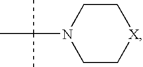
phenyl optionally substituted with (C₁-C₃)alkyl, (C₁-C₃)alkoxy, halo, CF₃, and CN, N[(C₁-C₄)alkyl]₂ where each of said alkyl groups are independently optionally substituted with OH, CN, N[(C₁-C₄)alkyl]₂, (C₁-C₄)alkoxy, S(O)₂-phenyl, S(O)₂(C₁-C₃)alkyl, phenyl, furyl, tetrahydrofuryl, (C₃-C₆)cycloalkyl, and pyridyl, and
pyrrolidinyl optionally substituted with N[(C₁-C₃)alkyl]₂,
and, only when 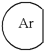
contains no N atoms, R⁹ is also selected from pyridyl, thienyl, and NHR¹⁰;
R¹⁰ is selected from H, indolyl,
(C₁-C₄)alkyl optionally substituted with up to two substituents independently selected from OH, F, phenyl, (C₁-C₄)alkoxy, NHC(O)(C₁-C₃)alkyl, S—(C₁-C₃)alkyl, benzimidazolyl, indolyl, thienyl, pyrazolyl, 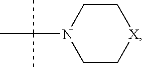
N[(C₁-C₄)alkyl]₂ where each alkyl is independently optionally substituted with up to two substituents independently selected from OH, (C₁-C₃)alkoxy, F, and phenyl,
phenyl optionally substituted with up to two substituents independently selected from (C₁-C₃)alkyl, (C₁-C₃)alkoxy, CN, halo, CF₃, S(O)₂(C₁-C₃)alkyl, S(O)₂phenyl, and S(O)₂NH₂,
pyridyl optionally substituted up to two times with CF₃,
imidazolyl optionally substituted up to two times with (C₁-C₃)alkyl,
furyl optionally substituted up to two times with (C₁-C₄)alkyl, and
pyrrolidinyl optionally substituted with up to two substituents independently selected from (C₁-C₄)alkoxy, (O), and
(C₁-C₄)alkyl optionally substituted with up to two substituents independently selected from OH, (C₁-C₃)alkoxy, F, and phenyl,
S(O)₂-phenyl optionally substituted with up to two substituents independently selected from (C₁-C₄)alkoxy, (C₁-C₃)alkyl, halo, and CN,
pyrazolyl optionally substituted with up to two substituents independently selected from (C₁-C₄)alkyl, (C₃-C₆)cycloalkyl, and
phenyl, said phenyl being optionally substituted with up to two substituents independently selected from (C₁-C₄)alkoxy, (C₁-C₄)alkyl, halo, CF₃, and CN,
benzothiazolyl optionally substituted up to two times with (C₁-C₄)alkyl,
thiazolyl, optionally substituted up to two times with (C₁-C₄)alkyl,
thiadiazolyl, optionally substituted with up to two substituents independently selected from CF₃, (C₃-C₆)cycloalkyl, and (C₁-C₆)alkyl,
phenyl optionally substituted with up to two substituents independently selected from CN, halo, CF₃, N[(C₁-C₄)alkyl]₂, indolyl, 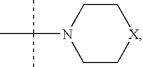
O-pyridyl optionally substituted with C(O)NH(C₁-C₄)alkyl,
(C₁-C₄)alkyl optionally substituted with up to two substituents independently selected from pyridyl, 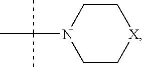
OH,
(C₁-C₃)alkoxy, F, and phenyl, and
(C₁-C₄)alkoxy optionally substituted with N[(C₁-C₄)alkyl]₂ where one alkyl group is optionally substituted with phenyl, or
(C₁-C₄)alkoxy optionally substituted with 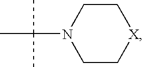
pyridyl optionally substituted with phenoxy where said phenoxy is optionally substituted with up to two substituents independently selected from (C₁-C₄)alkyl and (C₁-C₄)alkoxy, and
indazolyl optionally substituted up to two times with (C₁-C₄)alkyl;
R¹¹ and R¹² are each selected independently from H, F and Cl with the proviso that when one of R¹¹ and R¹² is F or Cl, the other must be H;
X is selected from O, S, CH₂, and NH, and
when X is NH, the H on NH is optionally replaced with pyridyl, pyrazinyl, phenyl,
or (C₁-C₄)alkyl optionally substituted with up to two substituents independently selected from OH, (C₁-C₃)alkoxy, N[(C₁-C₃)alkyl]₂, C(O)-pyrrolidinyl, N[(C₁-C₄)alkyl]₂, and phenyl said phenyl being optionally substituted with up to two substituents independently selected from CN and (C₁-C₃)alkoxy,
and when X is O, S, or CH₂, the 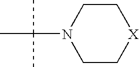
moiety is optionally substituted by replacing any H atom in the 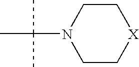
moiety with (C₁-C₄)alkyl;
or a pharmaceutically acceptable salt or ester thereof.
